(19) United States
(12) Patent Application Publication
(10) Pub. No.: US 2007/0072831 A1
(43) Pub. Date: Mar. 29, 2007

(54) Integrase inhibitor compounds

(75) Inventors: Zhenhong R. Cai, Foster City, CA (US); Salman Y. Jabri, San Francisco, CA (US); Haolun Jin, Foster City, CA (US); Choung U. Kim, San Carlos, CA (US); Rachael A. Lansdown, San Mateo, CA (US); Samuel E. Metobo, Newark, CA (US); Michael R. Mish, Foster City, CA (US); Richard M. Pastor, San Francisco, CA (US)

Correspondence Address: Intellectual Property Department;Gilead Sciences, Inc., 333 Lakeside Drive, Foster City, CA, 94404, US

(73) Assignee: GILEAD SCIENCES, INC.

(21) Appl. No.: 11/435,671

(22) Filed: May 16, 2006

Related U.S. Application Data
(60) Provisional application No. 60/681,690, filed on May 16, 2005.

(51) Int. Cl. A61K 31/4745; A61K 31/675; C07D 471/02; C07F 9/576

(52) U.S. Cl. 514/80; 514/291; 546/22; 546/84

(57) ABSTRACT

Tricyclic compounds, protected intermediates thereof, and methods for inhibition of HIV-integrase are disclosed.

### CROSS-REFERENCE TO RELATED APPLICATIONS

This application claims the benefit of U.S. Provisional Application 60/681,690, filed on May 16, 2005, which is hereby incorporated by reference in its entirety.

### FIELD OF THE INVENTION

The invention relates generally to compounds having antiviral activity, and more specifically, compounds having HIV-integrase inhibitory properties.

### BACKGROUND OF THE INVENTION

Human immunodeficiency virus (HIV) infection and related diseases are a major public health problem worldwide. A virally encoded integrase protein mediates specific incorporation and integration of viral DNA into the host genome. Integration is necessary for viral replication. Accordingly, inhibition of HIV integrase is an important therapeutic pursuit for treatment of HIV infection of the related diseases.

Human immunodeficiency virus type 1 (HIV-1) encodes three enzymes which are required for viral replication: reverse transcriptase, protease, and integrase. Although drugs targeting reverse transcriptase and protease are in wide use and have shown effectiveness, particularly when employed in combination, toxicity and development of resistant strains have limited their usefulness (Palella, etal N. Engl. J. Med. (1998) 338:853-860; Richman, D. D. Nature (2001) 410:995-1001). There is a need for new agents directed against alternate sites in the viral life cycle. Integrase has emerged as an attractive target, because it is necessary for stable infection and homologous enzymes are lacking in the human host (LaFemina, etal J. Virol. (1992) 66:7414-7419). The function of integrase is to catalyze integration of proviral DNA, resulting from the reverse transcription of viral RNA, into the host genome, by a stepwise fashion of endonucleolytic processing of proviral DNA within a cytoplasmic preintegration complex (termed 3′-processing or “3′-P”) with specific DNA sequences at the end of the HIV-1 long terminal repeat (LTR) regions, followed by translocation of the complex into the nuclear compartment where integration of 3′-processed proviral DNA into host DNA occurs in a “strand transfer” (ST) reaction (Hazuda, etal Science (2000) 287:646-650; Katzman, etal Adv. Virus Res. (1999) 52:371-395; Asante-Applah, etal Adv. Virus Res. (1999) 52:351-369). Although numerous agents potently inhibit 3′-P and ST in extracellular assays that employ recombinant integrase and viral long-terminal-repeat oligonucleotide sequences, often such inhibitors lack inhibitory potency when assayed using fully assembled preintegration complexes or fail to show antiviral effects against HIV-infected cells (Pommier, etal Adv. Virus Res. (1999) 52:427-458; Farnet, etal Proc. Natl. Acad. Sci. U.S.A. (1996) 93:9742-9747; Pommier, etal Antiviral Res. (2000) 47:139-148.

HIV integrase inhibitory compounds with improved antiviral and pharmacokinetic properties are desirable, including enhanced activity against development of HIV resistance, improved oral bioavailability, greater potency and extended effective half-life in vivo (Nair, V. “HIV integrase as a target for antiviral chemotherapy” Reviews in Medical Virology (2002) 12(3):179-193). Three-dimensional quantitative structure-activity relationship studies and docking simulations (Buolamwini, etal Jour. Med. Chem. (2002) 45:841-852) of conformationally-restrained cinnamoyl-type integrase inhibitors (Artico, etal Jour. Med. Chem. (1998) 41:3948-3960) have correlated hydrogen-bonding interactions to the inhibitory activity differences among the compounds.

Certain HIV integrase inhibitors have been disclosed which seek to block integration in extracellular assays and exhibit antiviral effects against HIV-infected cells (Anthony, etal WO 02/30426; Anthony, etal WO 02/30930; Anthony, etal WO 02/30931; WO 02/055079; Zhuang, etal WO 02/36734; U.S. Pat. No. 6,395,743; U.S. Pat. No. 6,245,806; U.S. Pat. No. 6,271,402; Fujishita, etal WO 00/039086; Uenaka etal WO 00/075122; Selnick, etal WO 99/62513; Young, etal WO 99/62520; Payne, etal WO 01/00578; Jing, etal Biochemistry (2002) 41:5397-5403; Pais, etal J. Med. Chem. (2002) 45:3184-94; Goldgur, etal Proc. Natl. Acad. Sci. U.S.A. (1999) 96:13040-13043; Espeseth, etal Proc. Natl. Acad. Sci. U.S.A. (2000) 97:11244-11249). Recent HIV integrase inhibitors are shown in WO 2005/016927, WO 2004/096807, WO 2004/035577, WO 2004/035576 and US 2003/0055071.

There exists a need to find better compounds for the treatment of HIV, particularly, improved integrase inhibitors having beneficial properties and good efficacy. The invention in part teaches compounds possessing improved anti-HIV and/or pharmaceutical properties compared to those disclosed in WO 2004/03577.

### SUMMARY OF THE INVENTION

One aspect the invention provides compounds represented by formula A:

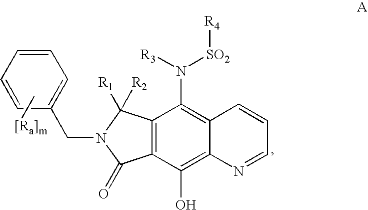

or pharmaceutically acceptable salts thereof,

where,

each Rₐ is independently selected from the group consisting of hydrogen, chloro, fluoro, CH₃HNC(O)—, (CH₃)₂NC(O)—, (CH₃)₂NS(0)₂—, CH₃S(O)₂—, cyano and amino;

m is zero, one, two, three, four or five;

R₁ and R₂ are independently selected from the group consisting of hydrogen and C₁₋₄ alkyl;

R₃ is selected from from the group consisting of hydrogen, methyl and ethyl; and

R₄ is C₁₋₄ alkyl, N-ethylamino or N,N-dimethylamino;

or R₃ and R₄ are cyclized to form, together with the nitrogen atom pendent to the R₃ group and the SO₂ group pendent to the R₄ group a heterocyclic or substituted heterocyclic group.

In certain embodiments, compounds of formula A are represented by formula I or la: 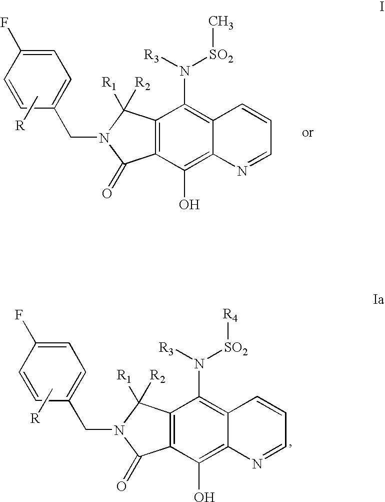
or pharmaceutically acceptable salts thereof,

where,

R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl;

R₃ is selected from from the group consisting of hydrogen, methyl and ethyl; and

R4 is N,N-dimethylamino;

or R₃ and R₄ are cyclized to form, together with the nitrogen atom pendent to the R₃ group and the SO₂ group pendent to the R₄ group a heterocyclic or substituted heterocyclic group.

In another embodiment, the compounds of this invention are represented by formula II: 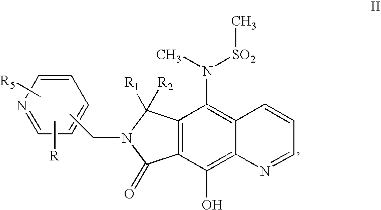
or pharmaceutically acceptable salts thereof,

where,

R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl; and

R₅ is selected from the group consisting of hydrogen and fluoro.

In another embodiment, the compounds of this invention are represented by formula Ill: 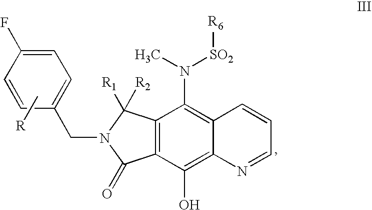
or pharmaceutically acceptable salts thereof,

where,

R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl; and

R₆ is selected from the group consisting of methyl, ethyl, isopropyl,1-methylimidazol-4-yl, 2,4-dimethylthiazol-5-yl, 2-(N,N-dimethylamino)eth-1-yl, 2-(N,N-diethylamino)eth-1-yl, 3-cyanoprop-1-yl, 3-(N-morpholino)prop-1-yl, 2-(N-morpholino)eth-1-yl, 3-(N,N-dimethylamino)prop-1-yl, amino, N-methylamino, N,N-dimethylamino, 2-(methylcarbonylamino)-4-methylthiazol-5-yl, 6-(N-morpholino)pyrid-3-yl, pyrid-2-yl, N-methyl-N-(pyrid-4-yl)methylamino, N-methyl-N-benzylamino, 2,2,2-trifluoroeth-1-yl, 2-(piperazin-2-yl)eth-1-yl, 2-(N-piperidinyl)eth-1-yl, 3-(imidazol-1-yl)-prop-1-yl, N-morpholino and 5-N,N-dimethylaminonaphth-1-yl.

In still another embodiment, the compounds of this invention are represented by formula IV: 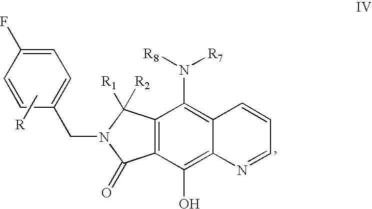
or pharmaceutically acceptable salts thereof,

where,

R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl;

R₇ is selected from the group consisting of hydrogen and methyl;

R₈ is selected from the group consisting of hydrogen, —C(O)OR₉, —C(O)R₁₀ and —C(O)C(O)NR₁₁R₁₁,

or R₇ and R₈, together with the nitrogen atom pendent thereto, form a heterocyclic or substituted heterocyclic group;

R₉ is selected from the group consisting of hydrogen, C₁-C₄ alkyl, phenyl and substituted phenyl;

R₁₀ is selected from the group consisting of amino, C₁-C₄ alkylamino, [C₁-C₄ alkyl]₂amino, C₁-C₄ alkyl, heterocyclic and substituted heterocyclic; and

each R₁₁ is independently selected from the group consisting of hydrogen and C₁-C₄ alkyl.

In yet another embodiment, the compounds of this invention are represented by formula V: 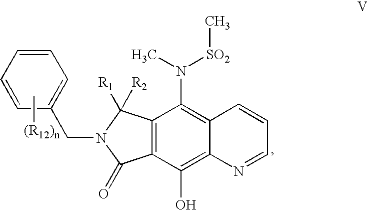
or pharmaceutically acceptable salts thereof,

where,

R₁ and R₂ are independently selected from the group consisting of hydrogen and methyl;

each R₁₂ is independently selected from the group consisting of halo, C₁-C₄ alkoxy, —C(O)OR₉, —C(O)NR₁₅R₁₆, amino, C₁-C₄ alkylamino, di(C₁-C₄ alkyl)amino, cyano, —SO₂—(C₁-C₄ alkyl) and —SO₂—NR₁₅R₁₆;

R₉ is selected from the group consisting of hydrogen and C₁-C₄ alkyl;

each R₁₅ and R₁₆ is independently selected from the group consisting of hydrogen and C₁-C₄ alkyl; and

n is one, two or three.

Yet another embodiment provides compounds represented by the formula VI: 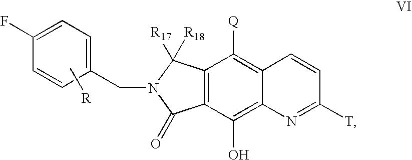
or pharmaceutically acceptable salts thereof,

where,

R is selected from the group consisting of hydrogen, CH₃HNC(O)—, (CH₃)₂NC(O)—, (CH₃)₂NS(O)₂—, CH₃S(O)₂—, cyano and amino;

R₁₇ and R₁₈ are independently selected from the group consisting of hydrogen and hydroxyl, provided that both R₁₇ and R₁₈ are not hydrogen, or R₁₇ and R₁₈, together with the carbon atom pendent thereto, form a carbonyl group;

Q is selected from the group consisting of amino, hydroxyl, 2-(trimethylsilyl)ethoxy, N-morpholino and —N(CH₃)SO₂CH₃; and

T is selected from the the group consisting of hydrogen, amino and halo.

Still another embodiment provides compounds represented by the formula VII: 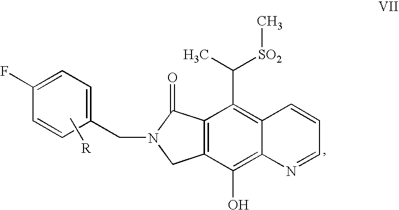
or pharmaceutically acceptable salts thereof,

where,

R is selected from the group consisting of hydrogen, CH₃HNC(O)—, (CH₃)₂NC(O)—, (CH₃)₂NS(O)₂—, CH₃S(O)₂—, cyano and amino;

In another embodiment, the compounds of this invention are represented by formula XXIV: 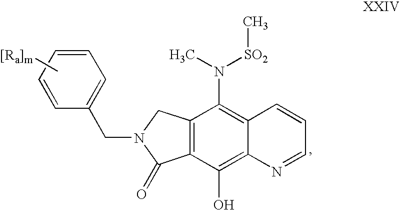
or pharmaceutically acceptable salts thereof,

where,

each Rₐ is independently halo; and

m is zero, one, two, three, four or five.

In still another embodiment, the compounds of this invention are represented by formula XXV: 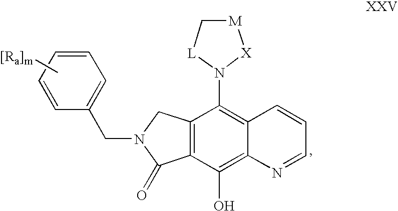
or pharmaceutically acceptable salts thereof,

where,

L is —CH₂—, —CH₂—CH₂— or —C(O)—;

X is —S(O)₂— or —C(O)—;

M is —N(R₂₀)— or —CH₂—;

R₂₀ is H or —C₁₋₄alkyl;

each Rₐ is independently halo; and

m is zero, one, two, three, four or five.

The invention also includes a pharmaceutical composition comprising a therapeutically effective amount of a compound of the invention, or a pharmaceutically acceptable salt thereof, in combination with a pharmaceutically acceptable diluent, excipient or carrier.

The invention also includes a pharmaceutical composition comprising a therapeutically effective amount of a compound of the invention, or a pharmaceutically acceptable salt thereof, in combination with a booster agent and/or a therapeutically effective amount of one or more of the following agents: another compound of the invention, an AIDS treatment agent, such as an HIV inhibitor agent, an anti-infective agent or an immunomodulator agent. The HIV inhibitor agent may include an HIV-protease inhibitor, a nucleoside reverse transcriptase inhibitor, a non-nucleoside reverse transcriptase inhibitor or a mixture thereof.

The invention also includes methods of treating (for example, preventing, mediating, inhibiting, etc.) the proliferation of HIV virus, treating AIDS, delaying the onset of AIDS or ARC symptoms and generally inhibiting HIV integrase. The methods comprise administering to a mammal infected with HIV (HIV positive) an amount of a compound of the invention, in a therapeutically effective dose or administration, to inhibit the growth of HIV infected cells of the mammal.

In another aspect of the invention, the activity of HIV integrase is inhibited by a method comprising the step of treating a mammal or sample suspected of containing HIV virus with a compound or composition of the invention.

The invention also includes processes and novel intermediates which are useful for preparing compounds of the invention. Some of the compounds of the invention are useful to prepare other compounds of the invention.

This invention also includes a method of increasing cellular accumulation, bioavailability or retention of drug compounds, thus improving their therapeutic and diagnostic value, by administering a phosphonate prodrug form of a compound of the invention.

Another aspect of the invention provides a method for inhibiting the activity of HIV integrase comprising the step of contacting a mammal or sample suspected of containing HIV virus with a composition of the invention.

In other aspects, methods for the synthesis, analysis, separation, isolation, crystallization, purification, characterization, resolution of isomers (including enantiomers and diastereomers) and testing of the compounds of the invention are provided.

### DEFINITIONS

Unless stated otherwise, the following terms and phrases as used herein are intended to have the following meanings:

The terms “phosphonate” and “phosphonate group” mean a functional group or moiety within a molecule that comprises at least one phosphorus-carbon bond, and at least one phosphorus-oxygen double bond. The phosphorus atom is further substituted with oxygen, sulfur, and nitrogen substituents. These substituents may be part of a prodrug moiety. As defined herein, “phosphonate” and “phosphonate group” include molecules with phosphonic acid, phosphonic monoester, phosphonic diester, phosphonamidate, phosphondiamidate, and phosphonthioate functional groups.

The term “prodrug” as used herein refers to any compound that when administered to a biological system generates the drug substance, i.e. active ingredient, as a result of spontaneous chemical reaction(s), enzyme catalyzed chemical reaction(s), photolysis, and/or metabolic chemical reaction(s). A prodrug is thus a covalently modified analog or latent form of a therapeutically-active compound.

“Pharmaceutically acceptable prodrug” refers to a compound that is metabolized in the host, for example hydrolyzed or oxidized, by either enzymatic action or by general acid or base solvolysis, to form an active ingredient. Typical examples, of prodrugs of the compounds of the invention have biologically labile protecting groups on a functional moiety of the compound. Prodrugs include compounds that can be oxidized, reduced, aminated, deaminated, esterified, deesterified, alkylated, dealkylated, acylated, deacylated, phosphorylated, dephosphorylated, photolyzed, hydrolyzed, or other functional group change or conversion involving forming or breaking chemical bonds on the prodrug.

“Prodrug moiety” means a labile functional group which separates from the active inhibitory compound during metabolism, systemically, inside a cell, by hydrolysis, enzymatic cleavage, or by some other process (Bundgaard, H., “Design and Application of Prodrugs” in Textbook of Drug Design and Development (1991), P. Krogsgaard-Larsen and H. Bundgaard, Eds. Harwood Academic Publishers, pp. 113-191). Enzymes which are capable of an enzymatic activation mechanism with the prodrug compounds of the invention include, but are not limited to, amidases, esterases, microbial enzymes, phospholipases, cholinesterases, and phosphases. Prodrug moieties can serve to enhance solubility, absorption and lipophilicity to optimize drug delivery, bioavailability and efficacy. A “prodrug” is thus a covalently modified analog of a therapeutically-active compound.

Exemplary prodrug moieties include the hydrolytically sensitive or labile acyloxymethyl esters —CH₂OC(═O) R²⁰ and acyloxymethyl carbonates —CH₂OC(═O)OR²⁰ where R²⁰ is C₁-C₆ alkyl, C₁-C₆ substituted alkyl, C₆-C₂₀ aryl or C₆-C₂₀ substituted aryl. The acyloxyalkyl ester was first used as a prodrug strategy for carboxylic acids and then applied to phosphates and phosphonates by Farquhar et al., (1983) J. Pharm. Sci. 72: 324; also U.S. Pat. Nos. 4,816,570, 4,968,788, 5,663,159 and 5,792,756, which are all incorporated by reference. In certain compounds of the invention, a prodrug moiety is part of a phosphonate group. Subsequently, the acyloxyalkyl ester was used to deliver phosphonic acids across cell membranes and to enhance oral bioavailability. A close variant of the acyloxyalkyl ester, the alkoxycarbonyloxyalkyl ester (carbonate), may also enhance oral bioavailability as a prodrug moiety in the compounds of the invention. An exemplary acyloxymethyl ester is pivaloyloxymethoxy, (POM) —CH₂OC(═O)C(CH₃)₃. An exemplary acyloxymethyl carbonate prodrug moiety is pivaloyloxymethylcarbonate (POC) —CH₂OC(═O)OC(CH₃)₃.

The phosphonate group may be a phosphonate prodrug moiety. The prodrug moiety may be sensitive to hydrolysis, such as, but not limited to a pivaloyloxymethyl carbonate (POC) or POM group. Alternatively, the prodrug moiety may be sensitive to enzymatic potentiated cleavage, such as a lactate ester or a phosphonamidate-ester group. Exemplary phosphonate prodrug moieties include by way of example and not limitation groups of the structure A³.

Aryl esters of phosphorus groups, especially phenyl esters, are reported to enhance oral bioavailability (DeLambert etal (1994) J. Med. Chem. 37: 498). Phenyl esters containing a carboxylic ester ortho to the phosphate have also been described (Khamnei and Torrence, (1996) J. Med. Chem. 39:4109-4115). Benzyl esters are reported to generate the parent phosphonic acid. In some cases, substituents at the ortho-or para-position may accelerate the hydrolysis. Benzyl analogs with an acylated phenol or an alkylated phenol may generate the phenolic compound through the action of enzymes, e.g. esterases, oxidases, etc., which in turn undergoes cleavage at the benzylic C—O bond to generate the phosphoric acid and the quinone methide intermediate. Examples of this class of prodrugs are described by Mitchell et al., (1992) J. Chem. Soc. Perkin Trans. I 2345; Brook et al., WO 91/19721. Still other benzylic prodrugs have been described containing a carboxylic ester-containing group attached to the benzylic methylene (Glazier et al., WO 91/19721). Thio-containing prodrugs are reported to be useful for the intracellular delivery of phosphonate drugs. These proesters contain an ethylthio group in which the thiol group is either esterified with an acyl group or combined with another thiol group to form a disulfide. Deesterification or reduction of the disulfide generates the free thio intermediate which subsequently breaks down to the phosphoric acid and episulfide (Puech et al., (1993) Antiviral Res., 22: 155-174; Benzaria et al., (1996) J. Med. Chem. 39: 4958). Cyclic phosphonate esters have also been described as prodrugs of phosphorus-containing compounds (Erion et al., U.S. Pat. No. 6,312,662).

“Protecting group” refers to a moiety of a compound that masks or alters the properties of a functional group or the properties of the compound as a whole. The chemical substructure of a protecting group varies widely. One function of a protecting group is to serve as intermediates in the synthesis of the parental drug substance. Chemical protecting groups and strategies for protection/deprotection are well known in the art. See: “Protective Groups in Organic Chemistry”, Theodora W. Greene (John Wiley & Sons, Inc., New York, 1991, which is incorporated herein by reference. Protecting groups are often utilized to mask the reactivity of certain functional groups, to assist in the efficiency of desired chemical reactions, e.g. making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

The term “hydroxyl protecting group,” as used herein, refers to an easily removable group which is known in the art to protect a hydroxyl group against undesirable reaction during synthetic procedures and/or during biodelivery and which group can be selectively removed. The use of hydroxy-protecting groups is well known in the art for protecting groups and many such protecting groups are known, for example, T. H. Greene and P. G. M. Wuts, Protective Groups in Organic Synthesis, 2nd edition, John Wiley & Sons, New York (1991). Examples of hydroxy-protecting groups include, but are not limited to,

Ethers (methyl);

Substituted methyl ethers (methoxymethyl, methylthiomethyl, t-butylthiomethyl, (phenyldimethylsilyl)methoxymethyl, benzyloxymethyl, p-methoxybenzyloxymethyl, (4-methoxyphenoxy)methyl, guaiacolmethyl, t-butoxymethyl, 4-pentenyloxymethyl, siloxymethyl, 2-methoxyethoxymethyl, 2,2,2-trichloroethoxymethyl, bis(2-chloroethoxy)methyl, 2-(trimethylsilyl)ethoxymethyl, tetrahydropyranyl, 3-bromotetrahydropyranyl, tetrahydrothiopyranyl, 1-methoxycyclohexyl, 4-methoxytetrahydropyranyl, 4-methoxytetrahydro-thiopyranyl, 4-methoxytetrahydropthiopyranyl S,S-dioxido, 1->(2-chloro-4-methyl)phenyl-4-methoxypiperidin-4-yl, 1 ,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl));

Substituted ethyl ethers (1-ethoxyethyl, 1-(2-chloroethoxy)ethyl, 1-methyl-i -methoxyethyl, 1-methyl-i -benzyloxyethyl, 1-methyl-i -benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl);

Substituted benzyl ethers (p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2- and 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxy)phenyldiphenylmethyl, 4,4′, 4″-tris(4,5-dichlorophthalimidophenyl)methyl, 4,4′, 4″-tris(levulinoyloxyphenyl)-methyl, 4,4′, 4″-tris(benxoyloxyphenyl)methyl, 3-(imidazol-1-ylmethyl)bis(4′, 4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-Dioxido);

Silyl ethers (trimethylsilyl, triethylsilyl, triisopropylsilyl, dimethylisopropylsilyl, diethylisopropylsily, dimethylthexylsilyl, t-butyldimethyl-silyl, t-butyldiphenylsilyl, tribenzylsilyl, tri-p-xylylsilyl, triphenylsilyl, diphenylmethylsilyl, t-butylmethoxyphenylsilyl);

Esters (formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, p-poly-phenylacetate, 3-phenyl-propionate, 4-oxopentanoate (Levulinate), 4,4-(ethylenedithio)pentanoate, pivaloate, adamantoate, crotonate, 4-methoxycrotonate, benzoate, p-phenyl-benzoate, 2,4,6-trimethylbenzoate (Mesitoate));

Carbonates (methyl, 9-fluorenylmethyl, ethyl, 2,2,2-trichloroethyl, 2-(trimethylsilyl)ethyl, 2-(phenylsulfonyl)ethyl, 2-(triphenylphosphonio)ethyl, isobutyl, vinyl, allyl, p-nitrophenyl, benzyl, p-methoxybenzyl, 3,4-dimethoxybenzyl, o-nitrobenzyl, p-nitrobenzyl, S-benzyl thiocarbonate, 4-ethoxy-1-naphthyl, methyl dithiocarbonate);

Groups with assisted cleavage (2-iodobenzoate, 4-azidobutyrate, 4-nitro-4-methylpentanoate, o-(dibromomethyl) benzoate, 2-formylbenzenesulfonate, 2-(methylthiomethoxy)ethyl carbonate, 4-(methylthiomethoxy)butyrate, 2-(methylthiomethoxymethyl)benzoate);

Miscellaneous Esters (2,6-dichloro-4-methylphenoxyacetate, 2,6-dichloro-4-(1, 1 ,3,3-tetramethylbutyl)phenoxyacetate, 2,4-bis(1, 1-dimethylpropyl)-phenoxyacetate, chlorodiphenylacetate, isobutyrate, monosuccinoate, (E)-2-methyl-2-butenoate (Tigloate), o-(methoxycarbonyl)benzoate, p-poly-benzoate, a-naphthoate, nitrate, alkyl N,N,N′,N′-tetramethylphosphorodiamidate, N-phenylcarbamate, borate, dimethylphosphinothioyl, 2,4-dinitrophenylsulfenate); and

Sulfonates (sulfate, methanesulfonate (Mesylate), benzylsulfonate, Tosylate).

More typically, hydroxy protecting groups include substituted methyl ethers, substituted benzyl ethers, silyl ethers, and esters including sulfonic acid esters, still more typically, trialkylsilyl ethers, tosylates and acetates.

The term “amino protecting group,” as used herein, refers to an easily removable group which is known in the art to protect an amino group against undesired reaction during synthetic procedures and/or during biodelivery and which group can be selectively removed. Such protecting groups are described by Greene at pages 315-385. They include:

Carbamates (methyl and ethyl, 9-fluorenylmethyl, 9(2-sulfo)fluoroenyl-methyl, 9-(2,7-dibromo)fluorenylmethyl, 2,7-di-t-buthyl-[9-(10,10-dioxo-10,10,10,1 0-tetrahydrothioxanthyl)]methyl, 4-methoxyphenacyl);

Substituted ethyl (2,2,2-trichoroethyl, 2-trimethylsilylethyl, 2-phenylethyl, 1-(1-adamantyl)-1-methylethyl, 1,1-dimethyl-2-haloethyl, 1,1-dimethyl-2,2-dibromoethyl, 1,1-dimethyl-2,2,2-trichloroethyl, 1-methyl-I -(4-biphenylyl)ethyl, 1-(3,5-di-t-butylphenyl)-1-methylethyl, 2-(2′- and 4′-pyridyl)ethyl, 2-(N,N-dicyclohexylcarboxamido)ethyl, t-butyl, 1-adamantyl, vinyl, allyl, 1-isopropylallyl, cinnamyl, 4-nitrocinnamyl, 8-quinolyl, N-hydroxypiperidinyl, alkyldithio, benzyl, p-methoxybenzyl, p-nitrobenzyl, p-bromobenzyl, p-chorobenzyl, 2,4-dichlorobenzyl, 4-methylsulfinylbenzyl, 9-anthrylmethyl, diphenylmethyl);

Groups With Assisted Cleavage (2-methylthioethyl, 2-methylsulfonylethyl, 2-(p-toluenesulfonyl)ethyl, [2-(1,3-dithianyl)]methyl, 4-methylthiophenyl, 2,4-dimethylthiophenyl, 2-phosphonioethyl, 2-triphenylphosphonioisopropyl, 1,1-dimethyl-2-cyanoethyl, m-choro-p-acyloxybenzyl, p-(dihydroxyboryl)benzyl, 5-benzisoxazolylmethyl, 2-(trifluoromethyl)-6-chromonylmethyl);

Groups Capable of Photolytic Cleavage (m-nitrophenyl, 3,5-dimethoxybenzyl, o-nitrobenzyl, 3,4-dimethoxy-6-nitrobenzyl, phenyl(o-nitrophenyl)methyl);

Urea-Type Derivatives (phenothiazinyl-(10)-carbonyl, N′-p-toluenesulfonylaminocarbonyl, N′-phenylaminothiocarbonyl);

Miscellaneous Carbamates (t-amyl, S-benzyl thiocarbamate, p-cyanobenzyl, cyclobutyl, cyclohexyl, cyclopentyl, cyclopropylmethyl, p-decyloxybenzyl, diisopropylmethyl, 2,2-dimethoxycarbonylvinyl, o-(N,N-dimethyl-carboxamido)benzyl, 1,1-dimethyl-3-(N,N-dimethylcarboxamido)propyl, 1,1-dimethylpropynyl, di(2-pyridyl)methyl, 2-furanylmethyl, 2-Iodoethyl, Isobornyl, Isobutyl, Isonicotinyl, p-(p′-Methoxyphenylazo)benzyl, 1-methylcyclobutyl, 1-methylcyclohexyl, 1-methyl-1-cyclopropylmethyl, 1-methyl-1-(3,5-dimethoxyphenyl)ethyl, 1-methyl-1-(p-phenylazophenyl)ethyl, 1-methyl-1-phenylethyl, 1-methyl-1-(4-pyridyl)ethyl, phenyl, p-(phenylazo)benzyl, 2,4,6-tri-t-butylphenyl, 4-(trimethylammonium)benzyl, 2,4,6-trimethylbenzyl);

Amides (N-formyl, N-acetyl, N-choroacetyl, N-trichoroacetyl, N-trifluoroacetyl, N-phenylacetyl, N-3-phenylpropionyl, N-picolinoyl, N-3-pyridylcarboxamide, N-benzoylphenylalanyl, N-benzoyl, N-p-phenylbenzoyl);

Amides With Assisted Cleavage (N-o-nitrophenylacetyl, N-o-nitrophenoxyacetyl, N-acetoacetyl, (N′-dithiobenzyloxycarbonylamino)acetyl, N-3-(p-hydroxyphenyl) propionyl, N-3-(o-nitrophenyl)propionyl, N-2-methyl-2-(o-nitrophenoxy)propionyl, N-2-methyl-2-(o-phenylazophenoxy)propionyl, N-4-chlorobutyryl, N-3-methyl-3-nitrobutyryl, N-o-nitrocinnamoyl, N-acetylmethionine, N-o-nitrobenzoyl, N-o-(benzoyloxymethyl)benzoyl, 4,5-diphenyl-3-oxazolin-2-one);

Cyclic Imide Derivatives (N-phthalimide, N-dithiasuccinoyl, N-2,3-diphenylmaleoyl, N-2,5-dimethylpyrrolyl, N-1,1,4,4-tetramethyldisilylazacyclopentane adduct, 5-substituted 1 ,3-dimethyl-1,3,5-triazacyclohexan-2-one, 5-substituted 1 ,3-dibenzyl-1 ,3-5-triazacyclohexan-2-one, 1-substituted 3,5-dinitro4-pyridonyl);

N-Alkyl and N-Aryl Amines (N-methyl, N-allyl, N-[2-(trimethylsilyl)ethoxy]methyl, N-3-acetoxypropyl, N-(1-isopropyl4-nitro-2-oxo-3-pyrrolin-3-yl),

Quaternary Ammonium Salts, N-benzyl, N-di(4-methoxyphenyl)methyl, N-5-dibenzosuberyl, N-triphenylmethyl, N-(4-methoxyphenyl)diphenylmethyl, N-9-phenylfluorenyl, N-2,7-dichloro-9-fluorenylmethylene, N-ferrocenylmethyl, N-2-picolylamine N′-oxide),

Imine Derivatives (N-1,1-dimethylthiomethylene, N-benzylidene, N-p-me thoxybenylidene, N-diphenylmethylene, N-[(2-pyridyl)mesityl]methylene, N,(N′,N′-dimethylaminomethylene, N,N′-isopropylidene, N-p-nitrobenzylidene, N-salicylidene, N-5-chlorosalicylidene, N-(5-chloro-2-hydroxyphenyl)phenyl-methylene, N-cyclohexylidene); Enamine Derivatives (N-(5,5-dimethyl-3-oxo-1-cyclohexenyl));

N-Metal Derivatives (N-borane derivatives, N-diphenylborinic acid derivatives, N-[phenyl(pentacarbonylchromium—or -tungsten)]carbenyl, N-copper or N-zinc chelate);

N-N Derivatives (N-nitro, N-nitroso, N-oxide); N-P Derivatives (N-diphenylphosphinyl, N-dimethylthiophosphinyl, N-diphenylthiophosphinyl, N-dialkyl phosphoryl, N-dibenzyl phosphoryl, N-diphenyl phosphoryl);

N-Si Derivatives; N-S Derivatives; N-Sulfenyl Derivatives (N-benzenesulfenyl, N-o-nitrobenzenesulfenyl, N-2,4-dinitrobenzenesulfenyl, N-pentachlorobenzenesulfenyl, N-2-nitro-4-methoxybenzenesulfenyl, N-triphenylmethylsulfenyl, N-3-nitropyridinesulfenyl); and

N-sulfonyl Derivatives (N-p-toluenesulfonyl, N-benzenesulfonyl, N-2,3 ,6-trimethyl-4-methoxybenzenesulfonyl, N-2,4,6-trimethoxybenzenesulfonyl, N-2,6-dimethyl-4-methoxybenzenesulfonyl, N-pentamethylbenzenesulfonyl, N-2,3,5,6,-tetramethyl-4-methoxybenzenesulfonyl, N-4-methoxybenzenesulfonyl, N-2,4,6-trimethylbenzenesulfonyl, N-2 ,6-dimethoxy-4-methyl benzenesulfonyl, N-2,2,5,7,8-pentamethylchroman-6-sulfonyl, N-methanesulfonyl, N-.beta.-trimethylsilyl-ethanesulfonyl, N-9-anthracenesulfonyl, N-4-(4′,8′-dimethoxynaphthyl-methyl)benzenesulfonyl, N-benzylsulfonyl, N-trifluoromethylsulfonyl, N-phenacylsulfonyl).

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g. alcohols, be physiologically acceptable, although in general it is more desirable if the products are pharmacologically innocuous. Exemplary protecting groups include by way of example and not limitation groups of the structure R^{x} other than hydrogen.

Reference to the compounds of the invention includes all physiologically acceptable salt thereof. Examples of physiologically acceptable salts of the compounds of the invention include salts derived from an appropriate base, such as an alkali metal (for example, sodium), an alkaline earth (for example, magnesium), ammonium and NX₄⁺ (wherein X is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound of an hydroxy group include the anion of said compound in combination with a suitable cation such as Na⁺ and NX₄⁺ (wherein X is independently selected from the group consisting of H and a C₁-C₄ alkyl group).

For therapeutic use, salts of active ingredients of the compounds of the invention will be physiologically acceptable, i.e. they will be salts derived from a physiologically acceptable acid or base. However, salts of acids or bases which are not physiologically acceptable may also find use, for example, in the preparation or purification of a physiologically acceptable compound. All salts, whether or not derived form a physiologically acceptable acid or base, are within the scope of the present invention. “Alkyl” is C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH3)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH3)CH₂CH₂CH₃), 3-pentyl (—CH(CH₂CH₃)₂), 2-methyl-2-butyl (—C(CH3)₂CH₂CH₃), 3-methyl-2-butyl (-CH(CH3)CH(CH3)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

“Alkenyl” is C₂-C₁₈ hydrocarbon containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂).

“Alkynyl” is C₂-C₁₈ hydrocarbon containing normal, secondary, tertiary or cyclic carbon atoms with at least one site of unsaturation, i.e. a carbon-carbon, sp triple bond. Examples include, but are not limited to: acetylenic (—C≡CH) and propargyl (—CH₂C≡CH),

The terms “alkylene” and “alkyldiyl” each refer to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene, i.e. double carbon-carbon bond moiety. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne, i.e. triple carbon-carbon bond moiety. Typical alkynylene radicals include, but are not limited to: acetylene (—C≡C—), propargyl (—CH₂C≡C—), and 4-pentynyl (—CH₂CH₂CH₂C≡CH—).

“Aryl” means a monovalent aromatic hydrocarbon radical of 6-20 carbon atoms derived by the removal of one hydrogen atom from a single carbon atom of a parent aromatic ring system. Typical aryl groups include, but are not limited to, radicals derived from benzene, substituted benzene, naphthalene, anthracene, biphenyl, and the like.

“Heteroaryl” means a monovalent aromatic radical of one or more carbon atoms and one or more atoms selected from the group consisting of N, O, S and P, derived by the removal of one hydrogen atom from a single atom of a parent aromatic ring system. Heteroaryl groups may be a monocycle having 3 to 7 ring members (2 to 6 carbon atoms and 1 to 3 heteroatoms selected from the group consisting of N, O, P and S) or a bicycle having 7 to 10 ring members (4 to 9 carbon atoms and 1 to 3 heteroatoms selected from the group consisting of N, O, P and S). Heteroaryl bicycles have 7 to 10 ring atoms (6 of N, O and S) arranged as a bicyclo [4,5], [5,5], [5,6], or [6,6] system; or 9 to 10 ring atoms (8 to 9 carbon atoms and 1 to 2 hetero atoms selected from the group consisting of N and S) arranged as a bicyclo [5,6] or [6,6] system. The heteroaryl group may be bonded to the drug scaffold through a carbon, nitrogen, sulfur, phosphorus or other atom by a stable covalent bond.

Heteroaryl groups include, for example: pyridyl, dihydropyridyl isomers, pyridazinyl, pyrimidinyl, pyrazinyl, s-triazinyl, oxazolyl, imidazolyl, thiazolyl, isoxazolyl, pyrazolyl, isothiazolyl, furanyl, thiofuranyl, thienyl, and pyrrolyl.

“Arylalkyl” refers to an acyclic alkyl radical in which one of the hydrogen atoms bonded to a carbon atom, typically a terminal or Sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms.

Substituted substituents such as “substituted alkyl”, “substituted aryl”, “substituted heteroaryl”, “substituted heterocyclic” and “substituted arylalkyl” mean alkyl, aryl, heteroaryl, heterocyclic and arylalkyl respectively, in which one or more hydrogen atoms are each independently replaced with a substituent. Typical substituents include, but are not limited to, —X, —R, ═O, —O⁻, —OR, —S³¹, —SR, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NRR —S(═O)₂O³¹, —S(═O)₂OH, —S(═O)₂R, —OS(═O)₂OR, —S(═O)₂NR, —S(═O)R, —OP(═O)₂RR, —P(═O)O₂RR—P(═O)(O⁻)₂, —P(═O)(OH)₂, —C(═O)R, —C(═O)X, —C(S)R, —C(O)OR, —C(O)O³¹, —C(S)OR, —C(O)SR, —C(S)SR, —C(O)NRR, —C(S)NRR, —C(NR)NRR, where each X is independently a halogen: F, Cl, Br, or l; and each R is independently H, alkyl, aryl, heterocycle, protecting group or prodrug moiety. Alkylene, alkenylene, and alkynylene groups may also be similarly substituted.

“Heterocycle” means a saturated, unsaturated or aromatic ring system including at least one N, O, S, or P. Heterocycle thus include heteroaryl groups. Heterocycle as used herein includes by way of example and-not limitation these heterocycles described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566.

Examples of heterocycles include by way of example and not limitation pyridyl, dihydropyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4H-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

One embodiment of the bis-tetrahydrofuranyl group is:

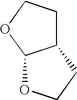

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” means a saturated, unsaturated or aromatic ring system having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g. arranged as a bicyclo [4,5], [5,5], [5,6] or [6,6] system, or 9 or 10 ring-atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl and naphthyl. Carbocycle thus includes some aryl groups.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities.. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and I or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

### DETAILED DESCRIPTION OF EXEMPLARY EMBODIMENTS

Certain compounds of the invention provide have the formula A:

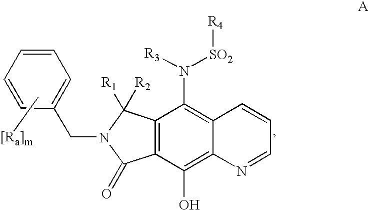

or pharmaceutically acceptable salts thereof,

where,

each Rₐ is independently selected from the group consisting of hydrogen, chloro, fluoro, CH₃HNC(O)—, (CH₃)₂NC(O)—, (CH₃)₂NS(O)₂—, CH₃S(O)₂—, cyano and amino;

m is zero, one, two, three, four or five;

R₁ and R₂ are independently selected from the group consisting of hydrogen and C₁₋₄ alkyl;

R₃ is selected from from the group consisting of hydrogen, methyl and ethyl; and

R₄ is C₁₋₄ alkyl, N-ethylamino or N,N-dimethylamino;

or R₃ and R4 are cyclized to form, together with the nitrogen atom pendent to the R₃ group and the SO₂ group pendent to the R₄ group a heterocyclic or substituted heterocyclic group.

In two embodiments, compounds of formula A are represented by formula I or la: 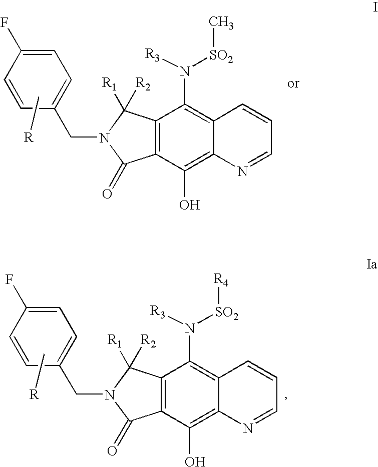
or pharmaceutically acceptable salts thereof,

where,

R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl;

R₃ is selected from from the group consisting of hydrogen, methyl and ethyl; and

R₄ is N,N-dimethylamino;

or R₃ and R₄ are cyclized to form, together with the nitrogen atom pendent to the R₃ group and the SO₂ group pendent to the R₄ group a heterocyclic or substituted heterocyclic group.

In one embodiment of formula I, R₃ is methyl. In another embodiment of formula I, R₃ is hydrogen. In still another embodiment of formula I, R₃ is ethyl.

In one embodiment of formula I, when R₃ is methyl or hydrogen, then R₁ and R₂ are hydrogen. In one embodiment of formula I, when R₃ is methyl, then R₁. hydrogen and R₂ is methyl. In still another embodiment of formula I, when R₃ is methyl, then R₁ and R₂ are methyl.

In one embodiment of formula Ia, when R, R₁, R₂ and R₃ are hydrogen, then R₄ is N,N-dimethylamino. In another embodiment of formula Ia, when R, R₁ and R₂ are each hydrogen, then R₃ and R4 are joined to form a 2-dioxoisothazolidine heterocyclic group.

Representative compounds of formula I and la are set forth in Tables I and 2 below:

TABLE 1

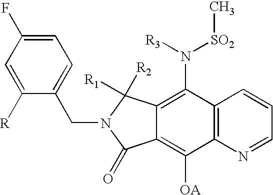

| R | R₁ | R₂ | R₃ |
|---|---|---|---|
| H | H | H | —CH₃ |
| H | H | —CH₃ | —CH₃ |
| H | —CH₃ | —CH₃ | —CH₃ |
| —C(O)NHCH₃ | H | H | —CH₃ |
| —C(O)N(CH₃)₂ | H | H | —CH₃ |
| —S(O)₂N(CH₃)₂ | H | H | —CH₃ |
| —S(O)₂CH₃ | H | H | —CH₃ |
| —CN | H | H | —CH₃ |
| —NH₂ | H | H | —CH₃ |
| H | H | H | H |
| H | H | H | —CH₂CH₃ |
| H | H | H | H |

### TABLE 2

TABLE 2

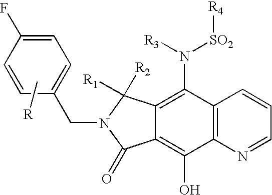

| R | R₁ | R₂ | R₃ | R₄ |
|---|---|---|---|---|
| H | H | H | H | (CH₃)₂N— |

| | | | |
|---|---|---|---|
| H | H | H | R3/R4 are joined to a |
| | | | 2-dioxoisothiazolidine |

In one embodiment, a pharmaceutically acceptable salt of formula I and formula Ia is represented by formula Ib and Ic: 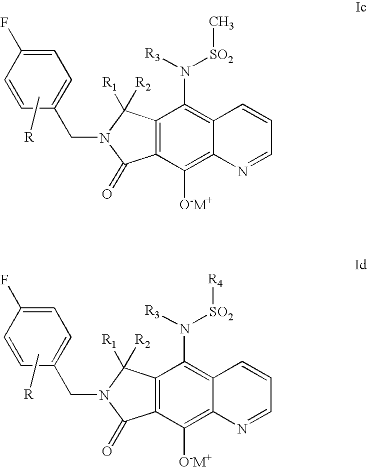
where R, R₁, R₂, R₃ and R₄ are as defined above and M⁺ is a pharmaceutically acceptable cation.

In one embodiment, M⁺ is selected from the group consisting of sodium and potassium.

In one embodiment of formula Ic, when R₃ is methyl, then M⁺ is potassium. In another embodiment, when R₃ is hydrogen, then M⁺ is potassium. In still another embodiment, when R₃ is ethyl, then M⁺ is potassium.

In one embodiment of formula Ic, when R₃ is methyl, then M⁺ is sodium. In another embodiment, when R₃ is hydrogen, then M⁺ is sodium. In still another embodiment, when R₃ is ethyl, then M⁺ is sodium.

In one embodiment of formula Ic, when R₃ is methyl or hydrogen, R₁, and R₂ are hydrogen, then M⁺ is potassium. In another embodiment, when R₃ is methyl, R₁ is hydrogen and R₂ is methyl, then M⁺ is potassium. In still another embodiment, when R₃ is methyl, R₁ and R₂ are methyl, then M⁺ is potassium.

In one embodiment of formula Ic, when R₃ is methyl or hydrogen, R₁ and R₂ are hydrogen, then M⁺ is sodium. In another embodiment, when R₃ is methyl, R₁, hydrogen and R₂ is methyl, then M⁺ is sodium. In still another embodiment, when R₃ is methyl, R₁ and R₂ are methyl, then M⁺ is sodium.

In one embodiment of formula Id, when R, R₁ , R₂ and R₃ are hydrogen, R₄ is N,N-dimethylamino, then M⁺ is potassium. In another embodiment of formula Id, when R, R₁ and R₂ are hydrogen and R₃ and R₄ are joined to form a 2-dioxoisothiazolidine heterocyclic group, then M⁺ is potassium.

In one embodiment of formula Id, when R, R₁, R₂ and R₃ are hydrogen and R₄ is N,N-dimethylamino, then M⁺ is sodium. In another embodiment of formula Id, when R, R. and R₂ are hydrogen and R₃ and R4 are joined to form a 2-dioxoisothiazolidine heterocyclic group, then M⁺ is sodium.

In another embodiment, the compounds of this invention are represented by formula II: 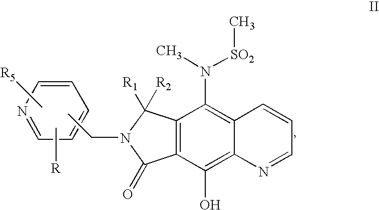
or pharmaceutically acceptable salts thereof,

where,

R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl; and

R₅ is selected from the group consisting of hydrogen and fluoro.

In one embodiment, when R, R₁ and R₂ are hydrogen and R₅ is 6-fluoro, then the pyridyl group is 3-pyridyl. In one embodiment, when R, R₁, R₂ and R₅ are hydrogen, then the pyridyl group is 2-pyridyl, 3-pyridyl or 4-pyridyl. In one embodiment, when R, Rand R₂ are hydrogen and R₅ is 5-fluoro, then the pyridyl group is 2-pyridyl.

Representative compounds of formula II are set forth in Table 3 below:

TABLE 3

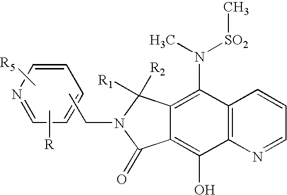

| R₁ | R₂ | R/R₅/pyridyl |
|---|---|---|
| H | H | 6-fluoropyrid-3-yl |
| H | H | pyrid-2-yl |
| H | H | pyrid-4-yl |
| H | H | 5-fluoropyrid-2-yl |

In one embodiment, a pharmaceutically acceptable salt of formula II is represented by formula IIa: 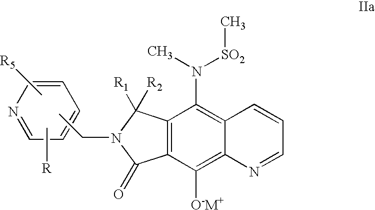
where R, R₁, R₂, and R₅ are as defined above and M⁺ is a pharmaceutically acceptable cation.

In one embodiment, when R, R₁ and R₂ are hydrogen, R₅ is 6-fluoro and the pyridyl group is 3-pyridyl, then M⁺ is potassium. In another embodiment, when R, R₁, R₂ and R₅ are hydrogen and the pyridyl group is 2-pyridyl, 3-pyridyl or 4-pyridyl, then M⁺ is potassium. In still another embodiment, when R, R₁ and R₂ are hydrogen, R₅ is 5-fluoro and the pyridyl group is 2-pyridyl, then M⁺ is potassium.

In one embodiment, when R, R₁ and R₂ are hydrogen, R₅ is 6-fluoro and the pyridyl group is 3-pyridyl, then M⁺ is sodium. In another embodiment, when R, R₁, R₂ and R₅ are hydrogen and the pyridyl group is either 3-pyridyl or 4-pyridyl, then M⁺ is sodium. In still another embodiment, when R, R₁ and R₂ are hydrogen, R₅ is 5-fluoro and the pyridyl group is 2-pyridyl, then M⁺ is sodium.

In another embodiment, the compounds of formula II are represented by formula IIb: 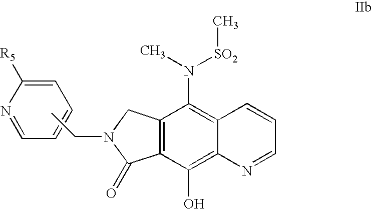
where R₅ is as defined above as well as pharmaceutically acceptable salts thereof.

In another embodiment, the compounds of this invention are represented by formula III: 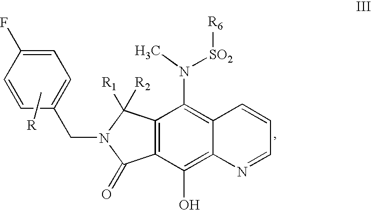
or pharmaceutically acceptable salts thereof,

where,

R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl; and

R₆ is selected from the group consisting of methyl, ethyl, isopropyl,1-methylimidazol-4-yl, 2,4-dimethylthiazol-5-yl, 2-(N,N-dimethylamino)eth-1-yl, 2-(N,N-diethylamino)eth-1-yl, 3-cyanoprop-1-yl, 3-(N-morpholino)prop-1-yl, 2(N-morpholino)eth-1-yl, 3-(N,N-dimethylamino)prop-1-yl, amino, N-methylamino, N,N-dimethylamino, 2-(methylcarbonylamino)-4-methylthiazol-5-yl, 6-(N-morpholino)pyrid-3-yl, pyrid-2-yl, N-methyl-N-(pyrid-4-yl)methylamino, N-methyl-N-benzylamino, 2,2,2-trifluoroeth-1-yl, 2-(piperazin-2-yl)eth-1-yl, 2-(N-piperidinyl)eth-1-yl, 3-(imidazol-1-yl)-prop-1-yl, N-morpholino and 5-N,N-dimethylaminonaphth-1-yl.

Representative compounds of formula III are set forth in Table 4 below:

TABLE 4

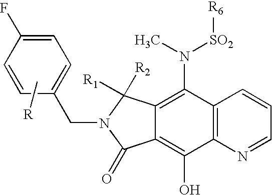

| R | R₁ | R₂ | R₆ |
|---|---|---|---|
| H | H | H | isopropyl |
| H | H | H | 1-methylimidazol-4-yl |
| H | H | H | 2,4-dimethylthiazol-5-yl |
| H | H | H | 2-(N,N-dimethylamino)eth-1-yl |
| H | H | H | 3-cyanoprop-1-yl |
| H | H | H | 3-(N-morpholino)prop-1-yl |
| H | H | H | 3-(N-imidazolyl)prop-1-yl |
| H | H | H | 3-(N,N-dimethylamino)prop-1-yl |
| H | H | H | N,N-dimethylamino |
| H | H | H | 2-(—NHC(O)CH₃)-4-methylthiazol-5-yl |
| H | H | H | 6-(N-morpholino)pyrid-5-yl |
| H | H | H | pyrid-2-yl |
| H | H | H | N-methyl-N-(pyrid-4-yl-CH₂—)amino |
| H | H | H | N-methyl-N-benzylamino |
| H | H | H | 2,2,2-trifluoroethyl |
| H | H | H | 2-(N-piperazinyl)eth-1-yl |
| H | H | H | 2-(N-morpholino)eth-1-yl |
| H | H | H | N-methylamino |
| H | H | H | 2-(N-piperidinyl)eth-1-yl |
| H | H | H | 2-(N,N-ethylamino)eth-1-yl |
| H | H | H | amino |
| H | H | H | 3-(N-imidazolyl)prop-1-yl |
| H | H | H | 5-(N,N-dimethylamino)naphth-1-yl |

In one embodiment, a pharmaceutically acceptable salt of formula III is represented by formula III:

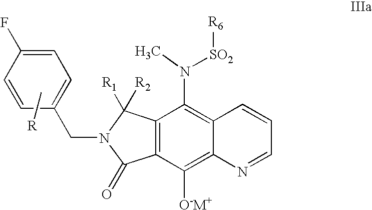

where R, R₁, R₂, and R₆ are as defined above and M⁺ is a pharmaceutically acceptable cation.

In one embodiment, M⁺ is selected from the group consisting of sodium and potassium.

In one embodiment, when R, R₁ and R₂ are hydrogen, then M⁺ is potassium.

In one embodiment, when R, R₁ and R₂ are hydrogen, then M⁺ is sodium.

When R, R₁ and R₂ are hydrogen, the compounds of formula III are represented by formula IIIb below:

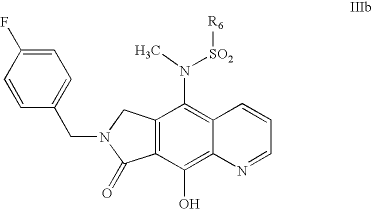

where R⁶ is as defined above as well as pharmaceutically acceptable salts thereof.

In still another embodiment, the compounds of this invention are represented by formula IV: 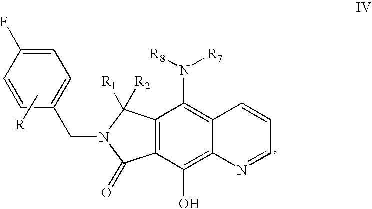
or pharmaceutically acceptable salts thereof,

where,

R is selected from the group consisting of hydrogen, CH₃HNC(O)—, (CH₃)₂NC(O)—, (C H₃)₂NS(O)₂—, CH₃S(O)₂—, cyano and amino;

R₁ and R₂ are independently selected from the group consisting of hydrogen and methyl;

R₇ is selected from the group consisting of the group consisting of hydrogen and methyl;

R₈ is selected from the group consisting of the group consisting of hydrogen, —C(O)OR₉, —C(O)R₁₀ and —C(O)C(O)NR₁₁R₁₁, or R₇ and R₈, together with the nitrogen atom pendent thereto, form a heterocyclic or substituted heterocyclic group;

R₉ is selected from the group consisting of hydrogen, C₁-C₄ alkyl, phenyl and substituted phenyl;

R₁₀ is selected from the group consisting of amino, C₁-C₄ alkylamino, [C₁-C₄ alkyl]₂amino, C₁-C₄ alkyl, heterocyclic and substituted heterocyclic; and

each R₁₁ is independently selected from the group consisting of hydrogen and C₁-C₄ alkyl.

In one embodiment, when R, R₁ and R₂ are each hydrogen, then R₇ and R₈, together with the nitrogen atom pendent thereto, form a group selected, from the group consisting of amino, N-methyl-N-ethoxycarbonylamino, N-methyl-N-(N,N-dimethylamino-carbonyl)carbonylamino, N-methyl-N-isopropylcarbonylamino, N-methyl-N-(N-morpholino)carbonylamino, N-methyl-N-(N-methylamino)carbonylamino and p-nitrophenoxycarbonylamino.

In one embodiment, when R and R₁ are hydrogen, and R₂ is methyl, then R₇ and R₈, together with the nitrogen atom pendent thereto, form N-methyl-N-ethoxycarbonylamino.

In one embodiment, when R is hydrogen, and R₁ and R₂ are methyl, then R₇ and R₈, together with the nitrogen atom pendent thereto, form N-methyl-N-ethoxycarbonylamino.

Representative compounds of formula IV are set forth in Table 5 below:

TABLE 5

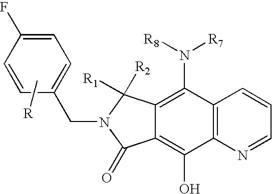

| R | R₁ | R₂ | R₈ | R₇ |
|---|---|---|---|---|
| H | H | H | —C(O)OCH₂CH₃ | —CH₃ |
| H | H | —CH₃ | —C(O)OCH₂CH₃ | —CH₃ |
| H | —CH₃ | —CH₃ | —C(O)OCH₂CH₃ | —CH₃ |
| H | H | H | —C(O)C(O)N(CH₃)₂ | —CH₃ |
| H | H | H | —C(O)CH(CH₃)₂ | —CH₃ |
| H | H | H | —C(O)CH(CH₃)₂ | H |
| H | H | H | —C(O)CH₃ | —CH₃ |
| H | H | H | H | H |
| H | H | H | —C(O)N(CH₃)₂ | —CH₃ |
| H | H | H | —C(O)-(N-morpholino) | —CH₃ |

In one embodiment, a pharmaceutically acceptable salt of formula IV is represented by formula IVa: 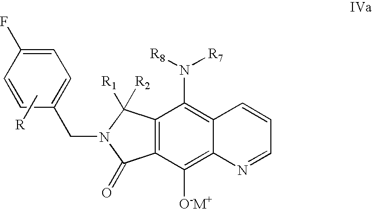
where R, R₁, R₂, R₇ and R₈ are as defined above and M⁺ is a pharmaceutically acceptable cation.

In one embodiment, M⁺ is selected from the group consisting of sodium and potassium.

In one embodiment, when R, R₁ and R₂ are hydrogen and R₇ and R₈, together with the nitrogen atom pendent thereto, form a group selected from the group consisting of amino, N-methyl-N-ethoxycarbonylamino, N-methyl-N-(N,N-dimethylamino-carbonyl)carbonylamino, N-methyl-N-isopropylcarbonylamino, N-methyl- N-(N-morpholino)carbonylamino, N-methyl-N-(N-methylamino)carbonylamino and p-nitrophenoxycarbonylamino, then M⁺ is potassium.

In one embodiment, when R, R₁ and R₂ are hydrogen and R₇ and R₈, together with the nitrogen atom pendent thereto, form a group selected from the group consisting of amino, N-methyl-N-ethoxycarbonylamino, N-methyl-N-(N,N-dimethylamino-carbonyl)carbonylamino, N-methyl-N-isopropylcarbonylamino, N-methyl-N-(N-morpholino)carbonylamino, N-methyl-N-(N-methylamino)carbonylamino and p-nitrophenoxycarbonylamino, then M⁺ is sodium.

In one embodiment, when R and R₁ are hydrogen, R₂ is methyl, and R₇ and R₈, together with the nitrogen atom pendent thereto, form N-methyl-N-ethoxycarbonylamino, then M⁺ is potassium.

In one embodiment, when R and R₁ are hydrogen, R₂ is methyl, and R₇ and R₈, together with the nitrogen atom pendent thereto, form N-methyl-N-ethoxycarbonylamino, then M⁺ is sodium.

In one embodiment, when R is hydrogen, R₁ and R₂ are methyl, and R₇ and R₈, together with the nitrogen atom pendent thereto, form N-methyl-N-ethoxycarbonylamino, then M⁺ is potassium.

In one embodiment, when R is hydrogen, R₁ and R₂ are methyl, and R₇ and R₈, together with the nitrogen atom pendent thereto, form N-methyl-N-ethoxycarbonylamino, then M⁺ is sodium.

In yet another embodiment, the compounds of this invention are represented by formula V: 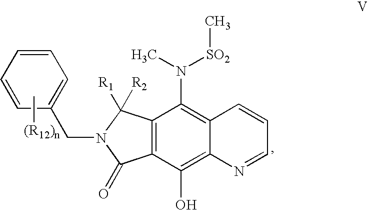
or pharmaceutically acceptable salts thereof,

where,

R₁ and R₂ are independently selected from the group consisting of hydrogen and methyl;

each R₁₂ is independently selected from the group consisting of halo, C₁-C₄ alkoxy, —C(O)OR₉, —C(O)NR₁₅R₁₆, amino, C₁-C₄ alkylamino, di(C₁-C₄ alkyl)amino, cyano, —SO₂—(C₁-C₄ alkyl) and —SO₂—NR₁₅R₁₆;

R₉ is selected from the group consisting of hydrogen and C₁-C₄ alkyl;

each R₁₅ and R₁₆ is independently selected from the group consisting of hydrogen and C₁-C₄ alkyl; and

n is one, two or three.

In one embodiment, the compounds of formula V are represented by formula Va: 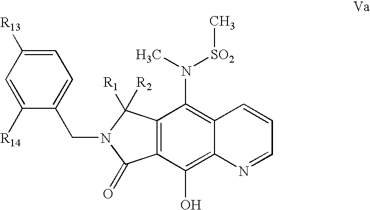
or pharmaceutically acceptable salts thereof,

where R₁ and R₂ are independently selected from the group consisting of hydrogen and methyl; and

R₁₃ and R₁₄ are independently selected from the group consisting of halo, C₁-C₄ alkoxy, —C(O)OR₉ where R₉ is hydrogen or C₁-C₄ alkyl, —C(O)NR₁₅R₁₆ where each R₁₅ and R₁₆ is independently selected from the group consisting of hydrogen and C₁-C₄ alkyl, amino, C₁-C₄ alkylamino, di(C₁-C₄ alkyl)amino, cyano, —SO₂—(C₁-C₄ alkyl), and —SO₂—NR₁₅R₁₆ where R₁₅ and R₁₆ are as defined above.

In one embodiment of formula Va, when R₁ and R₂ are hydrogen, then R₁₃ and R₁₄, together with the phenyl group pendent thereto, form a group selected from the group consisting of 2-chloro-4-fluorophenyl, 2,4-dimethoxyphenyl, 2,4-difluorophenyl, 2-amino-4-fluorophenyl, 2-cyano-4-fluorophenyl, 2-(N,N-dimethylamino)carbonyl-4-fluorophenyl, 2-methylsulfonyl-4-fluorophenyl, 2-(N,N-dimethyl)aminosulfonyl-4-fluorophenyl and 2-(N-methylamino)carbonyl-4-fluorophenyl.

Representative compounds of formula Va are set forth in Table 6 below:

TABLE 6

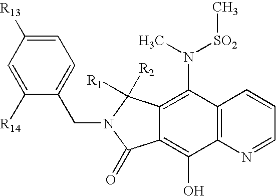

| R₁ | R₂ | R₁₄ | R₁₃ |
|---|---|---|---|
| H | H | —OCH₃ | —OCH₃ |
| H | H | F | F |
| H | H | —NH₂ | F |
| H | H | —CN | F |
| H | H | —C(O)N(CH₃)₂ | F |
| H | H | —S(O)₂CH₃ | F |
| H | H | —S(O)₂N(CH₃)₂ | F |
| H | H | —C(O)NHCH₃ | F |
| H | H | Cl | F |
| H | H | —OCH₃ | F |
| H | H | H | Cl |
| H | H | F | Cl |

In one embodiment, a pharmaceutically acceptable salt of formula V is represented by formula Vb:

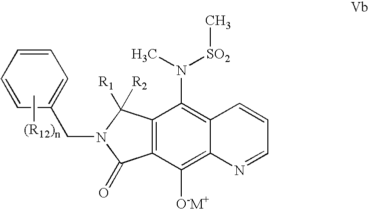

where R₁ and R₂ are independently selected from the group consiting of hydrogen and methyl;

R₁₂ is independently selected from the group consisting of halo, C₁-C₄ alkoxy, —C(O)OR₉ where R₉ is hydrogen or C₁-C₄ alkyl, —C(O)NR₁₅R₁₆ where each R₁₅ and R₁₆ is independently selected from the group consisting of hydrogen and C₁-C₄ alkyl, amino, C₁-C₄ alkylamino, di(C₁-C₄ alkyl)amino, cyano, —SO₂—(C₁-C₄ alkyl), and —SO₂—NR₁₅R₁₆ where R,₁₅ and R₁₆ are as defined above;

M⁺ is a pharmaceutically acceptable cation; and

n is one, two or three.

In one embodiment, a pharmaceutically acceptable salt of formula Va is represented by formula Vc:

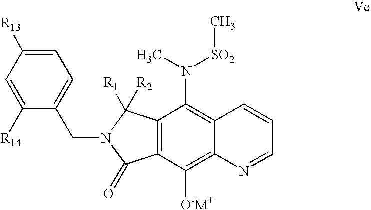

where R₁ and R₂ are independently selected from the group consisting of hydrogen and methyl;

R₁₃ and R₁₄ are independently selected from the group consisting of halo, C₁-C₄ alkoxy, —C(O)OR₉ where R₉ is hydrogen or C₁-C₄ alkyl, —C(O)NR₁₅R₁₆ where each R₁₅ and R₁₆ is independently selected from the group consisting of hydrogen and C₁-C₄ alkyl, amino, C₁-C₄ alkylamino, di(C₁-C₄ alkyl)amino, cyano, —SO₂—(C₁-C₄ alkyl), and —SO₂—NR₁₅R₁₆ where R₁₅ and R₁₆ are as defined above; and

M⁺ is a pharmaceutically acceptable cation.

In one embodiment of formula Vc, when R₁ and R₂ are hydrogen and R₁₃ and R₁₄, together with the phenyl group pendent thereto, form a group selected from the group consisting of 3-chloro-5-fluorophenyl, 3,5-dimethoxyphenyl, 3,5-difluorophenyl, 3-amino-5-fluorophenyl, 3-cyano-5-fluorophenyl, 3-(N,N-dimethylamino)carbonyl-5-fluorophenyl, 3-methylsulfonyl-5-fluorophenyl, 3-(N,N-dimethyl)aminosulfonyl-5-fluorophenyl and 3-(N-methylamino)carbonyl-5-fluorophenyl, then M⁺ is potassium.

In one embodiment of formula Vc, when R₁ and R₂ are hydrogen and R₁₃ and R₁₄, together with the phenyl group pendent thereto, form a group selected from the group consisting of 3,5-dimethoxyphenyl, 3,5-difluorophenyl, 3-amino-5-fluorophenyl, 3-cyano-5-fluorophenyl, 3-(N,N-dimethylamino)carbonyl-5-fluorophenyl, 3-methylsulfonyl-5-fluorophenyl, 3-(N,N-dimethyl)aminosulfonyl-5-fluorophenyl and 3-(N-methylamino)carbonyl-5-fluorophenyl, then M⁺ is sodium.

Yet another embodiment provides compounds represented by the formula VI: 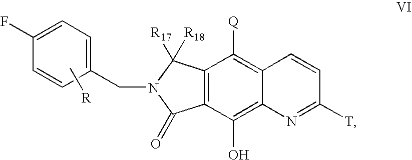
or pharmaceutically acceptable salts thereof,

where,

R is selected from the group consisting of hydrogen, CH₃HNC(O)—, (CH₃)₂NC(O)—, (CH₃)₂NS(O)₂—, CH₃S(O)₂—, cyano and amino;

R₁₇ and R₁₈ are independently selected from the group consisting of hydrogen and hydroxyl, provided that both R₁₇ and R₁₈ are not hydrogen, or

R₁₇ and R₁₈, together with the carbon atom pendent thereto, form a carbonyl group;

Q is selected from the group consisting of amino, hydroxyl, 2-(trimethylsilyl)ethoxy, N-morpholino and —N(CH₃)SO₂CH₃; and

T is selected from the the group consisting of hydrogen, amino and halo.

In one embodiment, when R is hydrogen, T is chloro and Q is hydroxyl, then R₁₇ and R₁₈, together with the carbon atom pendent thereto, form a carbonyl group.

In one embodiment, when R is hydrogen, T is amino and Q is 2-(trimethylsilyl)ethoxy, then R₁₇ and R₁₈, together with the carbon atom pendent thereto, form a carbonyl group.

In one embodiment, when R and T are hydrogen, and Q is —N(CH₃)SO₂CH₃, then R₁₇ and R₁₈, together with the carbon atom pendent thereto, form a carbonyl group, or R₁₇ is hydroxyl and R₁₈ is hydrogen (both R and S stereochemistry). In one embodiment, when R and T are hydrogen and Q is amino, then R₁₇ and R₁₈, together with the carbon atom pendent thereto, form a carbonyl group. In one embodiment, when R and T are hydrogen and Q is morpholino, then R₁₇ and R₁₈, together with the carbon atom pendent thereto, form a carbonyl group.

Representative compounds of formula VI are found in Table 7 below:

TABLE 7

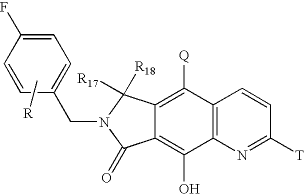

| | | | | |
|---|---|---|---|---|
| R | R₁₇ | R₁₈ | Q | T |

| | | | |
|---|---|---|---|
| H | ═O | —OH | Cl |
| H | ═O | —OCH₂CH₂Si(CH₃)₃ | —NH₂ |
| H | ═O | —N(CH₃)SO₂CH₃ | H |

| | | | | |
|---|---|---|---|---|
| H | H | —OH | —N(CH₃)SO₂CH₃ | H |
| H | —OH | H | —N(CH₃)SO₂CH₃ | H |

| | | | |
|---|---|---|---|
| H | ═O | —NH₂ | H |
| H | ═O | N-morpholino | H |

In one embodiment, a pharmaceutically acceptable salt of formula VI is represented by formula Vla:

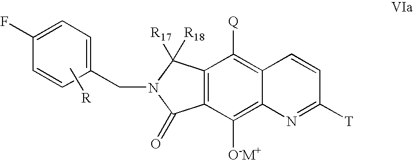

where R is selected from the group consisting of hydrogen, CH₃HNC(O)—, (CH₃)₂NC(O)—, (CH₃)₂NS(O)₂—, CH₃S(O)₂—, cyano and amino;

R₁₇ and R₁₈ are independently selected from the group consisting of hydrogen, hydroxyl or R₁₇ and R₁₈, together with the carbon atom pendent thereto, form a carbonyl group provided that both R₁₇ and R₁₈ are not hydrogen;

Q is amino, hydroxyl, 2-(trimethylsilyl)ethoxy; and —N(CH₃)SO₂CH₃;

T is hydrogen, amino or halo; and

M⁺ is a pharmaceutically acceptable cation.

In one embodiment, when R is hydrogen, T is chloro, Q is hydroxyl, and R₁₇ and R₁₈, together with the carbon atom pendent thereto, form a carbonyl group, then M⁺ is potassium.

In one embodiment, when R is hydrogen, T is chloro, Q is hydroxyl, and R₁₇ and R₁₈, together with the carbon atom pendent thereto, form a carbonyl group, then M⁺ is sodium.

In one embodiment, when R is hydrogen, T is amino, Q is 2-(trimethylsilyl)ethoxy, and R₁₇ and R₁₈, together with the carbon atom pendent thereto, form a carbonyl group, then M⁺ is potassium.

In one embodiment, when R is hydrogen, T is amino, Q is 2-(trimethylsilyl)ethoxy, and R₁₇ and R₁₈, together with the carbon atom pendent thereto, form a carbonyl group, then M⁺ is sodium.

In one embodiment, when R and T are hydrogen, Q is —N(CH₃)SO₂CH₃, and R₁₇ and R₁₈, together with the carbon atom pendent thereto, form a carbonyl group, or R₁₇ is hydroxyl and R₁₈ is hydrogen (both R and S stereochemistry). In one embodiment, when R and T are hydrogen and Q is amino, and R₁₇ and R₁₈, together with the carbon atom pendent thereto, form a carbonyl group then M⁺ is potassium. In one embodiment, when R and T are hydrogen and Q is morpholino, and R₁₇ and R₁₈, together with the carbon atom pendent thereto, form a carbonyl group then M⁺ is potassium.

In one embodiment, when R and T are hydrogen, Q is —N(CH₃)SO₂CH₃, and R₁₇ and R₁₈, together with the carbon atom pendent thereto, form a carbonyl group, or R₁₇ is hydroxyl and R₁₈ is hydrogen (both R and S stereochemistry). In one embodiment, when R and T are hydrogen and Q is amino, and R₁₇ and R₁₈, together with the carbon atom pendent thereto, form a carbonyl group then M⁺ is sodium. In one embodiment, when R and T are hydrogen and Q is morpholino, and R₁₇ and R₁₈, together with the carbon atom pendent thereto, form a carbonyl group then M⁺ is sodium.

Still another embodiment provides compounds represented by the formula VII: 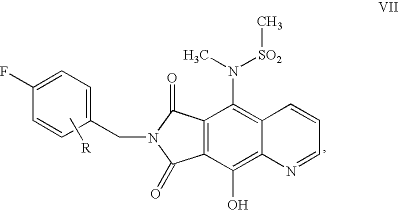
or pharmaceutically acceptable salts thereof,

where,

R is selected from the group consisting of hydrogen, CH₃HNC(O)—, (CH₃)₂NC(O)—, (CH₃)₂NS(O)₂—, CH₃S(O)₂—, cyano and amino;

In one embodiment, R is hydrogen.

A representative compound of formula VII is found in Table 8 below:

TABLE 8

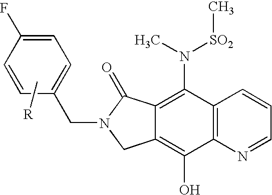

R
H

In one embodiment, a pharmaceutically acceptable salt of formula VII is represented by formula VIIa:

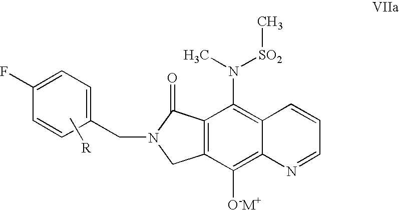

where R is as defined above and M⁺ is a pharmaceutically acceptable cation.

In one embodiment, R is hydrogen and M⁺ is sodium. In one embodiment, R is hydrogen and M⁺ is potassium.

In another embodiment, the compounds of this invention are represented by formula XXIV: 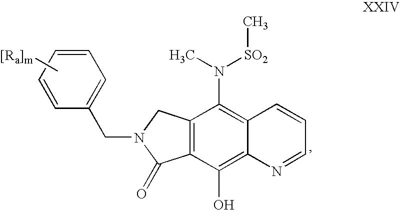
or pharmaceutically acceptable salts thereof,

where,

each Rₐ is independently halo; and

m is zero, one, two, three, four or five.

In certain preferred embodiments, each Rₐ is independently Cl or F, and m is one, two or three. For example, each Rₐ is independently Cl or F, and m is three.

In one embodiment, the compounds of this invention are represented by formula XXIVa: 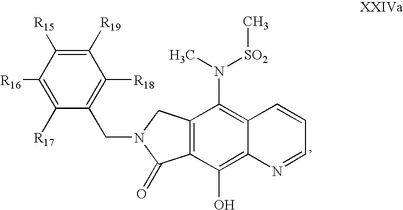
or pharmaceutically acceptable salts thereof,

where,

R₁₅, R₁₆, R₁₇, R₁₈ and R₁₉ are independently H, Cl or F.

Representative compounds of formula XXIVa are set forth in Table 9 below:

TABLE 9

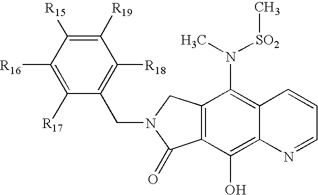

| R₁₅ | R₁₆ | R₁₇ | R₁₈ | R₁₉ |
|---|---|---|---|---|
| H | Cl | H | H | H |
| H | Cl | F | H | H |
| F | F | H | H | H |
| F | Cl | H | H | H |
| H | Cl | H | F | H |
| Cl | H | H | H | H |
| H | Cl | F | F | H |
| F | H | F | F | H |
| H | F | H | F | H |
| H | Cl | H | H | Cl |
| H | H | F | F | H |
| Cl | Cl | H | H | H |
| F | Cl | F | H | H |
| F | Cl | H | F | H |

In still another embodiment, the compounds of this invention are represented by formula XXV: 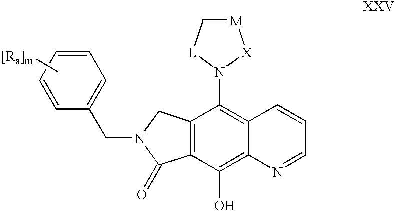
or pharmaceutically acceptable salts thereof,

where,

L is —CH₂—, —CH₂—CH₂—or —C(O)—;

X is —S(O)₂—or —C(O)—;

M is —N(R₂₀)—or —CH₂—;

R₂₀ is H or —C₁₋₄alkyl;

each Rₐ is independently halo; and

m is zero, one, two, three, four or five.

In certain embodiments, the compounds of this invention are represented by formula XXVa: 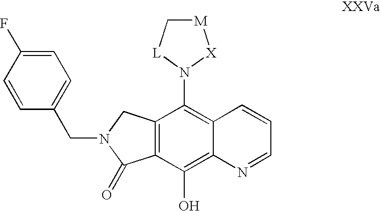
or pharmaceutically acceptable salts thereof,

where,

L is —CH₂—, —CH₂—CH₂—or —C(O)—;

X is —S(O)₂—or —C(O)—;

M is —N(R₂₀)—or —CH₂—; and

R₂₀ is H or —CH₃.

Representative compounds of formula XXVa are set forth in Table 10 below:

TABLE 10

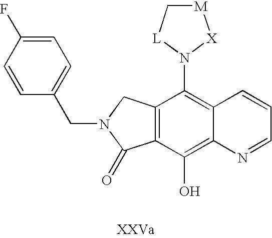

| L | X | M | R₂₀ |
|---|---|---|---|
| —CH₂—CH₂— | —S(O)₂— | —N(R₂₀)— | H |
| —CH₂— | —S(O)₂— | —N(R₂₀)— | H |
| —CH₂—CH₂— | —S(O)₂— | —N(R₂₀)— | —CH₃ |
| —CH₂—CH₂— | —C(O)— | —N(R₂₀)— | H |
| —CH₂—CH₂— | —C(O)— | —N(R₂₀)— | —CH₃ |
| —C(O)— | —C(O)— | —CH₂— | — |

Prodrugs of the compounds described above are also encompassed by this invention. In one embodiment, the prodrugs of formula I and Ia are represented by formula VIII and VIIIa:

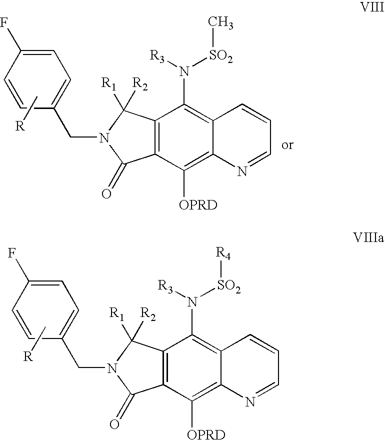

where R is selected from the group consisting of hydrogen, CH₃HNC(O)—, (CH₃)₂NC(O)—, (CH₃)₂NS(O)₂—, CH₃S(O)₂—, cyano and amino;

R, and R₂ are independently selected from the group consisting of hydrogen and methyl;

R₃ is selected from the group consisting of hydrogen, methyl and ethyl or R₃ and R₄ are cyclized to form, together with the nitrogen atom pendent to the R₃ group and the SO₂ group pendent to the R₄ group a heterocyclic or substituted heterocyclic group;

R₄ is N,N-dimethylamino; and

PRD is a pharmaceutically acceptable prodrug entity;

or pharmaceutically acceptable salts thereof.

In one embodiment of formula VIII, R₃ is methyl. In another embodiment of formula VIII, R₃ is hydrogen. In still another embodiment of formula VIII, R₃ is ethyl.

In one embodiment of formula VIII, when R₃ is methyl or hydrogen, then R₁ and R₂ are hydrogen. In one embodiment of formula VIII, when R₃ is methyl, then R. is hydrogen and R₂ is methyl. In still another embodiment of formula VIII, when R₃ is methyl, then R₁ and R₂ are methyl.

In one embodiment of formula VIIIa, when R, R₁, R₂ and R₃ are hydrogen, then R₄ is N,N-dimethylamino. In another embodiment of formula VIIIa, when R, R₁ and R₂ are hydrogen, then R₃ and R₄ are joined to form a 2-dioxoisothazolidine heterocyclic group.

In one embodiment, the prodrug entity, PRD, is selected from the group consisting of C₁-C₆ alkoxycarbonyloxymethylene, C₁-C₆ alkoxycarbonyl and C₃-C₇ cycloalkoxycarbonyloxymethylene.

In one embodiment, the prodrug entity, PRD is selected from the group consisting of isopropoxycarbonyl, cyclobutoxycarbonyloxymethylene, pent-3-oxycarbonyloxymethylene, cyclopentyloxycarbonyloxymethylene and isopropoxycarbonyloxymethylene.

In one embodiment, the prodrugs of formula II are represented by formula IX:

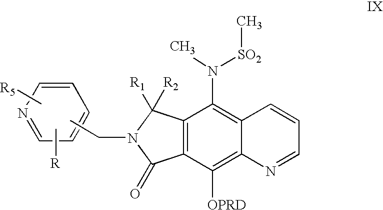

where R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl;

R₅ is selected from the group consisting of hydrogen and fluoro; and

PRD is a pharmaceutically acceptable prodrug entity;

or pharmaceutically acceptable salts thereof.

In one embodiment, when R, R₁ and R₂ are hydrogen and R₅ is 6-fluoro, then the pyridyl group is 3-pyridyl. In one embodiment, when R, R₁, R₂ and R₅ are hydrogen, then the pyridyl group is 2-pyridyl, 3-pyridyl or 4-pyridyl. In one embodiment, when R, R₁ and R₂ are hydrogen and R₅ is 5-fluoro, then the pyridyl group is 2-pyridyl.

In one embodiment, the prodrug entity, PRD, is selected from the group consisting of C₁-C₆ alkoxycarbonyl, C₁-C₆ alkoxycarbonyloxymethylene, and C₃-C₇ cycloalkoxycarbonyloxymethylene.

In one embodiment, the prodrug entity, PRD is selected from the group consisting of isopropoxycarbonyl, cyclobutoxycarbonyloxymethylene, pent-3-oxycarbonyloxymethylene, cyclopentyloxycarbonyloxymethylene and isopropoxycarbonyloxymethylene.

In one embodiment, the prodrugs of formula III are represented by formula X:

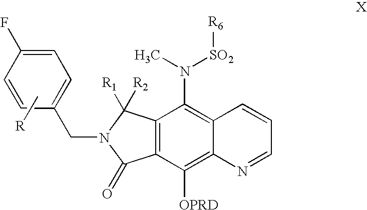

where R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl;

R₆ is selected from the group consisting of methyl, ethyl, isopropyl,1-methylimidazol-4-yl, 2,4-dimethylthiazol-5-yl, 2-(N,N-dimethylamino)eth-1-yl, 2-(N,N-diethylamino)eth-1-yl, 3-cyanoprop-1-yl, 3-(N-morpholino)prop-1-yl, 2-(N-morpholino)eth-1-yl, 3-(N,N-dimethylamino)prop-1-yl, amino, N-methylamino, N,N-dimethylamino, 2-(methylcarbonylamino)-4-methylthiazol-5-yl, 6-(N-morpholino)pyrid-3-yl, pyrid-2-yl, N-methyl-N-(pyrid-4-yl)methylamino, N-methyl-N-benzylamino, 2,2,2-trifluoroeth-1-yl, 2-(piperazin-2-yl)eth-1-yl, 2-(N-piperidinyl)eth-1-yl, 3-(imidazol-1-yl)-prop-1-yl, morpholino, and 5-N,N-dimethylaminonaphth-1-yl; and

PRD is a pharmaceutically acceptable prodrug entity;

and pharmaceutically acceptable salts thereof.

In one embodiment, the prodrug entity, PRD, is selected from the group consisting of C₁-C₆ alkoxycarbonyl, C₁-C₆ alkoxycarbonyloxymethylene, and C₃-C₇ cycloalkoxycarbonyloxymethylene.

In one embodiment, the prodrug entity, PRD is selected from the group consisting of isopropoxycarbonyl, cyclobutoxycarbonyloxymethylene, pent-3-oxycarbonyloxymethylene, cyclopentyloxycarbonyloxymethylene and isopropoxycarbonyloxymethylene.

In one embodiment, the prodrugs of formula IV are represented by formula XI:

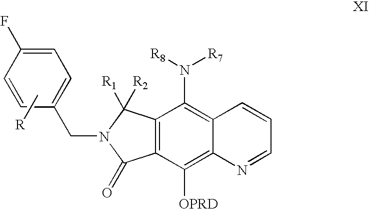

where R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl;

R₇ is selected from the group consisting of hydrogen and methyl;

R₈ is selected from the group consisting of hydrogen, —C(O)OR₉, —C(O)R₁₀, —C(O)C(O)NR₁₁R₁₁ where R₉ is hydrogen, C₁-C₄ alkyl, phenyl or substituted phenyl, R₁₀ is amino, C₁-C₄ alkylamino, [C₁-C₄ alkyl]₂amino, C₁-C₄ alkyl, heterocyclic or substituted heterocyclic, and each R₁₁ is independently hydrogen or C₁-C₄ alkyl;

or where R₇ and R₈, together with the nitrogen atom pendent thereto, form a heterocyclic or substituted heterocyclic group; and

PRD is a pharmaceutically acceptable prodrug entity;

or pharmaceutically acceptable salts thereof.

In one embodiment, when R, R₁ and R₂ are hydrogen, then R₇ and R₈, together with the nitrogen atom pendent thereto, form a group selected from the group consisting of amino, N-methyl-N-ethoxycarbonylamino, N-methyl-N-(N,N-dimethylamino-carbonyl)carbonylamino, N-methyl-N-isopropylcarbonylamino, N-methyl-N-(N-morpholino)carbonylamino, N-methyl-N-(N-methylamino)carbonylamino, and p-nitrophenoxycarbonylamino.

In one embodiment, when R and R₁ are hydrogen, and R₂ is methyl, then R₇ and R₈, together with the nitrogen atom pendent thereto, form N-methyl-N-ethoxycarbonylamino.

In one embodiment, when R is hydrogen, and R, and R₂ are methyl, then R₇ and R₈, together with the nitrogen atom pendent thereto, form N-methyl-N-ethoxycarbonylamino.

In one embodiment, the prodrug entity, PRD, is selected from the group consisting of C₁-C₆ alkoxycarbonyl, C₁-C₆ alkoxycarbonyloxymethylene, and C₃-C₇ cycloalkoxycarbonyloxymethylene.

In one embodiment, the prodrug entity, PRD is selected from the group consisting of isopropoxycarbonyl, cyclobutoxycarbonyloxymethylene, pent-3-oxycarbonyloxymethylene, cyclopentyloxycarbonyloxymethylene and isopropoxycarbonyloxymethylene.

In one embodiment, the prodrugs of formula V are represented by formula XII:

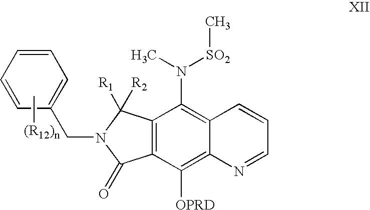

where R, and R₂ are independently selected from the group consisting of hydrogen and methyl;

R₁₂ is independently selected from the group consisting of halo, C₁-C₄ alkoxy, —C(O)OR₉ where R₉ is hydrogen or C₁-C₄ alkyl, —C(O)NR₁₅R₁₆ where each R₁₅ and R₁₆ is independently selected from the group consisting of hydrogen and C₁-C₄ alkyl, amino, C₁-C₄ alkylamino, di(C₁-C₄ alkyl)amino, cyano, —SO₂—(C₁-C₄ alkyl), and —SO₂—NR₁₅R₁₆ where R₁₅ and R₁₆ are as defined above;

PRD is a pharmaceutically acceptable prodrug entity; and

n is one, two or three;

or pharmaceutically acceptable salts thereof.

In one embodiment, the prodrug entity, PRD, is selected from the group consisting of C₁-C₆ alkoxycarbonyl, C₁-C₆ alkoxycarbonyloxymethylene, and C₃-C₇ cycloalkoxycarbonyloxymethylene.

In one embodiment, the prodrug entity, PRD is selected from the group consisting of isopropoxycarbonyl, cyclobutoxycarbonyloxymethylene, pent-3-oxycarbonyloxymethylene, cyclopentyloxycarbonyloxymethylene and isopropoxycarbonyloxymethylene.

In one embodiment, the prodrugs of formula Va are represented by formula XIII:

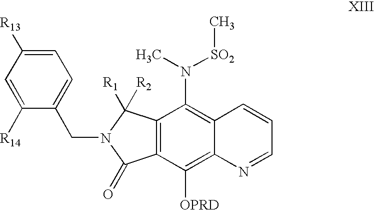

where R₁ and R₂ are independently selected from the group consisting of hydrogen and methyl;

R₁₃ and R₁₄ are independently selected from the group consisting of halo, C₁-C₄ alkoxy, —C(O)OR₉ where R₉ is hydrogen or C₁-C₄ alkyl, —C(O)NR₁₅R₁₆ where each R₁₅ and R₁₆ is independently selected from the group consisting of hydrogen and C₁-C₄ alkyl, amino, C₁-C₄ alkylamino, di(C₁-C₄ alkyl)amino, cyano, —SO₂—(C₁-C₄ alkyl), and —SO₂—NR₁₅R₁₆ where R₁₅ and R₁₆ are as defined above; and

PRD is a pharmaceutically acceptable prodrug entity;

or pharmaceutically acceptable salts thereof.

In one embodiment of formula XIII, when R₁ and R₂ are hydrogen, then R₁₃ and R₁₄, together with the phenyl group pendent thereto, form a group selected from the group consisting of 2-chloro-4-fluorophenyl, 2,4-dimethoxyphenyl, 2,4-difluorophenyl, 2-amino-4-fluorophenyl, 2-cyano-4-fluorophenyl, 2-(N,N-dimethylamino)carbonyl-4-fluorophenyl, 2-methylsulfonyl-4-fluorophenyl, 2-(N,N-dimethyl)aminosulfonyl-4-fluorophenyl and 2-(N-methylamino)carbonyl-4-fluorophenyl.

In one embodiment, the prodrug entity, PRD, is selected from the group consisting of C₁-C₆ alkoxycarbonyl, C₁-C₆ alkoxycarbonyloxymethylene, and C₃-C₇ cycloalkoxycarbonyloxymethylene.

In one embodiment, the prodrug entity, PRD is selected from the group consisting of isopropoxycarbonyl, cyclobutoxycarbonyloxymethylene, pent-3-oxycarbonyloxymethylene, cyclopentyloxycarbonyloxymethylene and isopropoxycarbonyloxymethylene.

In one embodiment, the prodrugs of formula VI are represented by formula XIV:

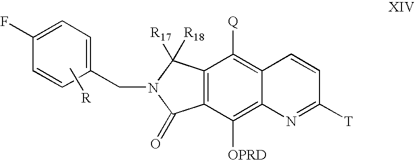

where R is selected from the group consisting of hydrogen, CH₃HNC(O)—, (CH₃)₂NC(O)—, (CH₃)₂NS(O)₂—, CH₃S(O)₂—, cyano and amino;

R₁₇ and R₁₈ are independently selected from the group consisting of hydrogen and hydroxyl, or R₁₇ and R₁₈, together with the carbon atom pendent thereto, form a carbonyl group, provided that both R₁₇ and R₁₈ are not hydrogen;

Q is amino, hydroxyl, 2-(trimethylsilyl)ethoxy, N-morpholino, and —N(CH₃)SO₂CH₃;

T is hydrogen, amino or halo; and

PRD is a pharmaceutically acceptable prodrug;

or pharmaceutically acceptable salts thereof.

In one embodiment, when R is hydrogen, T is chloro and Q is hydroxyl, then R₁₇ and R₁₈, together with the carbon atom pendent thereto, form a carbonyl group.

In one embodiment, when R is hydrogen, T is amino and Q is 2-(trimethylsilyl)ethoxy, then R₁₇ and R₁₈, together with the carbon atom pendent thereto, form a carbonyl group.

In one embodiment, when R and T are hydrogen, and Q is —N(CH₃)SO₂CH₃, then R₁₇ and R₁₈, together with the carbon atom pendent thereto, form a carbonyl group, or R₁₇ is hydroxyl and R₁₈ is hydrogen (both R and S stereochemistry). In one embodiment, when R and T are hydrogen and Q is amino, then R₁₇ and R₁₈, together with the carbon atom pendent thereto, form a carbonyl group. In one embodiment, when R and T are hydrogen and Q is morpholino, then R₁₇ and R₁₈, together with the carbon atom pendent thereto, form a carbonyl group.

In one embodiment, the prodrug entity, PRD, is selected from the group consisting of C₁-C₆ alkoxycarbonyl, C₁-C₆ alkoxycarbonyloxymethylen, and C₃-C₇ cycloalkoxycarbonyloxymethylene.

In one embodiment, the prodrug entity, PRD is selected from the group consisting of isopropoxycarbonyl, cyclobutoxycarbonyloxymethylene, pent-3-oxycarbonyloxymethylene, cyclopentyloxycarbonyloxymethylene and isopropoxycarbonyloxymethylene.

In one embodiment, the prodrugs of formula VII are represented by formula XV:

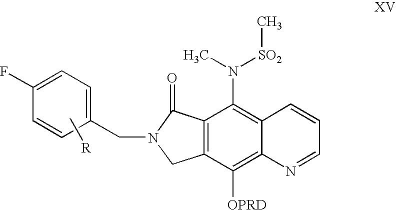

where R is selected from the group consisting of hydrogen, CH₃HNC(O)—, (CH₃)₂NC(O)—, (CH₃)₂NS(O)₂—, CH₃S(O)₂—, cyano and amino; and

PRD is a pharmaceutically acceptable prodrug entity;

or pharmaceutically acceptable salts thereof.

In one embodiment, R is hydrogen.

In one embodiment, the prodrug entity, PRD, is selected from the group consisting of C₁-C₆ alkoxycarbonyl, C₁-C₆ alkoxycarbonyloxymethylene, and C₃-C₇ cycloalkoxycarbonyloxymethylene.

In one embodiment, the prodrug entity, PRD is selected from the group consisting of isopropoxycarbonyl, cyclobutoxycarbonyloxymethylene, pent-3-oxycarbonyloxymethylene, cyclopentyloxycarbonyloxymethylene and isopropoxycarbonyloxymethylene.

Representative prodrugs of this invention are set forth in Table 11 below:

TABLE 11

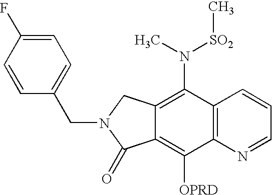

OPRD
—OC(O)OC(CH₃)₂
—OCH₂OC(O)O-cyclobutyl
—OCH₂OC(O)OC(CH₃)₂
—OCH₂OC(O)O-cyclopentyl
—OCH₂OC(O)OC(CH₂CH₃)₂

Also encompassed with the scope of this invention are intermediates in the preparation of compounds of this invention.

In one embodiment, intermediates for the preparation of compounds of formula I and Ia are represented by formula XVI, XVIa, XVIb and XVIc:

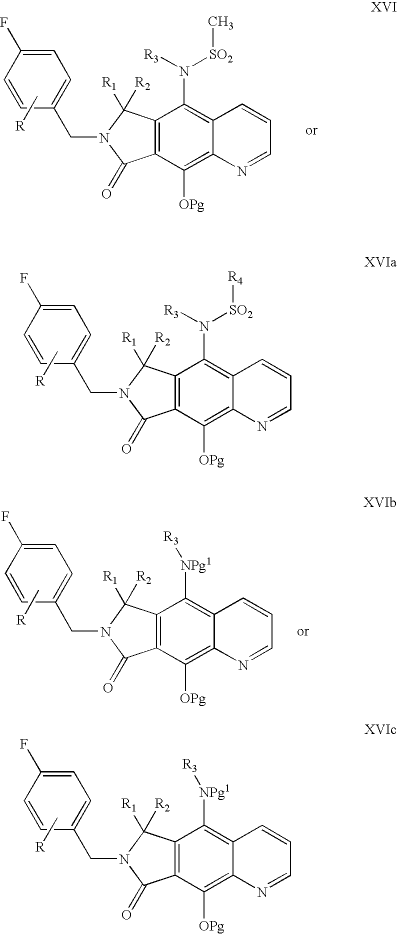

where R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl;

R₃ is selected from the group consisting of hydrogen, methyl and ethyl or R₃ and R4 are cyclized to form, together with the nitrogen atom pendent to the R₃ group and the SO₂ group pendent to the R₄ group a heterocyclic or substituted heterocyclic group;

R4 is N,N-dimethylamino;

Pg is a hydroxyl protecting group; and

Pg¹ is an amino protecting group.

In one embodiment of formula XVI, R₃ is methyl. In another embodiment of formula XVI, R₃ is hydrogen. In still another embodiment of formula XVI, R₃ is ethyl.

In one embodiment of formula XVI, when R₃ is methyl or hydrogen, then R₁ and R₂ are hydrogen. In one embodiment of formula XVI, when R₃ is methyl, then R₁ is hydrogen and R₂ is methyl. In still another embodiment of formula XVI, when R₃ is methyl, then R₁ and R₂ are methyl.

In one embodiment of formula XVIa, when R, R₁, R₂ and R₃ are hydrogen, then R₄ is N,N-dimethylamino. In another embodiment of formula XVIa, when R, R₁ and R₂ are hydrogen, then R₃ and R₄ are joined to form a 2-dioxisothiazolidine heterocyclic group.

In one embodiment, the hydroxyl protecting group is benzyl or triisopropylsilyl (TIPS).

In one embodiment, the amino protecting group is t-butoxycarbonyl (Boc or t-Boc).

In one embodiment, intermediates for the preparation of compounds of formula II are represented by formula XVII and XVIIa:

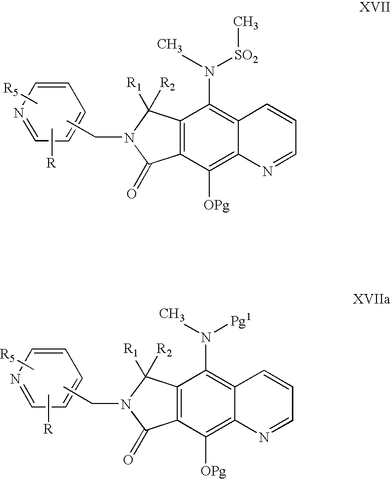

where R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl; and

R₅ is selected from the group consisting of hydrogen and fluoro;

Pg is a hydroxyl protecting group; and

Pg¹ is an amino protecting group.

In one embodiment, when R, R₁ and R₂ are hydrogen and R₅ is 6-fluoro, then the pyridyl group is 3-pyridyl. In one embodiment, when R, R₁, R₂ and R₅ are hydrogen, then the pyridyl group is 2-pyridyl, 3-pyridyl or 4-pyridyl. In one embodiment, when R, R₁ and R₂ are hydrogen and R₅ is 5-fluoro, then the pyridyl group is 2-pyridyl.

In one embodiment, the hydroxyl protecting group is benzyl or triisopropylsilyl (TIPS).

In one embodiment, the amino protecting group is t-butoxycarbonyl (Boc or t-Boc).

In one embodiment, intermediates for the preparation of compounds of formula III are represented by formula XVIII and XVIIIa: 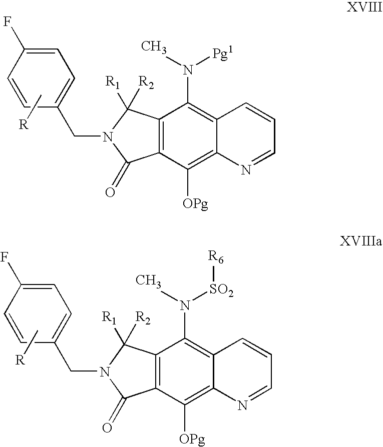
where R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl;

R₆ is selected from the group consisting of methyl, ethyl, isopropyl, 1-methylimidazol-4-yl, 2,4-dimethylthiazol-5-yl, 2-(N,N-dimethylamino)eth-1-yl, 2-(N,N-diethylamino)eth-1-yl, 3-cyanoprop-1-yl, 3-(N-morpholino)prop-1-yl, 2-(N-morpholino)eth-1-yl, 3-(N,N-dimethylamino)prop-1-yl, amino, N-methylamino, N,N-dimethylamino, 2-(methylcarbonylamino)-4-methylthiazol-5-yl, 6-(N-morpholino)pyrid-3-yl, pyrid-2-yl, N-methyl-N-(pyrid-4-yl)methylamino, N-methyl-N-benzylamino, 2,2,2-trifluoroeth-1-yl, 2-(piperazin-2-yl)eth-1-yl, 2-(N-piperidinyl)eth-1-yl, 3-(imidazol-1-yl)-prop-1-yl, N-morpholino, and 5-N,N-dimethylaminonaphth-1-yl;

Pg is a hydroxyl protecting group; and

Pg¹ is an amino protecting group.

In one embodiment, the hydroxyl protecting group is benzyl or triisopropylsilyl (TIPS).

In one embodiment, the amino protecting group is t-butoxycarbonyl (Boc or t-Boc).

In one embodiment, intermediates for the preparation of compounds of formula IV are represented by formula XIX and XIXa:

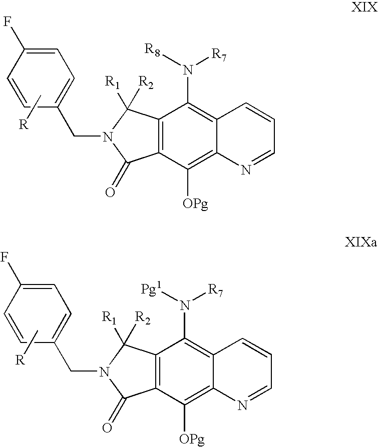

where R is selected from the group consisting of hydrogen, CH₃HNC(O)—, (CH₃)₂NC(O)—, (CH₃)₂NS(O)₂—, CH₃S(O)₂—, cyano and amino;

R₁ and R₂ are independently selected from the group consisting of hydrogen and methyl;

R₇ is selected from the group consisting of hydrogen and methyl;

R₈ is selected from the group consisting of hydrogen —C(O)OR₉, —C(O)R₁₀, —C(O)C(O)NR₁₁R₁₁ where R₉ is hydrogen, C₁-C₄ alkyl, phenyl or substituted phenyl, R₁₀ is amino, C₁-C₄ alkylamino, [C₁-C₄ alkyl]₂amino, C₁-C₄ alkyl, heterocyclic or substituted heterocyclic, and each R₁₁ is independently hydrogen or C₁-C₄ alkyl;

or where R₇ and R₈, together with the nitrogen atom pendent thereto, form a heterocyclic or substituted heterocyclic group;

Pg is a hydroxyl protecting group; and

Pg¹ is an amino protecting group.

In one embodiment, when R, R₁ and R₂ are hydrogen, then R₇ and R₈, together with the nitrogen atom pendent thereto, form a group selected from the group consisting of amino, N-methyl-N-ethoxycarbonylamino, N-methyl-N-(N,N-dimethylamino-carbonyl)carbonylamino, N-methyl-N-isopropylcarbonylamino, N-methyl-N-(N-morpholino)carbonylamino, N-methyl-N-(N-methylamino)carbonylamino, and p-nitrophenoxycarbonylamino.

In one embodiment, when R and R₁ are hydrogen, and R₂ is methyl, then R₇ and R₈, together with the nitrogen atom pendent thereto, form N-methyl-N-ethoxycarbonylamino.

In one embodiment, when R is hydrogen, and R₁ and R₂ are methyl, then R₇ and R₈, together with the nitrogen atom pendent thereto, form N-methyl-N-ethoxycarbonylamino.

In one embodiment, the hydroxyl protecting group is benzyl or triisopropylsilyl (TIPS).

In one embodiment, the amino protecting group is t-butoxycarbonyl (Boc or t-Boc).

In one embodiment, intermediates for the preparation of compounds of formula V are represented by formula XX and XXa:

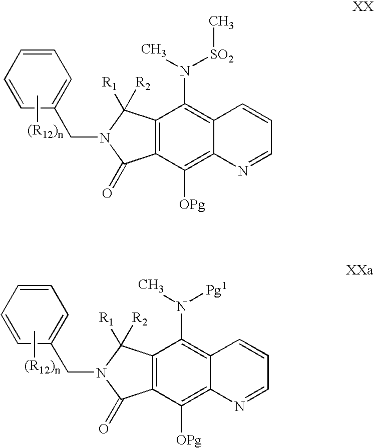

where R₁ and R₂ are independently selected from the group consisting of hydrogen and methyl;

R₁₂ is independently selected from the group consisting of halo, C₁-C₄ alkoxy, —C(O)OR₉ where R₉ is hydrogen or C₁-C₄ alkyl, —C(O)NR₁₅R₁₆ where each R₁₅ and R₁₆ is independently selected from the group consisting of hydrogen and C₁-C₄ alkyl, amino, C₁-C₄ alkylamino, di(C₁-C₄ alkyl)amino, cyano, —SO₂—(C₁-C₄ alkyl), and —SO₂—NR₁₅R₁₆ where R₁₅ and R₁₆ are as defined above; and

n is one, two or three;

Pg is a hydroxyl protecting group; and

Pg¹ is an amino protecting group.

In one embodiment, the hydroxyl protecting group is benzyl or triisopropylsilyl (TIPS).

In one embodiment, the amino protecting group is t-butoxycarbonyl (Boc or t-Boc).

In one embodiment, intermediates for the preparation of compounds of formula Va are represented by formula XXI and XXIa:

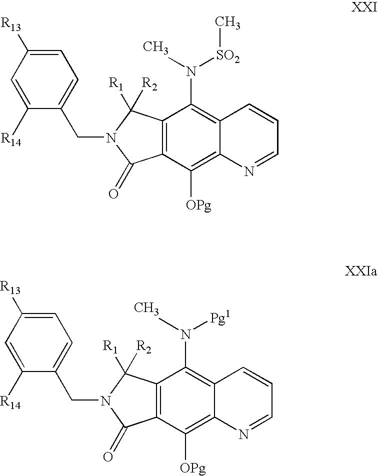

where R₁ and R₂ are independently selected from the group consisting of hydrogen and methyl;

R₁₃ and R₁₄ are independently selected from the group consisting of halo, C₁-C₄ alkoxy, —C(O)OR₉ where R₉ is hydrogen or C₁-C₄ alkyl, —C(O)NR₁₅R₁₆ where each R₁₅ and R₁₆ is independently selected from the group consisting of hydrogen and C₁-C₄ alkyl, amino, C₁-C₄ alkylamino, di(C₁-C₄ alkyl)amino, cyano, —SO₂—(C₁-C₄ alkyl), and —SO₂—NR₁₅R₁₆ where R₁₅ and R₁₆ are as defined above;

Pg is a hydroxyl protecting group; and

Pg¹ is an amino protecting group.

In one embodiment of formula Va, when R₁ and R₂ are hydrogen, then R₁₃ and R₁₄, together with the phenyl group pendent thereto, form a group selected from the group consisting of 2-chloro-4-fluorophenyl, 2,4-dimethoxyphenyl, 2,4-difluorophenyl, 2-amino-4-fluorophenyl, 2-cyano-4-fluorophenyl, 2-(N,N-dimethylamino)carbonyl-4-fluorophenyl, 2-methylsulfonyl-4-fluorophenyl, 2-(N,N-dimethyl)aminosulfonyl-4-fluorophenyl and 2-(N-methylamino)carbonyl-4-fluorophenyl.

In one embodiment, the hydroxyl protecting group is benzyl or triisopropylsilyl (TIPS).

In one embodiment, the amino protecting group is t-butoxycarbonyl (Boc or t-Boc).

In one embodiment, intermediates for the preparation of compounds of formula VI are represented by formula XXII and XXIIa:

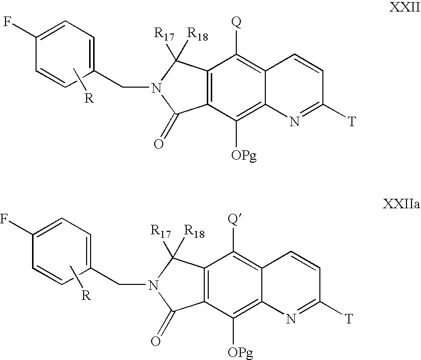

where R is selected from the group consisting of hydrogen, CH₃HNC(O)—, (CH₃)₂NC(O)—, (CH₃)₂NS(O)₂—, CH₃S(O)₂—, cyano and amino;

R₁₇ and R₁₈ are independently selected from the group consisting of hydrogen and hydroxyl, or R₁₇ and R₁₈, together with the carbon atom pendent thereto, form a carbonyl group, provided that both R₁₇ and R₁₈ are not hydrogen;

Q is amino, hydroxyl, 2-(trimethylsilyl)ethoxy, morpholino, or —N(CH₃)SO2CH₃;

Q′is —NHPg¹ or —N(CH3)Pg¹

T is hydrogen, amino or halo;

Pg is a hydroxyl protecting group; and

Pg¹ is an amino protecting group.

In one embodiment, when R is hydrogen, T is chloro and Q is hydroxyl, then R₁₇ and R₁₈, together with the carbon atom pendent thereto, form a carbonyl group.

In one embodiment, when R is hydrogen, T is amino and Q is 2-(trimethylsilyl)ethoxy, then R₁₇ and R₁₈, together with the carbon atom pendent thereto, form a carbonyl group.

In one embodiment, when R and T are hydrogen, and Q is —N(CH₃)SO₂CH₃, then R₁₇ and R₁₈, together with the carbon atom pendent thereto, form a carbonyl group, or R₁₇ is hydroxyl and R₁₈ is hydrogen (both R and S stereochemistry); or when R and T are hydrogen and Q is amino, then R₁₇ and R₁₈, together with the carbon atom pendent thereto, form a carbonyl group.

In one embodiment, the hydroxyl protecting group is benzyl or triisopropylsilyl (TIPS).

In one embodiment, the amino protecting group is t-butoxycarbonyl (Boc or t-Boc).

In one embodiment, intermediates for the preparation of compounds of formula VII are represented by formula XXIII and XXIIIa:

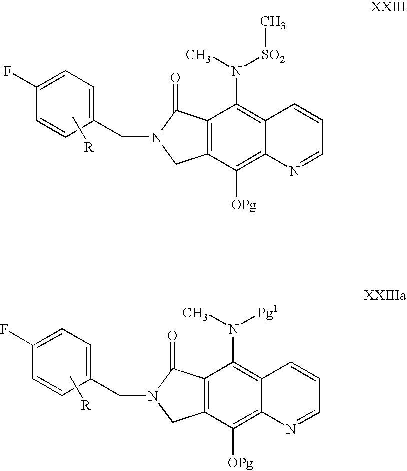

where R is selected from the group consisting of hydrogen, CH₃HNC(O)—, (CH₃)₂NC(O)—, (CH₃)₂NS(O)₂—, CH₃S(O)₂—, cyano and amino;

Pg is a hydroxyl protecting group; and

Pg¹ is an amino protecting group.

In one embodiment, R is hydrogen.

In one embodiment, the hydroxyl protecting group is benzyl or triisopropylsilyl (TIPS).

In one embodiment, the amino protecting group is t-butoxycarbonyl (Boc or t-Boc).

Novel tricyclic compounds with inhibitory activity against HIV integrase are described, including any pharmaceutically acceptable salts thereof. The salts, solvates, resolved enantiomers and purified diastereomers thereof are also contemplated. The compounds were named using the naming function in Chem Draw Ultra 9.0® (available from Cambridge Software, Cambridge Mass.).

Specific compounds included in this invention are disclosed in Table A below.

TABLE A

| | | EC₅₀ | | |
|---|---|---|---|---|
| Compound | IC₅₀ | Range | | |
| # | Range | MT-4 | Compound Structure | Name |
| 52 | A | A | 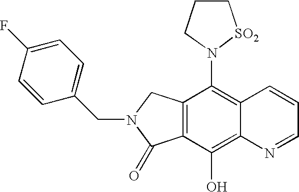 | N-(7-(4-fluorobenzyl)-9- hydroxy-8-oxo-7,8-dihydro-6H- pyrrolo[3,4-g]quinolin-5-yl)-N- 2-dioxoisothiazolidine |
| 29 | A | A | 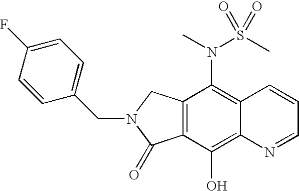 | N-(7-(4-fluorobenzyl)-9- hydroxy-8-oxo-7,8-dihydro-6H- pyrrolo[3,4-g]quinolin-5-yl)-N- methylmethanesulfonamide |
| 74 | A | A | 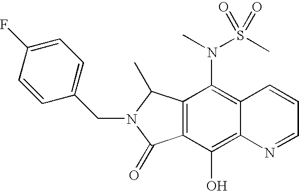 | N-(7-(4-fluorobenzyl)-9- hydroxy-6-(R and S)-methyl-8- oxo-7,8-dihydro-6H- pyrrolo[3,4-g]quinolin-5-yl)-N- methylmethanesulfonamide |
| 75 | A | A | 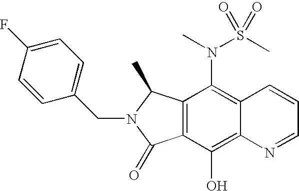 | N-(7-(4-fluorobenzyl)-9- hydroxy-6-(S)-methyl-8-oxo- 7,8-dihydro-6H-pyrrolo[3,4- g]quinolin-5-yl)-N- methylmethanesulfonamide |
| 76 | A | A | 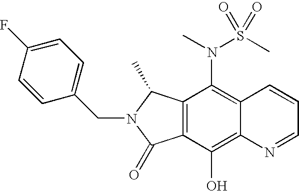 | N-(7-(4-fluorobenzyl)-9- hydroxy-6-(S)-methyl-8-oxo- 7,8-dihydro-6H-pyrrolo[3,4- g]quinolin-5-yl)-N- methylmethanesulfonamide |
| 71 | B | A | 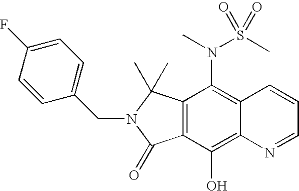 | N-(7-(4-fluorobenzyl)-9- hydroxy-6,6-dimethyl-8-oxo- 7,8-dihydro-6H-pyrrolo[3,4- g]quinolin-5-yl)-N- methylmethanesulfonamide |
| 88 | B | A | 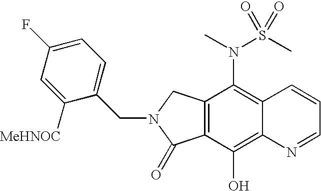 | 5-fluoro-2-((9-hydroxy-5-(N- methylmethylsulfonamido)-8- oxo-6H-pyrrolo[3,4-g]quinolin- 7(8H)-yl)methyl)-N- methylbenzamide |
| 90 | B | A | 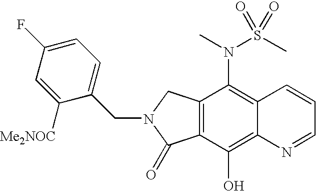 | 5-fluoro-2-((9-hydroxy-5-(N- methylmethylsulfonamido)-8- oxo-6H-pyrrolo[3,4-g]quinolin- 7(8H)-yl)methyl)-N,N- dimethylbenzamide |
| 109 | B | A | 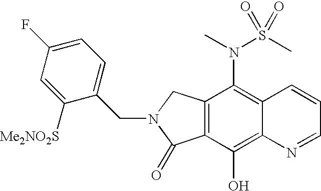 | 5-fluoro-2-((9-hydroxy-5-(N- methylmethylsulfonamido)-8- oxo-6H-pyrrolo[3,4-g]quinolin- 7(8H)-yl)methyl)-N,N- dimethylbenzenesulfonamide |
| 106 | B | A | 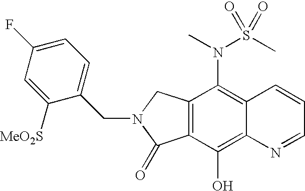 | N-(7-(4-fluoro-2- (methylsulfonyl)benzyl)-9- hydroxy-8-oxo-7,8-dihydro-6H- pyrrolo[3,4-g]quinolin-5-yl)-N- methylmethanesulfonamide |
| 100 | B | B | 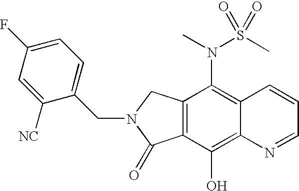 | N-(7-(2-cyano-4-fluorobenzyl)- 9-hydroxy-8-oxo-7,8-dihydro- 6H-pyrrolo[3,4-g]quinolin-5-yl)- N-methylmethanesulfonamide |
| 103 | A | A | 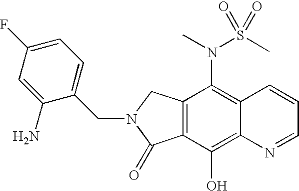 | N-(7-(2-amino-4-fluorobenzyl)- 9-hydroxy-8-oxo-7,8-dihydro- 6H-pyrrolo[3,4-g]quinolin-5-yl)- N-methylmethanesulfonamide |
| 97 | B | B | 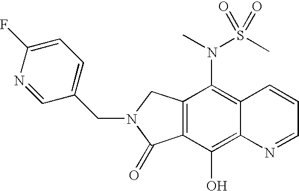 | N-(7-((6-fluoropyridin-3- yl)methyl)-9-hydroxy-8-oxo- 7,8-dihydro-6H-pyrrolo[3,4- g]quinolin-5-yl)-N- methylmethanesulfonamide |
| 94 | B | A | 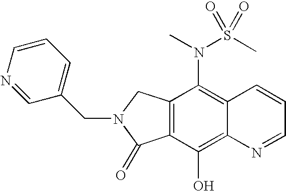 | N-(9-hydroxy-8-oxo-7-(pyridin- 2-ylmethyl)-7,8-dihydro-6H- pyrrolo[3,4-g]quinolin-5-yl)-N- methylmethanesulfonamide |
| 92 | C | C | 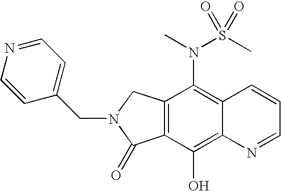 | N-(9-hydroxy-8-oxo-7-(pyridin- 4-ylmethyl)-7,8-dihydro-6H- pyrrolo[3,4-g]quinolin-5-yl)-N- methylmethanesulfonamide |
| 125 | A | B | 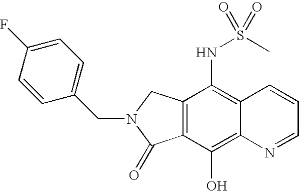 | N-(7-(4-fluorobenzyl)-9- hydroxy-8-oxo-7,8-dihydro-6H- pyrrolo[3,4-g]quinolin-5- yl)methanesulfonamide |
| 78 | A | A | 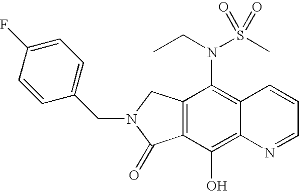 | N-ethyl-N-(7-(4-fluorobenzyl)- 9-hydroxy-8-oxo-7,8-dihydro- 6H-pyrrolo[3,4-g]quinolin-5- yl)methanesulfonamide |
| 54 | B | A | 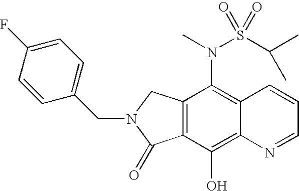 | N-(7-(4-fluorobenzyl)-9- hydroxy-8-oxo-7,8-dihydro-6H- pyrrolo[3,4-g]quinolin-5-yl)-N- methylpropane-2-sulfonamide |
| 56 | A | A | 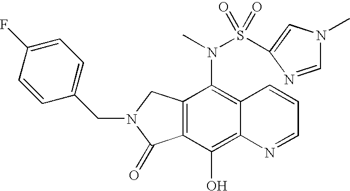 | N-(7-(4-fluorobenzyl)-9- hydroxy-8-oxo-7,8-dihydro-6H- pyrrolo[3,4-g]quinolin-5-yl)- N,1-dimethyl-1H-imidazole-4- sulfonamide |
| 58 | B | A | 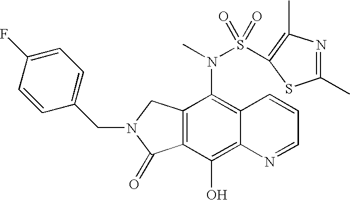 | N-(7-(4-fluorobenzyl)-9- hydroxy-8-oxo-7,8-dihydro-6H- pyrrolo[3,4-g]quinolin-5-yl)- N,2,4-trimethylthiazole-5- sulfonamide |
| 60 | A | A | 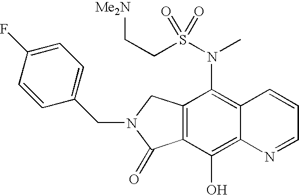 | N-(7-(4-fluorobenzyl)-9- hydroxy-8-oxo-7,8-dihydro-6H- pyrrolo[3,4-g]quinolin-5-yl)-N- methyl-2- dimethylaminoethane-1- sulfonamide |
| 48 | A | A | 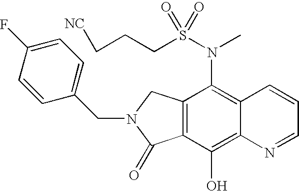 | 3-cyano-N-(7-(4-fluorobenzyl)- 9-hydroxy-8-oxo-7,8-dihydro- 6H-pyrrolo[3,4-g]quinolin-5-yl)- N-methylpropane-1- sulfonamide |
| 46 | A | A | 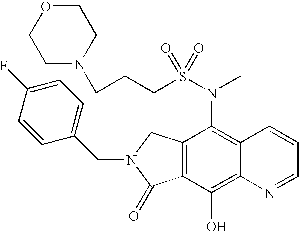 | N-(7-(4-fluorobenzyl)-9- hydroxy-8-oxo-7,8-dihydro-6H- pyrrolo[3,4-g]quinolin-5-yl)-N- methyl-3-morpholinopropane- 1-sulfonamide |
| 49 | B | A | 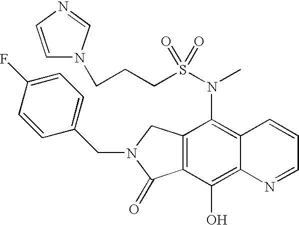 | N-(7-(4-fluorobenzyl)-9- hydroxy-8-oxo-7,8-dihydro-6H- pyrrolo[3,4-g]quinolin-5-yl)-3- (1H-imidazol-1-yl)-N- methylpropane-1-sulfonamide |
| 44 | A | A | 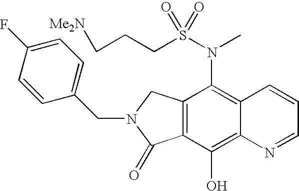 | N-(7-(4-fluorobenzyl)-9- hydroxy-8-oxo-7,8-dihydro-6H- pyrrolo[3,4-g]quinolin-5-yl)-N- methyl-3-(dimethylamino)- propane-1-sulfonamide |
| 38 | B | A | 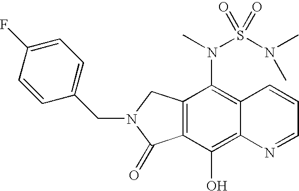 | N-(7-(4-fluorobenzyl)-9- hydroxy-8-oxo-7,8-dihydro-6H- pyrrolo[3,4-g]quinolin-5-yl)- N,N′,N′-trimethylsulfamide |
| 41 | B | A | 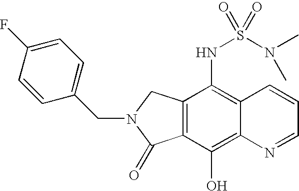 | N-(7-(4-fluorobenzyl)-9- hydroxy-8-oxo-7,8-dihydro-6H- pyrrolo[3,4-g]quinolin-5-yl)- N′,N′-dimethylsulfamide |
| 112 | A | A | 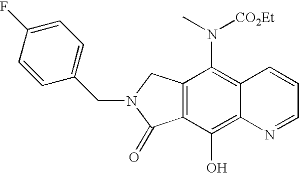 | Ethyl 7-(4-fluorobenzyl)-9- hydroxy-8-oxo-7,8-dihydro-6H- pyrrolo[3,4-g]quinolin-5- yl(methyl)carbamate |
| 114 | B | B | 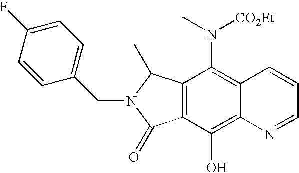 | Ethyl 7-(4-fluorobenzyl)-9- hydroxy-6-methyl-8-oxo-7,8- dihydro-6H-pyrrolo[3,4- g]quinolin-5- yl(methyl)carbamate |
| 115 | B | C | 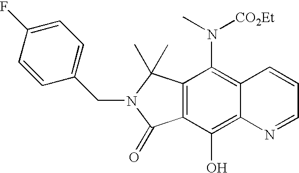 | Ethyl 7-(4-fluorobenzyl)-9- hydroxy-6,6-dimethyl-8-oxo- 7,8-dihydro-6H-pyrrolo[3,4- g]quinolin-5- yl(methyl)carbamate |
| 62 | B | A | 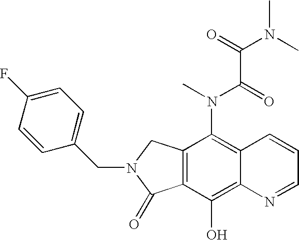 | N1-(7-(4-fluorobenzyl)-9- hydroxy-8-oxo-7,8-dihydro-6H- pyrrolo[3,4-g]quinolin-5-yl)- N1,N2,N2-trimethyloxalamide |
| 69 | A | A | 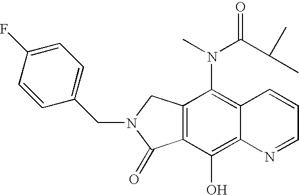 | N-(7-(4-fluorobenzyl)-9- hydroxy-8-oxo-7,8-dihydro-6H- pyrrolo[3,4-g]quinolin-5-yl)-N- methylisobutyramide |
| 67 | A | A | 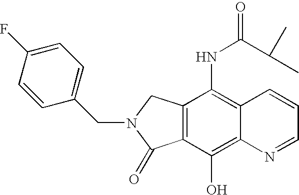 | N-(7-(4-fluorobenzyl)-9- hydroxy-8-oxo-7,8-dihydro-6H- pyrrolo[3,4-g]quinolin-5- yl)isobutyramide |
| 64 | A | A | 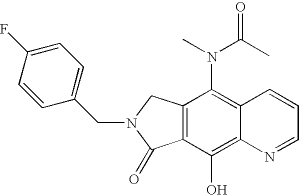 | N-(7-(4-fluorobenzyl)-9- hydroxy-8-oxo-7,8-dihydro-6H- pyrrolo[3,4-g]quinolin-5-yl)-N- methylacetamide |
| 8 | B | C | 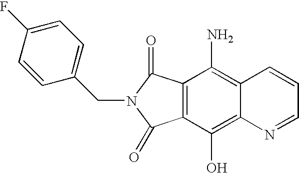 | 5-amino-7-(4-fluorobenzyl)-9- hydroxy-6H-pyrrolo[3,4- g]quinoline-6,8(7H)-dione |
| 127 | | | 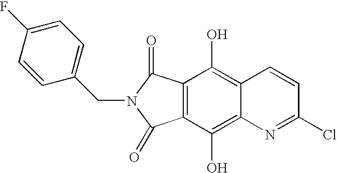 | 2-chloro-7-(4-fluorobenzyl)- 5,9-dihydroxy-6H-pyrrolo[3,4- g]quinoline-6,8(7H)-dione |
| 129 | | | 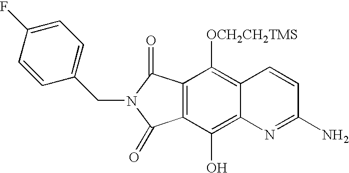 | 2-amino-7-(4-fluorobenzyl)-9- hydroxy-5-(2- (trimethylsilyl)ethoxy)-6H- pyrrolo[3,4-g]quinoline- 6,8(7H)-dione |
| 130 | | | 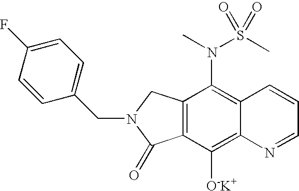 | Potassium 7-(4-fluorobenzyl)- 5-(N- methylmethylsulfonamido)-8- oxo-7,8-dihydro-6H- pyrrolo[3,4-g]quinolin-9-olate |
| 63 | | | 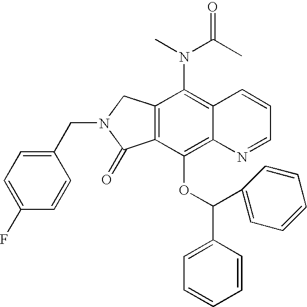 | N-(9-(benzhydryloxy)-7-(4- fluorobenzyl)-8-oxo-7,8- dihydro-6H-pyrrolo[3,4- g]quinolin-5-yl)-N- methylacetamide |
| 66 | | | 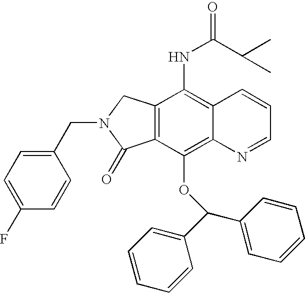 | N-(9-(benzhydryloxy)-7-(4- fluorobenzyl)-8-oxo-7,8- dihydro-6H-pyrrolo[3,4- g]quinolin-5-yl)isobutyramide |
| 68 | | | 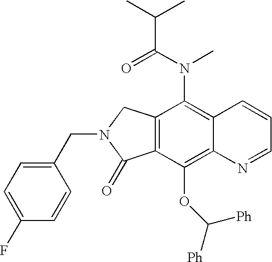 | N-(9-(benzhydryloxy)-7-(4- fluorobenzyl)-8-oxo-7,8- dihydro-6H-pyrrolo[3,4- g]quinolin-5-yl)-N- methylisobutyramide |
| 70 | | | 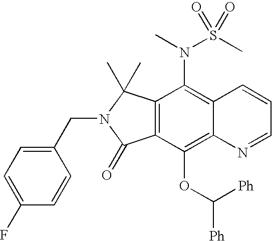 | N-(9-(benzhydryloxy)-7-(4- fluorobenzyl)-6,6-dimethyl-8- oxo-7,8-dihydro-6H- pyrrolo[3,4-g]quinolin-5-yl)-N- methylmethanesulfonamide |
| 73 | | | 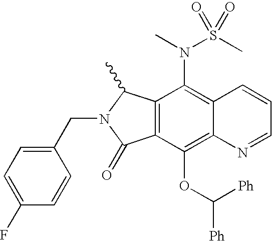 | N-(9-(benzhydryloxy)-7-(4- fluorobenzyl)-6-(R,S)-methyl- 8-oxo-7,8-dihydro-6H- pyrrolo[3,4-g]quinolin-5-yl)-N- methylmethanesulfonamide |
| 77 | | | 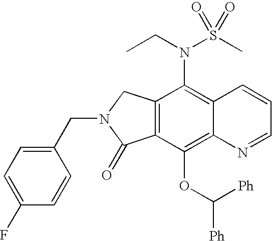 | N-ethyl-N-(9-(benzhydryloxy)- 7-(4-fluorobenzyl)-8-oxo-7,8- dihydro-6H-pyrrolo[3,4- g]quinolin-5- yl)methanesulfonamide |
| 87 | | | 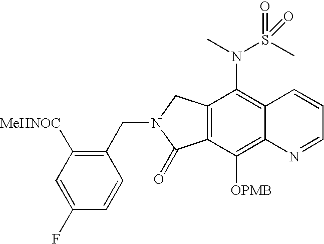 | 5-fluoro-2-((9-(4- methoxybenzyloxy)-5-(N- methylmethylsulfonamido)-8- oxo-6H-pyrrolo[3,4-g]quinolin- 7(8H)-yl)methyl)-N- methylbenzamide |
| 89 | | | 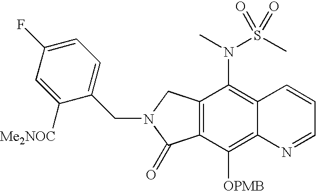 | 5-fluoro-2-((9-(4- methoxybenzyloxy)-5-(N- methylmethylsulfonamido)-8- oxo-6H-pyrrolo[3,4-g]quinolin- 7(8H)-yl)methyl)-N,N- dimethylbenzamide |
| 91 | | | 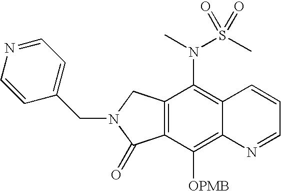 | N-(9-(4-methoxybenzyloxy)-8- oxo-7-(pyridin-4-ylmethyl)-7,8- dihydro-6H-pyrrolo[3,4- g]quinolin-5-yl)-N- methylmethanesulfonamide |
| 96 | | | 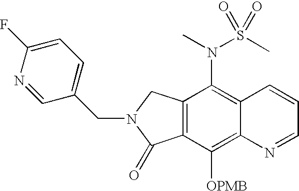 | N-(7-((6-fluoropyridin-3- yl)methyl)-9-(4- methoxybenzyloxy)-8-oxo-7,8- dihydro-6H-pyrrolo[3,4- g]quinolin-5-yl)-N- methylmethanesulfonamide |
| 99 | | | 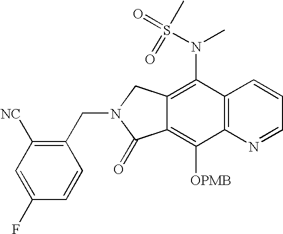 | N-(7-(2-cyano-4-fluorobenzyl)- 9-(4-methoxybenzyloxy)-8- oxo-7,8-dihydro-6H- pyrrolo[3,4-g]quinolin-5-yl)-N- methylmethanesulfonamide |
| 105 | | | 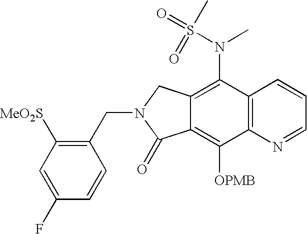 | N-(7-(4-fluoro-2- (methylsulfonyl)benzyl)-9-(4- methoxybenzyloxy)-8-oxo-7,8- dihydro-6H-pyrrolo[3,4- g]quinolin-5-yl)-N- methylmethanesulfonamide |
| 108 | | | 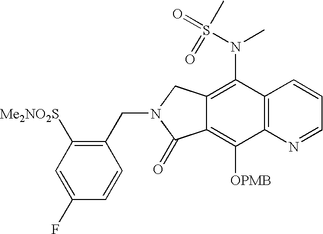 | 5-fluoro-2-((9-(4- methoxybenzyloxy)-5-(N- methylmethylsulfonamido)-8- oxo-6H-pyrrolo[3,4-g]quinolin- 7(8H)-yl)methyl)-N,N- dimethylbenzenesulfonamide |
| | | | 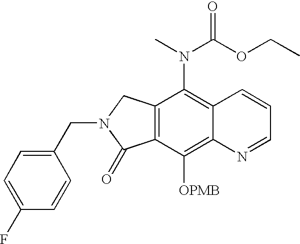 | ethyl 7-(4-fluorobenzyl)-9-(4- methoxybenzyloxy)-8-oxo-7,8- dihydro-6H-pyrrolo[3,4- g]quinolin-5- yl(methyl)carbamate |
| 113 | | | 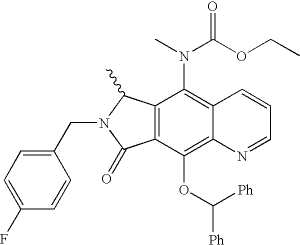 | ethyl 9-(benzhydryloxy)-7-(4- fluorobenzyl)-6-methyl-8-oxo- 7,8-dihydro-6H-pyrrolo[3,4- g]quinolin-5- yl(methyl)carbamate |
| 131 | | | 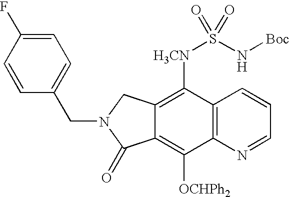 | tert-butyl N-(9- (benzhydryloxy)-7-(4- fluorobenzyl)-8-oxo-7,8- dihydro-6H-pyrrolo[3,4- g]quinolin-5-yl)-N- methylsulfamoylcarbamate |
| 132 | A | A | 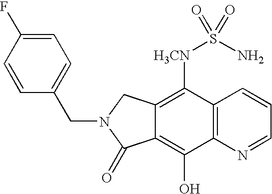 | N-(7-(4-fluorobenzyl)-9- hydroxy-8-oxo-7,8-dihydro-6H- pyrrolo[3,4-g]quinolin-5-yl)-N- methylsulfamide |
| 133 | | | 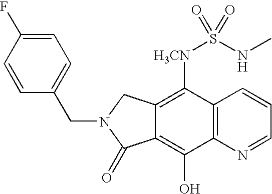 | N-(7-(4-fluorobenzyl)-9- hydroxy-8-oxo-7,8-dihydro-6H- pyrrolo[3,4-g]quinolin-5-yl)-N- methyl-N′-methylsulfamide |
| | | | 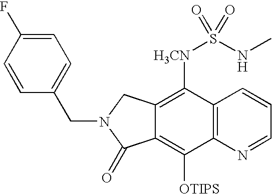 | N-(7-(4-fluorobenzyl)-9- (triisopropylsiloxy)-8-oxo-7,8- dihydro-6H-pyrrolo[3,4- g]quinolin-5-yl)-N-methyl-N′- methylsulfamide |
| 135 | B | B | 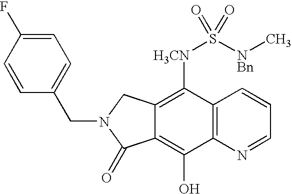 | N′-benzyl-N′-methyl-N-(7-(4- fluorobenzyl)-9-hydroxy-8-oxo- 7,8-dihydro-6H-pyrrolo[3,4- g]quinolin-5-yl)-N- methylsulfamide |
| 136 | B | B | 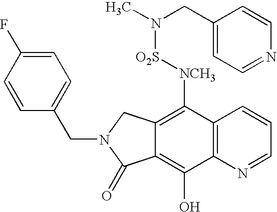 | N-(7-(4-fluorobenzyl)-9- hydroxy-8-oxo-7,8-dihydro-6H- pyrrolo[3,4-g]quinolin-5-yl)-N- methyl-N′-methyl-N′-(pyridin-4- ylmethyl)sulfamide |
| 143 | B | B | 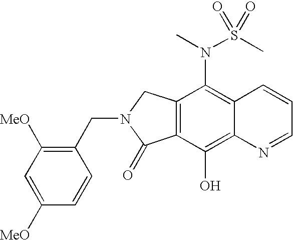 | N-(7-(2,4-dimethoxybenzyl)-9- hydroxy-8-oxo-7,8-dihydro-6H- pyrrolo[3,4-g]quinolin-5-yl)-N- methylmethanesulfonamide |
| 145 | A | A | 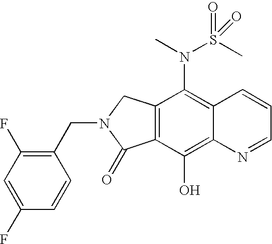 | N-(7-(2,4-difluorobenzyl)-9- hydroxy-8-oxo-7,8-dihydro-6H- pyrrolo[3,4-g]quinolin-5-yl)-N- methylmethanesulfonamide |
| 147 | A | A | 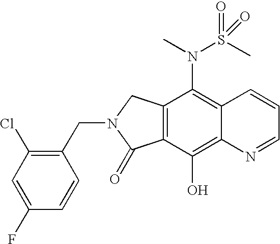 | N-(7-(2-chloro-4-fluorobenzyl)- 9-hydroxy-8-oxo-7,8-dihydro- 6H-pyrrolo[3,4-g]quinolin-5-yl)- N-methylmethanesulfonamide |
| 151 | B | B | 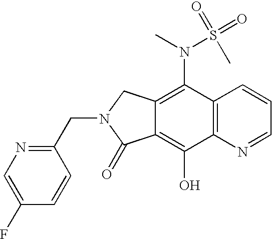 | N-(7-((5-fluoropyridin-2- yl)methyl)-9-hydroxy-8-oxo- 7,8-dihydro-6H-pyrrolo[3,4- g]quinolin-5-yl)-N- methylmethanesulfonamide |
| 152 | A | C | 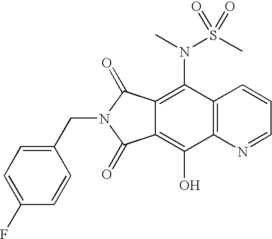 | N-(7-(4-fluorobenzyl)-9- hydroxy-6,8-dioxo-7,8-dihydro- 6H-pyrrolo[3,4-g]quinolin-5-yl)- N-methylmethanesulfonamide |
| 154 | B | C | 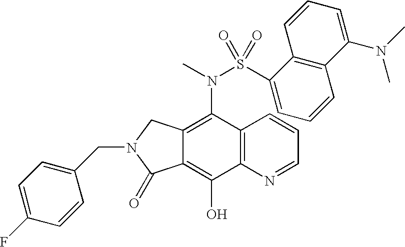 | 5-(dimethylamino)-N-(7-(4- fluorobenzyl)-9-hydroxy-8-oxo- 7,8-dihydro-6H-pyrrolo[3,4- g]quinolin-5-yl)-N- methylnaphthalene-1- sulfonamide |
| 156 | A | B | 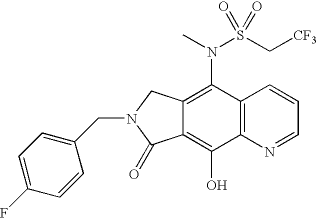 | 2,2,2-trifluoro-N-(7-(4- fluorobenzyl)-9-hydroxy-8-oxo- 7,8-dihydro-6H-pyrrolo[3,4- g]quinolin-5-yl)-N- methylethane-1-sulfonamide |
| 158 | B | A | 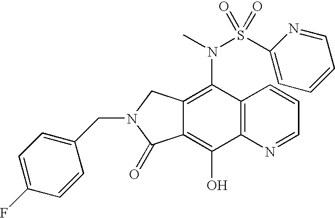 | N-(7-(4-fluorobenzyl)-9- hydroxy-8-oxo-7,8-dihydro-6H- pyrrolo[3,4-g]quinolin-5-yl)-N- methylpyridine-2-sulfonamide |
| 159 | B | B | 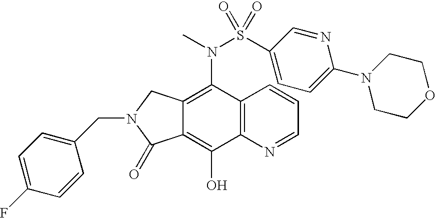 | N-(7-(4-fluorobenzyl)-9- hydroxy-8-oxo-7,8-dihydro-6H- pyrrolo[3,4-g]quinolin-5-yl)-N- methyl-6-morpholinopyridine- 3-sulfonamide |
| 160 | B | B | 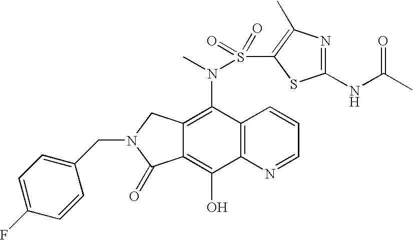 | N-(5-(N-(7-(4-fluorobenzyl)-9- hydroxy-8-oxo-7,8-dihydro-6H- pyrrolo[3,4-g]quinolin-5-yl)-N- methylsulfamoyl)-4- methylthiazol-2-yl)acetamide |
| 161 | B | A | 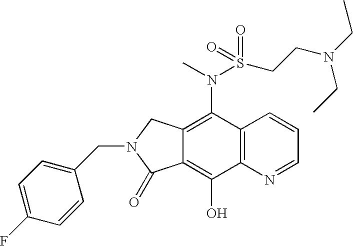 | 2-(diethylamino)-N-(7-(4- fluorobenzyl)-9-hydroxy-8-oxo- 7,8-dihydro-6H-pyrrolo[3,4- g]quinolin-5-yl)-N- methylethanesulfonamide |
| 162 | B | B | 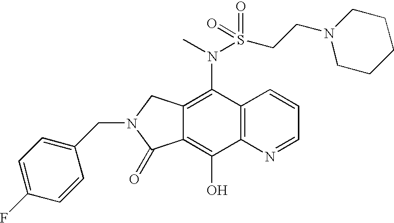 | N-(7-(4-fluorobenzyl)-9- hydroxy-8-oxo-7,8-dihydro-6H- pyrrolo[3,4-g]quinolin-5-yl)-N- methyl-2-(piperidin-1- yl)ethanesulfonamide |
| 163 | A | A | 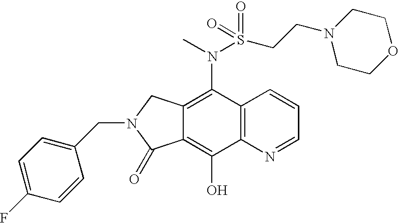 | N-(7-(4-fluorobenzyl)-9- hydroxy-8-oxo-7,8-dihydro-6H- pyrrolo[3,4-g]quinolin-5-yl)-N- methyl-2- morpholinoethanesulfonamide |
| 164 | B | B | 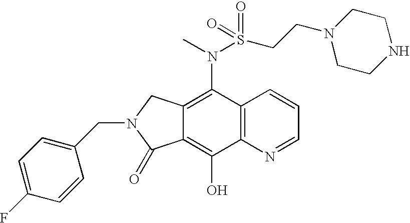 | N-(7-(4-fluorobenzyl)-9- hydroxy-8-oxo-7,8-dihydro-6H- pyrrolo[3,4-g]quinolin-5-yl)-N- methyl-2-(piperazin-1- yl)ethanesulfonamide |
| 166 | A | A | 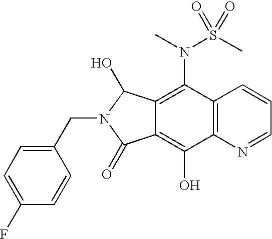 | N-(7-(4-fluorobenzyl)-6,9- dihydroxy-8-oxo-7,8-dihydro- 6H-pyrrolo[3,4-g]quinolin-5-yl)- N-methylmethanesulfonamide |
| 168 | | | 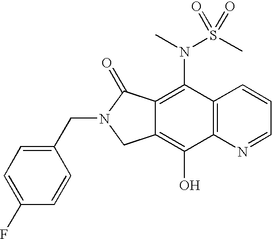 | N-(7-(4-fluorobenzyl)-9- hydroxy-6-oxo-7,8-dihydro-6H- pyrrolo[3,4-g]quinolin-5-yl)-N- methylmethanesulfonamide |
| 169 | | | 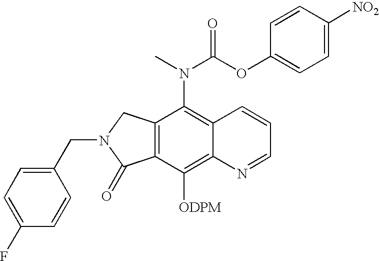 | 4-nitrophenyl 9- (benzhydryloxy)-7-(4- fluorobenzyl)-7,8-dihydro-8- oxo-6H-pyrrolo[3,4-g]quinolin- 5-ylmethylcarbamate |
| 171 | A | A | 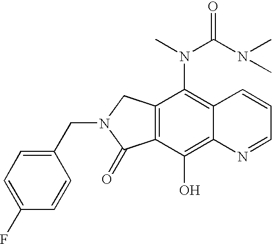 | 1-(7-(4-fluorobenzyl)-9- hydroxy-8-oxo-7,8-dihydro-6H- pyrrolo[3,4-g]quinolin-5-yl)- 1,3,3-trimethylurea |
| 170 | | | 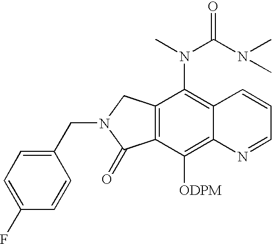 | 1-(9-(benzhydryloxy)-7-(4- fluorobenzyl)-8-oxo-7,8- dihydro-6H-pyrrolo[3,4- g]quinolin-5-yl)-1,3,3- trimethylurea |
| 173 | B | B | 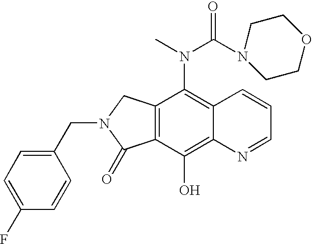 | N-(7-(4-fluorobenzyl)-9- hydroxy-8-oxo-7,8-dihydro-6H- pyrrolo[3,4-g]quinolin-5-yl)-N- methylmorpholine-4- carboxamide |
| 172 | | | 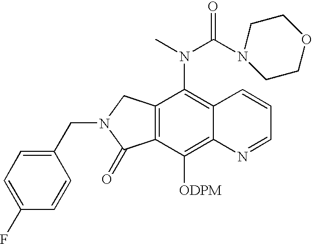 | N-(9-(benzhydryloxy)-7-(4- fluorobenzyl)-8-oxo-7,8- dihydro-6H-pyrrolo[3,4- g]quinolin-5-yl)-N- methylmorpholine-4- carboxamide |
| 34 | C | A | 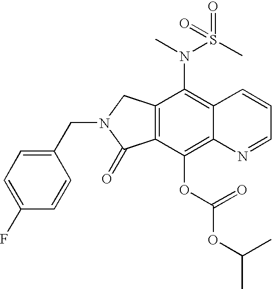 | 7-(4-fluorobenzyl)-5-(N- methylmethan-5- ylsulfonamido)-8-oxo-7,8- dihydro-6H-pyrrolo[3,4- g]quinolin-9-yl isopropyl carbonate |
| 31 | C | A | 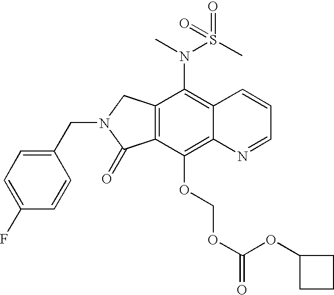 | (7-(4-fluorobenzyl)-5-(N- methylmethan-5- ylsulfonamido)-8-oxo-7,8- dihydro-6H-pyrrolo[3,4- g]quinolin-9-yloxy)methyl cyclobutyl carbonate |
| 32 | C | B | 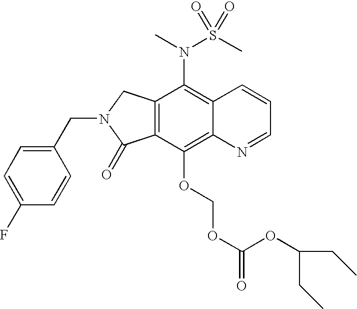 | (7-(4-fluorobenzyl)-5-(N- methylmethan-5- ylsulfonamido)-8-oxo-7,8- dihydro-6H-pyrrolo[3,4- g]quinolin-9-yloxy)methyl pentan-3-yl carbonate |
| 30 | C | B | 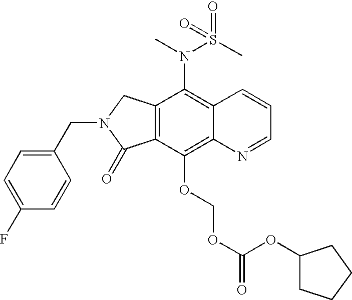 | (7-(4-fluorobenzyl)-5-(N- methylmethan-5- ylsulfonamido)-8-oxo-7,8- dihydro-6H-pyrrolo[3,4- g]quinolin-9-yloxy)methyl cyclopentyl carbonate |
| 33 | C | A | 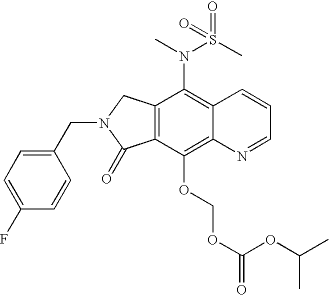 | (7-(4-fluorobenzyl)-5-(N- methylmethan-5- ylsulfonamido)-8-oxo-7,8- dihydro-6H-pyrrolo[3,4- g]quinolin-9-yloxy)methyl isopropyl carbonate |
| 227 | | | 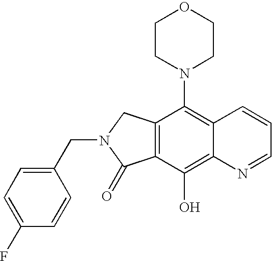 | 7-(4-fluorobenzyl)-9-hydroxy- 5-morpholino-6,7- dihydropyrrolo[3,4-g]quinolin- 8-one |
| 210 | B | A | 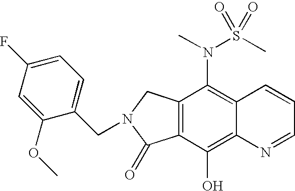 | N-(7-(4-fluoro-2- methoxybenzyl)-9-hydroxy-8- oxo-7,8-dihydro-6H- pyrrolo[3,4-g]quinolin-5-yl)-N- methylmethanesulfonamide |
| 175 | B | A | 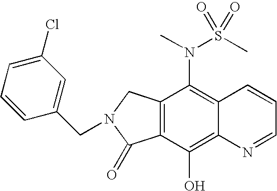 | N-(7-(3-chlorobenzyl)-9- hydroxy-8-oxo-7,8-dihydro-6H- pyrrolo[3,4-g]quinolin-5-yl)-N- methylmethanesulfonamide |
| 177 | B | A | 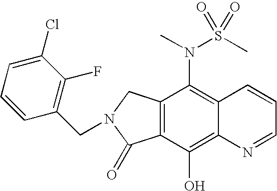 | N-(7-(3-chloro-2-fluorobenzyl)- 9-hydroxy-8-oxo-7,8-dihydro- 6H-pyrrolo[3,4-g]quinolin-5-yl)- N-methylmethanesulfonamide |
| 179 | A | A | 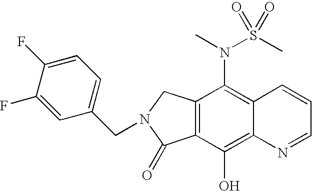 | N-(7-(3,4-difluorobenzyl)-9- hydroxy-8-oxo-7,8-dihydro-6H- pyrrolo[3,4-g]quinolin-5-yl)-N- methylmethanesulfonamide |
| 181 | A | A | 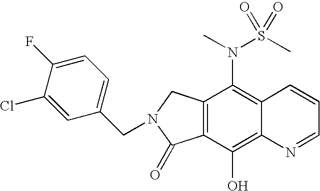 | N-(7-(3-chloro-4-fluorobenzyl)- 9-hydroxy-8-oxo-7,8-dihydro- 6H-pyrrolo[3,4-g]quinolin-5-yl)- N-methylmethanesulfonamide |
| 211 | B | A | 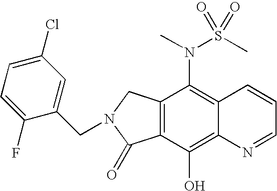 | N-(7-(5-chloro-2-fluorobenzyl)- 9-hydroxy-8-oxo-7,8-dihydro- 6H-pyrrolo[3,4-g]quinolin-5-yl)- N-methylmethanesulfonamide |
| 183 | B | A | 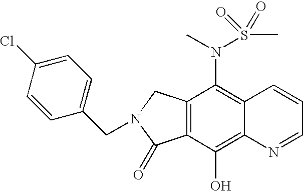 | N-(7-(4-chlorobenzyl)-9- hydroxy-8-oxo-7,8-dihydro-6H- pyrrolo[3,4-g]quinolin-5-yl)-N- methylmethanesulfonamide |
| 185 | B | A | 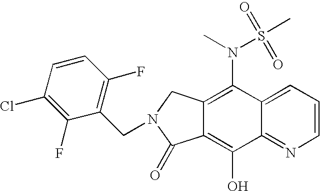 | N-(7-(3-chloro-2,6- difluorobenzyl)-9-hydroxy-8- oxo-7,8-dihydro-6H- pyrrolo[3,4-g]quinolin-5-yl)-N- methylmethanesulfonamide |
| 187 | B | A | 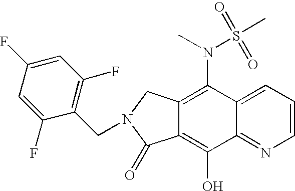 | N-(7-(2,4,6-trifluorobenzyl)-9- hydroxy-8-oxo-7,8-dihydro-6H- pyrrolo[3,4-g]quinolin-5-yl)-N- methylmethanesulfonamide |
| 189 | B | A | 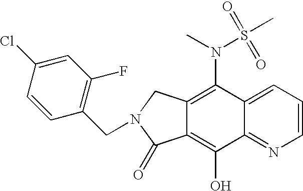 | N-(7-(4-chloro-2-fluorobenzyl)- 9-hydroxy-8-oxo-7,8-dihydro- 6H-pyrrolo[3,4-g]quinolin-5-yl)- N-methylmethanesulfonamide |
| 191 | B | A | 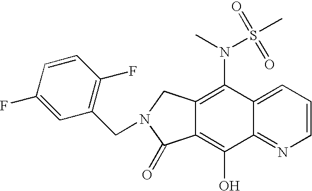 | N-(7-(2,5-difluorobenzyl)-9- hydroxy-8-oxo-7,8-dihydro-6H- pyrrolo[3,4-g]quinolin-5-yl)-N- methylmethanesulfonamide |
| 193 | B | A | 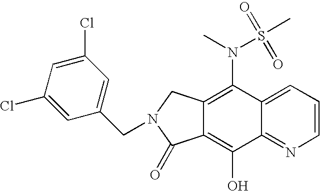 | N-(7-(3,5-dichlorobenzyl)-9- hydroxy-8-oxo-7,8-dihydro-6H- pyrrolo[3,4-g]quinolin-5-yl)-N- methylmethanesulfonamide |
| 195 | B | B | 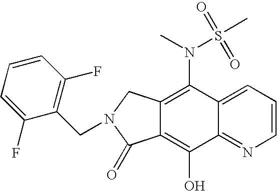 | N-(7-(2,6-difluorobenzyl)-9- hydroxy-8-oxo-7,8-dihydro-6H- pyrrolo[3,4-g]quinolin-5-yl)-N- methylmethanesulfonamide |
| 197 | B | A | 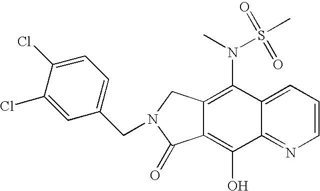 | N-(7-(3,4-dichlorobenzyl)-9- hydroxy-8-oxo-7,8-dihydro-6H- pyrrolo[3,4-g]quinolin-5-yl)-N- methylmethanesulfonamide |
| 199 | B | A | 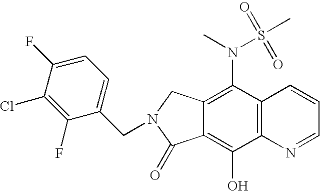 | N-(7-(3-chloro-2,4- difluorobenzyl)-9-hydroxy-8- oxo-7,8-dihydro-6H- pyrrolo[3,4-g]quinolin-5-yl)-N- methylmethanesulfonamide |
| 204 | A | A | 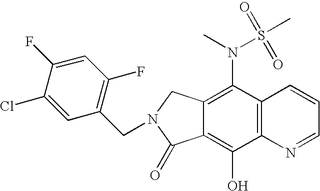 | N-(7-(5-chloro-2,4- difluorobenzyl)-9-hydroxy-8- oxo-7,8-dihydro-6H- pyrrolo[3,4-g]quinolin-5-yl)-N- methylmethanesulfonamide |
| 215 | B | A | 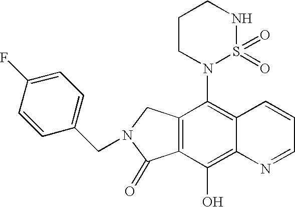 | |
| 214 | A | A | 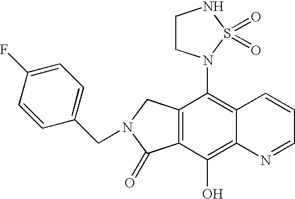 | |
| 218 | A | A | 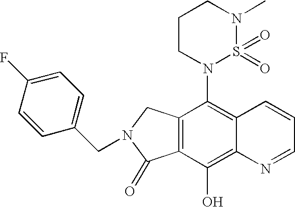 | |
| 222 | B | A | 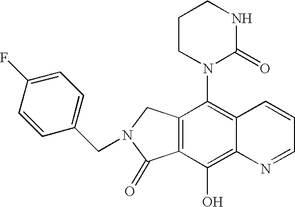 | 7-(4-fluorobenzyl)-9-hydroxy- 5-(2-oxo-tetrahydropyrimidin- 1(2H)-yl)-6,7- dihydropyrrolo[3,4-g]quinolin- 8-one |
| 224 | A | B | 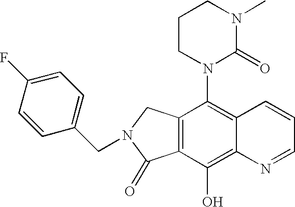 | 7-(4-fluorobenzyl)-9-hydroxy- 5-(3-methyl-2-oxo- tetrahydropyrimidin-1(2H)-yl)- 6,7-dihydropyrrolo[3,4- g]quinolin-8-one |
| 221 | A | A | 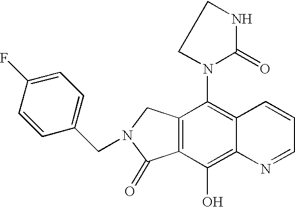 | 7-(4-fluorobenzyl)-9-hydroxy- 5-(2-oxoimidazolidin-1-yl)-6,7- dihydropyrrolo[3,4-g]quinolin- 8-one |
| 226 | A | A | 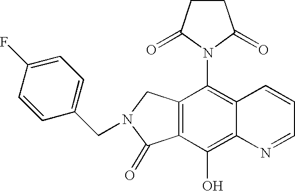 | 1-(7-(4-fluorobenzyl)-9- hydroxy-8-oxo-7,8-dihydro-6H- pyrrolo[3,4-g]quinolin-5- yl)pyrrolidine-2,5-dione |
| 232 | | A | 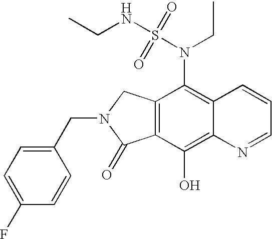 | |

A is from >0 nM to about 60 nM.
B is about 60 nM to about 1 μM.
C is > about 1 μM.
[See Biological Activity infra.]

Compounds of the invention bearing one or more prodrug moieties may increase or optimize the bioavailability of the compounds as therapeutic agents. For example, bioavailability after oral administration may be beneficial and may depend on resistance to metabolic degradation in the gastrointestinal tract or circulatory system, and eventual uptake inside cells. Prodrug moieties are considered to confer said resistance by slowing certain hydrolytic or enzymatic metabolic processes. Lipophilic prodrug moieties may also increase active or passive transport of the compounds of the invention across cellular membranes (Darby, G. Antiviral Chem. & Chemotherapy (1995) Supp. 1, 6:54-63).

Exemplary embodiments of the invention includes phosphonamidate and phosphoramidate (collectively “amidate”) prodrug compounds. General formulas for phosphonamidate and phosphoramidate prodrug moieties include:

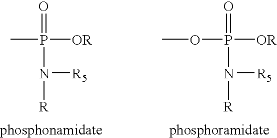

The phosphorus atom of the phosphonamidate group is bonded to a carbon atom. The nitrogen substituent R₅ may include an ester, an amide, or a carbamate functional group. For example, R₅ may be —CR₂C(═O)OR′ where R′ is H, C₁-C₆ alkyl, C₁-C₆ substituted alkyl, C₆-C₂₀ aryl, C₆-C₂₀ substituted aryl, C₂-C₂₀ heteroaryl, or C₂-C₂₀ substituted heteroaryl.

Exemplary embodiments of phosphonamidate and phosphoramidate prodrugs include:

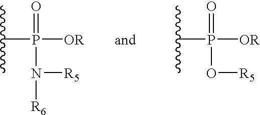

wherein R₅ is —CR₂CO₂R₇ where R₆ and R₇ are independently H or C₁-C₈ alkyl.

The nitrogen atom may comprise an amino acid residue within the prodrug moiety, such as a glycine, alanine, or valine ester (e.g. valacyclovir, see: Beauchamp, etal Antiviral Chem. Chemotherapy (1992) 3:157-164), such as the general structure: 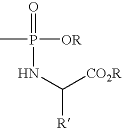
where R′ is the amino acid side-chain, e.g. H, CH₃, CH(CH₃)₂, etc.

An exemplary embodiment of a phosphonamidate prodrug moiety is:

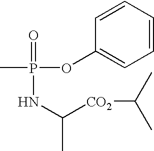

Another embodiment of the invention is directed toward an HIV integrase inhibitor tricyclic compound of the invention which is capable of accumulating in human PBMC (peripheral blood mononuclear cells). PBMC refer to blood cells having round lymphocytes and monocytes. Physiologically, PBMC are critical components of the mechanism against infection. PBMC may be isolated from heparinized whole blood of normal healthy donors or buffy coats, by standard density gradient centrifugation and harvested from the interface, washed (e.g. phosphate-buffered saline) and stored in freezing medium. PBMC may be cultured in multi-well plates. At various times of culture, supernatant may be either removed for assessment, or cells may be harvested and analyzed (Smith R. etal (2003) Blood 102(7):2532-2540). The compounds of this embodiment may further comprise a phosphonate or phosphonate prodrug. Typically, the phosphonate or phosphonate prodrug has the structure A³ as described herein.

Optionally, the compounds of this embodiment demonstrate improved intracellular half-life of the compounds or intracellular metabolites of the compounds in human PBMC when compared to analogs of the compounds not having the phosphonate or phosphonate prodrug. Typically, the half-life is improved by at least about 50%, more typically at least in the range 50-100%, still more typically at least about 100%, more typically yet greater than about 100%.

In another embodiment, the intracellular half-life of a metabolite of the compound in human PBMCs is improved when compared to an analog of the compound not having the phosphonate or phosphonate prodrug. In such embodiments, the metabolite may be generated intracellularly, or it is generated within human PBMC. The metabolite may be a product of the cleavage of a phosphonate prodrug within human PBMCs. The phosphonate prodrug may be cleaved to form a metabolite having at least one negative charge at physiological pH. The phosphonate prodrug may be enzymatically cleaved within human PBMC to form a phosphonate having at least one active hydrogen atom of the form P-OH.

Those of skill in the art will also recognize that the compounds of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state—any and all protonated forms of the compounds are intended to fall within the scope of the invention.

The compounds of this invention optionally comprise salts of the compounds herein, especially pharmaceutically acceptable non-toxic salts containing, for example, Na⁺, Li⁺, K⁺, Ca⁺² and Mg⁺². Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the invention, and that the invention encompasses the compounds in association with any type of counter ion. Moreover, as the compounds can exists in a variety of different forms, the invention is intended to encompass not only forms of the compounds that are in association with counter ions (e.g., dry salts), but also forms that are not in association with counter ions (e.g., aqueous or organic solutions).

Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids, e.g., HCI, HBr, H₂SO₄, H₃PO₄ or organic sulfonic acids, to basic centers, typically amines, or to acidic groups. Finally, it is to be understood that the compositions herein comprise compounds of the invention in their unionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention can also exist as tautomeric, resonance isomers in certain cases. Typically, the structures shown herein exemplify only one tautomeric or resonance form of the compounds. For example, hydrazine, oxime, hydrazone groups may be shown in either the syn or anti configurations. The corresponding alternative configuration is contemplated as well. All possible tautomeric and resonance forms are within the scope of the invention.

One enantiomer of a compound of the invention can be separated substantially free of its opposing enantiomer by a method such as formation of diastereomers using optically active resolving agents (Stereochemistry of Carbon Compounds (1962) by E. L. Eliel, McGraw Hill; Lochmuller, C. H., (1975) J. Chromatogr., 113:(3) 283-302). Separation of diastereomers formed from the racemic mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers. Alternatively, enantiomers can be separated directly under chiral conditions, method (3).

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched xanthene. A method of determining optical purity involves making chiral esters, such as a menthyl ester or Mosher ester, α-methoxy-α-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO 96/15111).

By method (3), a racemic mixture of two asymmetric enantiomers can be separated by chromatography using a chiral stationary phase (Chiral Liquid Chromatoqraphy (1989) W. J. Lough, Ed. Chapman and Hall, New York; Okamoto, (1990) “Optical resolution of dihydropyridine enantiomers by High-performance liquid chromatography using phenylcarbamates of polysaccharides as a chiral stationary phase”, J. of Chromatogr. 513:375-378).

Enantiomers can be distinguished by methods used to distinguish other chiral molecules with asymmetric carbon atoms, such as optical rotation and circular dichroism.

Improving the delivery of drugs and other agents to target cells and tissues has been the focus of considerable research for many years. Though many attempts have been made to develop effective methods for importing biologically-active molecules into cells, both in vivo and in vitro, none has proved to be entirely satisfactory. Optimizing the association of the inhibitory drug with its intracellular target, while minimizing intercellular redistribution of the drug, e.g. to neighboring cells, is often difficult or inefficient.

Most agents currently administered parenterally to a patient are not targeted, resulting in systemic delivery of the agent to cells and tissues of the body where it is unnecessary, and often undesirable. This may result in adverse drug side effects, and often limits the dose of a drug (e.g., cytotoxic agents and other anti-cancer or anti-viral drugs) that can be administered. By comparison, although oral administration of drugs is generally recognized as a convenient and economical method of administration, oral administration can result in either (a) uptake of the drug through the cellular and tissue barriers, e.g. blood/brain, epithelial, cell membrane, resulting in undesirable systemic distribution, or (b) temporary residence of the drug within the gastrointestinal tract. Accordingly, a major goal has been to develop methods for specifically targeting agents to cells and tissues. Benefits of such treatment include avoiding the general physiological effects of inappropriate delivery of such agents to other cells and tissues, such as uninfected cells. Intracellular targeting may be achieved by methods and compositions which allow accumulation or retention of biologically active agents inside cells.

Preparation of Compounds of the Invention

The compounds of the invention may be prepared by a variety of synthetic routes and methods known to those skilled in the art. The invention also relates to methods of making the compounds of the invention. The compounds may be prepared by any of the applicable techniques of organic synthesis. For example, known techniques are elaborated in: “Compendium of Organic Synthetic Methods”, John Wiley & Sons, New York, Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, jr., 1980; Vol. 5, Leroy G. Wade, Jr., 1984; and Vol. 6, Michael B. Smith; as well as March, J., “Advanced Organic Chemistry”, Third Edition, John Wiley & Sons, New York, 1985; “Comprehensive Organic Synthesis. Selectivity, Strategy & Efficiency in Modern Organic Chemistry” (9 Volume set) Barry M. Trost, Editor-in-Chief, Pergamon Press, New York, 1993.

Intermediates useful in preparing compounds of the invention are provided in Example 1-A (compound series 4000).

A number of exemplary methods for the preparation of the compounds of the invention are provided herein. These methods are intended to illustrate the nature of such preparations and are not intended to limit the scope of applicable methods. For instance, see Example 1 (compound series 5002, 5003, and 5004), Example 2 (compound series 5006), Example 3 (compound series 5008) and Example 4 (compound series 5010).

Deliberate use may be made of protecting groups to mask reactive functionality and direct reactions regioselectively (Greene, etal (1991) “Protective Groups in Organic Synthesis”, 2nd Ed., John Wiley & Sons). For example, useful protecting groups for the 8-hydroxyl group and other hydroxyl substituents include methyl, MOM (methoxymethyl), trialkylsilyl, benzyl, benzoyl, trityl, and tetrahydropyranyl. Certain aryl positions may be blocked from substitution, such as the 2-position as fluorine.

Protection of Reactive Substituents.

Depending on the reaction conditions employed, it may be necessary to protect certain reactive substituents from unwanted reactions by protection before the sequence described, and to deprotect the substituents afterwards, according to the knowledge of one skilled in the art. Protection and deprotection of functional groups are described, for example, in Protective Groups in Organic Synthesis, by T. W. Greene and P. G. M Wuts, Wiley, Second Edition 1990. Reactive substituents which may be protected are shown in the accompanying schemes as, for example, [OH], [SH], etc.

Preparation of Carboalkoxy-Substituted Phosphonate Bisamidates, Monoamidates, Diesters and Monoesters.

A number of methods are available for the conversion of phosphonic acids into amidates and esters. In one group of methods, the phosphonic acid is either converted into an isolated activated intermediate such as a phosphoryl chloride, or the phosphonic acid is activated in situ for reaction with an amine or a hydroxy compound.

The conversion of phosphonic acids into phosphoryl chlorides is accomplished by reaction with thionyl chloride, for example as described in J. Gen. Chem. USSR, 1983, 53, 480, Zh. Obschei Khim., 1958, 28, 1063, or J. Org. Chem., 1994, 59, 6144, or by reaction with oxalyl chloride, as described in J. Am. Chem. Soc., 1994, 116, 3251, or J. Org. Chem., 1994, 59, 6144, or by reaction with phosphorus pentachloride, as described in J. Org. Chem., 2001, 66, 329, or in J. Med. Chem., 1995, 38, 1372. The resultant phosphoryl chlorides are then reacted with amines or hydroxy compounds in the presence of a base to afford the amidate or ester products.

Phosphonic acids are converted into activated imidazolyl derivatives by reaction with carbonyl diimidazole, as described in J. Chem. Soc., Chem. Comm., 1991, 312, or Nucleosides Nucleotides 2000, 19, 1885. Activated sulfonyloxy derivatives are obtained by the reaction of phosphonic acids with trichloromethylsulfonyl chloride, as described in J. Med. Chem. 1995, 38, 4958, or with triisopropylbenzenesulfonyl chloride, as described in Tet. Lett., 1996, 7857, or Bioorg. Med. Chem. Lett., 1998, 8, 663. The activated sulfonyloxy derivatives are then reacted with amines or hydroxy compounds to afford amidates or esters.

Alternatively, the phosphonic acid and the amine or hydroxy reactant are combined in the presence of a diimide coupling agent. The preparation of phosphonic amidates and esters by means of coupling reactions in the presence of dicyclohexyl carbodiimide is described, for example, in J. Chem. Soc., Chem. Comm., 1991, 312, or J. Med. Chem., 1980, 23, 1299 or Coll. Czech. Chem. Comm., 1987, 52, 2792. The use of ethyl dimethylaminopropyl carbodiimide for activation and coupling of phosphonic acids is described in Tet. Lett., 2001, 42, 8841, or Nucleosides Nucleotides, 2000, 19, 1885.

A number of additional coupling reagents have been described for the preparation of amidates and esters from phosphonic acids. The agents include Aldrithiol-2, and PYBOP and BOP, as described in J. Org. Chem., 1995, 60, 5214, and J. Med. Chem., 1997, 40, 3842, mesitylene-2-sulfonyl-3-nitro-1,2,4-triazole (MSNT), as described in J. Med. Chem., 1996, 39, 4958, diphenylphosphoryl azide, as described in J. Org. Chem., 1984, 49, 1158, 1-(2,4,6-triisopropylbenzenesulfonyl-3-nitro- 1,2,4-triazole (TPSNT) as described in Bioorg. Med. Chem. Lett., 1998, 8, 1013, bromotris(dimethylamino)phosphonium hexafluorophosphate (BroP), as described in Tet. Lett., 1996, 37, 3997, 2-chloro-5,5-dimethyl-2-oxo-1,3,2-dioxaphosphinane, as described in Nucleosides Nucleotides 1995, 14, 871, and diphenyl chlorophosphate, as described in J. Med. Chem., 1988, 31, 1305.

Phosphonic acids are converted into amidates and esters by means of the Mitsonobu reaction, in which the phosphonic acid and the amine or hydroxy reactant are combined in the presence of a triaryl phosphine and a dialkyl azodicarboxylate. The procedure is described in Org. Lett., 2001, 3, 643, or J. Med. Chem., 1997, 40, 3842.

Phosphonic esters are also obtained by the reaction between phosphonic acids and halo compounds, in the presence of a suitable base. The method is described, for example, in Anal. Chem., 1987, 59, 1056, or J. Chem. Soc. Perkin Trans., I, 1993, 19, 2303, or J Med. Chem., 1995, 38, 1372, or Tet. Lett., 2002, 43, 1161.

Biological Activity of HIV-Integrase Inhibitor Compounds

Representative compounds of the invention were tested for biological activity by methods including anti-HIV assay, measuring inhibition of HIV-integrase strand transfer catalysis, and cytotoxicity. See: Wolfe, etal J. Virol. (1996) 70:1424-1432; Hazuda, etal Nucleic Acids Res. (1994) 22:1121-22; Hazuda, etal J. Virol. (1997) 71:7005-7011; Hazuda, etal Drug Design and Discovery (1997) 15:17-24; and Hazuda, etal Science (2000) 287:646-650. The antiviral activity of a compound of the invention can be determined using pharmacological models which are well known in the art. While many of the compounds of the present invention demonstrate inhibition of integration of HIV reverse-transcribed DNA, there may be other mechanisms of action whereby HIV replication or proliferation is affected. The compounds of the invention may be active via inhibition of HIV-integrase or other enzymes associated with HIV infection, AIDS, or ARC. Furthermore, the compounds of the invention may have significant activity against other viral diseases. Thus, the specific assays embodied herein are not intended to limit the present invention to a specific mechanism of action.

HIV Integrase Assay (IC₅₀ Determination)

The HIV Integrase assay is carried out in Reacti-Bind High Binding Capacity Streptavidin coated plates (Pierce # 15502) in 100 μl reactions. The wells of the plate are rinsed once with PBS. Each well is then coated at room temperature for 1 h with 100 μl of 0.14 μM Donor DNA with the following sequence:

5′Biotin-
ACC CTT TTA GTC AGT GTG GAA AAT CTC TAG CAG T-3′
3′-GAA AAT CAG TCA CAC CTT TTA GAG ATC GTC A-5′

After coating, the plate was washed twice with PBS. 3′processing of the Donor DNA is started by adding 80 μl of Integrase/buffer mixture (25 mM HEPES, pH 7.3, 12.5 mM DTT, 93.75 mM NaCl, 12.5 mM MgCl₂, 1.25% Glycerol, 0.3125 uM integrase) to each well. 3′processing is allowed to proceed for 30 min at 37° C., after which, 10 μl of test compound and 10 μl of 2.5 uM DIG-labeled Target DNA with the following sequence:

5′-TGA CCA AGG GCT AAT TCA CT-3′DIG
3′DIG-ACT GGT TCC CGA TTA AGT GA-5′

are added to each well to allow strand transfer to proceed for 30 min at 37° C. The plate is then washed three times with 2×SSC for 5 min and rinsed once with PBS. For detection of integrated product, 100 μl of a 1/2000 dilution of HRP-conjugated anti-DIG antibody (Pierce #31468) are added to each well and incubated for 1 hour. The plate was then washed three times for 5 min each, with 0.05% Tween-20 in PBS. For signal development and amplification, 100 μl of SuperSignal ELISA Femto Substrate (Pierce #37075) are added to each well. Chemiluminescence (in relative light units) is read immediately at 425 nm in the SPECTRAmax GEMINI Microplate Spectrophotometer using the end point mode at 5 sec per well.

For IC₅₀ determinations, eight concentrations of test compounds in a 1/2.2 dilution series are used.

Antiviral Assays in MT2 and MT4 Cells

For the antiviral assay utilizing MT-2 cells, 50 μl of 2× test concentration of 5-fold serially diluted compound in culture medium with 10% FBS was added to each well of a 96-well plate (9 concentrations) in triplicate. MT-2 cells were infected with HIV-IIIb at a multiplicity of infection (m.o.i) of 0.01 for 3 hours. Fifty microliters of infected cell suspension in culture medium with 10% FBS (˜1.5×10⁴ cells) was then added to each well containing 50 μl of diluted compound. The plates were then incubated at 37° C. for 5 days. For the antiviral assay utilizing MT-4 cells, 20 μl of 2× test concentration of 5-fold serially diluted compound in culture medium with 10% FBS was added to each well of a 384-well plate (7 concentrations) in triplicate. MT-4 cells were next mixed with HIV-IIIb at an m.o.i. of 0.1 and 20 μl of virus/cell mixture (˜2000 cells) was immediately added to each well containing 20 μl of diluted compound. The plates were then incubated at 37° C. for 5 days. After 5 days of incubation, 100 μl of CellTiter-Glo™ Reagent (catalog # G7571, Promega Biosciences, Inc., Madison, Wis.) was added to each well containing MT-2 cells and 40 μl to each well containing MT-4 cells. Cell lysis was carried out by incubating at room temperature for 10 min and then chemiluminescence was read.

Cytotoxicity Assays in MT-2 and MT-4 Cells

For compound cytotoxicity assessment in MT-2 cells, the protocol was identical to that of the antiviral assay in MT-2 cells, except that uninfected cells and a 3-fold serial dilution of compounds were used. For cytotoxicity assessment in MT-4 cells, the protocol is identical to that of the antiviral assay in MT-4 cells, except that no virus was added.

The compounds of the invention preferably have an IC₅₀ of less than or equal to about 1 μM. More preferably, the compounds of the invention have an IC₅₀ of less than or equal to about 60 nM. Even more preferably, the inventive compounds have an IC₅₀ of less than or equal to about 25 nM. The compounds of the invention preferably have an EC₅₀ of less than or equal to about 1 μM, and more preferably, an EC₅₀ of less than or equal to about 60 nM. Even more preferably, the inventive compounds have an IC₅₀ of less than or equal to about 25 nM. Certain compounds of the invention have an IC₅₀ of between >0 μM and about 1 μM, and an EC₅₀ of between >0 μM and about 1 μM. More preferably, certain compounds of the invention have an IC₅₀ of between >0 μM and about 60 nM and an EC₅₀ of between >0 μM and about 60 nM. Even more preferably, certain compounds of the invention have an IC₅₀ of between >0 μM and about 25 nM and an EC₅₀ of between >0 μM and about 25 nM.

Pharmaceutical Formulations and Routes of Administration

Examples of pharmaceutically acceptable carriers and methods of manufacture for various compositions may be found in Remington's Pharmaceutical Sciences, 18^{th} Ed., Mack Publishing Co. (1990), which is incorporated in its entirety by reference herein.

The compounds of the invention may be formulated with conventional carriers, diluents and excipients, which will be selected in accord with ordinary practice. Tablets will contain excipients, glidants, fillers, binders, diluents and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Compounds of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

While it is possible for the active ingredients to be administered alone it is preferably to present them as pharmaceutical formulations. The formulations, both for veterinary and for human use, of the present invention comprise at least one active ingredient, as above defined, together with one or more pharmaceutically acceptable carriers (excipients, diluents, etc.) thereof and optionally other therapeutic ingredients. The carrier(s) must be “acceptable” in the sense of being compatible with the other ingredients of the formulation and not deleterious to the recipient thereof.

The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

For infections of the eye or other external tissues e.g. mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the present invention include Tween™ 60, Span™ 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties, since the solubility of the active compound in most oils likely to be used in pharmaceutical emulsion formulations is very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents such as pentamidine for treatment of pneumocystis pneumonia.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

The present invention further provides veterinary compositions comprising at least one active ingredient as above defined together with a veterinary carrier □yclospor. Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods. Controlled release formulations may be employed for the treatment or prophylaxis of various microbial infections particularly human bacterial, human parasitic protozoan or human viral infections caused by microbial species including Plasmodium, Pneumocystis, herpes viruses (CMV, HSV 1, HSV 2, VZV, and the like), retroviruses, adenoviruses and the like. The controlled release formulations can be used to treat HIV infections and related conditions such as tuberculosis, malaria, pneumocystis pneumonia, CMV retinitis, AIDS, AIDS-related complex (ARC) and progressive generalized lymphadeopathy (PGL), and AIDS-related neurological conditions such as multiple sclerosis, and tropical spastic paraparesis. Other human retroviral infections that may be treated with the controlled release formulations according to the invention include Human T-cell Lymphotropic virus (HTLV)-I and IV and HIV-2 infections. The invention accordingly provides pharmaceutical formulations for use in the treatment or prophylaxis of the above-mentioned human or veterinary conditions and microbial infections.

Combination Therapy

The compounds of the invention may be employed in combination with other therapeutic agents for the treatment or prophylaxis of the infections or conditions indicated above. Examples of such further therapeutic agents include agents that are effective for the treatment or prophylaxis of viral, parasitic or bacterial infections or associated conditions or for treatment of tumors or related conditions include 3′-azido-3′-deoxythymidine (zidovudine, AZT), 2′-deoxy-3′-thiacytidine (3TC), 2′,3′-dideoxy-2′,3′-didehydroadenosine (D4A), 2′,3′-dideoxy-2′,3′-didehydrothymidine (D4T), carbovir (carbocyclic 2′,3′-dideoxy-2′,3′-didehydroguanosine), 3′-azido-2′,3′-dideoxyuridine, 5-fluorothymidine, (E)-5-(2-bromovinyl)-2′-deoxyuridine (BVDU), 2-chlorodeoxyadenosine, 2-deoxycoformycin, 5-fluorouracil, 5-fluorouridine, 5-fluoro-2′-deoxyuridine, 5-trifluoromethyl-2′-deoxyuridine, 6-azauridine, 5-fluoroorotic acid, methotrexate, triacetyluridine, 1-(2′-deoxy-2′-fluoro-1-β-arabinosyl)-5-iodocytidine (FIAC), tetrahydro-imidazo(4,5, 1 jk)-(1,4)-benzodiazepin-2(1H)-thione (TIBO), 2′-nor-cyclicGMP, 6-methoxypurine arabinoside (ara-M), 6-methoxypurine arabinoside 2′-O-valerate, cytosine arabinoside (ara-C), 2′,3′-dideoxynucleosides such as 2′,3′-dideoxycytidine (ddC), 2′,3′-dideoxyadenosine (ddA) and 2′,3′-dideoxyinosine (ddl), acyclic nucleosides such as acyclovir, penciclovir, famciclovir, ganciclovir, HPMPC, PMEA, PMEG, PMPA, PMPDAP, FPMPA, HPMPA, HPMPDAP, (2R, 5R)-9>tetrahydro-5-(phosphonomethoxy)-2-furanyladenine, (2 R, 5R)- 1 -)tetrahydro-5-(phosphonomethoxy)-2-furanylthymine, other antivirals including ribavirin (adenine arabinoside), 2-thio-6-azauridine, tubercidin, aurintricarboxylic acid, 3-deazaneoplanocin, neoplanocin, rimantidine, adamantine, and foscarnet (trisodium phosphonoformate), antibacterial agents including bactericidal fluoroquinolones (ciprofloxacin, pefloxacin and the like), aminoglycoside bactericidal antibiotics (streptomycin, gentamicin, amicacin and the like) β-lactamase inhibitors (cephalosporins, penicillins and the like), other antibacterials including tetracycline, isoniazid, rifampin, cefoperazone, claithromycin and azithromycin, antiparasite or antifungal agents including pentamidine (1,5-bis(4′-aminophenoxy)pentane), 9-deaza-inosine, sulfamethoxazole, sulfadiazine, quinapyramine, quinine, fluconazole, ketoconazole, itraconazole, Amphotericin B, 5-fluorocytosine, clotrimazole, hexadecylphosphocholine and nystatin, renal excretion inhibitors such as probenicid, nucleoside transport inhibitors such as dipyridamole, dilazep and nitrobenzylthioinosine, immunomodulators such as FK506, □yclosporine A, thymosin α-1, cytokines including TNF and TGF-β, interferons including IFN-α, IFN-β, and IFN-γ, interleukins including various interleukins, macrophage/granulocyte colony stimulating factors including GM-CSF, G-CSF, M-CSF, cytokine antagonists including anti-TNF antibodies, anti-interleukin antibodies, soluble interleukin receptors, protein kinase C inhibitors and the like.

The compounds of the invention may be employed in combination with booster agents. One aspect of the invention provides the use of an effective amount of a booster agent to boost the pharmacokinetics of a compound of the invention. An effective amount of a booster agent, for example, the amount required to boost an HIV integrase inhibitor of the invention, is the amount necessary to improve the pharmacokinetic profile of the inventive compound when compared to its profile when used alone. The inventive compound possesses a better efficacious pharmacokinetic profile than it would without the addition of the boosting agent. The amount of booster agent used to boost the integrase inhibitor potency of the inventive compound is, preferably, subtherapeutic (e.g., dosages below the amount of booster agent conventionally used for therapeutically treating HIV infection in a patient). A boosting dose for the compounds of the invention is subtherapeutic for treating HIV infection, yet high enough to effect modulation of the metabolism of the compounds of the invention, such that their exposure in a patient is boosted by increased bioavailability, increased blood levels, increased half life, increased time to peak plasma concentration, increased/faster inhibition of HIV integrase and/or reduced systematic clearance. An example of a boosting agent is Ritonavir® (ABBOTT Laboratories).

The compounds of the invention are preferably administered in an oral dosage form. The inventive compounds (or pharmaceutically acceptable salts thereof) are useful for the treatment of AIDS. The inventive compounds (or pharmaceutically acceptable salts thereof) are useful for therapy. They are useful as a medicament. The compounds or pharmaceutically acceptable salts of the invention are useful in the manufacture of a medicament for the treatment of HIV. The pharmaceutical compositions of the invention may be used in the treatment of AIDS.

Still another aspect of this invention is to provide a kit for the treatment of disorders, symptoms and diseases where integrase inhibition plays a role, comprising two or more separate containers in a single package, wherein an inventive compound, salt or composition thereof is placed in combination with one or more of the following: a pharmaceutically acceptable carrier (excipient, diluent, etc.), a booster agent, and a therapeutically effective amount of another inventive compound, salt or composition thereof, an AIDS treatment agent, such as an HIV inhibitor agent, an anti-infective agent or an immunomodulator agent.

The compounds can be made though a variety of synthetic routes. Generic procedures known in the art, such as those disclosed in WO/2004035577, which is hereby incorporated herein in its entirety, may be applied to synthesize a number of compounds of the invention. Specific procedures for representative compounds follow below.

### EXAMPLES

### Example 1

Intermediates useful in synthesizing compounds of the invention can be prepared by the following methodology. It should be noted that after every step, the product may be recovered and optionally purified by conventional methods such as precipitation, filtration, evaporation, crystallization, chromatography and the like. Alternatively, the products can be used directly in the next step without purification and/or isolation.

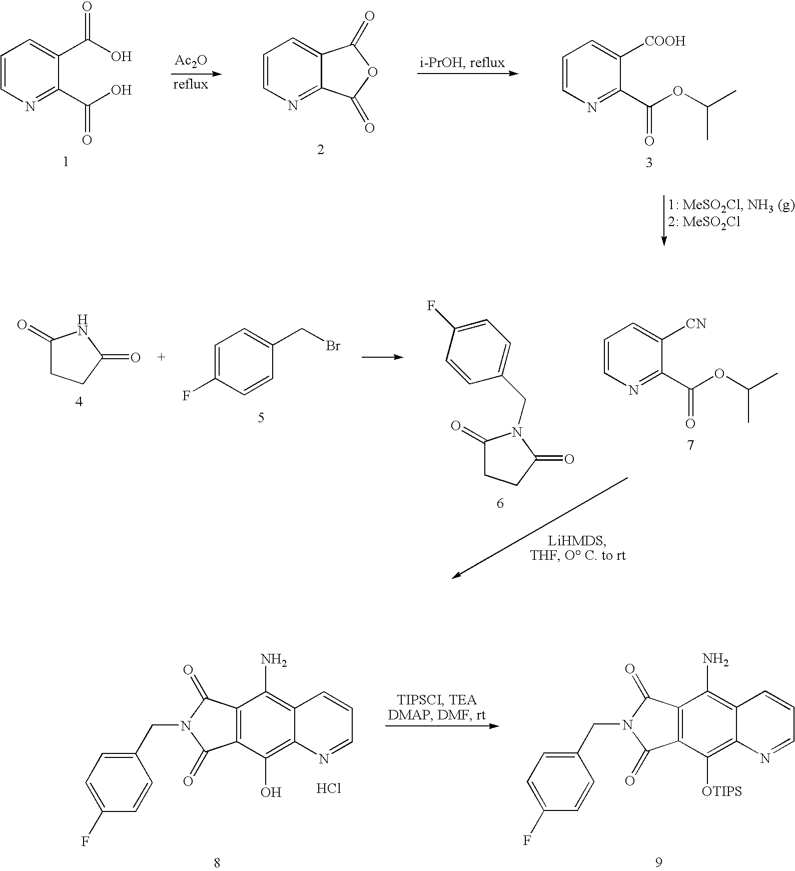

Compound 1 is converted under conventional conditions to the corresponding anhydride 2. Specifically, compound 1 is refluxed in a suitable solvent, such as acetone, methyl ethyl ketone in the presence of an excess of 15 acetic anhydride to provide the anhydride 2. Compound 2 is then refluxed in the presence of an approximately single equivalent of isopropanol for about 2 to about 20 hours to provide for the mono-carboxy, mono-isopropoxy derivative, compound 3. Compound 3 is then condensed under conventional conditions with methylsulfonyl chloride in a suitable base such as ammonia, to provide for the 3-cyanopyridine 7.

Separately succinimide 4 is condensed with a slight excess of 4-fluorobenzylbromide 5 to provide for N-(4-fluorobenzyl)succinimide, compound 6. In turn, approximately stoichiometric amount of compound 6 and 7 are condensed in the presence of LiHMDS to provide for HCI of compound 8. The reaction is conducted in a suitable inert solvent such as THF, dioxane and the like at a temperature from 0 to 30° C. The reaction is continued until substantial completion. The hydroxyl group of compound 8 is then protected under conventional conditions using an excess of triisopropylsilylchloride in the presence of a suitable base (e.g., triethylamine/DMAP) to scavenge the acid generated. The reaction is conducted in a suitable solvent DMF and maintained at room temperature until substantial completion to provide for compound 9.

### Example 2

### Preparation of N-(7-(4-fluorobenzyl)-9-hydroxy-8-oxo-7,8-di hydro-6H-pyrrolo[3,4-g]quinolin-5-yl)-N-methylmethanesulfonamide

Procedure:

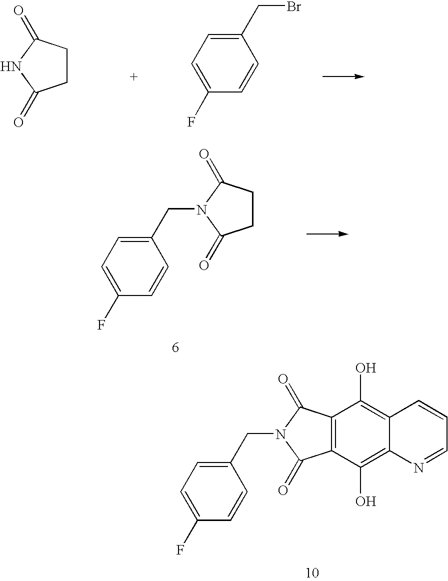

Freshly ground K₂CO₃ (31 g, 225 mmol) was added to dry acetone (200 mL) in a 3-necked flask equipped with drying tube, condenser, and mechanical stirrer. To this was added succinimide (7.43 g, 75 mmol) and 4-fluorobenzylbromide (11.21 mL, 90 mmol). Refluxed for 19 hours. Mixture filtered through Celite, then acetone removed under vacuum, diluted with EtOAc, washed with saturated aqueous sodium bicarbonate and also with brine, dried (MgSO₄), filtered and concentrated to give crude. Crude product chromatographed (EtOAc/hexane) on silica gel to give 6 as white solid (13.22 g, 85%). ¹H NMR (CDCI₃) δ 7.4 (dd, 2H), 7.0 (t, 2H), 4.6 (s, 1H), 2.7 (s, 4 H).

Compound 6 (8 g, 38.6 mmol) and 2,3-pyridine carboxylic acid dimethyl ester (7.9 g, 40.6 mmol) were dissolved in dry THF (78 mL) and dry MeOH (1.17 mL) in a 3-necked flask with mechanical stirrer and condenser. To this was added NaH (60% in mineral oil, 3.4 g, 85 mmol) slowly in four portions. Mixture stirred until bubbling ceased, then refluxed for 24 hours. 30 mL 6 M HCI was then added to the mixture while in an ice bath, stirring for 15 minutes. 100 mL diethyl ether was added, and the precipitate was filtered, and washed with diethyl ether and H₂O, and dried under vacuum at 100° C. Crude product was then recrystallized from 1 L refluxing dioxane and then dried under vacuum at 100° C. to give pure solid 10 (8.6 g, 66%). ¹H NMR (CD₃SOCD₃) δ 9.05 (d, 1H), 8.75 (d, 1H), 7.79 (dd, 1 H), 7.37 (dd, 2 H), 7.17 (t, 2H), 4.73 (s, 2 H). mp: 281.9-284.0.

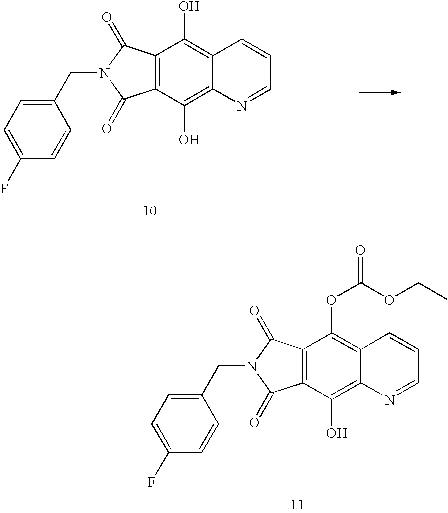

Bis-phenol 10 (3 g, 8.87 mmol) was suspended in 420 mL of dioxane and sonicated. To this was added 180 mL H₂O and again sonicated. After cooling in an ice-bath to 8° C., one equivalent of 0.675 M NaOH solution (13.14 mL) and solution turned red and clear. At this temperature was then added ethyl chloroformate (1.017 mL, 10.644 mmol) and then stirred at room temperature for one hour. Dioxane was concentrated off, mixture diluted with dichloromethane, aqueous layer acidified with 1 M HCI and NaCl added, organics dried (MgSO₄), concentrated to give crude as a 2:1 mixture of product and starting material. Triturated with dichloromethane (2×), collecting filtrate gives pure product 11 (2.27 g, 5.59 mmol, 63%). ¹H NMR (CDCI₃) δ 9.01 (dd, 1H), 8.55 (dd, 2H), 7.74 (dd, 1H), 7.49 (dd, 2H), 7.03 (dd, 2H), 4.84 (s, 2H), 4.49 (q, 2H), 1.50 (t, 3H.) MS: 411 (M+1), 409 (M−1).

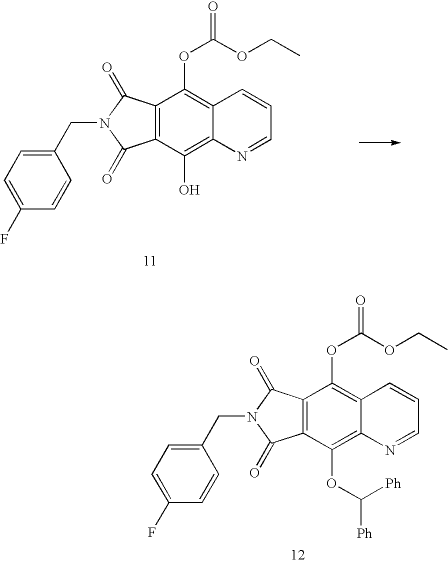

Mono-carbonate 11 (0.2 g, 0.4878mmol) was dissolved in 9 mL of dichloroethane. To this was added diphenyldiazomethane (0.189 g, 0.9756 mmol) and stirred at 70° C. for two hours. After starting material consumed, concentrated off some solvent, and chromatographed (25% ethyl acetate/hexanes) to give pure product 12 (0.2653 g, 0.4598 mmol, 94%). ¹H NMR (CDCI₃) δ 9.14 (d, 1H), 8.47 (d, 1H), 7.99 (s, 1H), 7.61 (m, 5H), 7.43 (dd, 2H), 7.27 (m, 6H), 7.02 (dd, 2H), 4.82 (s, 2H), 4.45 (q, 2H), 1.47 (t, 3H.) MS: 577 (M+1).

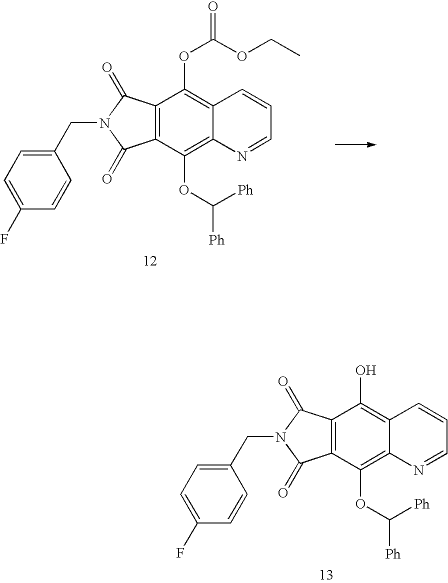

Ethyl Carbonate 12 (0.2653 g, 0.4598 mmol) was dissolved in 23 mL tetrahydrofuran and 15 mL H₂O. To this was added K₂CO₃ (0.633 g, 4.59 mmol) and dimethylaminopyridine (0.109 g, 0.9 mmol). Reaction stirred twelve hours at room temperature. Concentrated off solvent, dilute with dichloromethane, acidified aqueous layer with 1M HCl and added NaCl, concentrated organics to give crude. Triturate with 1:1 diethyl ether/hexanes to give pure product 13 (0.1807 g, 0.3586 mmol, 78%.) ¹H NMR (CDCl₃) δ 9.14 (d, 1H), 8.60.

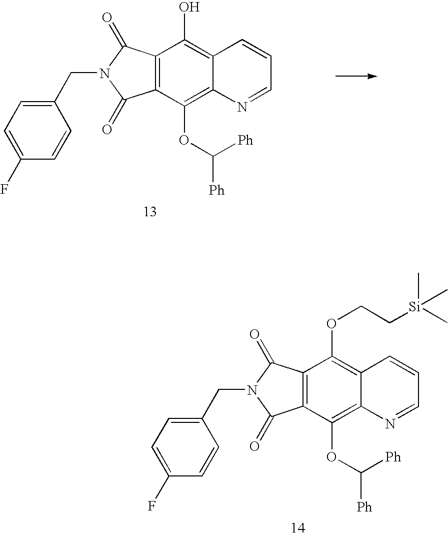

To the phenol 13 (3.37 g, 6.7 mmol) in anhydrous THF (70 mL) was added 2-(trimethylsilyl) ethanol (2.4 mL, 16.7 mmol), triphenylphosphine (3.5 g, 13.4 mmol) and diethyl azodicarboxylate (92.1 mL, 13.4 mmol). The solution was stirred at room temperature for 3 hours under nitrogen. TLC indicated the completion of the reaction. The solvent was evaporated off. The residue oil was purified by silica gel chromatography, eluting with EtOAc/hexane to afford the desired product 14 (3.3 g, 82%). ¹H NMR (CDCl₃): δ 9.1 (d, 1H, 8.6 (d, 1H), 7.9 (s, 1H), 7.6 (dd, 1H), 7.6 (d, 4H), 7.4 (d, 2H), 7.2-7.3 (m, 6H), 7.0 (t, 2H), 4.8 (s, 2H), 4.6 (t, 2H), 1.2 (t, 2H). MS: 605 (M+1), 627 (M+23).

An alternate route to 14:

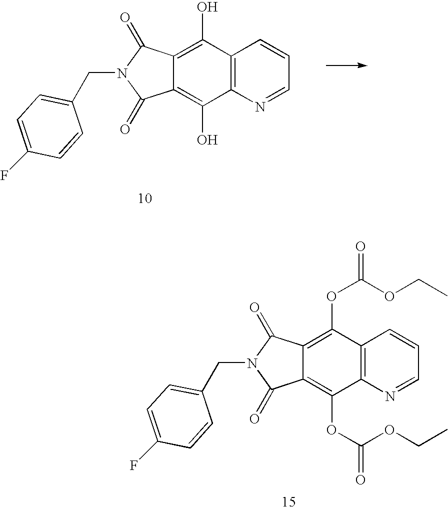

Into a flask containing bisphenol 10 (2.24 g, 6.63 mmol) was added DMF (10 mL). Under a nitrogen atmosphere, this was followed by addition of ethyl chloroformate (1.33 mL, 16.57 mmol, 2.5 eq). The addition of pyridine (1.61 mL, 19.88 mmol, 3.0 eq) made the reaction homogeneous. After several minutes precipitation occurred. The reaction was allowed to stir for 1 h before being quenched with 1 N HCl (20 mL). The solid was filtered and washed thoroughly with water and allowed to air dry to give 15 as an off white powder in 98% yield (3.12 g, 6.49 mmol).

¹H NMR (300 MHz) CDCl₃ δ: 9.15 (dd, J₁=1.5 Hz, J₂=4.2 Hz, 1H), 8.59 (dd, J₁=1.8 Hz, J₂=9.0 Hz, 1H), 7.71 (dd, J₁=4.2 Hz, J₂=8.7 Hz, 1H), 7.47-7.42 (m, 2H), 7.03-6.98 (m, 2H), 4.82 (s, 2H), 4.51-4.41 (m, 4H), 1.51-1.46 (m, 6H). MS: 482.1 (M+1), 505.0 (M+23). R_{f} (1/1 hexanes/EtAOc) 0.5.

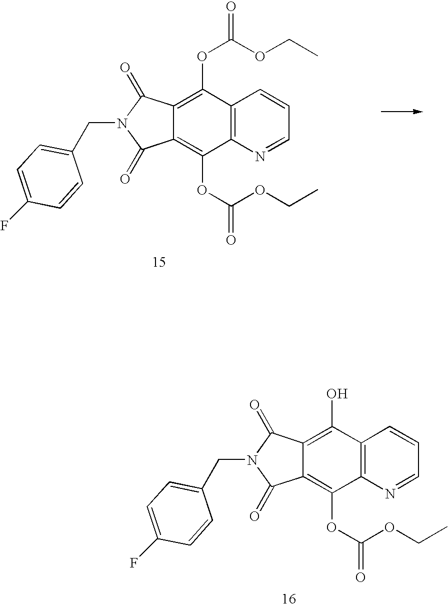

Into a flask containing the biscarbonate 15 (4.77 g, 9.89 mmol) was added THF (100 mL, 0.1 M). Under a nitrogen atmosphere was added DMAP (1.21 g, 9.89 mmol, 1 eq) and the reaction stirred for 26 h. The reaction was quenched with 1N HCl (50 mL) and extracted with EtOAC (2×50 mL). The organic extracts were combined and washed with water (2×45 mL) followed by brine solution (50 mL). The organic layer was then dried over Na₂SO₄, filtered and concentrated in vacuo to obtain the monocarbonate 16 (95%, 3.86 g, 9.40 mmol).

¹H NMR (300 MHz) CDCl₃ δ: 9.11 (dd, J₁=1.8 Hz, J₂=4.5 Hz, 1H), 8.59 (dd, J₁=1.8 Hz, J₂=8.7 Hz, 1H), 7.64 (dd, J₁=4.2 Hz, J₂=8.7 Hz, 1H), 7.61-7.42 (m, 2H), 7.05-6.98 (m, 2H), 4.82 (s, 2H), 4.44 (q, J=7.2 Hz 2H), 1.47 (t, J=7.2 Hz, 3H). MS: 411.0 (M+1), 433.0 (M+23).

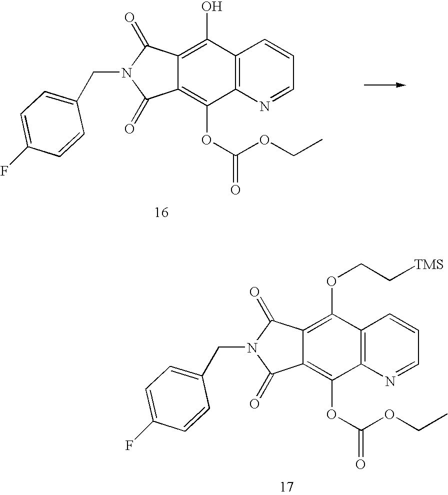

Into a flask containing the monocarbonate 16 (3.85 g, 9.4 mmol) was added THF (94 mL, 0.1 M) to form a suspension. Under a nitrogen atmosphere was sequentially added 2-(trimethylsilyl)ethanol (3.4 mL, 23.51 mmol, 2.5 eq), DEAD (7.41 mL, 18.81 mmol, 2.0 eq, 40% solution) and PPh₃ (4.94 g, 18.80 mmol, 2 eq). The mixture appears as a light brown homogeneous solution which was allowed to stir for 20 h. The mixture was concentrated in vacuo and loaded and purified by flash column chromatography with 85/15 (petroleum ether/ethyl acetate). A white solid 17 (93 %, 4.5 g, 8.82 mmol) was obtained.

¹H NMR (300 MHz) CDCl₃ δ: 9.08 (dd, J₁=1.5 Hz, J₂=4.2 Hz, 1H), 8.75 (dd, J₁=1.5 Hz, J₂=8.5 Hz, 1H), 7.64 (dd, J₁=4.2 Hz, J₂=8.5 Hz, 1H), 7.48-7.43 (m, 2H), 7.03-6.97 (m, 2H), 4.82 (m, 4H), 4.45 (q, J=7.5 Hz, 2H), 1.47 (t, J=7.5 Hz, 3H), 0.06 (s, 9H). MS: 510.1 (M+1), 533.0 (M+23). R_{f} (7/3 hexanes/EtAOc) 0.30.

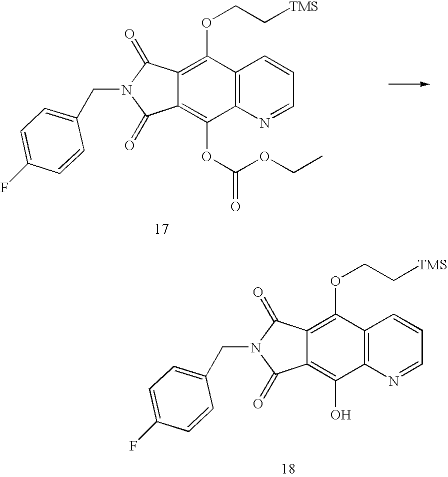

Into flask containing the carbonate 17 (4.5 g, 8.82 mmol) was dissolved in THF (50 mL) along with DMAP (2.15 g, 17.73 mmol, 2 eq). A solution of K₂CO₃ (6.09 g, 41.0 mmol, 5 eq) was dissolved separately in H₂O (40 mL) before transferring to the reaction mixture. The reaction was allowed to stir for 18 h and quenched with 1 N HCl (20 mL) and extracted with EtOAc (2×30 mL). The organic layer was washed with saturated NH₄Cl solution (25 mL), brine solution (25 mL) and dried over Na₂SO₄ and concentrated in vacuo to yield the phenol 18 (98%, 3.82 g, 9.78 mmol).

¹H NMR (300 MHz) CDCl₃ δ: 8.98 (d, J=4.5 Hz, 1H), 8.75 (d, J=8.2 Hz, 1H), 7.64 (dd, J¹=4.2 Hz, J₂=8.2 Hz, 1H), 7.51-7.46 (m, 2H) 7.03-6.97 (m, 2H), 4.85 (s, 2H), 4.71 (t, J=9.0 Hz, 2H), 1.28 (t, J=9.0 Hz, 3H), 0.08 (s, 9H). MS: 439.0 (M+1), 461.0 (M+23).

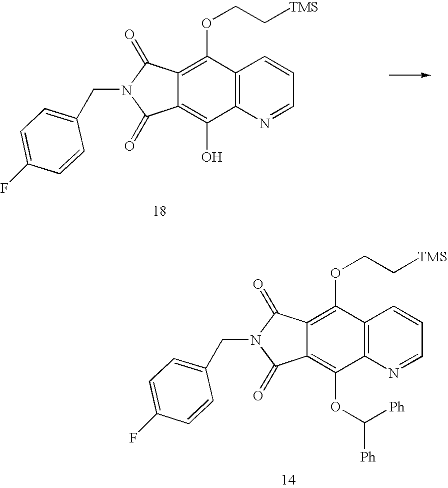

Into flask containing the phenol 18 (500 mg, 1.14 mmol) was dissolved in 1,2 dichloroethane (11 mL, 0.1 M). To this was added diphenyldiazomethane (114. mg, 0.59 mmol). The reaction was allowed to stir for 14 h during which time the reaction seems complete. It was concentrated in vacuo. The mixture was loaded purified by flash column chromatography and eluted by 9/1 PE/EtOAc. 14 was obtained as an off-white foam in 92% yield (639 mg, 1.06 mmol).

¹H NMR (300 MHz) CDCl₃ δ: 9.10 (dd, J₁=1.5 Hz, J₂=4.5 Hz, 1H), 8.64 (d, J₁=1.8 Hz, J₂=8.7 Hz, 1H), 7.90 (s, 1H), 7.84 (dd, J₁=1.8 Hz, J₂=8.7 Hz, 1H), 7.63-7.51 (m, 4H), 7.51-7.46 (m, 3H), 7.20-7.02 (m, 5H), 7.03-6.97 (m, 2H), 4.84 (s, 2H), 4.71 (t, J=8.4 Hz, 2H), 1.28 (t, J=8.4 Hz, 3H), 0.06 (s, 9H). MS: 439.0 (M+1), 461.0 (M+23). R_{f} (9/1 hexanes/EtOAc) 0.3.

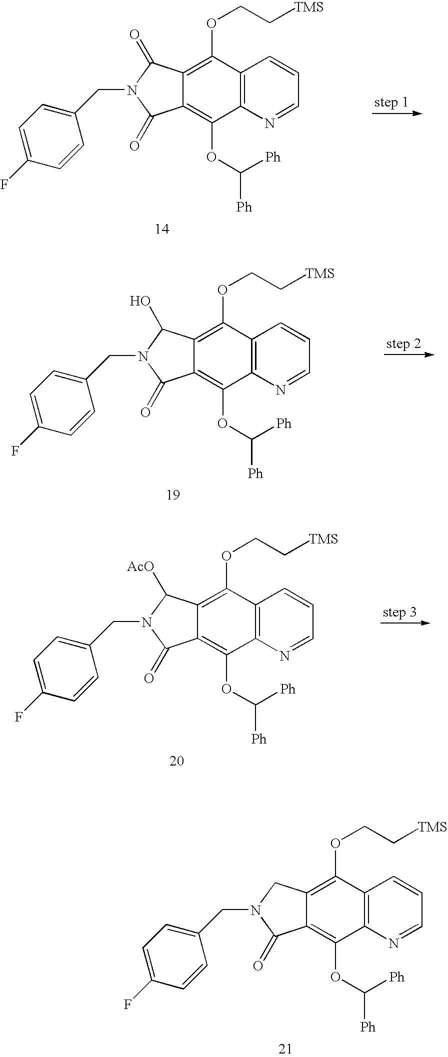

Step 1: The compound 14 (3.3 g, 5.46 mmol) was dissolved in the mixture of THF (40 mL), isopropanol (20 mL) and water (10 mL) and cold to 0° C. in an ice-bath. To this was added lithium borohydrate (373.0 mg, 16.4 mmol) slowly. The mixture was stirred at 0° C. for 1 h and at room temperature for 1 h under nitrogen. TLC indicated the completion of the reaction. It was added to 1N HCl (30 mL) and extracted with CH₂Cl₂ twice (2×50 mL). The organic layer was washed with sat'd NaHCO₃ and dried over Mg₂SO₄. It was then evaporated to dryness to give an oily crude product of 19 (3.3 g).

Step 2: The crude product 19 was dissolved in anhydrous dichloromethane (50 mL). To this solution was added N-dimethylaminopyridine (66.7 mg, 0.546 mmol), N, N-diisopropylethylamine (2.85 mL, 16.4 mmol) and acetic anhydride (1.03 mL, 109 mmol). The mixture was stirred at room temperature under nitrogen overnight. TLC indicated the completion of the reaction. It was quenched with 1N HCl (30 mL) and extracted with CH₂Cl₂ twice (2×50 mL). The organic layer was washed with sat'd NaHCO₃, dried (Mg₂SO₄) and concentrated to give a crude product of 20 (3.5 g).

Step 3: The crude product 20 was dissolved in anhydrous dichloromethane (60 mL) under nitrogen. To this solution was added 2,6-lutidine (3.2 mL, 23.7 mmol), triethylsiliane (10 mL), then trimethylsilyl trifrate (1.5 mL, 8.2 mmol) slowly. The mixture was stirred at room temperature for 3 h. TLC indicated the completion of the reaction. It was quenched with 1N HCl (30 mL) and extracted with CH₂Cl₂ twice (2×50 mL). The organic layer was washed with sat'd NaHCO₃, dried (Mg₂SO₄) and concentrated. The residue was chromatographied on a silica gel column, eluting with EtOAc/hexane to afford the clean desired 21 (1.4 g, 43.4% in 3 steps.).

¹H NMR (CDCl₃): δ 9.0 (d, 1H), 8.4 (d, 1H), 8.0 (s, 1H), 7.7 (d, 4H), 7.4 (dd, 1H), 7.1-7.3 (m, 8H), 7.0 (t, 2H), 4.8 (s, 2H), 4.2 (s, 2H), 4.1 (t, 2H), 1.1 (t, 2H), 0.1 (s, 9H). MS: 591 (M+1).

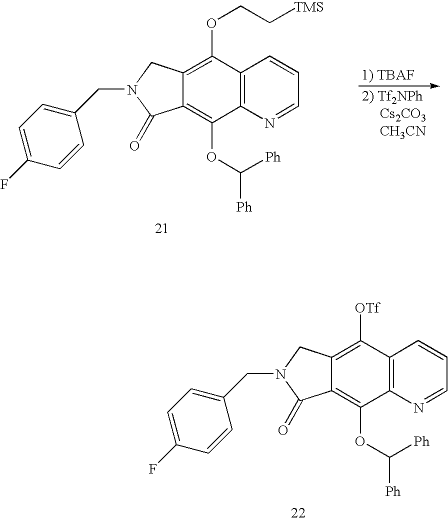

To a solution of TMS ethyl ether lactam 21 (18.9 g, 32.0 mmol) dissolved in THF (350 mL) was added TBAF hydrate (16.7 g, 46.4 mmol) over 5 min at room temperature. The reaction mixture was stirred at room temperature for 1 h under an inert atmosphere. TLC showed no starting materials. It was diluted with 500 mL of dichloromethane, and quenched with ice cold HCl solution (100 mL of 1N HCl plus 700 mL of ice water). The layers were separated. The aqueous layer was extracted with another 300 mL of dichloromethane. The organic layers were combined and dried over MgSO₄, then concentrated in vacuo. (It may result as C5-phenol precipitate out from solution during work up).

To the residue obtained from above, was dissolved in acetonitrile (300 mL) at room temperature. It was added cesium carbonate (20.8 g, 64 mmol) and N-phenyltrifluoromethanesulfonimide (13.7 g, 38.3 mmol). The reaction mixture was stirred at room temperature overnight (16 h). Filtered off the solid, the filtrate was diluted with EtOAc (500 mL), washed with 0.1 N of HCl, brine, and dried (MgSO₄). The concentrated crude mixture was purified by flash chromatography on silicon gel with EtOAc/hexane from 1/9 to 2/8). It yielded 17.6 g of triflite 22 (88%).

¹H NMR (CDCl₃) δ 9.1 (d, 1H), 8.3 (d, 1H), 8.2 (s, 1H), 7.7 (d, 2H), 7.6 (dd, 1H), 7.4-7.0 (m, 12H), 4.8 (s, 2H), 4.4 (s, 2H); MS: 623 (M+1).

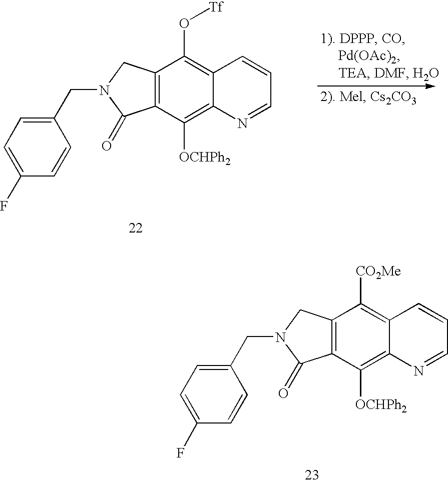

To trifluoro-methanesulfonic acid 9-benzhydryloxy-7-(4-fluoro-benzyl)-8-oxo-7,8-dihydro-6H-pyrrolo[3,4-g]quinolin-5-yl ester 22 (1.48 g, 2.39 mmol) and 1,3-bis(diphenylphosphino)propane (DPPP) (295 mg, 0.7 mmol) in DMF (20 mL) and water (1 mL) in a two-necked round bottom flask were added Pd(OAc)₂ (107 mg, 0.48 mmol). The solution was degassed under high vacuum and flushed with carbon monoxide from a balloon. The flushing was repeated five times. TEA (0.733 mL, 3.26 mmol) was introduced. The mixture was heated under CO atmosphere for 2.5 hours and cooled down to the room temperature. Mel (0.74 mL, 12 mmol) and Cs₂CO₃ were added and stirring was continued under a nitrogen atmosphere for 45 minutes. The mixture was diluted with EtOAc (300 mL), washed with water, 1N aqueous HCl and brine, dried over MgSO₄ and concentrated. The crude product was purified by chromatography on a silica gel column eluting with 15% to 35% of EtOAc in hexane to afford 9-benzhydryloxy-7-(4-fluoro-benzyl)-8-oxo-7,8-dihydro-6H-pyrrolo[3,4-g]quinoline-5-carboxylic acid methyl ester 23, (0.9 g, 1.69 mmol, 70%) as a yellow solid. ¹H NMR (CDCl₃): δ 9.25 (d, 1H), 9.05 (m, 1H), 7.80 (d, 4H), 7.56 (dd, 1H), 7.0-7.4 (m, 11H), 4.85 (s, 2H), 4.55 (s, 2H), 3.95 (s, 3H); MS: 555 (M+Na).

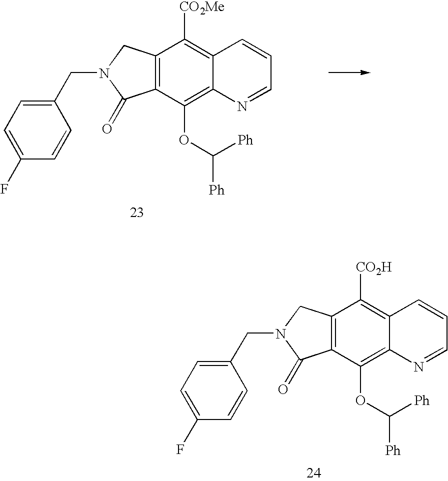

Methyl ester 23 (0.071 g, 0.1334 mmol) was dissolved in 2.4 mL of tetrahydrofuran and 0.6 mL of Dl H₂O. To this was added LiOH (0.013 g, 0.5338 mmol) and mixture stirred at room temperature. After 15 hours, starting material consumed. Diluted with dichloromethane, washed with 1M HCl solution, dried (Na₂SO₄), concentrated to give clean product 24 (0.068 g, 0.1313 mmol, 98%.)

¹H NMR (CD₃SOCD₃) δ 9.25 (d, 1H), 9.12 (dd, 1H), 8.17 (s, 1H), 7.75 (d, 5H), 7.37 (dd, 2H), 7.24 (m, 6H), 4.82 (s, 2H), 4.59 (s, 2H.) MS: 517 (M−1.)

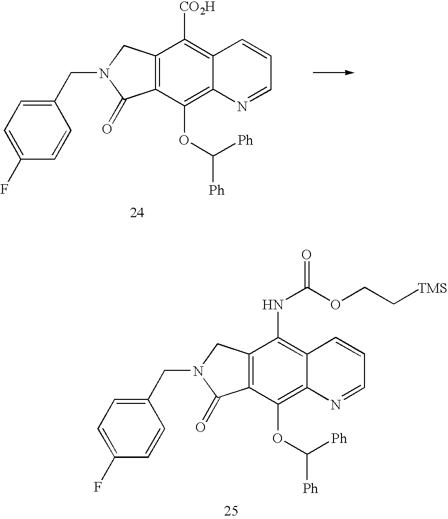

Into a flask containing toluene (15 mL, 0.2 M) was added carboxylic acid 24 (2.50 g, 4.83 mmol) followed by triethylamine (1.35 mL, 9.65 mmol, 2 eq) and phosphorazidic acid diphenyl ester (1.15 mL, 4.83 mmol, 1 eq) under inert atmosphere. The reaction was stirred at room temperature for 5 h before 2-trimethylsilyl ethanol (10 mL) was added and the reaction warmed to 60° C. for 26 h. The reaction was then concentrated in vacuo to a brown oil and re-dissolved in EtOAc (100 mL) and washed with saturated NH₄Cl, water (2×40 mL) and brine (40 mL). The organic layer was dried over Na₂SO₄, filtered and concentrated in vacuo. Flash column chromatography was then used to purify the product using hexanes/ethyl acetate (3/2) as eluent. Carbamate 25 was obtained as a white solid (2.14 g, 70% yield).

¹H NMR (300 MHz) CDCl₃ δ: 9.00 (d, J=2.7 Hz, 1H), 8.17 (d, J=8.4 Hz, 1H), 8.07 (s, 1H), 7.74-7.65 (m, 4H), 7.45 (dd, J₁=8.1 Hz, J₂=4.2 Hz, 1H), 7.30-7.12 (m, 8H), 7.12-7.00 (m, 2H), 6.40 (bs, 1H), 4.76 (s, 2H), 4.19 (s, 2H), 4.18 (s, 2H), 4.85 (s, 2H), 0.10 (s, 9H). ¹⁹F NMR (300 MHz) CDCl₃ δ: 115.24. MS: 634.1 (M+1).

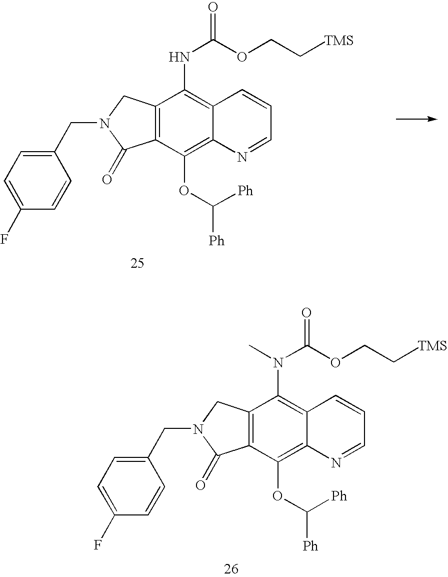

Into a flask containing carbamate 25 (100 mg, 0.158 mmol, 1 eq) was added DMF (1 mL) and cooled in an ice bath to 0° C. before sodium hydride (2.5 mg, 0.056 mmol, 60% mineral oil, 1.3 eq) was added and stirred for 10 minutes under inert atmosphere. Iodomethane (10 μl, 0.14 mmol, 3 eq) was added and the reaction allowed to stir for 45 minutes. The reaction was quenched with saturated NH₄Cl and dissolved in EtOAc (20 mL). It was washed with water (2×40 mL) and brine (40 mL). The organic layer was dried over Na₂SO₄, filtered and concentrated in vacuo. No further purification was carried out. Carbamate 26 was obtained (25 mg, 80% yield).

¹H NMR (300 MHz) CDCl₃ δ: 9.06 (d, J=2.4 Hz, 1H), 8.16 (s, 1H), 8.03 (d, J=9 Hz, 1H), 7.77 (d, J=7.5 Hz, 4H), 7.52 (dd, J₁=8.7 Hz, J₂=3.9 Hz, 1H), 7.37-7.23 (m, 6H), 7.20-7.13 (m, 2H), 7.09-7.02 (m, 2H) 4.94 (d, J=14.4 Hz, 1H), 4.73 (d, J=14.4 Hz), 4.13 (s, 2H), 4.05-3.93 (m, 2H), 3.19 (s, 3H), 0.49 (t, J=8.4 Hz, 2H), −0.26 (s, 9H). ¹⁹F NMR (300 MHz) CDCl₃ δ: −115.06. MS: 647.8 (M+1).

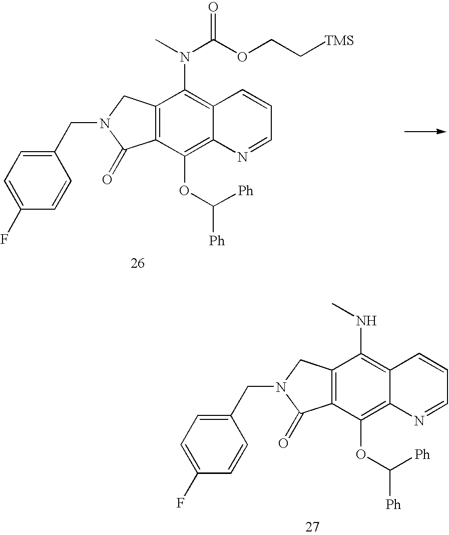

Into a flask containing carbamate 26 (25 mg, 0.038 mmol, 1 eq) was added THF (4 mL,) and cooled in an ice bath to 0° C. before tetra-butyl ammonium fluoride (100 μL, 0.096 mmol, 2.5 eq). The reaction was allowed to warm up to ambient temperature and stirred overnight. The reaction was quenched with saturated NH₄Cl and dissolved in EtOAc (20 mL). It was washed with water (2×40 mL) and brine (40 mL). The organic layer was dried over Na₂SO₄, filtered and concentrated in vacuo. No further purification was carried out and the material was used as is. 27 was obtained as a deep yellow solid (16 mg, 84 % yield).

¹H NMR (300 MHz) CDCl₃ δ: 9.00 (d, J=2.3 Hz, 1H), 8.23 (d, J=8.2 Hz, 1H), 7.85 (s, 1H), 7.77 (d, J=7.5 Hz, 4H), 7.42 (dd, J₁₌8.7 Hz, J₂=3.9 Hz, 1H), 7.37-7.23 (m, 6H), 7.20-7.13 (m, 2H), 7.09-7.02 (m, 2H), 4.81 (s, 2H), 4.27 (s, 2H), 2.95 (s, 3H), 2.73 (bs, 1H). MS: 504.0 (M+1).

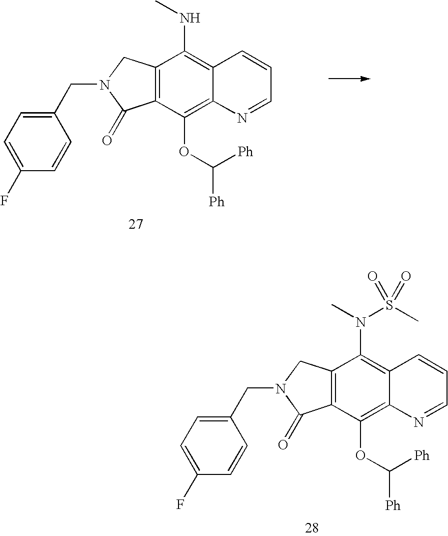

Into a flask containing aniline 27 (400 mg, 0.79 mmol, 1 eq) was added pyridine (3 mL, 0.2 M) and cooled in an ice bath to 0° C. before methanesulfonyl chloride (185 μl, 2.38 mmol, 3 eq) was added and the reaction allowed to warm up to room temperature overnight. The reaction was quenched with saturated NH₄Cl and dissolved in EtOAc (20 mL). It was washed with water (2×40 mL) and brine (40 mL). The organic layer was dried over Na₂SO₄, filtered and concentrated in vacuo. Flash column chromatography was then used purify the product using hexanes/rthyl acetate (3/2) as eluent. Sulfonylamide 28 was obtained as a white solid (398 mg, 86% yield).

¹H NMR (300 MHz) CDCl₃ δ: 9.05 (dd, J₁=3.9 Hz, J₂=1.5 Hz, 1H), 8.19 (s, 1H), 8.16 (d, J=1.5 Hz, 1H), 7.78-7.70 (m, 4H), 7.60 (dd, J₁=8.7 Hz, J₂=4.2 Hz, 1H), 7.37-7.15 (m, 10H), 7.07-7.04 (m, 2H), 5.05 (d, J=15.9 Hz, 1H), 4.62 (d, J=2.8 Hz), 4.57 (d, J=2.8 Hz, 1H), 4.27 (d, J=15.9 Hz, 1H), 3.25 (s, 3H), 3.02 (s, 3H). ¹⁹F NMR (300 MHz) CDCl₃ δ: −115.05. MS: 581.9 (M+1).

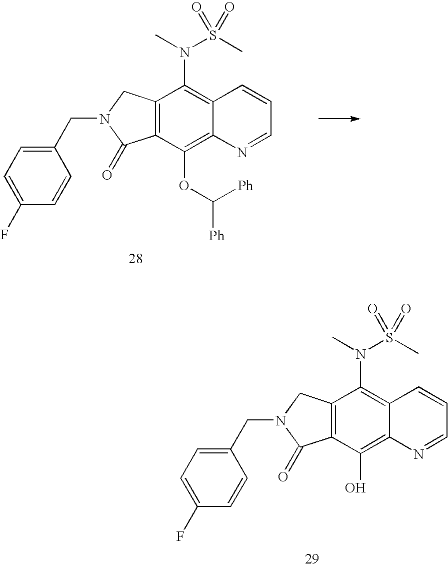

Into a flask containing sulfonamide 28 (395 mg, 0.68 mmol, 1 eq) was added dichloromethane (7 mL, 0.1 M) and cooled in an ice bath to 0° C. before triethylsilane (1.5 mL, 10.2 mmol, 15 eq) and trifluoroacetic acid (525 μl, 6.8 mmol, 10 eq) and the reaction carried out until the starting material was consumed. It was then concentrated in vacuo and dried thoroughly. A solution of hexanes/ethyl ether (30 mL, 1/1) was added to it and washed thoroughly via triturating (3×15 mL). Sonication was used to aid this washing. The residue was filtered on a sintered funnel and air dried thoroughly. An off white solid 29 (261 mg, 0.62 mmol, 93%) was obtained.

¹H NMR (300 MHz) DMSO-d₆ δ: 10.91 (bs, 1H), 8.95 (s, 1H), 8.43 (s, 1H), 7.78 (s, 1H), 7.39-7.33 (m, 2H), 7.21-7.13 (m, 2H), 4.71 (s, 2H), 4.53 (s, 2H), 3.25 (s, 3H), 3.18 (s, 3H). ¹⁹F NMR (300 MHz) DMSO-d₆ ∂: −115.87. MS: 416.1 (M+H).

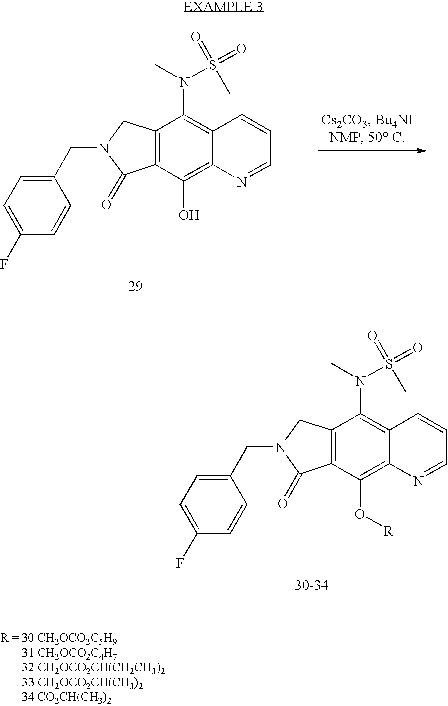

The following is a representative procedure for generating species 30-34. The free 8-phenol scaffold 29 (15 mg, 0.04 mmol) was dissolved in N-Methyl Pyrrolidinone (1 mL, 0.04M), and cesium carbonate (5 eq, 65 mg) and catalytic tetrabutylammonium iodide were added. The suspension stirred for 5 minutes and carbonic acid chloromethyl ester cyclopentyl ester (3 eq, 22 mg) was added. The reaction mixture-was placed under nitrogen and heated to 50° C. in an oil bath with condenser for three hours. The reaction mixture was cooled to room temperature, diluted with isopropyl acetate, and washed with water (3×). The aqueous layer was back extracted with isopropyl acetate (1×). The combined organic layers were washed with brine, dried over sodium sulfate, and concentrated to an oil. The residue was re-dissolved in minimal dichloromethane and purified by flash chromatography. Elution of the product with 3:1 ethyl acetate:hexanes afforded pure 30 (5.2 mg, 36% yield). 300 MHz ¹H NMR (CDCl₃) δ (ppm): 9.0 (d, 1H); 8.16 (d, 1H); 7.43 (m, 1H); 7.12 (t, 2H); 7.03 (t, 2H); 6.12 (m, 1H); 5.05 (m, 1H); 4.58-4.92 (dd, 2H); 4.05-4.45 (dd, 2H); 3.98 (m, 1H); 3.25 (s, 3H); 3.05 (s, 3H); 1.75 (m, 4H); 1.15 (m, 4H). MS=558.6 (M+H);

Using carbonic acid chloromethyl ester cyclobutyl ester, 31 was obtained (23% yield) Purified using reverse phase HPLC. MS=544.6 (M+H); 300 MHz ¹H NMR (CDCl₃) δ (ppm): 6.12 (m, 1H), 4.82 (m, 1H), 3.25 (s, 3H), 3.05 (s, 3H), 2.20 (m, 4H), 1.96 (m, 2H);

Using carbonic acid chloromethyl ester 2-pentyl ester, 32 was obtained (40% yield) Purified using reverse phase HPLC. MS=560.6 (M+H); 300 MHz ¹H NMR (CDCl₃) δ (ppm): 6.12 (m, 1H), 4.32 (m, 1H), 3.25 (s, 3H), 3.05 (s, 3H), 1.57 (m, 4H), 0.96 (t, 6H);

Using carbonic acid chloromethyl ester 2-propyl ester 33, was obtained (55% yield) FCC eluting with 2:1 ethyl acetate:hexanes resulted in pure material. MS=532.6 (M+H); 300 MHz ¹H NMR (CDCl₃) δ (ppm): 6.12 (m, 1H), 4.82 (m, 1H), 3.25 (s, 3H), 3.05 (s, 3H), 1.35 (d, 6H);

Using isopropylchlorocarbonate, 34 was obtained (98% yield) FCC eluting with 2:1 ethyl acetate:hexanes resulted in pure material. MS=532.6 (M+H); 300 MHz ¹H NMR (CDCl₃) δ (ppm): 5.12 (m, 1H), 3.25 (s, 3H), 3.05 (s, 3H), 1.35 (d, 6H).

An alternate route to 28:

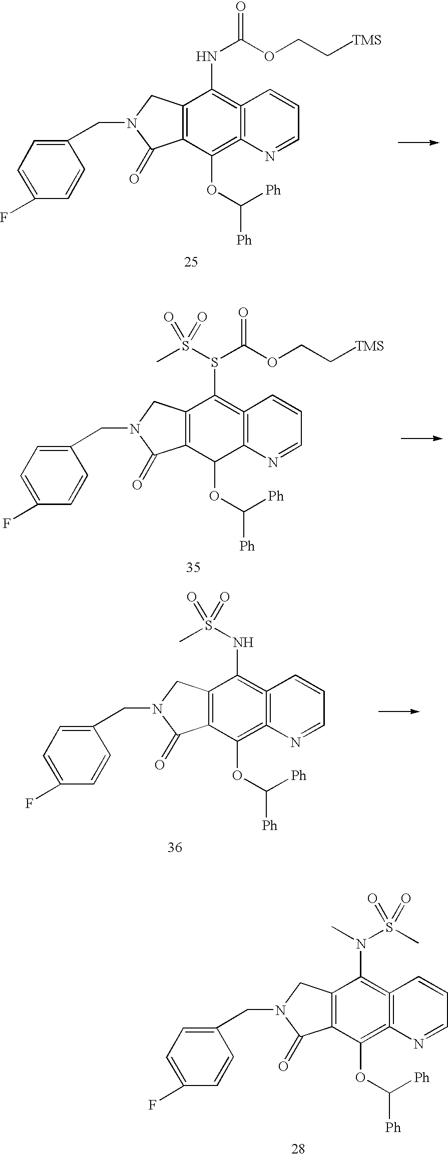

25 (80 mg, 130 μmol) was dissolved in 300 μL of DMF and cooled to 0° C. Sodium Hydride (15 mg, 390 μmol) was then added and the reaction was allowed to stir at 0° C. for 2 minutes. Methane Sulfonyl Chloride (45 mg, 390 μmol ) was then added and the reaction was allowed to warm to room temperature. After stirring at room temperature for 15 minutes, acetic acid (60 mg, 1 mmol) was added and the reaction was diluted with ethyl acetate. The organic phase was then washed once with 0.25 M citric acid, once with 5% LiCl, twice with water, and once with brine. The organic was then dried over MgSO₄ and concentrated in vacuo. The crude residue was then purified by silca gel chromatography (35% ethyl acetate in hexane) to afford intermediate 35 (62 mg, 66%).

Intermediate 35 (20 mg, 27 mol) was dissolved in 100 μL of THF and treated with 81 μL of 1.0M TBAF in THF. After stirring at room temperature for 15 minutes, 30 μL of acetic acid was added and the reaction was diluted with ethyl acetate. The organic was then washed once with 0.25 M citric acid, twice with water, and once with brine. The organic was dried over MgSO₄ and concentrated in vacuo to afford a crude compound 36 (27 mg, 48 μmol) which was dissolved in 150 μL of DMF and cooled to 0° C. Sodium hydride (4.6 mg, 120 μmol) as a 60% dispersion in mineral oil was added and the reaction was stirred at 0° C. for 2 minutes. Iodomethane (17 mg, 120 μmol) was then added and the reaction was allowed to warm to room temperature. After stirring at room temperature for 30 minutes, 30 μL of acetic acid was added and the reaction was then diluted with ethyl acetate. The organic phase was washed once with 0.25 M citric acid, twice with water, and once with brine. The organic was then dried over MgSO₄ and concentrated in vacuo. The residue was then purified by silica gel chromatography (1:1—hexane:ethyl acetate) to afford compound 28 (28 mg, 95%).

### Example 5

### Preparation of N-(7-(4-fluorobenzyl)-9-hydroxy-8-oxo-7,8-dihydro-6H-pyrrolo[3,4-g]quinolin-5-yl)-N,N′,N′-trimethylsulfamide

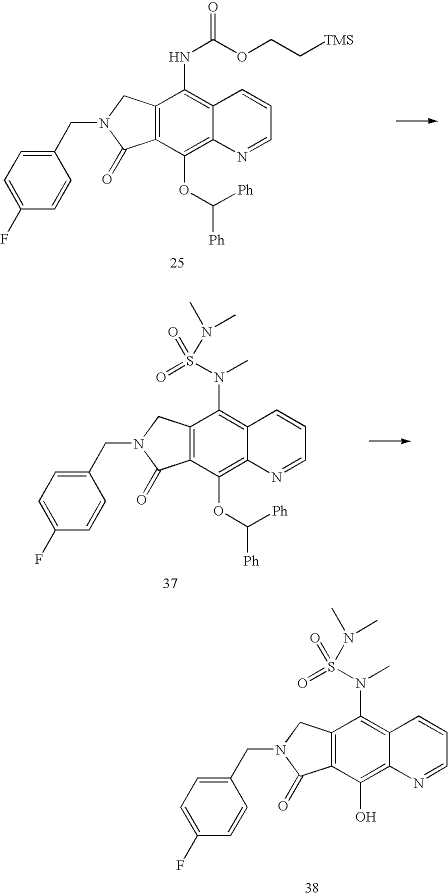

Intermediate 37 was synthesized from 25 in a manner similar to intermediate 28. Intermediate 37 ( 22 mg, 38 μmol) was dissolved in 200 μL of DCM and treated with TFA (22 mg, 190 μmol) and triethylsilane (9.0 mg, 76 μmol ). After stirring for 15 minutes at room temperature, the reaction mixture was azeotroped with toluene three times. The residue was then triturated with 3:1 hexane:ether to provide 38. 300 MHz 1H NMR (CDCl₃) δ (ppm): 9.11 (d, 1H); 8.65 (d, 1H); 7.71 (m, 1H); 7.30 (t, 2H); 7.02 (t, 2H); 4.90 (d, 2H); 4.72 (d, 1H); 4.58 (d, 1H); 4.35 (d, 1H); 3.1 (s, 3H); 2.9 (s, 3H). MS=445.5 ( M +1). Example 6

### Alternate Preparation of N-(7-(4-fluorobenzyl)-9-hydroxy-8-oxo-7,8-dihydro-6H-pyrrolo[3,4-g]quinolin-5-yl)-N,N′,N′-trimethylsulfamide

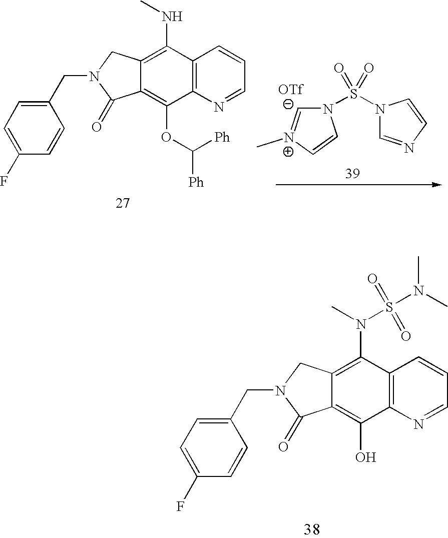

To the amine 27 (50 g, 0.099 mmol, 1 eq) contained in a microwave vial was added acetonitrile (2 mL, 0.05 M) and to it added the triflate 39 (J. Org Chem., 2003, 68, 115-119, 100 mg, 0.29 mmol, 3 eq). The reaction was microwaved at 120° C. for 90 min. HPLC purification then furnished phenol 38.

### Example 7

### Preparation of N-(7-(4-fluorobenzyl)-9-hydroxy-8-oxo-7,8-dihydro-6H-pyrrolo[3,4-g]quinolin-5-yl)-N′,N′-dimethylsulfamide

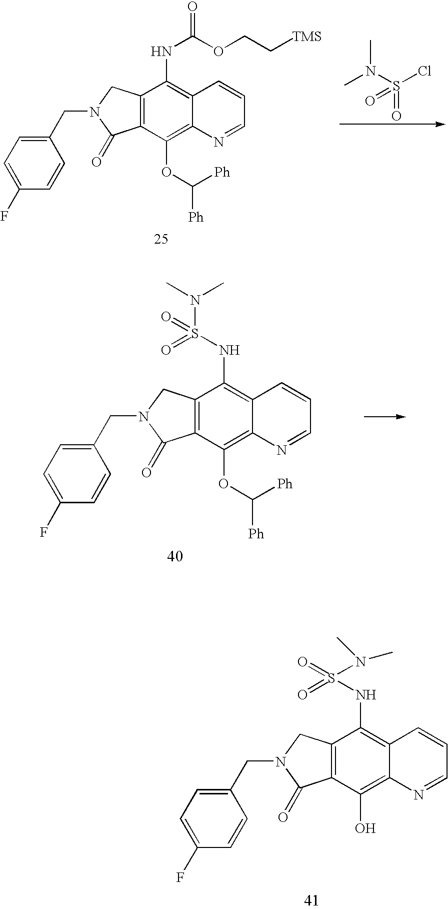

Intermediate 40 ( 6.5 mg, 11 μmol, prepared similarly to 36 from 25) was dissolved in 100 μL of DCM and treated with TFA (6.3 mg , 55 μmol) and triethylsilane (2.5 mg, 22 μmol ). After stirring for 15 minutes at room temperature, the reaction mixture was azeotroped with toluene three times. The residue was then triturated with 3:1—hexane:ether. The resulting residue was then purified by reverse-phase prep HPLC to provide 41 as the TFA salt. 300 MHz ¹H NMR (CDCl₃) δ (ppm): 8.9 (d, 1H); 8.65 (d, 1H); 7.71 (m, 1H); 7.36 (t, 2H); 7.07 (t, 2H); 4.76 (s, 2H); 4.58 (s, 2H); 2.78 (s, 6H). ¹⁹F NMR (CDCl₃) δ (ppm): −77.5; −117.3. MS=431.5 (M+1)

### Example 8

### Preparation of N-(7-(4-fluorobenzyl)-9-hydroxy-8-oxo-7,8-dihydro-6H-pyrrolo[3,4-g]quinolin-5-yl)-N-methyl-3-(dimethylamino)-propane-1-sulfonamide

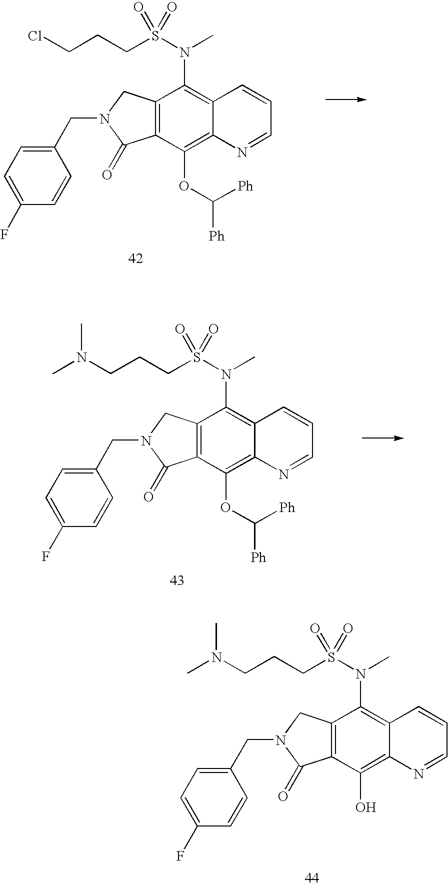

Intermediate 42 (52 mg, 81 μmol, synthesized in a manner similar to intermediate 28 from 25) was dissolved in 100 μl of THF and the reaction mixture was cooled to 0C. To this mixture, 1.0 mL of dimethylamine was added by condensation addition. The reaction was then placed under a reflux condenser and was allowed to warm to room temperature. The reaction was then stirred at room temperature for 2 days. The reaction was then concentrated in vacuo and purified by silica-gel chromatography (9:1—ethyl acetate:methanol) to afford 43 (17 mg, 32%).

43 (24 mg, 38 μmol) was dissolved in 200 μL of DCM and treated with TFA (22 mg, 190 μmol) and triethylsilane (9.0 mg, 76 μmol). After stirring for 15 minutes at room temperature, the reaction mixture was azeotroped with toluene three times. The residue was then triturated with 3:1—hexane:ether to provide 44. 300 MHz ¹H NMR (CDCl₃) δ ppm): 8.88 (d, 1H); 8.47 (d, 1H); 7.71 (m, 1H); 7.36 (t, 2H); 7.03 (t, 2H); 4.65 (m, 4H); 3.48 (m, 1H); 3.37 (b, 1H); 3.30 (s, 3H); 3.2 (m, 2H); 2.83 (s, 6H); 2.20 (b, 2H). MS=487.5 (M+1).

### Example 9

### Preparation of N-(7-(4-fluorobenzyl)-9-hydroxy-8-oxo-7,8-dihydro-6H-pyrrolo[3,4-g]quinolin-5-yl)-N-methyl-3-morpholinopropane-1-sulfonamide

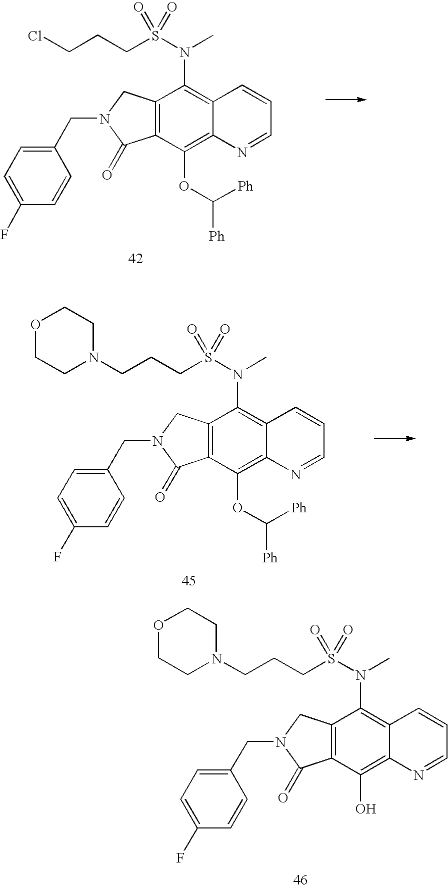

Intermediate 42 (31 mg, 48 μmol) was dissolved in 2 mL of morpholine and allowed to stir at room temperature for 2 days. The mixture was then concentrated in vacuo and azeotroped two times with toluene. The resulting residue was then purified by silica-gel chromatography (6:2:1:1—ethyl acetate methanol:acetic Acid:water) at afford 45 (20 mg, 60%). 45 (24 mg, 38 μmol) was dissolved in 200 μL of DCM and treated with TFA (22 mg, 190 μmol) and triethylsilane (9.0 mg, 76 μmol). After stirring for 15 minutes at room temperature, the reaction mixture was azeotroped with toluene three times. The residue was then triturated with 3:1—hexane:ether to provide 46. 300 MHz ¹H NMR (CDCl₃) δ ppm): 8.88 (d,1H); 8.47 (d, 1H); 7.71 (m, 1H); 7.36 (t, 2H); 7.03 (t, 2H); 4.65 (m, 4H); 3.90 (b, 2H); 3.70 (b , 2H); 3.40 (b, 4H); 3.27 (b, 7H); 2.24 (b, 2H). MS=529.5 (M+1)

### Example 10

### Preparation of 3-cyano-N-(7-(4-fluorobenzyl)-9-hydroxy-8-oxo-7,8-dihydro-6H-pyrrolo[3,4-g]quinolin-5-yl)-N-methylpropane-1 -sulfonamide

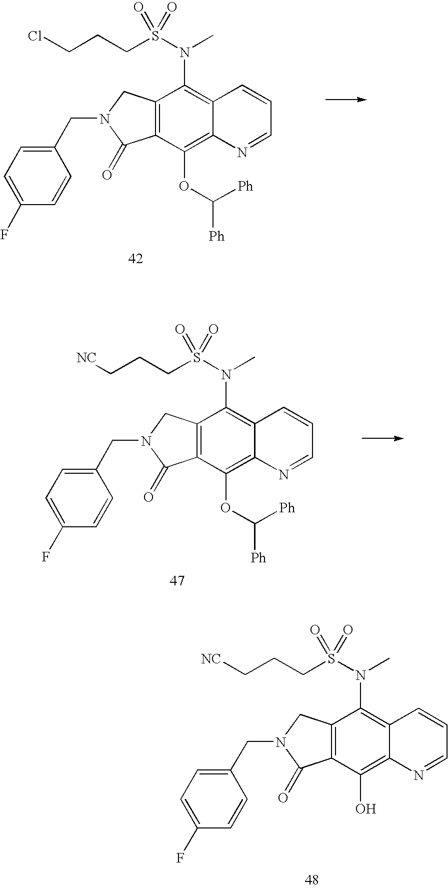

Intermediate 42 (52 mg, 80 μmol) dissolved in 200 μl of DMF was added 12 mg (240 μmol) of NaCN while the mixture stirred at room temperature. The reaction was then warmed to 80° C. and was stirred at 80° C. for 30 minutes. The reaction was the cooled to 0° C. and quenched by adding 60 μl of acetic acid. The mixture was the diluted with ethyl acetate and the organic was washed once with 1 M citric acid, twice with water, and once with brine. The organic layer was then dried over MgSO₄ and concentrated in vacuo. The residue was then purified by silica-gel chromatography (3:1—ethyl acetate:hexane) to afford 47 (35.3 mg, 70%).

47 (24 mg, 38 μmol) was dissolved in 200 μL of DCM and treated with TFA (22 mg, 190 μmol) and triethylsilane (9.0 mg, 76 μmol). After stirring for 15 minutes at room temperature, the reaction mixture was azeotroped with toluene three times. The residue was then triturated with 3:1—hexane:ether to provide 48. 300 MHz ¹H NMR (CDCl₃) δ ppm): 9.02 (d, 1H); 8.30.(d, 1H); 7.70 (m,1H); 7.37 (t, 2H); 7.07 (t, 2H); 4.96 (d, 2H); 4.72 (d,1H); 4.58 (d, 1H); 4.39 (d,1H); 3.34 (m, 5H); 2.62 (m, 2H); 2.24 (m, 3H). MS=469.5 (M +1)

### Example 11

### Preparation of N-(7-(4-fluorobenzyl)-9-hydroxy-8-oxo-7,8-dihydro-6H-pyrrolo[3,4-g]quinolin-5-yl)-3-(1 H-imidazol-1-yl)-N-methylpropane-1-sulfonamide

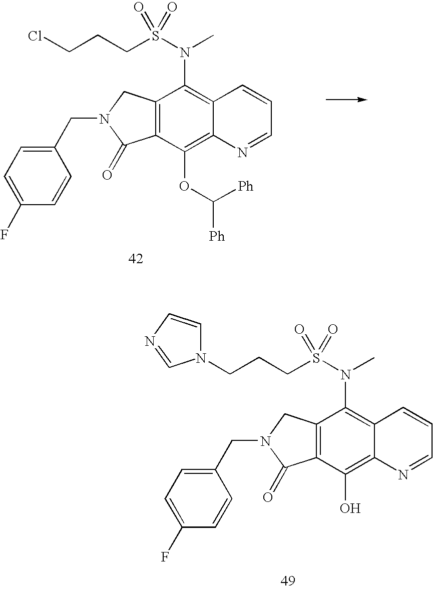

Intermediate 42 from Example 8 (30 mg, 46 μmol) with imidazole (64 mg, 923 μmol) were dissolved in 1 mL of DMF at room temperature. It was heated up to 80° C. for 30 h (none or little progress at lower temperature). After cooled to room temperature, it was quenched with 10% citric acid and extracted with ethyl acetate. The aqueous layer contained desired compound and was concentrated in vacuo. The residue was purified by reverse-phase prep HPLC to provide 15 mg (44% yield) of 49 as the bis-TFA salt. 300 MHz ¹H NMR (CD₃OD) δ ppm): 9.1-8.8 (m, 3H); 8.0 (m, 1H); 7.7 (s, 1H); 7.6 (s, 1H); 7.2 (m, 2H); 7.1 (m, 2H); 4.8-4.4 (m, 6H); 3.4-3.6 (m, 2H); 3.3 (s, 3H); 2.4 (m, 2H). ¹⁹F NMR (CDCl₃) δ (ppm):-78.11, 78.13;-117.2. m/z=510 (M+1).

### Example 12

### Preparation of N-(7-(4-fluorobenzyl)-9-hydroxy-8-oxo-7,8-dihydro-6H-pyrrolo[3,4-g]quinolin-5-yl)-N-2-dioxoisothiazolidine

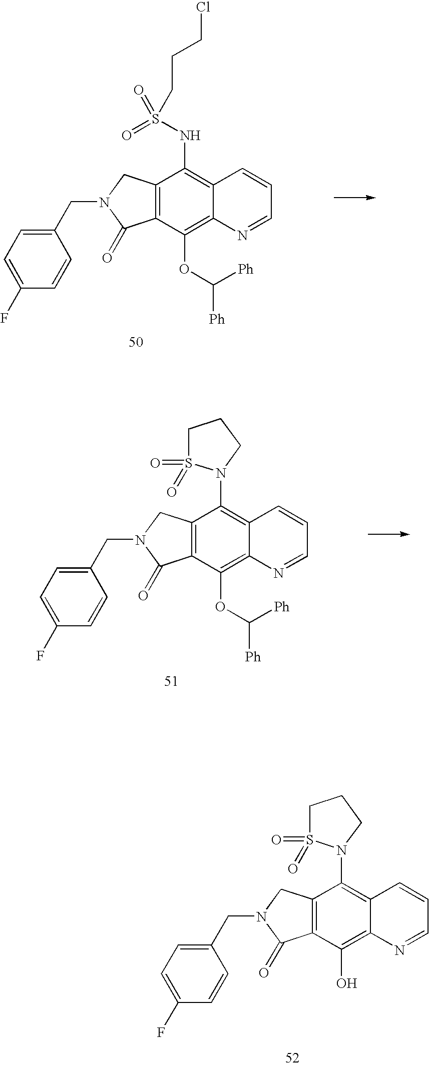

Intermediate 50 (52 mg, 80 μmol, prepared similarly to 36 from 17) was dissolved in 0.4 mL of DMF and cooled to 0° C. Sodium hydride (10 mg, 260 μmol) as a 60% dispersion in mineral oil was added and the reaction was immediately warmed to 75° C. where it was stirred for 7 minutes. The reaction was then cooled to room temperature and quenched with acetic acid (60 μL). The reaction mixture was then diluted with ethyl acetate. The organic phase was then washed once with 0.25 M citric acid, twice with water, and once with brine. The organic phase was then dried over MgSO₄ and concentrated in vacuo. The residue was purified using silica gel chromatography (3:1—ethyl acetate:hexane) to afford 51 (57 mg, 57%).

51 (22 mg, 38 μmol) was dissolved in 200 μL of DCM and treated with TFA (22 mg, 190 μmol) and triethylsilane (9.0 mg, 76 μmol). After stirring for 15 minutes at room temperature, the reaction mixture was azeotroped with toluene three times. The residue was then triturated with 3:1—hexane:ether to afford 52. 300 MHz ¹H NMR (CDCl₃) δ ppm): 9.0 (d, 1H); 8.48 (d, 1H); 7.65 (m, 4H); 7.28 (t, 2H); 7.02 (t, 2H); 4.95 (d, 1H); 4.60 (m, 2H); 4.33 (d, 1H); 3.68 (m, 2H); 3.43 (m, 2H); 2.63 (m, 2H). ¹⁹F NMR (CDCl₃) δ (ppm):−76.36; −114.46. MS=428.5 (M +1).

Example 13

### Preparation of N-(7-(4-fluorobenzyl)-9-hydroxy-8-oxo-7,8-dihydro-6H-pyrrolo[3,4-g]quinolin-5-yl)-N-methylpropane-2-sulfonamide

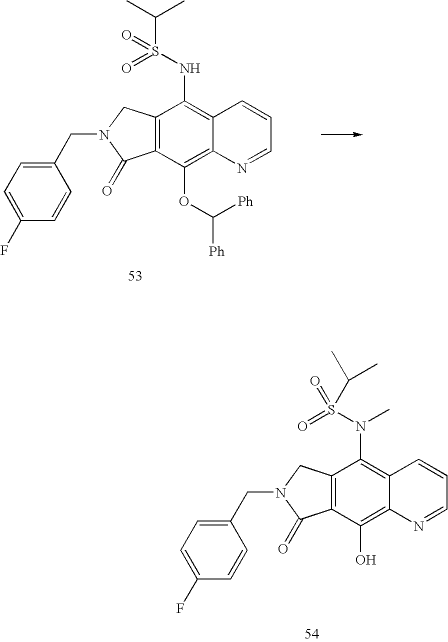

Intermediate 53 (13 mg, 21 μmol), was prepared similarly to 36 from 25 to afford 54. 300 MHz ¹H NMR (CDCl₃) δ ppm: 9.01 (d, 1H); 8.46 (d, 1H); 7.69 (m, 1H); 7.30 (t, 2H); 7.02 (t, 2H); 4.90 (d, 2H); 4.72 (d, 1H); 4.58 (d, 1H); 4.35 (d, 1H); 3.3 (m, 5H); 1.48 (d, 3H); 1.38 (d, 3H). MS=444.5 (M +1).

Example 14

### Preparation of N-(7-(4-fluorobenzyl)-9-hydroxy-8-oxo-7,8-dihydro-6H-pyrrolo[3,4-g]quinolin-5-yl)-N,1-dimethyl-1 H-imidazole-4-sulfonamide

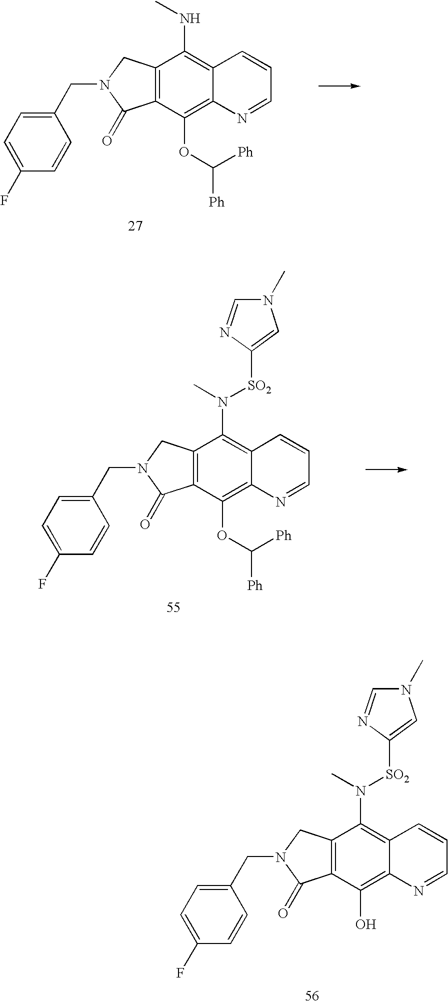

Intermediate 27 (40 mg, 79 μmol) was dissolved in 1 mL of pyridine and flushed with nitrogen, It was cold to 0° C. and added 1-methyl-1 H-imidazole-4-sulfonyl chloride (43,mg, 240 μmol). The mixture was allowed to warm to room temperature and stirred for 20 h under nitrogen. The reaction was diluted with 10 mL of EtOAc, washed with brine, dried over Na₂SO₄ and concentrated in vacuum to give crude product which was then purified by flash chromatography on silica gel (20% to 50% ethyl acetate in hexane, then 5/5/1 of EtOAc/hexane/MeOH) to provide 55 (24 mg, 47%). m/z=648 (M+1).

The deprotection of the DPM group at C₈—OH was carried out as in the conversion of 28 to 29. The resulting residue was then purified by reverse-phase prep HPLC to provide 13 mg (59% in yield) of 56 as a TFA salt. 300 MHz ¹H NMR (CDCl₃) δ (ppm): 9.0 (d, 1H); 8.4 (d, 1H); 7.5 (m, 1H); 7.4 (s, 1H); 7.3 (m, 2H); 7.2 (s, 1H); 7.0 (t, 2H); 4.8 (s, 2H); 4.3-4.5 (m, 2H); 3.6 (s, 3H); 3.4 (s, 3H). m/z=482 (M +1).

### Example 15

### Preparation of N-(7-(4-fluorobenzyl)-9-hydroxy-8-oxo-7,8-dihydro-6H-pyrrolo[3,4-g]quinolin-5-yl)-N,2,4-trimethylthiazole-5-sulfonamide

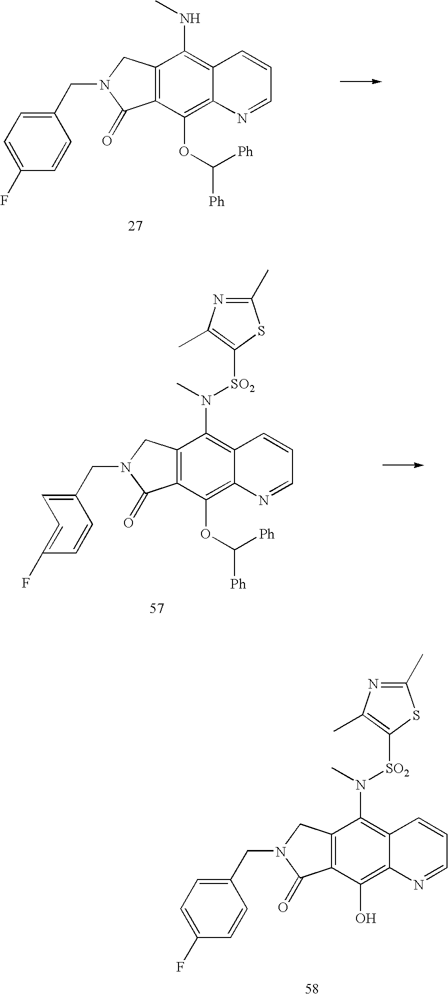

Intermediate 27 (20 mg, 40 μmol) was dissolved in 1 mL of pyridine and flashed with nitrogen, it was cold to 0° C. and added 2,4-dimethyl-thiazole-5-sulfonyl chloride (46.5 mg, 160 μmol) and catalytic amount of DMAP. The mixture was allowed to warm to room temperature and stirred for 20 hours under nitrogen. The reaction was diluted with 10 mL of EtOAc, washed with brine, dried over Na₂SO₄ and concentrated in vacuum to give crude product which was then purified flash chromatography on silica gel (20% to 50% ethyl acetate in hexane) to provide 57 (25 mg, with SM). m/z=679 (M +1).

The deprotection of the DPM group at C₈—OH was carried out as in the conversion of 28 to 29. The resulting residue was then purified by reverse-phase prep HPLC to provide 6 mg (18% in yield for two steps) of 58 as the tris-TFA salt. 300 MHz ¹H NMR (CD₃OD) δ (ppm): 9.0 (d, 1H); 8.2 (d, 1H); 7.6 (m, 1H); 7.4 (m, 2H); 7.1 (t, 2H); 4.9-4.1 (m, 4H); 2.6 (s, 3H); 2.1 (s, 3H). ¹⁹F NMR (CD₃OD) δ (ppm): −78.11, −117.2. m/z=513 (M +1).

### Example 16

### Preparation of N-(7-(4-fluorobenzyl)-9-hydroxy-8-oxo-7,8-dihydro-6H-pyrrolo[3,4-g]quinolin-5-yl)-N-methyl-2-dimethylaminoethane-1-sulfonamide

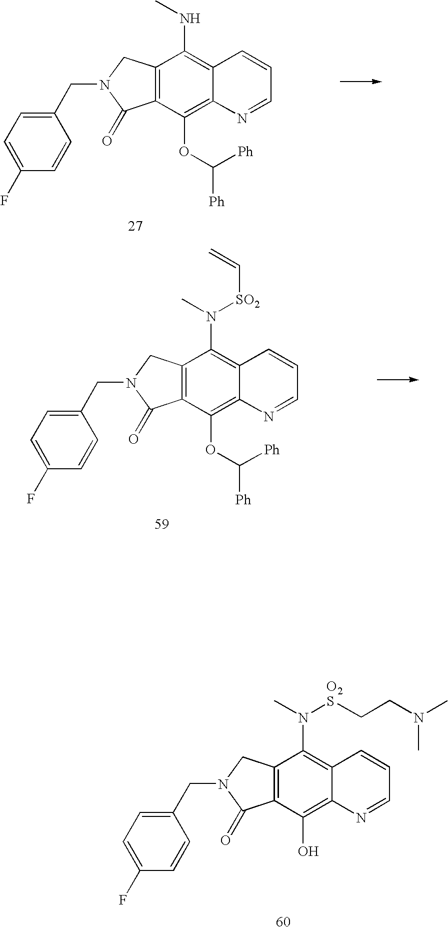

Intermediate 27 (300 mg, 596 μmol) was dissolved in 6 mL of pyridine and flashed with nitrogen. It was cold to 0° C. and added chloroethyl-sulfonyl chloride (188 μl, 1.8 mmol). The mixture was stirred for 10 min under nitrogen. The reaction was diluted with cold water and extracted with EtOAc. The organic phase was washed with 0.1 N HCl and brine, dried over Na₂SO₄ and concentrated in vacuum to give crude product which was precipitated out from ether/DCM. After drying, clean product 59 was obtained as pale colored solid (443mg). m/z=594. The intermediate 59 was treated according to the methods described in example 58 using dimethylamine as the dialkylamine component to afford 60.

### Example 17

### Preparation of N1-(7-(4-fluorobenzyl)-9-hydroxy-8-oxo-7,8-dihydro-6H-pyrrolo[3,4-g]quinolin-5-yl)-N1,N2,N2-trimethyloxalamide

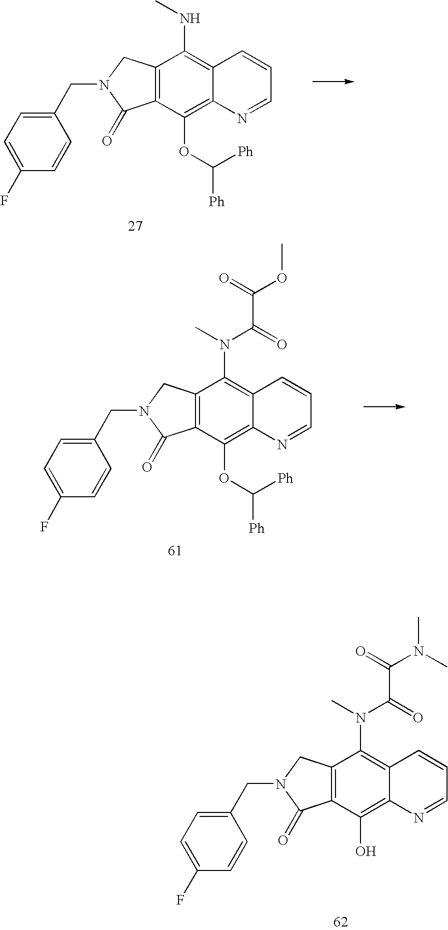

To 27 (crude, 150 μmol) was added 1.0 mL of DCM, followed by TEA (600 μmol, 4.0 eq) and methyl chlorooxoacetate (600 μmol, 4.0 eq). After the reaction mixture was stirred at room temperature for 30 minutes, it was diluted with ethyl acetate. The organic was then washed once with 0.25 M citric acid, twice with water, and once with brine. The organic was then dried over Magnesium Sulfate and concentrated in vacuo. The crude residue was then purified on silica gel (60% ethyl acetate in hexane) to provide intermediate 61 (67 mg, 76%).

61 was dissolved in 200 μL of DCM in a pressure tube. 500 μL of neat Dimethylamine was added, the reactor was sealed, and the reaction was stirred at room temperature for 10 minutes. The reaction was then concentrated in vacuo, followed by a dilution with ethyl acetate. The organic was then washed once with 0.25 M citric acid, twice with water, and once with brine. The organic was then dried over Magnesium Sulfate and concentrated in vacuo.

The residue was dissolved in 200 μL of DCM and treated with TFA (22 mg, 190 μmol) and triethylsilane (9.0 mg, 76 μmol). After stirring for 15 minutes at room temperature, the reaction mixture was azeotroped with toluene three times. The residue was then triturated with 3:1—hexane:ether. The resulting residue was then purified by reverse-phase prep HPLC to provide 62 as the TFA salt. 300 MHz ¹H NMR (CDCl₃) δ (ppm): 9.0 (d,1H); 8.0 (d, 1H); 7.6 (m, 1H); 7.12 (t, 2H); 7.03 (t, 2H); 5.0 (m, 2H); 4.25 (m, 2H); 3.20 (s, 3H); 2.70 (s, 6H). MS=437.5 (M +1)

### Example 18

### Preparation of N-(7-(4-fluorobenzyl)-9-hydroxy-8-oxo-7,8-dihydro-6H-pyrrolo[3,4-g]quinolin-5-yl)-N-methylacetamide and N-(9-(benzhydryloxy)-7-(4-fluorobenzyl)-8-oxo-7,8-dihydro-6H-pyrrolo[3,4-g]quinolin-5-yl)-N-methylacetamide

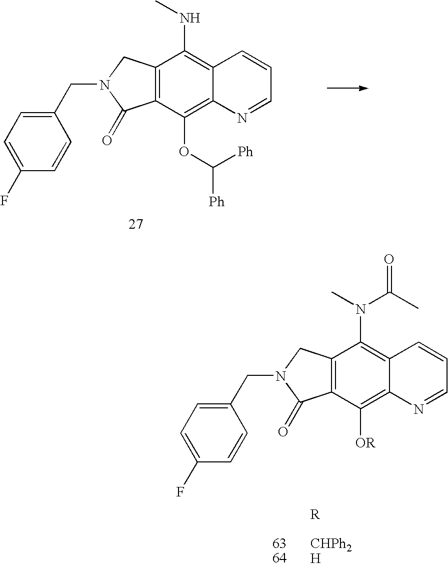

Into a flask containing 27 (16 mg, 0.032 mmol, 1 eq) was added DMF (0.5 mL) and cooled in an ice bath to 0° C. before sodium hydride (1.9 mg, 0.048 mmol, 60% mineral oil, 1.5 eq) was added and stirred for 10 minutes under inert atmosphere. Acetyl chloride (7 μl, 0.095 mmol, 5 eq) was added and the reaction allowed to stir for 2 h at 0° C. The reaction was quenched with saturated NH₄Cl and dissolved in EtOAc (20 mL). It was washed with water (2×40 mL) and brine (40 mL). The organic layer was dried over Na₂SO₄, filtered and concentrated in vacuo. Flash column chromatography was then used purify the product using hexanes/Ethyl acetate (1/4) as eluent. Acylamide 63 was obtained (15 mg, 86% yield).

¹H NMR (300 MHz) CDCl₃ δ: 9.11 (dd, J₁=3.9 Hz, J₂=1.5 Hz, 1H), 8.14 (s, 1H), 8.02 (dd, J₁=8.4 Hz, J₂=1.5 Hz, 1H), 7.78-7.70 (m, 4H, 7.60 (dd, J₁=8.7 Hz, J₂=4.2 Hz, 1H), 7.37-7.15 (m, 10H), 7.07-7.04 (m, 2H), 4.83 (dd, J_{AB}=13.2, 2H), 4.14 (d, J=4.8 Hz, 2H), 3.19 (s, 3H), 1.53 (s, 3H). ¹⁹F NMR (300 MHz) CDCl₃ δ: −115.02. MS: 704.0 (M+1).

Deprotection of DPM group at C₈—OH was carried out as in the conversion of 28 to 29 and 64 was purified by HPLC. ¹H NMR (300 MHz) DMSO-d₆ δ: 8.99 (d, J=2.7 Hz, 1H), 8.21 (d, J=8.4 Hz, 1H), 7.81 (dd, J₁=8.4 Hz, J₂=1.5 Hz, 1H), 7.42-7.33 (m, 2H), 7.21-7.13 (m, 2H), 4.77 (d, j =15.0 Hz, 1H), 4.60 (d, J=15.0 Hz, 1H), 4.42 (s, 2H), 3.13 (s, 3H), 1.58 (s, 3H). ¹⁹F NMR (300 MHz) CDCl₃ δ: −75.56, −114.51 MS: 380.2 (M+H).

### Example 19

### Preparation of N-(7-(4-fluorobenzyl)-9-hydroxy-8-oxo-7,8-dihydro-6H-pyrrolo[3,4-g]quinolin-5-yl)isobutyramide and N-(9-(benzhydryloxy)-7-(4-fluorobenzyl)-8-oxo-7,8-dihydro-6H-pyrrolo[3,4-g]quinolin-5-yl)isobutyramide

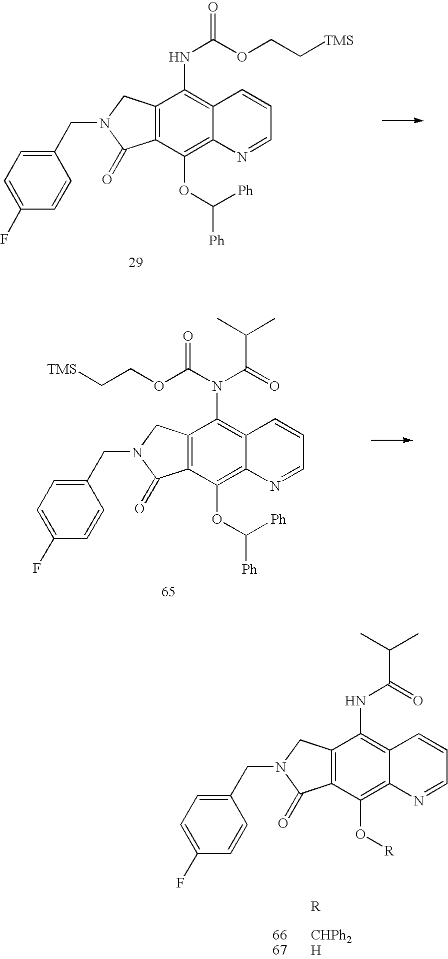

Into a flask containing carbamate 29 (125 mg, 0.20 mmol, 1 eq) was added DMF (2 mL) and cooled in an ice bath to 0° C. before sodium hydride (11 mg, 0.26 mmol, 60% mineral oil, 1.5 eq) was added and stirred for 10 minutes under inert atmosphere. Isobutyryl chloride (31 μl, 0.30 mmol, 1.5 eq) was added and the reaction allowed to stir for 2 h at 0° C. The reaction was quenched with saturated NH₄Cl and dissolved in EtOAc (20 mL). It was washed with water (2×40 mL) and brine (40 mL). The organic layer was dried over Na₂SO₄, filtered and concentrated in vacuo. Flash column chromatography was then used purify the product using hexanes/Ethyl acetate (7/3) as eluent. Acylamide 65 was obtained as a white solid (115 mg, 82% yield).

¹H NMR (300 MHz) CDCl₃ δ: 9.05 (dd, J=3.9 Hz, J₂=1.5 Hz, 1H), 8.13 (s, 1H), 7.88 (dd, J₁=8.7 Hz, J₂=1.5 Hz, 1H), 7.78 -7.70 (m, 4H), 7.52 (dd, J₁=8.7 Hz, J₂=4.2 Hz, 1H), 7.37-7.15 (m, 9H), 7.07-7.04 (m, 2H), 4.95 (d, J=14.7 Hz, 1H), 4.62 (d, J=14.7 Hz, 1H), 4.00 (d, 2H), 4.05-3.98 (m, 2H), 3.85-3.78 (m, 1H), 1.28 (d, J=6.6 Hz, 3H), 1.18 (d, J=6.6 Hz, 3H), 0.49 (t, J=8.4 Hz, 2H), −0.16 (s, 9H). MS: 545.8 (M+1).

Into a flask containing carbamate 65 (71 mg, 0.11 mmol, 1 eq) was added THF (2 mL, 0.05 M) and cooled in an ice bath to 0° C. before tetra-butyl ammonium fluoride (230 μl, 0.23 mmol, 2.2 eq). The reaction was allowed to warm up to ambient temperature and stirred overnight. The reaction was quenched with saturated NH₄Cl and dissolved in EtOAc (20 mL). It was washed with water (2×40 mL) and brine (40 mL). The organic layer was dried over Na₂SO₄, filtered and concentrated in vacuo. Flash column chromatography was then used purify the product using hexanes/ethyl acetate (1/4) as eluent. Acylamide 66 was obtained as an off-white solid (95 mg, 92% yield).

¹H NMR (300 MHz) CDCl₃ δ: 8.95 (s, 1H), 8.04-7.99 (d, J=8.4 Hz, 1H), 8.02 (s, 1H), 7.80-7.74 (m, 4H), 7.27 (dd, J₁=8.4 Hz, J₂=1.5 Hz, 1H), 7.30-7.05 (m, 10H), 7.09-7.02 (m, 1H), 8.07 (s, 1H), 7.74-7.65 (m, 4H), 4.71 (s, 2H), 4.05 (s, 2H), 2.55-2.50 (bs, 1H), 1.27. (d, J=6.9 Hz, 6H). ¹⁹F NMR (300 MHz) CDCl₃ δ: −115.06 MS: 559.9 (M+1).

Deprotection of DPM group at C8—OH was carried out as in Example 18 and 67 was purified by HPLC.

¹H NMR (300 MHz) DMSO-d₆ δ: 9.71 (s, 1H), 8.94 (d, J=2.7 Hz, 1H), 8.28 (d, J=8.4 Hz, 1H), 7.81 (dd, J₁=8.4 Hz, J₂=1.5 Hz, 1H), 7.42 -7.33 (m, 2H), 7.21-7.13 (m, 2H), 4.70 (d J=15.0 Hz, 1H), 4.70 (d, J=15.0 Hz, 1H), 4.26 (d, J=15.0 Hz, 1H), 3.15 (s, 3H), 2.72 (m, 1H), 1.17 (d, J=6.6 Hz, 6H).

### Example 20

### Preparation of N-(7-(4-fluorobenzyl)-9-hydroxy-8-oxo-7,8-dihydro-6H-pyrrolo[3,4-g]quinolin-5-yl)-N-methylisobutyramide and N-(9-(benzhydryloxy)-7-(4-fluorobenzyl)-8-oxo-7,8-dihydro-6H-pyrrolo[3,4-g]quinolin-5-yl)-N-methylisobutyramide

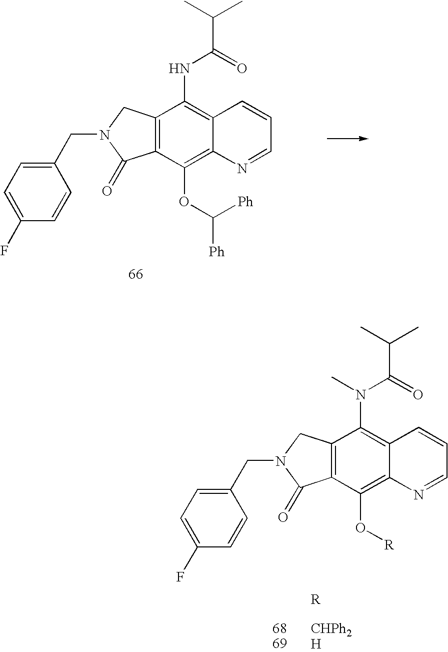

Into a flask containing acylamide 66 (47 mg, 0.084 mmol, 1 eq) was added DMF (1.5 mL) and cooled in an ice bath to 0° C. before sodium hydride (4 mg, 0.10 mmol, 60% mineral oil, 1.2 eq) was added and stirred for 10 minutes under inert atmosphere. Iodomethane (16 μl, 0.29 mmol, 3 eq) was added and the reaction allowed to stir for an hour at 0° C. The reaction was quenched with saturated NH4Cl and dissolved in EtOAc (20 mL). It was washed with water (2×40 mL) and brine (40 mL). The organic layer was dried over Na₂SO₄, filtered and concentrated in vacuo. No further purification was carried out. Amide 68 was obtained (50 mg, 103% yield). MS: 573.9 (M+1).

Deprotection of DPM group at C8—OH was carried out as in Example 18 and 69 was purified by HPLC.

¹H NMR (300 MHz) DMSO-d₆ δ: 8.99 (d, J=2.7 Hz, 1H), 8.19 (d, J=8.4 Hz, 1H), 7.81 (dd, J₁=8.4 Hz, J₂=1.5 Hz, 1H), 7.42 -7.33 (m, 2H), 7.21-7.13 (m, 2H), 4.70 (d J=15.0 Hz, 1H), 4.50 (d, J=15.0 Hz, 1H), 4.23 (d, J=15.0 Hz, 1H), 3.15 (s, 3H), 2.06 (m, 1H), 0.79 (d, J=6.9 Hz, 3H), 0.71 (d, J=6.9 Hz, 3H). ¹⁹F NMR (300 MHz) CDCl₃ δ: −75.79,-114.69. MS: 394.3 (M+H).

### Example 21

### Preparation of N-(7-(4-fluorobenzyl)-9-hydroxy-6,6-dimethyl-8-oxo-7,8-dihydro-6H-pyrrolo[3,4-g]quinolin-5-yl)-N-methylmethanesulfonamide and N-(9-(benzhydryloxy)-7-(4-fluorobenzyl)-6,6-dimethyl-8-oxo-7,8-dihydro-6H-pyrrolo[3,4-g]quinolin-5-yl)-N-methylmethanesulfonamide

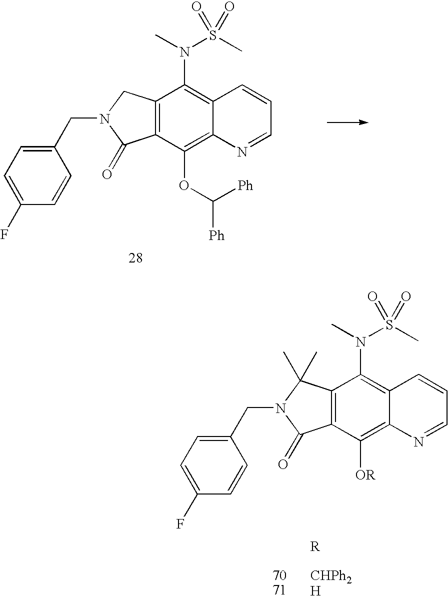

28 (64 mg, 110 μmol) was dissolved in 1 mL of DMF and cooled to 0° C. Sodium hydride (25 mg, 660 μmol) as a 60% dispersion in mineral oil was added and the reaction was stirred at 0° C. for 2 minutes. Iodomethane (94 mg, 660 μmol) was then added and the reaction was allowed to warm to room temperature. After stirring at room temperature for 30 minutes, 30 μL of acetic acid was added and the reaction was then diluted with ethyl acetate. The organic phase was washed once with 0.25 M citric acid, twice with water, and once with brine. The organic was then dried over Mg₂SO₄ and concentrated in vacuo. The residue was then purified by silica gel chromatography (3:1—hexane:ethyl acetate) to afford intermediate 70 (25 mg, 37%).

70 (24 mg, 38 μmol) was dissolved in 200 μL of DCM and treated with TFA (22 mg, 190 μmol) and triethylsilane (9.0 mg, 76 μmol). After stirring-for 15 minutes at room temperature, the reaction mixture was azeotroped with toluene three times. The residue was then triturated with 3:1—hexane:ether to afford a crude residue which was purified by reverse-phase prep HPLC to afford 71. 300 MHz ¹H NMR (CDCl₃) δ (ppm): 8.98 (d, 1H); 8.63 (d, 1H); 7.72 (m, 1H); 7.40 (t, 2H); 7.03 (t, 2H); 4.70 (m, 2H); 3.30 (s, 3H); 3.26 (s, 3H); 1.68 (s, 3H); 1.57 (s, 3H). MS=444.5 (M +1)

### Example 22

### Preparation of N-(7-(4-fluorobenzyl)-9-hydroxy-6-(R and S)-methyl-8-oxo-7,8-dihydro-6H-pyrrolo[3,4-g]quinolin-5-yl)-N-methylmethanesulfonamide and N-(9-(benzhydryloxy)-7-(4-fluorobenzyl)-6-(R,S)-methyl-8-oxo-7,8-dihydro-6H-pyrrolo[3,4-g]quinolin-5-yl)-N-methylmethanesulfonamide

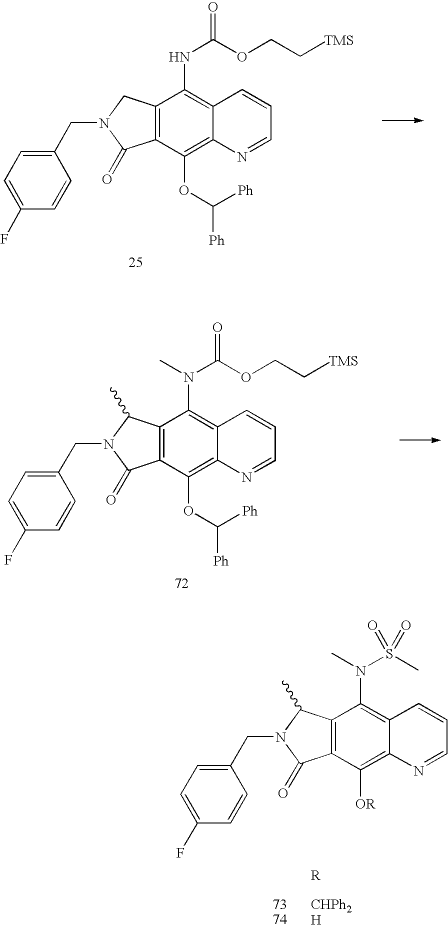

Into a flask containing carbamate 25 (60 mg, 95 μmol, 1 eq) was added DMF (300 μl) and cooled in an ice bath to 0° C. before sodium hydride (5.3 mg, 400 μmol), as a 60% mineral oil dispersion was added and stirred for 5 minutes under inert atmosphere. Iodomethane (135 mg, 950 μmol) was added and the reaction allowed to stir for 45 minutes at room temperature. The reaction was quenched with saturated NH₄Cl and dissolved in EtOAc (20 mL). It was washed with water (2×40 mL) and brine (40 mL). The organic layer was dried over Na₂SO₄, filtered and concentrated in vacuo. No further purification was carried out. Carbamate 72 was obtained (33 mg, 50% yield).

Into a flask containing carbamate 72 (25 mg, 0.038 mmol, 1 eq) was added THF (4 mL,) and cooled in an ice bath to 0° C. before tetra-butyl ammonium fluoride (100 μl, 0.096 mmol, 2.5 eq). The reaction was allowed to warm up to ambient temperature and stirred overnight. The reaction was quenched with saturated NH₄Cl and dissolved in EtOAc (20 mL). It was washed with water (2×40 mL) and brine (40 mL). The organic layer was dried over Na₂SO₄, filtered and concentrated in vacuo.

To the resulting residue (161 mg, 0.31 mmol, 1 eq) was added pyridine (3 mL, 0.2 M) and cooled in an ice bath to 0° C. before methanesulfonyl chloride (72 μl, 0.93 mmol, 3 eq) was added and the reaction allowed to warm up to room temperature overnight. The reaction was quenched with saturated NH₄Cl and dissolved in EtOAc (20 mL). It was washed with water (2×40 mL) and brine (40 mL). The organic layer was dried over Na₂SO₄, filtered and concentrated in vacuo. Flash column chromatography was then used purify the product using hexanes/Ethyl acetate (3/2) as eluent. Sulfonylamide 73 was obtained as a white solid (158 mg, 86% yield).

73 (24 mg, 38 μmol) was dissolved in 200 μL of DCM and treated with TFA (22 mg, 190 μmol) and triethylsilane (9.0 mg, 76 μmol). After stirring for 15 minutes at room temperature, the reaction mixture was azeotroped with toluene three times. The residue was then triturated with 3:1—hexane:ether to afford a crude residue which was purified by reverse-phase prep HPLC to afford 74. 300 MHz ¹H NMR (CDCl₃) δ (ppm): 8.98 (d, 1H); 8.14 (d, 1H); 7.60 (m, 1H); 7.28 (m, 2H); 7.00 (t, 2H); 5.05 (d,1H); 4.96 (q, 1H); 4.38 (d, 1H); 3.35 (s, 3H); 2.98 (s, 3H); 1.51 (d, 3H). MS=430.5 (M+1).

### Example 23

### Preparation of N-(7-(4-fluorobenzyl)-9-hydroxy-6-(R)-methyl-8-oxo-7,8-dihydro-6H-pyrrolo[3,4-g]quinolin-5-yl)-N-methylmethanesulfonamide and N-(7-(4-fluorobenzyl)-9-hydroxy-6-(S)-methyl-8-oxo-7,8-dihydro-6H-pyrrolo[3,4-g]quinolin-5-yl)-N-methylmethanesulfonamide

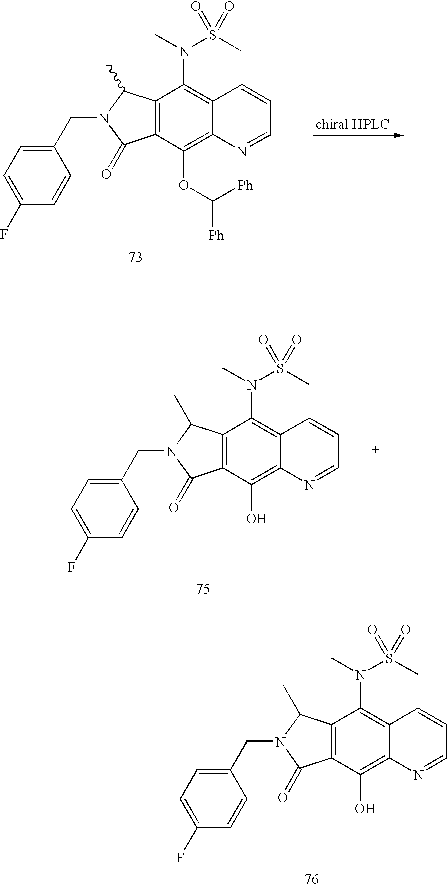

The enantiomers of 73 (100 mg) were separated by chiral preparatory purification (Chiralpac Chiralcel OD-H 250×4.6 mm, 5 micron stationary phase, 1:1—Methanol:Ethanol mobile phase). Enantiomer Retention Times=11.76 min., 12.69 min. Each enantiomer was then treated with TFA and TES according to the following representative procedure in order to provide 75 and 76 respectively. The enantiomer (24 mg, 38 μmol) was dissolved in 200 μL of DCM and treated with TFA (22 mg, 190 μmol) and triethylsilane (9.0 mg, 76 μmol). After stirring for 15 minutes at room temperature, the reaction mixture was azeotroped with toluene three times. The residue was then triturated with 3:1—hexane:ether to afford 75 (from the precursor first eluted by chiral HPLC) and 76 (from the precursor second eluted by chiral HPLC). Data for 75: 300 MHz ¹H NMR (CDCl₃) δ (ppm): 8.98 (d, 1H); 8.14 (d, 1H); 7.60 (m, 1H); 7.28 (m, 2H); 7.00 (t, 2H); 5.05 (d, 1H); 4.96 (q, 1H); 4.38 (d, 1H); 3.35 (s, 3H); 2.98 (s, 3H); 1.51 (d, 3H). MS=430.5 (M +1). Data for 63: 300 MHz ¹H NMR (CDCl₃) δ (ppm): 8.98 (d, 1H); 8.14 (d, 1H); 7.60 (m, 1H); 7.28 (m, 2H); 7.00 (t, 2H); 5.05 (d, 1H); 4.96 (q, 1H); 4.38 (d,1H); 3.35 (s, 3H); 2.98 (m, 3H); 1.51 (d, 3H). MS=430.5 (M +1).

### Example 24

### Preparation of N-ethyl-N-(7-(4-fluorobenzyl)-9-hydroxy-8-oxo-7,8-dihydro-6H-pyrrolo[3,4]quinolin-5-yl)methanesulfonamide and N-ethyl-N-(9-(benzhydryloxy)-7-(4-fluorobenzyl)-8-oxo-7,8-dihydro-6H-pyrrolo[3,4-g]quinolin-5-yl)methanesulfonamide

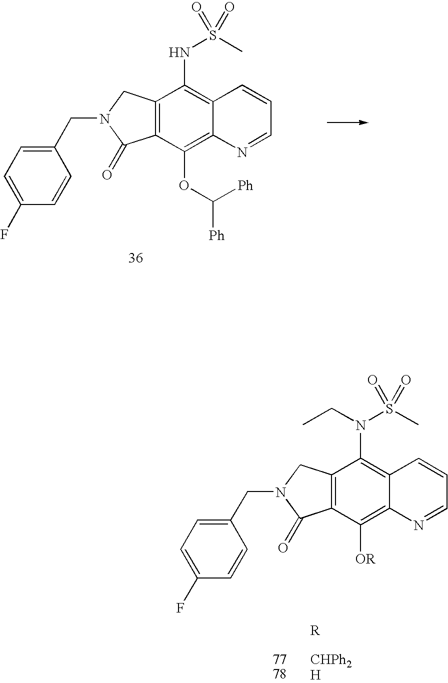

Intermidiate 77 was synthesized in a manner similar to that of 28 from 77 (24 mg, 38 μmol) was then dissolved in 200 μL of DCM and treated with TFA (22 mg, 190 μmol) and triethylsilane (9.0 mg, 76 μmol). After stirring for 15 minutes at room temperature , the reaction mixture was azeotroped with toluene three times. The residue was then triturated with 3:1—hexane:Ether to provide 78 300 MHz ¹H NMR (CD₃OD) δ (ppm): 8.92 (d, 1H); 8.57 (d, 1H); 7.76 (m, 1H); 7.36 (t, 2H); 7.03 (t, 2H); 4.60 (m, 4H); 3.73 (m, 2H); 3.10 (s, 3H); 1.04 (t, 3H). MS=430.5 (M+1).

### Example 25

### Preperation of 5-fluoro-2-((9-hydroxy-5-(N-methylmethylsulfonamido)-8-oxo-6H-pyrrolo[3,4]quinolin- 7(8H)-N-methylbenzamide and 5-fluoro-2-((9-(4-methoxybenzyloxy)-5-(N-methylmethylsulfonamido)-8-oxo-6H-pyrrolo[3,4-g]quinolin-7(8H)-yl)methyl)-N-methylbenzamide

Procedure:

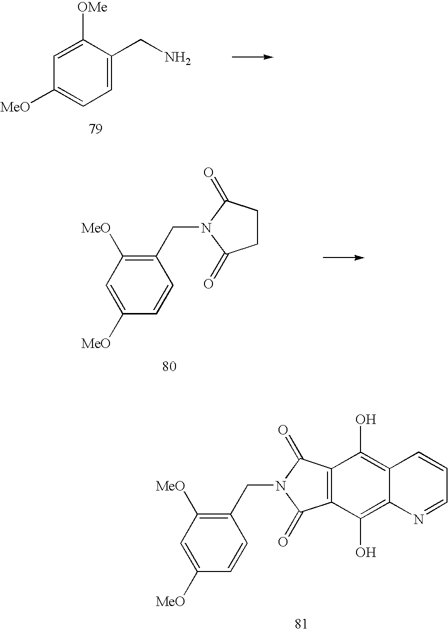

To a solution of the commercially available succinic anhydride 79(16.1 g, 161mmol) in N,N-dimethylformamide (DMF) (40.6 mL) was added 2,4-dimethoxybenzyl amine (27.1 g, 161 mmol). The reaction mixture was heated to 150° C. under nitrogen atmosphere and stirred for 2 days at which point the reaction was complete. The reaction mixture was diluted with ethyl acetate, washed with saturated NH₄Cl, brine (twice), and aqueous LiCI (twice), then dried (over Na₂SO₄), filtered and concentrated in vacuo. The residue was purified by chromatography on silica gel (2/1- ethyl acetate/hexane) to afford the desired product 80 (33.1 g, 82%): 300 MHz ¹H NMR (CDCl₃) δ (ppm) 7.15 (d,1H), 6.4 (m, 2H), 4.65 (s, 2H), 3.85 (s, 3H), 3.8 (s, 3H), 2.7 (s, 4H); MS: 250 (M+1)

80 (33.1 g, 132.8 mmol) and 2,3-pyridine carboxylic acid dimethyl ester (20.2 g, 159.3 mmol) were dissolved in dry THF (1000 mL) and dry methanol (100 mL) in a 3-necked flask with a mechanical stirrer and condenser. To this was added NaH (60% in mineral oil, 7.01 g, 292.1 mmol) slowly in four portions. The mixture stirred until bubbling ceased, then refluxed for 24 hours. 50 mL 6 M HCl was added to the mixture while in an ice bath, stirring for 15 minutes. 200 mL diethyl ether was added, and the precipitate was filtered, and washed with diethyl ether and H₂O , then dried under vacuum at 100° C. with no further purification to afford the desired product 81 (23.7 g, 50%) as a solid: 300 MHz ¹H NMR (CD₃SOCD₃) δ 9.05 (d, 1H), 8.75 (d, 1H), 7.8 (dd, 1H), 7.95 (d, 1H), 6.55 (s,1H), 6.45 (d, 1H), 4.65 (s, 2H), 3.8 (s, 3H), 3.7 (s, 3H); MS: 381 (M+1).

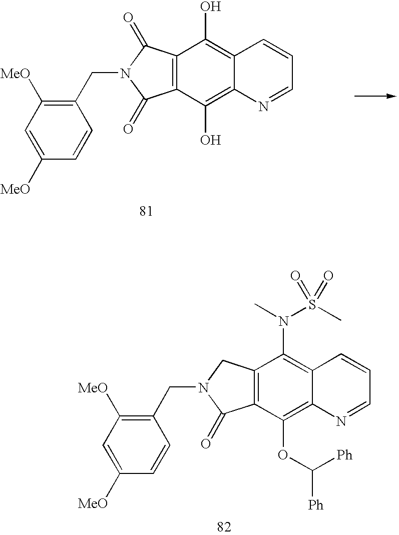

Compound 82 was synthesized in multi-steps from 81 in a manner similar to 28 from 10 as a solid: 300 MHz ¹H NMR (CDCl₃) δ (ppm) 9.05 (dd, 1H), 8.22 (s, 1H), 8.19 (dd, 1H), 7.75 (m, 4H), 7.55 (dd, 1H), 7.3-7.1 (m, 7H), 6.45 (m, 2H), 4.9 (d, 1H), 4.75 (d, 1H), 4.6 (d, 1H), 4.25 (d, 1H), 3.85(s, 3H), 3.82 (s, 3H), 3.25 (s, 3H), 3.05 (s, 3H); MS: 624 (M+1).

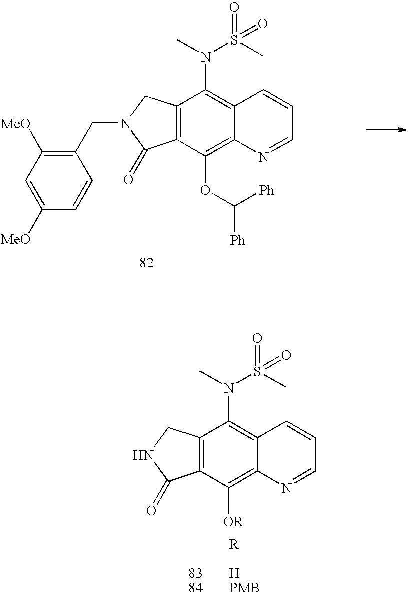

To a solution of 82 (1.86 g, 2.98 mmol) dissolved in anhydrous dichloromethane (60 mL) was added triethylsilane (4.76 mL, 29.8 mmol) and trifluoroacetic acid (2.3 mL, 29.8 mmol). The reaction mixture was stirred for 1 hour under nitrogen atmosphere then concentrated in vacuo. The residue was redissolved in trifluoroacetic acid (45 mL) and triethylsilane (1 mL) was added. The reaction mixture was stirred at room temperature under nitrogen atmosphere for 1 hour then heated to 75° C. for 2 hours upon which the mixture was azeotroped with toluene repeatedly. The crude residue was suspended in a solution of hexanes/ethyl ether (30 mL, 1/1) washed thoroughly via triturating (3×15 mL). Sonication was used to aid this washing. The solid was filtered on a sintered funnel and air dried thoroughly. An off-white brownish solid 83 (982 mg, 78 %) was obtained as the TFA salt; MS: 308 (M+1).

The lactam 83 (982 mg, 3.19 mmol) was dissolved in DMF (32 mL) and treated with Cs₂CO₃ (4.15 g, 12.8 mmol), para-methoxybenzyl chloride (1.30 mL, 9.58 mmol) and tetrabutylammonium iodide (590 mg, 1.6 mmol). The reaction was stirred under nitrogen atmosphere at 65° C. for 2 hours upon which half of the volume of DMF was removed in vacuo then diluted with ethyl acetate. The reaction was quenched with water and the organic layer was washed with water (twice), aqueous LiCl (twice), and brine, then dried (over Na₂SO₄), filtered and concentrated in vacuo. The crude residue was purified by chromatography on silica gel (eluting with 0-10% methanol in ethyl acetate) in order to obtain a light brownish solid 84 (800mg, 80%): 300 MHz ¹H NMR (DMSO) δ (ppm) 9.05 (dd, 1H), 8.75 (s, 1H), 8.44 (dd, 1H), 7.75 (dd, 1H), 7.62 (d, 2H), 6.92 (d, 2H), 5.52 (s, 2H), 4.55 (s, 2H), 3.75 (s, 3H), 3.3 (m, 6H); MS: 428 (M+1).

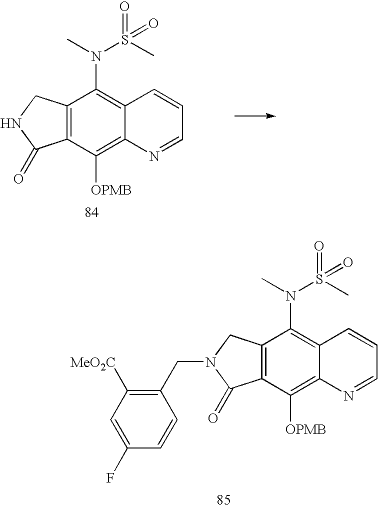

To a solution of lactam 84 (32 mg, 0.075 mmol) dissolved in DMF (1 mL) and cooled in an ice bath to 0° C. was added sodium hydride (3.9 mg, 0.097 mmol, 60% mineral oil) and stirred for 5 minutes under nitrogen atmosphere. Methyl 2-bromomethyl-5-fluoro-benzoate (27.8 mg, 0.112 mmol) and tetrabutylammonium iodide (8.3 mg, 0.022 mmol) was added and the reaction was allowed to stir for 30 minutes at 0° C. The reaction was quenched with H₂O and diluted with ethyl acetate. The organic layer was washed with H₂O , aqueous LiCl (twice), and brine, then dried (over Na₂SO₄), filtered and concentrated in vacuo. The crude residue was purified by chromatography on silica gel (4/1- ethyl acetate/hexane) to afford the desired product 85 (25 mg, 56%): 300 MHz ¹H NMR (CDCl₃) δ (ppm) 9.07 (dd, 1H), 8.3 (dd, 1H), 7.65 (m, 4H), 7.45 (m, 1H), 7.22 (m, 1H), 6.88 (d, 2H), 5.79 (m, 2H), 5.22 (m, 2H), 4.75 (d, 1H), 4.45 (d, 1H), 3.98 (s, 3H), 3.79 (s, 3H), 3.35 (m, 3H), 3.2 (s, 3H); MS: 594 (M+1).

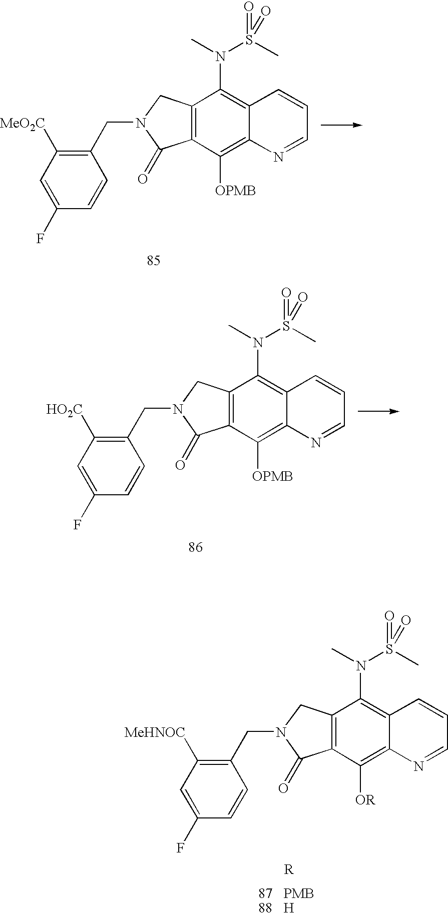

To a solution of methyl ester 85 (14 mg, 0.024 mmol) dissolved in THF (0.236 mL) and water (0.060 mL) was added DMAP (catalytic) and LiOH (4 mg, 0.0944 mmol). The reaction was stirred at room temperature for 2.5 hours upon which diluted with ethyl acetate (5 mL). The mixture was acidified with 1N HCl (until soln pH=3) and the product was extracted with ethyl acetate. The organic layer was washed with brine then dried (over Na₂SO₄), filtered and concentrated in vacuo to give clean product 86 (15 mg, 100%) with no further purification; MS: 580 (M+1).

A solution of carboxylic acid 86 (15 mg, 0.024 mmol) in DMF (0.236 mL) that had been stirred with HATU (0.018 g, 0.047 mmol) and DIPEA (0.0125 mL, 0.071 mmol) for 5 minutes was treated with TEA (0.050 mL, 0.354 mmol) and methylamine hydrochloride (16 mg, 0.236 mmol). The reaction mixture was stirred overnight under nitrogen atmosphere upon which diluted with ethyl acetate, washed with saturated NH₄Cl, brine, and aqueous LiCl (twice), then dried (NaSO₄), filtered and concentrated. The residue was purified by chromatography on silica gel (0-10% - methanol/ethyl acetate) to afford the desired product 87 (8.4 mg, 60%): 300 MHz ¹H NMR (CDCl₃)δ (ppm) 9.06 (dd, 1H), 8.35 (dd, 1H), 7.62 (m, 4H), 7.47 (m, 1H), 7.13 (m, 2H), 6.86 (m, 2H), 6.5 (bs, 1H), 5.78 (m, 2H), 4.92 (m, 2H), 4.75 (d, 1H), 4.53 (d, 1H), 3.80 (s, 3H), 3.35 (s, 3H), 3.14 (m, 3H), 2.98 (d, 3H); MS: 593 (M+1).

A solution of the amide 87 (8.4 mg, 0.014 mmol) in dichloromethane (0.5 mL) was treated with trifluoroacetic acid (0.1 mL) and triethylsilane (0.05 mL). The reaction mixture was stirred at room temperature under an inert atmosphere for 20 minutes. The volatiles were removed in vacuo with toluene. The solid was triturated in diethyl ether/hexane to afford the desired product 88 (6 mg, 100%) as the parent solid: 300 MHz ¹H NMR (CDCl₃) δ (ppm) 8.98 (dd, 1H), 8.35 (dd, 1H), 7.66 (m, 1H), 7.48 (m, 1H), 7.13 (m, 2H), 6.56 (bs, 2H), 4.92 (s, 2H), 4.8 (d, 1H), 4.6 (d, 1H), 3.35 (s, 3H), 3.11 (s, 3H), 3.01 (d, 3H); 300 MHz ¹⁹F NMR (CDCl₃) δ (ppm) -113.25; MS: 473 (M+1).

### Example 26

### Preparation 5-fluoro-2-((9-hydroxy-5-(N-methylmethylsulfonamido)-8-oxo-6H-pyrrolo[3,4-g]quinolin-7(8H)-yl)methyl)-N,N-dimethylbenzamide and 5-fluoro-2-((9-(4-methoxybenzyloxy)-5-(N-methylmethylsulfonamido)-8-oxo-6H-pyrrolo[3,4-g]quinolin-7(8H)-yl)methyl)-N, N-dimethylbenzamide

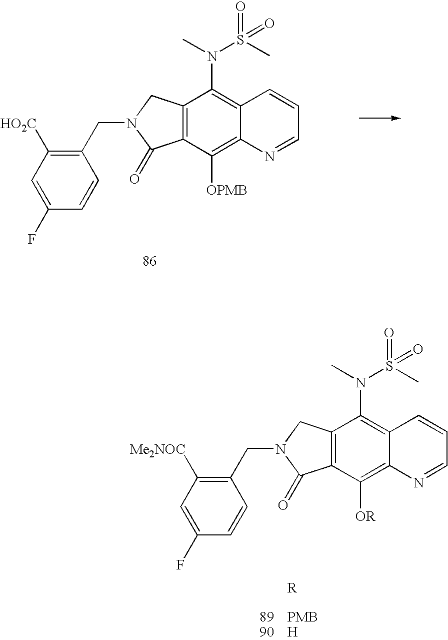

A solution of carboxylic acid 86 from example 25 (27.3 mg, 0.047 mmol) in DMF (0.250 mL) that had been stirred with HATU (54 mg, 0.142 mmol) and DIPEA (0.041 mL, 0.236 mmol) for 5 minutes was treated with dimethylamine (2M THF soln, 0.472 mL, 0.944 mmol). The reaction mixture was stirred overnight under nitrogen atmosphere upon which diluted with ethyl acetate, washed with saturated NH₄Cl, brine, and aqueous LiCl (twice), then dried (NaSO₄), filtered and concentrated. The residue was purified by chromatography on silica gel (0-10% - methanol/ethyl acetate) to afford the desired product 89 (16 mg, 57%): 300 MHz ¹H NMR (CDCl₃) δ (ppm) 9.05 (dd, 1H), 8.35 (dd, 1H), 7.7-7.5 (m, 3H), 7.45 (m, 1H), 7.1 (m, 1H), 6.95 (m, 1H), 6.94 (d, 2H), 5.76 (m, 2H), 4.95 (d, 1H), 4.65 (d, 1H), 4.55 (d, 1H), 4.4 (d, 1H), 3.80 (s, 3H), 3.32 (s, 3H), 3.05 (s, 3H), 2.91 (s, 3H); MS: 607 (M+1)

The compound 90 was made from 89 in a similar fashion as compound 88 in example 25 to afford the desired product 90 (12 mg, 76%) as the TFA salt: 300 MHz ¹H NMR (CDCl₃) δ (ppm) 9.02 (dd, 1H), 8.42 (dd, 1H), 7.68 (dd, 1H), 7.45 (m, 1H), 7.12 (m, 1H), 6.97 (m, 1H), 4.95 (d, 1H), 4.6 (m, 2H), 4.45 (d, 1H), 4.4 (d, 1H), 3.32 (s, 3H), 3.1 (s, 3H), 3.06 (s, 3H), 2.93 (s, 3H); 300 MHz ¹⁹F NMR (CDCl₃) δ (ppm) -76.37, -113.25; MS: 487 (M+1).

### Example 27

### Preparation of N-(9-hydroxy-8-oxo-7-(pyridin-4-ylmethyl)-7,8-dihydro-6H-pyrrolo[3,4-g]quinolin-5-yl)-N-methylmethanesulfonamide and N-(9-(4-methoxybenzyloxy)-8-oxo-7-(pyridin-4-ylmethyl)-7,8-dihydro-6H-pyrrolo[3,4-g]quinolin-5-yl)-N-methylmethanesulfonamide

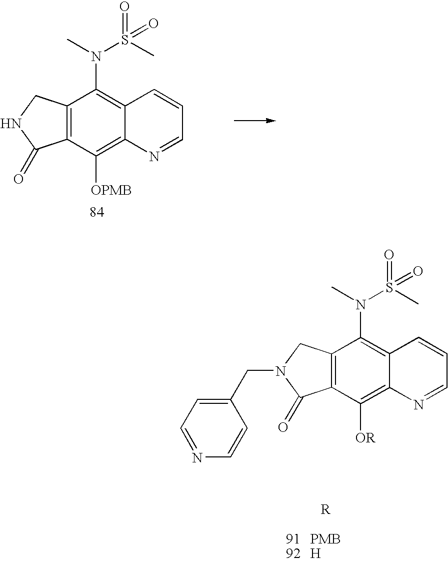

In procedures similar to those exemplified in example 25, compound 91 was prepared from 84 using the commercially available 4-bromomethyl pyridine. ¹H NMR (300 MHz) CDCl₃ δ: partial list 5.81 (d, J=3.9 Hz, 2H), 5.07 (d, J=15.6 Hz, 1H), 4.75 (d, J=18 Hz, 1H), 4.65 (d, J=15.6 Hz, 1H), 4.37 (d, J=18 Hz, 1H). MS: 519.11 (M+1).

In procedures similar to those exemplified above, compound 92 was prepared. ¹H NMR (300 MHz) CD₃OD δ: partial 5.08 (s, 2H), 3.37 (s, 3H), 3.20 (s, 3H). ¹⁹F NMR (300 MHz) CDCl₃ δ: -77.62, -80.66. MS: 399.12 (M+1).

### Example 28

### Preparation of N-(9-hydroxy-8-oxo-7-(pyridin-2-ylmethyl)-7,8-dihydro-6H-pyrrolo[3,4-g]quinolin-5-yl)-N-methylmethanesulfonamide and N-(9-(4-methoxybenzyloxy)-8-oxo-7-(pyridin-2-ylmethyl)-7,8-dihydro-6H-pyrrolo[3,4-g]quinolin-5-yl)-N-methylmethanesulfonamide

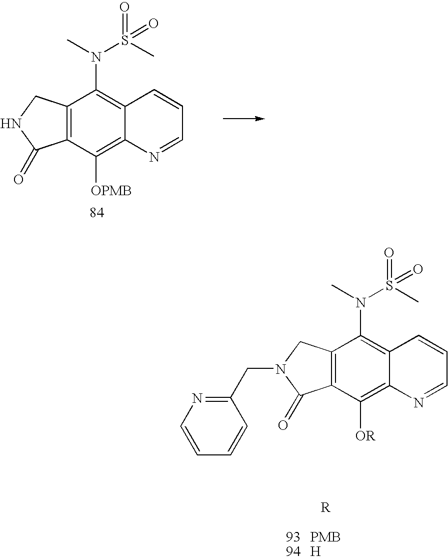

In procedures similar to those exemplified in example 23, compound 93 was prepared from 84 using the commercially available 2-bromomethyl pyridine. ¹H NMR (300 MHz) CDCl₃ δ: partial list 5.81 (AB, J=10.50 Hz, 2H), 5.07 (d, J=15.6 Hz, 1H), 4.75 (d, J=18 Hz, 1H), 4.65 (d, J=15.6 Hz, 1H), 4.37 (d, J=18 Hz, 1H). MS: 519.11 (M+1), 541.12 (M=23).

In procedures similar to those exemplified above, compound 94 was prepared. ¹H NMR (300 MHz) CDCl₃ δ: partial 4.63 (s, 2H). ¹⁹F NMR (300 MHz) CDCl₃ δ: -77.89. MS: 399.09 (M+1).

### Example 29

### Preparation of N-(7-((6-fluoropyridin-3-yl)methyl)-9-hydroxy-8-oxo-7,8-dihydro-6H-pyrrolo[3,4-g]quinolin-5-yl)-N-methylmethanesulfonamide and N-(7-((6-fluoropyridin-3-yl)methyl)-9-(4-methoxybenzyloxy)-8-oxo-7,8-dihydro-6H-pyrrolo[3,4-g]quinolin-5-yl)-N-methylmethanesulfonamide

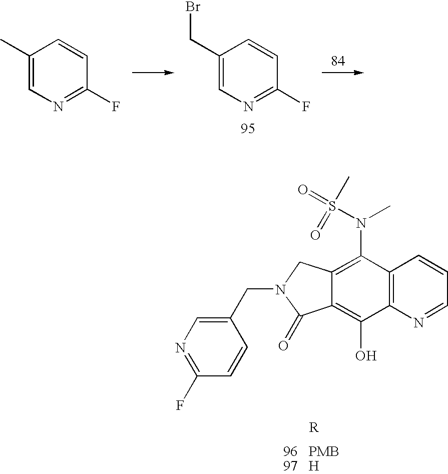

Into a flask containing 2-fluoro-5-methylpyridine (1g, 10.30 mmol, 1 eq) was added carbon tetrachloride (100 mL, 0.1 M), N-Bromosuccinimide (2.01 g, 11.33 mmol, 1.1 eq) and benzoyl peroxide (125 mg, 0.052 mmol, 0.05 eq). The reaction was refluxed at 80° C. for 16 h. The reaction was cooled and the solid filtered off. The filtrate was concentrated in vacuo. Flash column chromatography was used to purify the product using hexanes/ethyl acetate (4/1) as eluent. Bromide 95 was obtained. ¹H NMR (300 MHz) CDCl₃ δ: 8.24 (s, 1H), 7.85 (dt, J₁=8.4, J₂=2.4 Hz, 1H), 6.95 (dd, J₁=8.4 Hz, J₂=2.4 Hz, 1H).

To a flask containing lactam 84 from example 25 (30 mg, 0.070 mmol, 1 eq) was added DMF (1.2 mL, 0.1 M). At 80° C., sodium hydride (3.5 mg, 1.3 eq, 0.1 eq, 60 % mineral oil) was added. The reaction was allowed to stir for 5 minutes before the aryl bromide 95 (20 mg, 0.11 mmol, 1.5 eq) was added. TLC was used to indicate when the reaction was complete. The reaction was quenched with saturated NH₄Cl and dissolved in EtOAc (10 mL). It was washed with water (2×5mL) and brine (10 mL). The organic layer was dried over Na₂SO₄, filtered and concentrated in vacuo. Flash column chromatography was used to purify the product using hexanes/Ethyl acetate (1/4) as eluent. Sulfonamide 96 was obtained as a clear oil (30 mg, 80% yield).

Into a flask containing sulfonamide 96 (35 mg, 0.057 mmol, 1 eq) was added dichloromethane (5 mL) and cooled in an ice bath to 0° C. before triethylsilane (135 μL, 0.86 mmol, 15 eq) and trifluoroacetic acid (44 μL, 0.57 mmol, 10 eq) and the reaction carried out until the starting material was consumed. It was then concentrated in vacuo and dried thoroughly. A solution of hexanes/Ethyl ether (20 mL, 1/1) was added to it and washed thoroughly via triturating (3×10 mL). Sonication was used to aid this washing. The residue was filtered on a sintered funnel and air dried thoroughly. An off white solid 97 was obtained.

¹H NMR (300 MHz) CDCl₃ δ: 9.12 (d, J₁=3.6 Hz, 1H), 8.42 (d, J=8.7 Hz, 1H), 8.27 (s, 1H), 7.91 (t, J =8.9 Hz, 1H), 7.75 -7.50 (m, 1H), 7.02 (dd, 1H), 5.03 (d, J=15.6 Hz, 1H), 4.79 (d, J=18 Hz, 1H), 4.68 (d, J=15.6 Hz, 1H), 4.47 (d, J =18 Hz, 1H), 3.34 (s, 3H), 3.10 (s, 3H). ¹⁹ F NMR (300 MHz) CDCl₃ δ: -68.91, -75.54, -76.34 MS: 417.14 (M+1).

### Example 30

### Preparation of N-(7-(2-cyano-4-fluorobenzyl)-9-hydroxy-8-oxo-7,8-dihydro-6H-pyrrolo[3,4-g]quinolin-5-yl)-N-methylmethanesulfonamide and N-(7-(2-cyano-4-fluorobenzyl)-9-(4-methoxybenzyloxy)-8-oxo-7,8-dihydro-6H-pyrrolo[3,4-g]quinolin-5-yl)-N-methylmethanesulfonamide

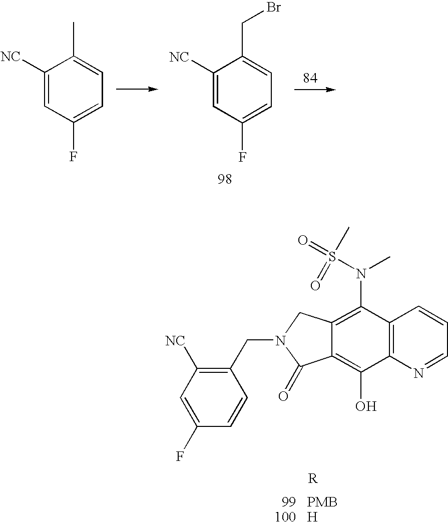

In procedures similar to those used to obtain 95 of example 29, compound 98 was prepared.

¹H NMR (300 MHz) CDCl₃ δ: partial 4.63 (s, 2H). ¹⁹F NMR (300 MHz) CDCl₃ δ: -77.89. MS: 399.09 (M+1).

In procedures similar to those exemplified above, compound 99 was prepared.

¹H NMR (300 MHz) CDCl₃ δ: partial 5.78 (d, J=1.8 Hz, 2H), 5.03 (AB, J=3.9 Hz, 2H), 4.75 (d, J=18 Hz, 1H), 4.53 (d, J=15.6 Hz, 1H). MS: 561.04 (M+1).

In procedures similar to those exemplified above, compound 100 was prepared.

¹H NMR (300 MHz) CDCl₃ δ: partial 4.99 (s, 2H), 3.53 (s, 3H), 3.11 (s, 3H). ¹⁹ F NMR (300 MHz) CDCl₃ δ: -76.26, -111.14. MS: 441.14 (M+1).

### Example 31

### Preparation of N-(7-(2-amino-4-fluorobenzyl)-9-hydroxy-8-oxo-7,8-dihydro-6H-pyrrolo[3,4-g]quinolin-5-yl)-N-methylmethanesulfonamide

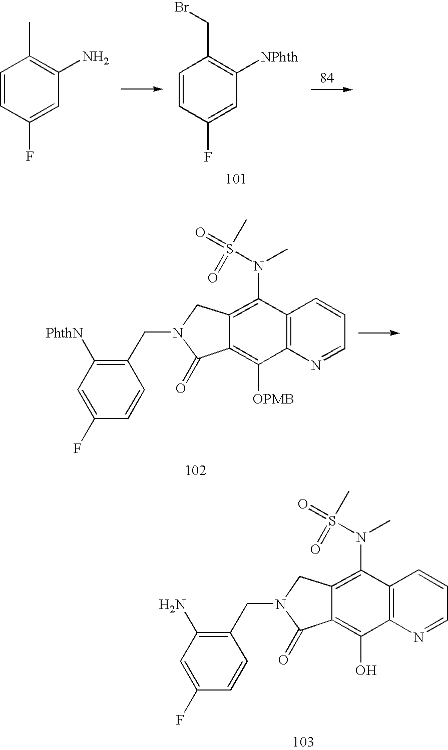

Using standard precedence, aniline was protected as its phthalamido toluene and brominated to furnish 101 in similar fashion as exemplified above.

¹H NMR (300 MHz) CDCl₃ δ: partial 4.42 (s, 2H).

In procedures similar to those exemplified above, compound 103 was prepared.

¹H NMR (300 MHz) CDCl₃ δ: partial 5.34 (d, J=4.2 Hz, 2H), 5.17 (d, J=14.7 Hz, 1H) 4.51 (d, J=16.8 Hz, 1H), 4.42 (d, J=14.7 Hz, 1H), 4.23 (d, J=14.7 Hz, 1H). MS: 680.94 (M+1).

### Example 32

### Preparation of N-(7-(4-fluoro-2-(methylsulfonyl)benzyl)-9-hydroxy-8-oxo-7,8-dihydro-6H-pyrrolo[3,4-g]quinolin-5-yl)-N- methylmethanesulfonamide and N-(7-(4-fluoro-2-(methylsulfonyl)benzyl)-9-(4-methoxybenzyloxy)-8-oxo-7,8-dihydro-6H-pyrrolo[3,4-g]quinolin-5-yl)-N-methylmethanesulfonamide

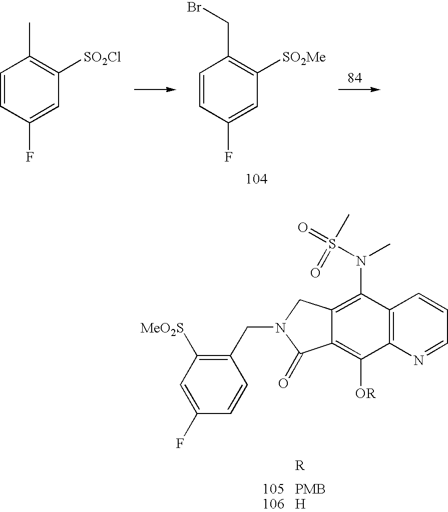

Following the published method of Grunewald et al. (J. Med. Chem. 1999, 42, 3220) for the conversion of sulfonyl chlorides to the corresponding alkylsulfonates, to 800 mg, 3.8 mmol of the commercially available sulfonyl chloride in 20 mL THF at 0° C. was added 0.5 mL, ca. 2.2 eq of hydrazine. After 16h, the reaction solvent was removed to give a white solid. At this time, the reaction was taken up in 10 mL EtOH, and excess sodium acetate (10 eq) and methyl iodide (5 eq) were added. The reaction was then heated to reflux for 16h. At this time, the reaction was concentrated and the residue chromatographed on silica gel (4:1 hexanes/EtOAc) to give 0.4 g of (73%) pure product.

The 0.4 g of intermediate sulfone thus obtained was treated to bromination conditions disclosed in WO 03/086319 to give 60 mg of 104 (10% yield) after column chromatography in 4:1 hexanes/EtOAc. ¹H NMR (300MHz, CDCl₃) shows diagnostic peaks at δ 5.05 (s, 2H) and 3.30 (s, 3H) ppm, MS=269.1 (M+H).

To a flask containing lactam 84 from example 25 (35mg, 0.082 mmol, 1 eq) was added DMF (0.5 mL, 0.1 M). At 0° C., sodium hydride (4 mg, 1.3 eq, 60% mineral oil) was added. The reaction was allowed to stir for 5 minutes before 104 (33 mg, 0.13 mmol, 1.5 eq) was added. TLC was used to indicate when the reaction was complete. The reaction was quenched with saturated NH₄Cl and dissolved in EtOAc (10 mL). It was washed with water (2×5 mL) and brine (10 mL). The organic layer was dried over Na₂SO₄, filtered and concentrated in vacuo. Flash column chromatography was used to purify the product using hexanes/Ethyl acetate (1/4) as eluent. Sulfonate 105 was obtained as an off white solid (35 mg, 70% yield).

¹H NMR (300 MHz) CDCl₃ δ: 9.05 (d, J₁=3.9 Hz, 1H), 8.33 (d, J=8.7 Hz, 1H), 7.65-7.50 (m, 5H), 7.27-7.23 (m, 1H), 6.89 (d, J=8.7 Hz), 5.78 (d, J=1.8 Hz, 2H), 5.22 (s, 2H), 4.63 (d, J=17.1 Hz, 1H), 4.45 (d, J=15.9 Hz, 1H), 3.79 (s, 3H), 3.32 (s, 3H), 3.22 (s, 3H), 3.12 (s, 3H). ¹⁹F NMR (300 MHz) CDCl₃ δ: -110.80. MS: 614.11(M+1).

Into a flask containing sulfonate 105 (35 mg, 0.057 mmol, 1 eq) was added dichloromethane (5 mL) and cooled in an ice bath to 0° C. before triethylsilane (135 μL, 0.86 mmol, 15 eq) and trifluoroacetic acid (44μL, 0.57 mmol, 10 eq) and the reaction carried out until the starting material was consumed. It was then concentrated in vacuo and dried thoroughly. A solution of hexanes/Ethyl ether (20 mL, 1/1) was added to it and washed thoroughly via triturating (3×10 mL). Sonication was used to aid this washing. The residue was filtered on a sintered funnel and air dried thoroughly to obtain phenol 106.

¹H NMR (300 MHz) CDCl₃ δ: 9.00 (d, J₁3.9 Hz, 1H), 8.40 (d, J=8.7 Hz, 1H), 7.83 (d, J=8.9 Hz, 1H), 7.65-7.50 (m, 2H), 7.37-7.30 (m, 1H), 5.26 (s, 2H), 4.73 (d, J=17.1 Hz, 1H), 4.55 (d, J=15.9 Hz, 1H), 3.34 (s, 3H), 3.23 (s, 3H), 3.10 (s, 3H), 2.89 (s, 6H).

### Example 33

### Preparation of 5-fluoro-2-((9-(hydroxy)-5-(N-methylmethylsulfonamido)-8-oxo-6H-pyrrolo[3,4-g]quinolin-7(8H)-yl)methyl)-N,N-dimethylbenzenesulfonamide and

5-fluoro-2-((9-(4-methoxybenzyloxy)-5-(N-methylmethylsulfonamido)-8-oxo-6H-pyrrolo[3,4-g]quinolin-7(8H)-yl)methyl)-N,N-dimethylbenzenesulfonamide

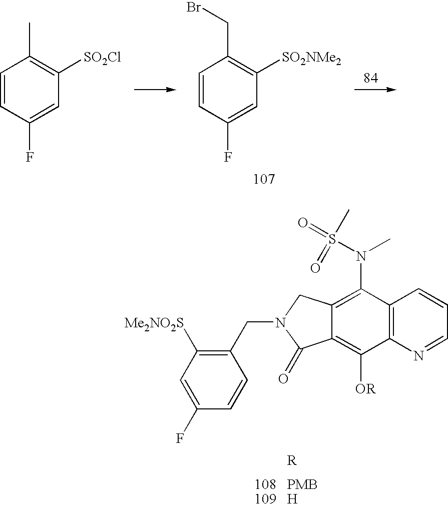

To 2g, 9 mmol of the commercially available sulfonyl chloride in 20 mL THF was added 8 mL, ca. 3 eq of a 2.0 M THF solution of dimethylamine. Then, ca. 5 mg 4-DMAP was added. After 16h, the reaction solvent was removed and the residue chromatographed on silica gel (4:1 hexanes/EtOAc) to give 0.5 g (25%) pure product. MS (M+H) 218.1, ¹H NMR (300MHz, CDCl₃) shows diagnostic peaks at δ 2.85 (s, 6H) and 2.60 (s, 3H) ppm.

The 0.5 g intermediate sulfonamide thus obtained was treated to bromination conditions disclosed in WO 03/086319 to give 50 mg 107 (10% yield) after column chromatography in 4:1 hexanes/EtOAc. ¹H NMR (300MHz, CDCl₃) shows diagnostic peaks at δ 4.85 (s, 2H) and 2.90 (s, 6H) ppm, MS=296.0 (M+H).

To a flask containing lactam 84 from example 25 (33mg, 0.075 mmol, 1 eq) was added DMF (1.2 mL, 0.1 M). At 0° C, sodium hydride (4 mg, 1.3 eq, 0.1 eq, 60% mineral oil) was added. The reaction was allowed to stir for 5 minutes 107 (34 mg, 0.12 mmol, 1.5 eq) was added. TLC was used to indicate when the reaction was complete. The reaction was quenched with saturated NH₄Cl and dissolved in EtOAc (10 mL). It was washed with water (2+5 mL) and brine (10 mL). The organic layer was dried over Na₂SO₄, filtered and concentrated in vacuo. Flash column chromatography was used to purify the product using hexanes/Ethyl acetate (1/4) as eluent. Sulfonamide 108 was obtained as an off white solid (20 mg, 43 % yield).

¹H NMR (300 MHz) CDCl₃ δ: 9.05 (d, J₁=3.9 Hz, 1H), 8.33 (d, J=8.7 Hz, 1H), 7.65-7.50 (m, 5H), 7.27-7.23 (m, 1H), 6.89 (d, J=8.7 Hz), 5.78 (d, J=1.8 Hz, 2H), 5.22 (s, 2H), 4.63 (d, J=17.1 Hz, 1H), 4.45 (d, J=15.9 Hz, 1H), 3.80 (s, 3H), 3.32 (s, 3H), 3.13 (s, 3H), 2.89 (s, 6H). ¹⁹F NMR (300 MHz) CDCl₃ δ: -111.92. MS: 643.11(M+1).

Into a flask containing sulfonamide 108 (20 mg, 0.031 mmol, 1 eq) was added dichloromethane (5 mL) and cooled in an ice bath to 0° C. before triethylsilane (100 μL, 0.63 mmol, 20 eq) and trifluoroacetic acid (36 μL, 0.47 mmol, 15 eq) and the reaction carried out until the starting material was consumed. It was then concentrated in vacuo and dried thoroughly. A solution of hexanes/Ethyl ether (20 mL, 1/1) was added to it and washed thoroughly via triturating (3×10 mL). Sonication was used to aid this washing. The residue was filtered on a sintered funnel and air dried thoroughly. A light yellow solid 109 (7 mg, 0.62 mmol, 93 %) was obtained.

¹H NMR (300 MHz) CDCl₃ δ: 9.02 (d, J₁=3.9 Hz, 1H), 8.40 (d, J=8.7 Hz, 1H), 7.83 (d, J=8.9 Hz, 1H), 7.65-7.50 (m, 2H), 7.37-7.30 (m, 1H), 5.21 (s, 2H), 4.73 (d, J=17.1 Hz, 1H), 4.55 (d, J=15.9 Hz, 1H), 3.34 (s, 3H), 3.23 (s, 3H), 3.10 (s, 3H), 2.89 (s, 6H). 19 F NMR (300 MHz) CDCl₃ δ: -76.27, -111.49. MS: 523.0 (M+1).

### Example 34

### Preparation of ethyl 7-(4-fluorobenzyl)-9-(hydroxy)-8-oxo-7,8-dihydro-6H-pyrrolo[3,4-g]quinolin-5-yl(methyl)carbamate and ethyl 7-(4-fluorobenzyl)-9-(4-methoxybenzyloxy)-8-oxo-7,8-dihydro-6H-pyrrolo[3,4-g]quinolin-5-yl(methyl)carbamate

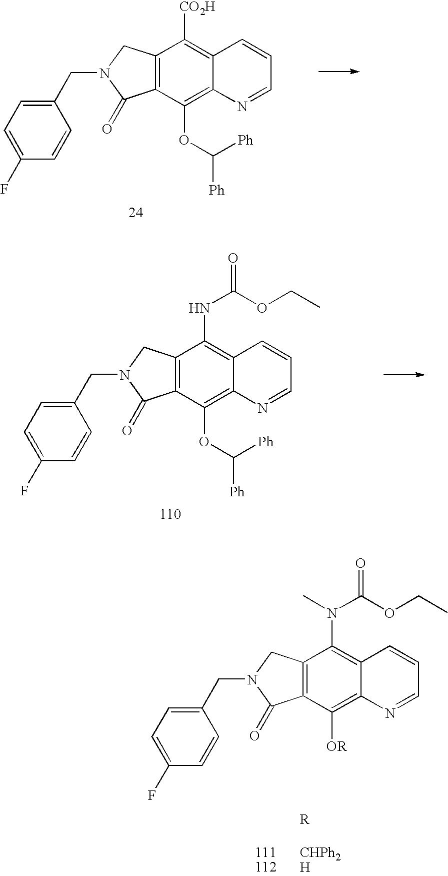

To the suspension of 0.5g (0.965 mmol) of acid 24 from example 1 in 5 mL of anhydrous Toluene was added 0.27 mL (1.93 mmol) of triethylamine and 0.23 mL (1.06 mmol) of diphenylphosphorylazide. After which the reaction was stirred at room temperature and flashed with nitrogen three times. To this mixture, a 5 mL of ethanol was then added and the reaction was heated to 65° C. for 2 days. The reaction was cooled to room temperature. It was diluted with ethyl acetate and washed with 10% citric acid, Sat'd NaHCO₃ and brine. The residue was then dried over Magnesium Sulfate and concentrated in vacuum. The crude residue was purified by flash chromatography (10% to 30% ethyl acetate in hexane) to afford 300 mg (0.534 mmol, 55% yield) of 110. 300 MHz ¹H NMR (CDCl₃) δ (ppm): 9.0(d, 1H); 8.2 (d, 1H); 8.0 (s, 1H); 7.7 (m, 4H); 7.4 (m, 1H); 7.23 (m, 6H); 7.1 (t, 2H); 7.0 (t, 2H); 6.5 (s, 1H, NH); 4.8 (s, 2H); 4.2 (s, 2H); 4.1 (m, 2H); 1.2 (t, 3H). m/z=562 (M+1).

A 300 mg (0.534 mmol) of 110 was dissolved in 5 mL of DMF and flashed with nitrogen. It was cooled to 0° C. To this was added 15 mg (0.587 mmol) NaH and the reaction was stirred at 0° C. for 5 minutes. It was then added 166 pL (2.67 mmol) of iodomethane. After stirring at 0° C. for 20 min, the reaction was quenched with the addition of 20mL sat'd NaHCO₃ and extracted with ethyl acetate. The organic layer was then washed once with 2% LiCl, and once with brine. The organic was then dried over Mg₂SO₄ and concentrated in vacuum. The crude residue was then purified on silica gel (20% to 50% ethyl acetate in hexane ) to provide 111(254 mg, 83%). 300 MHz ¹H NMR (CDCl₃) δppm): 9.1 (d, 1H); 8.1 (d, 1H); 8.0 (d, 1H); 7.8 (m, 4H); 7.5 (m, 1H); 7.3 (m, 7H); 7.12 (t, 2H); 7.03 (t, 2H); 4.7-4.9 (m, 2H); 4.1 (m, 2H); 3.9 (m, 2H); 3.20 (s, 3H); 0.8 (m, 3H). m/z=576 (M+1).

Compound 111 (31 mg, 54 μmol) was dissolved in 100 μL of DCM and treated with TFA (100 μL) and triethylsilane (200 μL). After stirring for 30 minutes at room temperature, the reaction mixture was azeotroped with toluene three times. The resulting residue was then purified by reverse-phase prep HPLC to provide 23 mg (81.5% in yield) of 112 as the TFA salt. 300 MHz ¹H NMR (CDCl₃) δ (ppm): 9.1 (d, 1H); 8.3 (d, 1H); 7.7 (m, 1H); 7.3 (t, 2H); 7.1 (t, 2H); 4.8 (s, 2H); 4.3 (s, 2H); 4.0 (m, 2H); 3.2 (s, 3H); 0.9 (m, 3H). ^{·}F NMR (CDCl₃) δ (ppm): -76.3; -114.2. m/z=410 (M+1).

### Example 35

### Preparation of ethyl 7-(4-fluorobenzyl)-9-hydroxy-6-methyl-8-oxo-7,8-dihydro-6H-pyrrolo[3,4-g]quinolin-5-yl(methyl)carbamate and ethyl 9-(benzhydryloxy)-7-(4-fluorobenzyl)-6-methyl-8-oxo-7,8-dihydro-6H-pyrrolo[3,4-g]quinolin-5-yl(methyl)carbamate

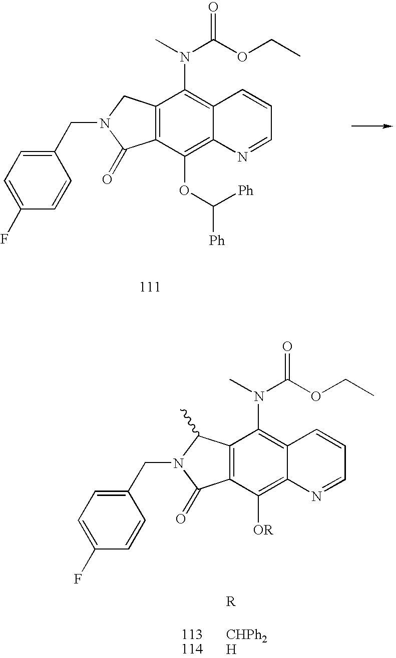

Compound 111 from Example 34 (41.6 mg, 72 μmol) was dissolved in 1 mL of DMF and cooled to 0° C. Iodomethane (22 μL, 360 μmol) and sodium hydride (5.4 mg, 217 μmol) were then added and the reaction was allowed to stir at 0° C. for 120 minutes. (The reaction was monitored by LC/MS; TLC did not show the difference between SM and product.). The reaction was quenched with 10 mL sat'd NaHCO₃ and extracted with ethyl acetate. The organic layer was then washed once with 2% LiCl, and once with 10% citric acid and brine. The organic was then dried over Na₂SO₄ and concentrated in vacuum to give crude product 113.

The compound 113 was dissolved in 100 μL of DCM and treated with TFA (100 μL) and triethylsilane (200 μL). After stirring for 30 minutes at room temperature, the reaction mixture was azeotroped with toluene three times. The resulting residue was then purified by reverse-phase prep HPLC to provide 17 mg (44 % in yield) of 114 as the TFA salt. 300 MHz ¹H NMR (CDCl₃) 67 (ppm): 9.1 (d, 1H); 8.2 (d, 1H); 7.7 (m, 1H); 7.3 (t, 2H); 7.1 (t, 2H); 5.3-5.2 (m, 1H); 4.5-3.8 (m, 4H); 3.3 (s, 3H); 3.2 (s, 3H); 1.5 & 1.0 (m, 3H). m/z=424 (M+1).

### Example 36

### Preparation of ethyl 7-(4-fluorobenzyl)-9-hydroxy-6,6-dimethyl-8-oxo-7,8-dihydro-6H-pyrrolo[3,4-g]quinolin-5-yl(methyl)carbamate

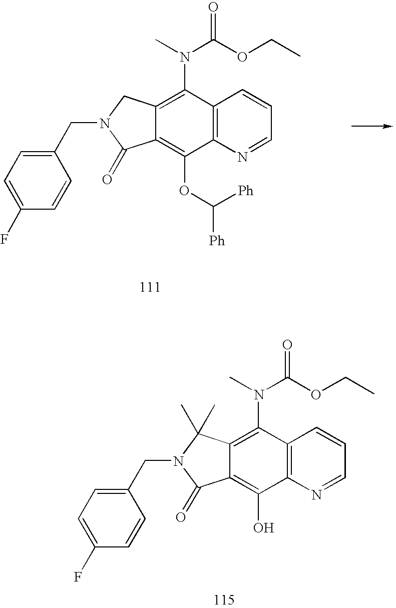

Compound 111 from example 32 (43 mg, 75 μmol) was dissolved in 1 mL of THF and flashed with nitrogen, it was added iodomethane (47 mg, 750 μmol) and cooled to 0° C. followed by LiHMDS (1M in hexane, 374 μL, 374 μmol). The reaction was stirred and allowed to warm to room temperature overnight. The reaction was quenched with 10 mL sat'd NaHCO₃ and extracted with ethyl acetate. The organic layer was then washed once with 10% citric acid and brine and concentrated in vacuum to give crude product. The resulting residue was then purified by reverse-phase prep HPLC to provide 11 mg (27 % in yield) of 115 as the TFA salt. 300 MHz ¹H NMR (CDCl₃) δ (ppm): 9.2 (d, 1H); 8.2 (d, 1H); 7.8 (m, 1H); 7.4 (t, 2H); 7.0 (t, 2H); 4.8 (s, 2H); 4.0 (m, 2H); 3.3 (s, 3H); 1.5 (s, 3H); 1.4 (s, 3H); 1.0(m, 3H).). ¹⁹F NMR (CDCl₃) δ (ppm): -76.4; -115.0. m/z=438 (M+1).

### Example 37

### Preparation of 5-amino-7-(4-fluorobenzyl)-9-hydroxy-6H-pyrrolo[3,4-g]quinoline-6,8(7H)-dione and other intermediates useful for preparing compounds of the invention

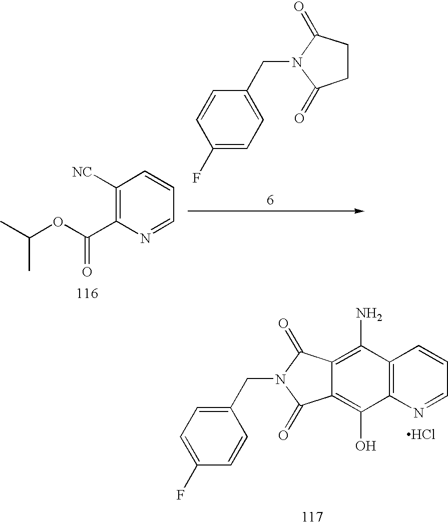

To 116 (Dunn, A. D.; Mills, M. J.; Henry, W. Org. Prep. Proced. Int. 1982, 14, 396-399; 10 g, 52.6 mmol, 1 eq) and imide 1 (11.98 gm, 57.88 mmol, 1.1 eq) was added THF (170 mL, 0.3 M). The flask was cooled to 0° C. and NaHMDS (131 mL, 131 mmol, 2.5 eq, 0.1 M THF) diluted in THF (90 mL, overall 0.2 M) was added dropwise via an addition funnel over 10 min. The ice-bath was removed and the reaction allowed to stir for an hour. The flask was cooled to 0° C. and slowly quenched with HCl (6 N, 55 mL) before being concentrated in vacuo to a red paste. Ethyl ether (400 mL) was added to the flask along with water (50 mL). It was allowed to stir vigorously for 15 min before being filtered over a sintered funnel. The red residue was washed with water (2×15 mL) and ether (3×50 mL) and allowed to air dry in a vacuum oven at 65° C. for several hours to afford 117 (16.7 gm, 95%) of a red powder.

¹H NMR (DMSO-d₆, 300 MHz) 8.96 (d, 2H), 7.77 (m, 1H), 7.29 (m, 2H), 7.12 (m, 2H), 6.63 (br s, 2H), 4.65 (s, 2H); MS (ESI) m/z 338 [M +H]⁺.

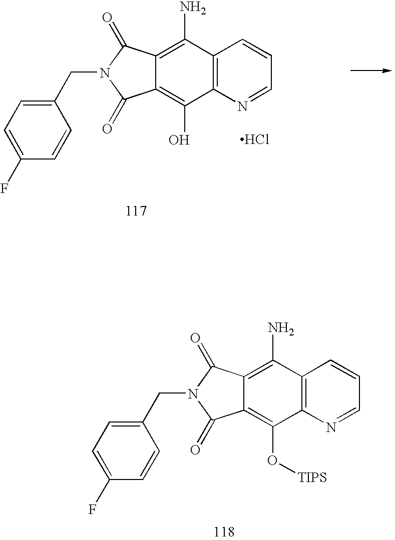

117 (8.85 g, 26.2 mmol) in DMF (260 mL, 0.1 M) was treated with TEA (10.97 mL, 78.7 mmol), DMAP (321 mg, 2.62 mmol), and TIPSCI (11.68 mL, 55.1 mmol) under Ar and stirred at room temperature for 3 h. The reaction mixture was quenched with 1 N HCl (100 mL) and extracted with EtOAc-(2 ×250 mL). The organic layer was washed with 1 N HCl (100 mL), saturated NaHCO₃ (2×100 mL), and brine (2×100 mL). The organic layer was dried (Na₂SO₄), concentrated, and the remaining solid was filtered and washed with hexanes (5×100 mL) to afford 118 (10.81 g, 85%) as a yellow solid: R_{f}=0.63 (50% EtOAc-hexanes): ¹H NMR (CDCl₃, 300 MHz) 8.87 (d, 1H), 8.19 (d, 1H), 7.47 (m, 1H), 7.40 (m, 2H), 6.95 (t, 2H), 5.70 (br s, 2H), 4.77 (s, 2H), 1.45 (m, 3H), 1.07 (d, 18H); MS (ESI) m/z494 [M+H]⁺.

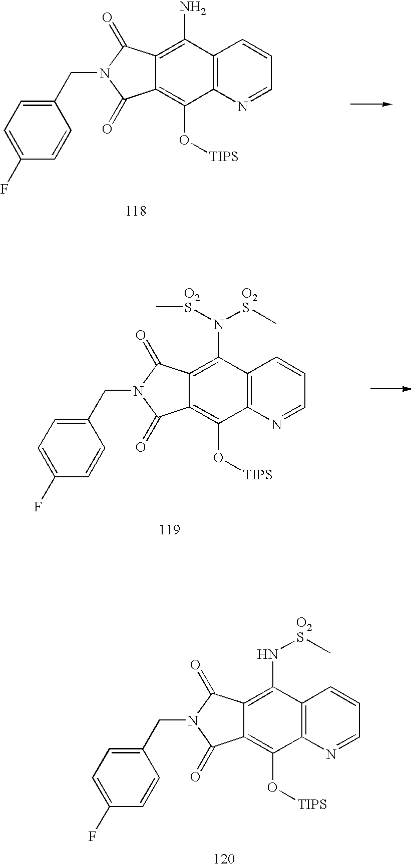

A mixture of 11.00 g (20.26 mmol) of 118 and 32 mL (229.6 mmol) of NEt₃ in 550 mL of dichloromethane was stirred at −10° C. as a solution of 8.7 mL (112.4 mmol) of methanesulfonyl chloride in 78 mL of dichloromethane was added over 45 min. After addition, the mixture was stirred for 1 h at −10° C. and treated with 200 mL of saturated aq. NH₄Cl. After the mixture was diluted with 200 mL of water, the product was extracted with ethyl acetate (3.3 L×1; 500 mL×2) and the extracts were washed with water (500 mL×1), dried (MgSO₄), and concentrated to ˜300 mL. The concentrated solution was dried (MgSO₄) again and filtered through celite before further concentration. The residue was dried in vacuum to give 16.024 g of the crude bis-mesylate 119: R_{f}=0.30 (THF/hexanes=½); ¹H NMR (CDCl₃, 300 MHz) 8.98 (dd, 1H, J=4.2 and 1.5 Hz), 8.53 (dd, 1H, J=8.4 and 1.5 Hz), 7.74 (dd, 1H, J=8.4 and 4.2 Hz), 7.47 (appt dd, 2H, J=8.7 and 5.4 Hz), 7.02 (appt t, 2H, J=8.7 Hz), 4.86 (s, 2H), 3.56 (s, 6H), 1.55 (m, 1H, J=7.5 Hz) 1.13 (d, 18H, J=7.5 Hz); MS (ESI) m/z 650 [M+H]⁺.

A solution of 14.58 g of the crude 119 in 50 mL of THF was stirred in 0° C. as 41 mL (41 mmol) of 1.0 M solution of potassium t-butoxide in THF was added over ˜10 min. After 5 min, the solution was diluted with a solution of 2.4 mL of acetic acid in 300 mL of water and the product was extracted with ethyl acetate (300 mL×2). The extracts were washed with water (300 mL×1), dried (Mg SO₄), and concentrated.

The residue was dissolved in ˜100 mL of dichloromethane with warm heating and filtered. The filtrate was passed through 50 g SiO₂ column, which was washed with 450 mL of 0.05% NEt₃ in dichloromethane. The column was eluted with of 0.05% NEt₃ in ethyl acetate/hexane (½) until the product was eluted. The eluent was concentrated and dried to afford 120 (8.9894 g, 77% from 105) as a tan-yellow solid: R_{f}=0.40 (THF/hexanes=½); ¹H NMR (CDCl₃, 300 MHz) 8.95 (s, 1H), 8.92-8.94 (m,1H), 7.62-7.68 (m, 2H), 7.46 (appt dd, 2H, J=8.7 and 5.1 Hz), 7.02 (appt t, 2H, J=8.7 Hz), 4.84 (s, 2H), 3.02 (s, 3H), 1.54 (m, 1H, J=7.2 Hz) 1.12 (d, 18H, J=7.2 Hz); MS (ESI) m/z 570 [M−H]⁻.
Procedure for the Conversion of 120 to 29 via 121:

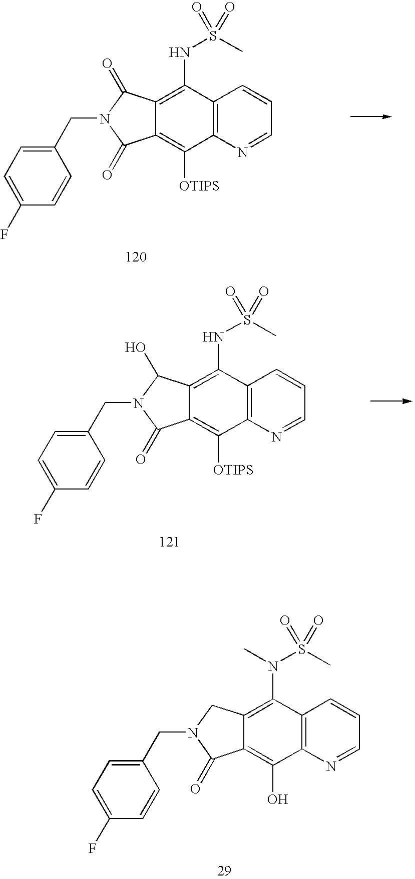

120 (11.2 g, 19.41 mmol) in THF-water (10:1, 260 mL, 0.1 M) was cooled to 0° C., treated with LiBH₄ (2.0 M in THF, 78 mL, 155.3 mmol), and stirred for 15 min. The reaction mixture was warmed to room temperature and stirred for 12 h. The reaction mixture was treated with 5% citric acid (100 mL), filtered, and THF was removed in vacuo. The resulting solution was diluted with EtOAc (250 mL), washed with saturated NaHCO₃ (50 mL) and brine (50 mL). The solution was dried (Na₂SO₄) and concentrated to afford crude 121 (9.84 g, 88% recovery) as a yellow solid (mixture of diastereomers at CHOH, data for major isomer): R_{f}=0.33 (50% EtOAc-hexanes); ¹H NMR (CDCl₃, 300 MHz) 8.80 (d, 1H), 8.51 (d, 1H), 7.48 (m, 1H), 7.24 (m, 2H), 6.93 (m, 2H), 6.77 (br s, 1H), 4.85 (d,1H), 4.25 (d, 1H), 3.02 (s, 3H), 1.48 (m, 3H), 1.10 (d, 18H); MS (ESI) m/z 575 [M+H]⁺.

121 (9.75 g, 16.99 mmol) in DMF (170 mL, 0.1 M) was cooled to 0° C., treated with NaH (60% dispersion in oil, 1.70 g, 42.48 mmol), and stirred for 5 min. The reaction mixture was treated with MeI (2.65 mL, 42.48 mmol) and stirred for 45 min. The reaction mixture was quenched with water (25 mL) and extracted into EtOAc (500 mL). The organic layer was washed with brine (3×100 mL), dried (Na₂SO₄), and concentrated to afford crude O,N-dimethylated derivative 122 as a yellow solid (10.22 g, mixture of stereomers at MeO, data for major isomer): R_{f}=0.47 (50% EtOAc-hexanes); ¹H NMR (CDCl₃, 300 MHz) 8.85 (d, 1H), 8.27 (d, 1H), 7.55 (m, 1H), 7.30 (m, 2H), 6.97 (m, 2H), 5.93 (s, 1H), 5.05 (d, 1H), 4.23 (d, 1H), 3.31 (s, 3H), 3.01 (s, 3H), 2.90 (s, 3H), 1.50 (m, 3H), 1.09 (d, 18H); MS (ESI) m/z 602 [M+H]⁺.

Crude O,N-dimethylated 122 (10.22 g, 16.99 mmol) in CH₂Cl₂ (170 mL, 0.1 M) was treated with TES (16.28 mL, 101.94 mmol) and TFA (5.24 mL, 67.96 mmol) and stirred for 30 min at room temperature. TMSOTf (12.30 mL, 67.96 mmol) was added and stirred for 2 h. The solvent was removed in vacuo and the resulting residue was dissolved in DMF (22 mL) and purified by reverse-phase preparative HPLC (5-95% MeCN-H₂O gradient) to provide 29, (1.75 g, 25%, 2 steps) as a white solid: ¹H NMR (CDCl₃, 300 MHz) 8.92 (d, 1H), 8.23 (d, 1H), 7.59 (m, 1H), 7.29 (m, 2H), 6.99 (m, 2H), 4.90 (d, 1H), 4.67 (d, 1H), 4.53 (d, 1H), 4.32 (d, 1H), 3.27 (s, 3H), 3.01 (s, 3H); ¹⁹F NMR (CDCl₃, 282 MHz) −114.3; MS (ESI) m/z 416 [M+H]⁺.

120 to 29 via 123 and 124:

Alternatively, a solution of 10.003 mg (17.50 mmol) of the reactant 120 in 75 mL DMF was stirred at 0° C. as powder (˜325 mesh) of K₂CO₃ (3.630 g, 26.26 mmol) followed by 1.65 mL (26.50 mmol) of methyl iodide were added. After stirring for 1 h at 0° C., the mixture was diluted with ethyl acetate (100 mL), treated with 5% aq. citric acid (100 mL) (CO₂ gas was evolved! pH was ˜4.), and transferred to 1 L seperatory funnel with ethyl acetate (400 mL) and water (500 mL). The separated aq. fraction was extracted with ethyl acetate (500 mL×1). The organic fractions were washed with a mixture of aq. NaHCO₃ solution (100 mL) and water (500 mL), followed by water (500 mL×2), dried (MgSO₄), and concentrated. The residue was dissolved in CH₂Cl₂, concentrated, and dried in vacuum for 30 min to give ˜10.623 g (104%) of the crude 123. ¹H and ¹⁹F NMR spectral data were obtained from the purified sample obtained from the separate trial: ¹H NMR (CDCl₃, 300 MHz) δ 8.94 (dd, 1H, J=3.9 and 1.5 Hz), 8.72 (dd, 1H, J=8.4 and 1.5 Hz), 7.68 (dd, 1H, J=8.4 and 4.5 Hz), 7.47 (m, 2H), 7.03 (m, 2H), 4.85 (s, 2H), 3.44 (s, 3H), 3.16 (s, 3H), 1.55 (m, 3H, 7.8 Hz), 1.13 (dd, 18H, J=7.8 and 1.5 Hz); ¹⁹F NMR (CDCl₃, 282 MHz) δ −114.7 (m).

A solution of the crude 123 (˜10.623 g , obtained from 10.003 g of 120) in THF (52.5 mL) was stirred at 0° C. as 17.5 mL (35.0 mmol) of 2.0 M LiBH₄ in THF was added. (H₂ gas was evolved a little bit) The solution was stirred at 0° C. as a solution of 5.0 mL (123.44 mmol) of methanol in THF (20 mL) over 1 h using a syringe drive. After the addition, the solution was further stirred at 0° C. for 1 h and diluted with ethyl acetate (200 mL) before adding 5% aq. citric acid solution (100 mL). After the mixture was transferred to seporatory funnel with ethyl acetate (300 mL) and water (400 mL), the two layers were separated and the aq. layer was extracted with ethyl acetate (500 mL×1). The two organic fractions were washed with water (500 mL×1), dried (MgSO₄), and concentrated. The residue was dissolved in CH₂Cl₂, concentrated, and dried in vacuum for 20 min to give ˜11.1835 g (109%) of the crude diastereomeric mixture (˜9:1) of 124, along with the regioisomer (3-4%) and TIPS deleted impurities (5-10%) . The analytical samples of the two diastereomers were obtained by flash chromatography from a separate trial:

The major (non-polar isomer): R_{f}=0.46 (ethyl acetate/hexanes=1/1); ¹H NMR (CDCl₃, 300 MHz) δ 8.89 (dd, 1H, J=3.9 and 1.5 Hz), 8.46 (dd, 1H, J=8.4 and 1.5 Hz), 7.61 (dd, 1H, J=8.4 and 3.9 Hz), 7.38 (appt dd, 2H, J=8.4 and 5.4 Hz), 7.03 (appt t, 2H, J=8.7 Hz), 5.64 (d, 1H, J=11.1 Hz), 5.14 (d, 1H, J=15.0 Hz), 4.33 (d, 1H, J=15.0 Hz), 3.29 (s, 3H), 3.27 (s, 3H), 3.25 (d, 1H, J=11.1 Hz), 1.56 (m, 3H, 7.4 Hz), 1.15 (d, 18H, J=7.4 Hz); ¹⁹F NMR (CDCl₃, 282 MHz) δ −115.4 (m); MS (ESI) m/z 586 [M−H]⁻.

The minor (polar isomer): R_{f}=, 0.26 (ethyl acetate/hexanes=1/1); ¹H NMR (CDCl₃, 300 MHz) δ 8.90 (dd, 1H, J=4.2 and 1.5 Hz), 8.31 (dd, 1H, J=8.4 and 1.5 Hz), 7.61 (dd, 1H, J=8.4 and 4.2 Hz), 7.31 (appt dd, 2H, J=8.4 and 6.0 Hz), 7.01 (appt t, 2H, J=8.6 Hz), 5.97 (d, 1H, J=9.9 Hz), 4.83 (d, 1H, J=15.0 Hz), 4.40 (d, 1H, J=15.0 Hz), 3.40 (s, 3H), 3.16 (d, 1H, J=9.9 Hz), 3.10 (s, 3H), 1.55 (m, 3H, 7.4 Hz), 1.15 (dd, 18H, J=7.4 and 5.5 Hz); ¹⁹F NMR (CDCl₃, 282 MHz) δ −115.3 (m); MS (ESI) m/z 586 [M−H]⁻.

A solution of the crude 124 (11.1835 g, obtained from 10.003 g of 120) in dichloromethane (75 mL) and Et₃SiH (28.0 mL, 175.3 mmol) was stirred at 0° C. as 75 mL (973.5 mmol) of trifluoroacetic acid was added. (The solution became red.) After the red solution was stirred at rt for 11.5 h, it was cooled at 0° C. and methanol (50 mL) was added. The solution was stirred at rt for 2 h before the solution was concentrated.

The remaining viscous oil (two immiscible oils) was dried in vacuum for 1.5 h. The remaining residue was dissolved in methanol (50 mL), diluted with ethyl ether (50 mL), and the solution was sonicated for 5 min while the solids were precipitated. The resulting mixture was stirred at 0° C. for 1 h and filtered through medium glass filter, washed with small amount (˜7 mL) of methanol followed by ethyl ether (˜7 mL). The obtained solids were dried in vacuo for 10 min to afford 5.51 g of 29 (75.8%).

121 to 124:

Crude 121 (9.84 g, 17.15 mmol) in DMF (85 mL, 0.2 M) was treated with K₂CO₃ (3.56 g, 25.73 mmol) and stirred for 5 min. The reaction mixture was treated with MeI (1.6 mL, 25.73 mmol) and stirred for 1 h. The reaction mixture was quenched with 5% citric acid (50 mL) and extracted into EtOAc (250 mL). The organic layer was washed with saturated NaHCO₃ (2×50 mL) and brine (1×50 mL). The solution was dried (Na₂SO₄) and concentrated to afford crude 124 (10.22 g, >100% recovery) as a yellow solid.

### EXAMPLE 38

### Preparation of N-(7-(4-fluorobenzyl)-9-hydroxy-8-oxo-7,8-dihydro-6H-pyrrolo[3,4-g]quinolin-5-yl)methanesulfonamide

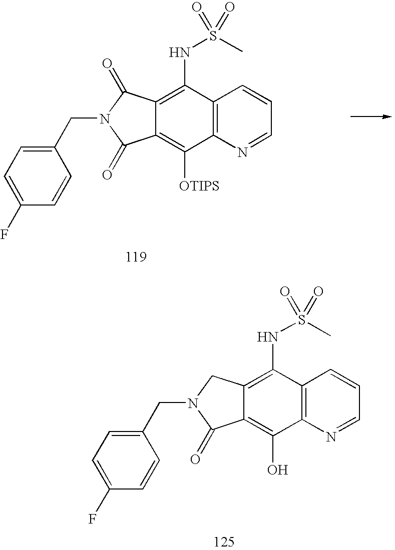

119 from example 37 (405 mg, 0.709 mmol) in THF/water (7:1, 35 mL, 0.02 M) was cooled to 0° C., treated with LiBH₄ (175 mg, 8.05 mmol), and stirred for 15 min. The reaction mixture was warmed to room temperature and stirred for 1 h. The reaction mixture was treated with saturated 1 N HCl (25 mL) and solvent removed in vacuo. The resulting residue was dissolved in Et₂O (200 mL) and the organic layer was washed with water (100 mL), dried (Na₂SO₄), and concentrated to afford a crude mixture of hydroxy-lactam (aminal).

Hydroxy-lactam in CH₂Cl₂ (5 mL, 0.14 M) was treated with triethylsilane (1.5 mL, 9.39 mmol) and TFA (320 μL, 4.15 mmol) at room temperature. After 15 min, the solvent was removed in vacuo and residual TFA was removed via toluene azeotrope (2×5 mL). Tritiration with hexanes/CH₂Cl₂ afforded 125 (142 mg, 50% over 2 steps) as a brown solid (data for TFA salt): ¹H NMR (CD₃OD, 300 MHz) 8.94 (s, 1H), 8.79 (d, 1H), 7.80 (m,1H), 7.36 (m, 2H), 7.05 (m, 2H), 4.76 (s, 2H), 4.62 (s, 2H), 3.01 (s, 3H); MS (ESI) m/z 402 [M+H]⁺.

### EXAMPLE 39

### Preparation of 2-chloro-7-(4-fluorobenzyl)-5,9-dihydroxy-6H-pyrrolo[3,4-g]quinoline-6,8(7H)-dione

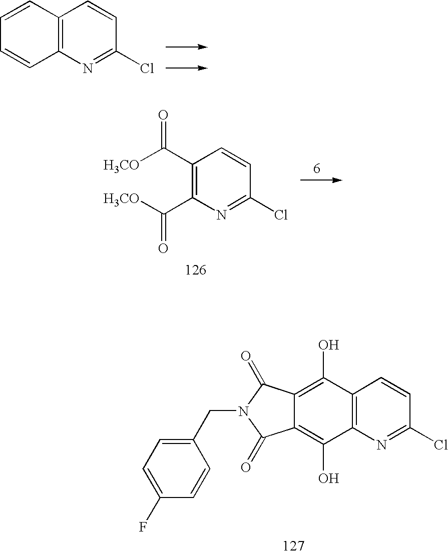

Following the literature procedure of M.-D. Le Bas et al. (Synthesis 2001, 16, p. 2495), 100 mL CCl₄ was mixed with 250 mL of an aqueous NaOCl solution. To this mixture was added 40 mg of RuO₂, followed by 3g 2-chloroquinoline dissolved in 50 mL CCl₄. Additional 30 mL portions of bleach were added at 2, 4, and 6 h. After 24 h, the aqueous layer was collected and acidified to pH 1 with 3N HCl. The aqueous layer was then extracted with ethyl acetate, dried over Na₂SO₄ and volatiles removed by evaporation to give the 1.7 g product as a yellow resin, (48% yield). ¹H NMR and MS data matched that reported in the literature.

The diacid, 1.7 g (8.5 mmol) was taken up in 100 mL of a 1:1 mixture of toluene/methanol. To this solution was added dropwise, at room temperature, a 2M solution of TMS diazomethane until a persistent deep yellow color was observed. Volatiles were removed to provide the product 126 as a light yellow solid in quantitative yield.

To 50 mg of 126 in 2 mL THF was added 1.2 eq of 6. The reaction was cooled to 0° C. and NaHMDS (3 eq, 0.6 mmol, 0.6 mL of a 1M THF solution) was added dropwise. A deep red color was observed immediately upon addition of the NaHMDS. The reaction was allowed to continue stirring overnight. LC/MS analysis showed reaction had gone completely to product at this time. The product was precipitated from the reaction mixture by addition of 6N HCl. Approximately 50 mg (62%) of unpurified product was obtained as a yellow solid which was further refined by trituration with diethyl ether to provide 2 mg highly pure bis-phenol product 127. ¹H NMR (300 MHz, d₆-DMSO) shows diagnostic peaks at δ 8.70 (d, 1H), 7.82 (d, 1H) and 4.65 (s, 2H) ppm, MS=373.1 (M+H).

### EXAMPLE 40

### Preparation of 2-amino-7-(4-fluorobenzyl)-9-hydroxy-5-(2-(trimethylsilyl)ethoxy)-6H-pyrrolo[3,4-g]quinoline-6,8(7H)-dione

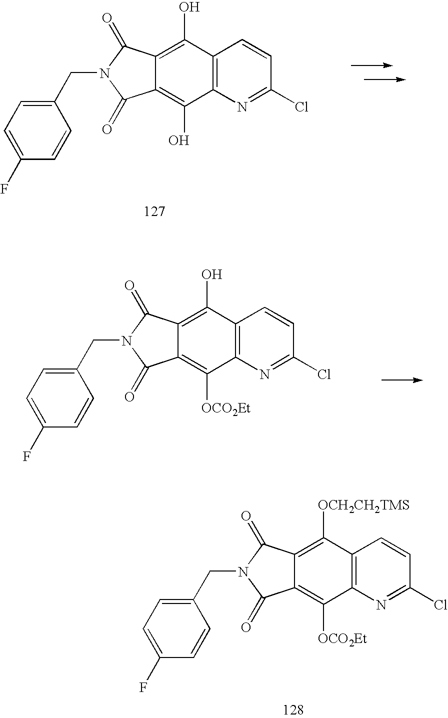

Using a sequence of phenol protecting chemistry procedures that have been described herein, 1.4 g (3.7 mmol) of bis-phenol 127 was converted to the 5-trimethylsilylethyl ether, 8-ethyl carbonate compound 128, obtained in 24% overall yield across the three chemical steps performed after purification via silica gel chromatography with 4:1 hexanes/ethyl acetate as eluent. ¹H NMR (300 MHz, CDCl₃) shows diagnostic peaks at δ 8.65 (d, 1H), 7.60 (d, 1H), 4.82 (s, 2H), and 0.02 (9H, s) ppm, MS=545.1 (M+H).

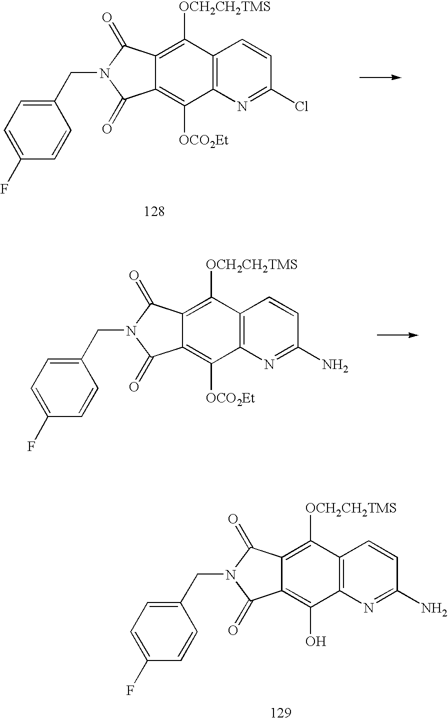

Following the procedure reported by Buchwald et al. (Org. Lett. 2001, 3, 3417.) for the use of LiHMDS as an ammonia equivalent in the Pd₂(dba)₃/2-dicylohexyl phosphinobiphenyl catalyzed amination of aryl halides, 50 mg (0.9mmol, 1 eq) of starting 2-chloroquinoline 128 was dissolved in 1 mL of THF, which was then purged with argon for a period of 5 minutes. Then, Pd₂(dba)₃ (4.6 mg, 5.0 pmol) and 2-dicyclohexylphosphinobiphenyl ligand (4.2 mg, 12 μmol) were added. LiHMDS (1.2 mL, 1 M solution in THF, 1.2 mmol) was then added via syringe.

The reaction was then placed in an oil bath at 65° C. under an atmosphere of argon for 16 h. After cooling of the reaction mixture to room temperature, aqueous HCl (5 mL, 1 M) was added, and the mixture was stirred at room temperature for 5 min. The solution was then neutralized by the addition of aqueous NaOH. The aqueous phase was extracted with CH₂Cl₂ three times. The combined organic layers were combined, dried over Na₂SO₄, filtered, and concentrated in vacuo. The product was purified by flash chromatography on silica gel, to give ca. 10 mg (ca. 20% yield) of intermediate amine product as a yellow film. This residue was subjected to carbonate hydrolysis in a 5:1 solution of THF/1 M aq. NaOH to give, after neutralization of the reaction mixture with dilute HCl followed by extraction with ether and concentration of the organic layer under vacuum, 5 mg (51% yield) 2-aminoquinoline product 129.

¹H NMR (300 MHz, CD₃CN) shows diagnostic peaks at δ 8.22 (d, 1H), 6.75 (d, 1H), 4.78 (s, 2H), 0.04 (9H, s) ppm, MS=454.1 (M+H).

### EXAMPLE 41

### Preparation of Potassium 7-(4-fluorobenzyl)-5-(N-methylmethylsulfonamido)-8-oxo-7,8-dihydro-6H-pyrrolo[3,4-g]quinolin-9-olate

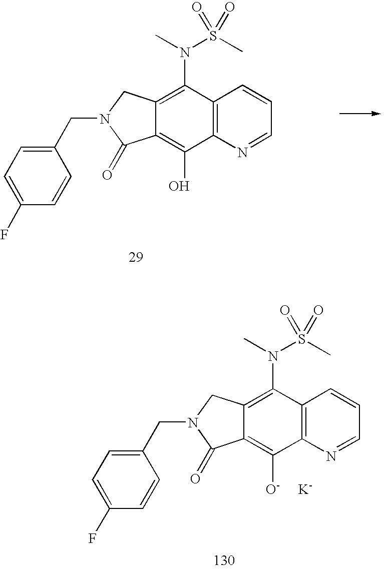

To 29 from example 1 (340 mg, 0.819 mmol) suspended in anhydrous THF (12 mL) under N₂ was added a solution of potassium t-butoxide in THF (1 M, 0.78 mL) dropwise at room temperature. The yellow solution formed was concentrated and the residue was triturated with dichloromethane and ether and filtered to afford 130 as a yellow power (304 mg, 82%).

¹H NMR (300 MHz) DMSO-d₆ δ: 8.5 (d, 2H), 8.00 (d, 1H), 7.38 (dd, 1 H), 7.39-7.33 (m, 2H), 7.21-7.12 (m, 2H), 4.58 (dd, 2H), 4.22 (dd, 2H), 3.13 (s, 3H), 3.05 (s, 3H). MS: 438.8 (M+Na).

### EXAMPLE 42

### Preparation of tert-butyl N-(9-(benzhydryloxy)-7-(4-fluorobenzyl)-8-oxo-7,8-dihydro-6H-pyrrolo[3,4-g]quinolin-5-yl)-N-methylsulfamoylcarbamate

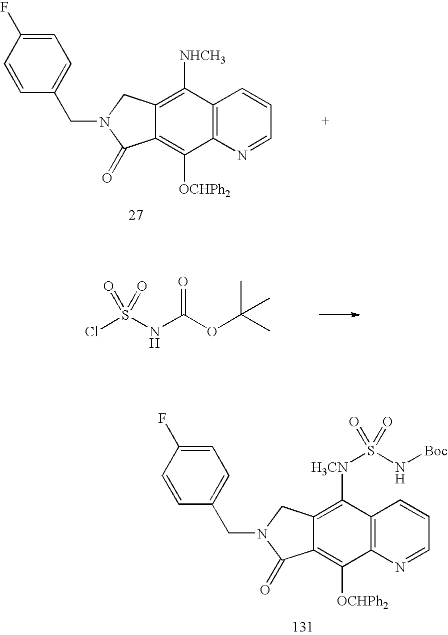

To 25 mg of aniline 27 in 1 ml CH₂Cl₂ at rt was added 100 mL DIEA, followed by a 0.3 M solution of the N-(tert-butoxycarbonyl)sulfamoyl chloride reagent reported in Winum et al. Organic Letters 2001, 3, p. 2241-2243 (0.2 mL, 1.5 eq). At the completion of this addition, the original orange color of the reaction solution had faded to a colorless appearance. The reaction was diluted with 100 mL dichloromethane and washed with 25 mL water and then 25 mL aq. brine solution. After drying over Na₂SO₄, solvent was removed by rotary evaporation to give quantitative yield of the N-Boc protected sulfonyl urea 131.

¹H NMR (300 MHz, CDCl₃) of 131 shows diagnostic peaks at δ 8.95 (m, 1H), 7.70 (d, 1H), 3.42 (s, 2H), and 1.12 (9H, s) ppm, MS=683.1 (M+H).

### EXAMPLE 43

### Preparation of N-(7-(4-fluorobenzyl)-9-hydroxy-8-oxo-7,8-dihydro-6H-pyrrolo[3,4-g]quinolin-5-yl)-N-methylsulfamide

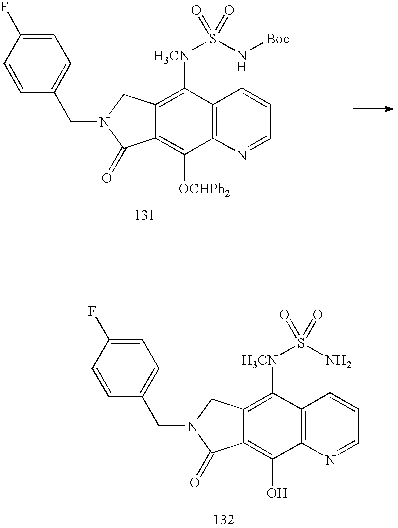

A 28 mg quantity of sulfonyl urea 131 was subjected to the previously reported diphenylmethane ether cleavage conditions to effect global deprotection. After stirring with excess TFA/TES for 4 h in 3 mL dichloromethane, the solvent was removed and the resulting residue was subjected to purification by trituration to provide 10 mg (53%) of sulfonyl urea 132 as the TFA salt.

¹H NMR (300 MHz, CDCl₃) of 132 shows diagnostic peaks at δ 8.95 (d, 1H), 8.50 (d, 1H), and 3.35 (s, 3H) ppm, MS=439.1 (M+Na).

### EXAMPLE 44

### Preparation of N-(7-(4-fluorobenzyl)-9-hydroxy-8-oxo-7,8-dihydro-6H-pyrrolo[3,4-g]quinolin-5-yl)-N-methyl-N′-methylsulfamide

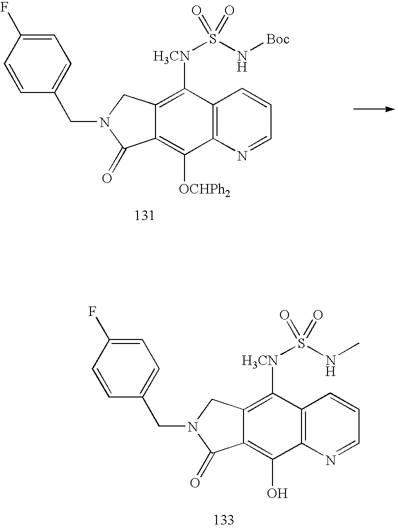

A 300 mg quantity of sulfonyl urea 131 was dissolved in 10 mL acetonitrile. 231 μL of DIEA was added, followed by 100 μL (1.54 mmol, 3.5 eq) MeI. After 1 h at room temperature, LC/MS analysis showed approximately 50% conversion to product, and another 60 μL MeI was added. LC/MS analysis showed no additional starting material remained at that time. The reaction was diluted with ethyl acetate and washed with water & brine solution, followed by drying over Na₂SO₄. Solvent removal via rotary evaporation provided the crude residue which was purified by chromatography on silica gel (1:1 hexanes/EtOAc) to give 232 mg pure (76% yield) as an orange solid. Treatment of this product material to the previously reported diphenylmethane ether cleavage effected global deprotection to provide 150 mg (82% yield) of the mono-methyl sulfonyl urea product 133 as its TFA salt after purification by trituration from hexanes/diethyl ether.

¹H NMR (300 MHz, CDCl₃) of 133 shows diagnostic peaks at δ 8.88 (d, 1H), 8.48 (d,1H), 3.22 (s, 3H), and 2.80 (s, 3H) ppm, MS=432.1 (M+H).

### EXAMPLE 45

### Preparation of N-(7-(4-fluorobenzyl)-9-hydroxy-8-oxo-7,8-dihydro-6H-pyrrolo[3,4-g]quinolin-5-yl)-N-methyl-N′-methylsulfamide

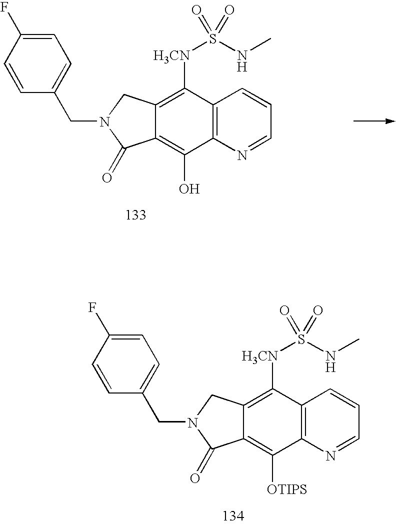

To 150 mg 133 in 5 mL DMF was added 310 μL DIEA, followed by 220 μL TIPS-Cl. A catalytic amount of 4-DMAP was added to the mixture. After 2 h, LC/MS analysis showed a 5:1 ratio of product to starting material, and an additional 200 μL TIPS-Cl was added. The reaction was diluted with EtOAc and washed with water, then brine, followed by drying over Na₂SO₄. Solvent removal gave the product 134 as an orange oil that was carried forward in the analog series in examples 46-47 without additional purification.

### EXAMPLE 46

### Preparation of N′-benzyl-N′-methyl-N-(7-(4-fluorobenzyl)-9-hydroxy-8-oxo-7,8-dihydro-6H-pyrrolo[3,4-g]quinolin-5-yl)-N-methylsulfamide

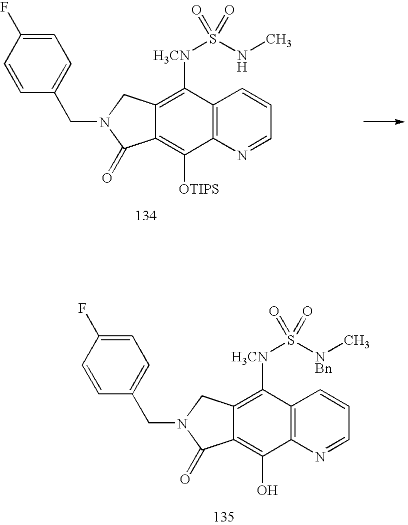

To 20 mg of the TIPS-ether protected intermediate 134, dissolved in 1 mL acetonitrile, is added 55 mg (0.17 mmol, 5 eq) Cs₂CO₃. Two equivalents (0.7 mmol, 8 μL) of benzyl bromide were added and the reaction stirred at room temperature with monitoring by LC/MS and TLC analysis. The reaction was observed to have gone to complete conversion after 1 h, at which time the reaction was diluted with EtOAc, washed with brine, and dried over Na₂SO₄. Concentration via rotary evaportation provided the intermediate alkylation product, which was purified by chromatography on silica gel using 2:1 hexanes/EtOAc. The resulting product was then submitted to TIPS removal via treatment in a 0.3M solution of 2:1 THF/TFA, which furnished 8 mg pure product (38% overall yield from TIPS-protected 135) after trituration from diethyl ether/hexanes.

¹H NMR (300 MHz, CDCl₃) of 135 shows diagnostic peaks at δ 9.05 (m, 1H), 8.52 (d, 1H), 7.35-7.25 (m, 5H), 3.18 (s, 3H), and 2.78 (3H, s) ppm, MS=521.5 (M+H).

### EXAMPLE 47

### Preparation of N-(7-(4-fluorobenzyl)-9-hydroxy-8-oxo-7,8-dihydro-6H-pyrrolo[3,4-g]quinolin-5-yl)-N-methyl-N′-methyl-N′-(pyridin-4-ylmethyl)sulfamide

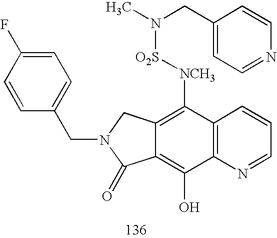

Using the representative procedure from example 44, the TIPS protected intermediate 134 was alkylated to give the N-methylpyridiyl analog 136, 5 mg, obtained after TIPS ether cleavage in 26% overall yield after trituration from diethyl ether/hexanes.

¹H NMR (300 MHz, CDCl₃) of 136 shows diagnostic peaks at δ 8.98 (bs, 1H), 7.94 (d, 1H), 3.26 (s, 3H), and 2.82 (3H, s) ppm, MS=521.7.1 (M+H).

### EXAMPLE 48

### Preparation of N-(7-(2,4-dimethoxybenzyl)-9-hydroxy-8-oxo-7,8-dihydro-6H-pyrrolo[3,4-g]quinolin-5-yl)-N-methylmethanesulfonamide

Procedure:

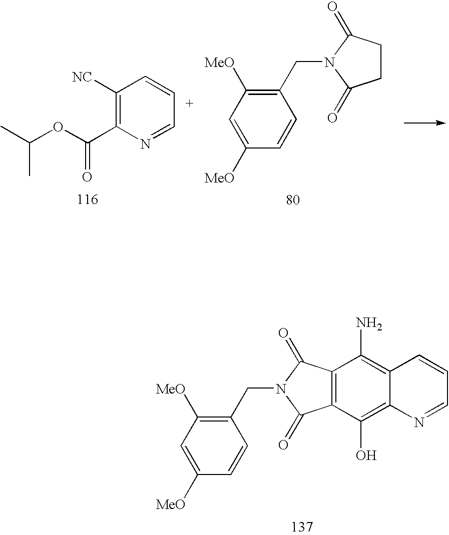

116 (12 g, 63.1 mmol) and 80 from example 23(17.3 g, 69.4 mmol) in THF (216 mL) were cooled to 0° C. and treated with LiHMDS (158 mL, 158 mmol, 1.0 M in THF) predissolved in THF (100 mL) under Ar. The solution was gradually warmed to room temperature for 2 h. The reaction mixture was cooled to 0° C. and 6N HCl (66 mL) was slowly added. THF was removed in vacuo then the crude mixture was suspended in diethyl ether (400 mL) and H₂O (40 mL). The product was filtered then dried using an oven vacuum to afford 137 (22.3 g, 93%) as a red solid (data for HCl salt): 300 MHz ¹H NMR (DMSO) δ (ppm) 9.01 (d, 1H), 8.90 (m, 1H), 7.78 (m, 1H), 6.87 (m, 2H), 6.56 (br s, 1H), 6.42 (m, 1H), 4.62 (s, 2H), 3.81 (s, 3H), 3.71 (s, 3H); MS: 380 (M+1).

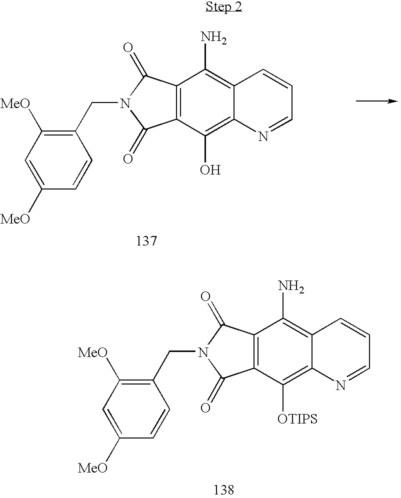

137 (22.3 g, 58.8 mmol) in DMF (295 mL, 0.2 M) was treated with TEA (24.6 mL, 176.5 mmol) and DMAP (719 mg, 5.88 mmol). TIPSCl (17.5 mL, 82.4 mmol) was slowly added over 15 minutes and the reaction mixture was stirred at room temperature for 2 h under nitrogen atmosphere. The reaction mixture was diluted with ethyl acetate (2 L) and quenched with H₂O (800 mL). The layers were separated and the aqueous layer was extracted with ethyl acetate (600 mL). The combined organic layer was washed with aqueous LiCl (twice), citric acid (5% solution) and brine then dried (over Na₂SO₄), filtered and concentrated in vacuo. The crude product was triturated in hexane and filtered to afford the desired product 138 (28.3 g, 90%) as a yellow solid: 300 MHz ¹H NMR (CDCl₃) δ (ppm) 8.95 (dd, 1H), 8.22 (dd, 1H), 7.55 (m, 1H), 7.15 (m, 1H), 6.42 (m, 2H), 4.58 (s, 2H), 3.82 (s, 3H), 3.78 (s 3H), 1.5 (m, 3H), 1.15 (d, 18H); MS: 536 (M+1).

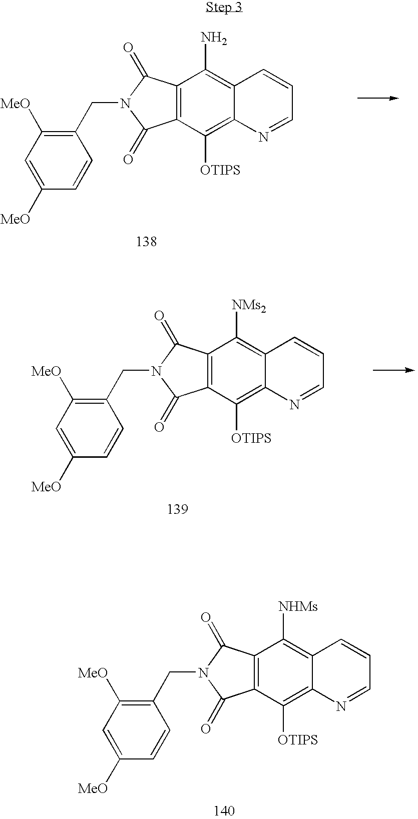

138 (28.3 g, 52.9 mmol) in CH₂Cl₂ (350 mL) was treated with TEA (58.9 mL, 423 mmol) and stirred at −10° C. as a solution of methanesulfonyl chloride (16.4 mL, 211 mmol) in predissolved in CH₂Cl₂ (170 mL) was added dropwise over 45 min. After addition, the mixture was stirred for 3 h while warming to 0° C. The volatiles were removed in vacuo then the residue was redissolved in ethyl acetate (1 L) then quenched with H₂O (400 mL). The layers were separated and the aqueous layer was extracted with ethyl acetate (300 mL). The combined organic layer was washed with H₂O (3×), citric acid (5% solution) and brine then dried (over Na₂SO₄), filtered and concentrated in vacuo with no further purification to yield the crude intermediate bis-mesylate 139 (36.6 g); MS: 692 (M+1).

A solution of bis-mesylate 139 (36.5 g, 52.8 mmol) in THF (360 mL) was stirred at −10° C. as potassium t-butoxide (1.0 M solution in THF, 89.7 mL, 89.7 mmol) predissolved in THF (170 mL) was added dropwise over 30 min. After 20 min, the solution was diluted with ethyl acetate (850 mL) and quenched with H₂O (300 mL). The layers were separated and the aqueous layer was extracted with ethyl acetate (300 mL, 2×). The combined organic layer was washed with H₂O (3×), saturated NH₄Cl and brine then dried (over Na₂SO₄), filtered and concentrated in vacuo. 80% of the crude residue was dissolved in CH₂Cl₂ (100 mL) then passed through a SiO₂ plug, which was pre-washed with 0.05% TEA+9/1—ethyl acetate/hexane. The short column was eluted with 0.05% TEA+9/1—ethyl acetate/hexane to afford the mono-mesylate 140 (17.6 g, 68% from 138) as a tan-yellow solid: 300 MHz ¹H NMR (CDCl₃) δ (ppm) 8.95 (m, 2H), 7.65 (m, 2H), 7.20 (m, 1H), 6.42 (m, 2H), 4.85 (s, 2H), 3.8 (s, 3H), 3.78 (s, 3H), 3.02 (s, 3H), 1.55 (m, 3H), 1.15 (d, 18H); MS: 614 (M+1).

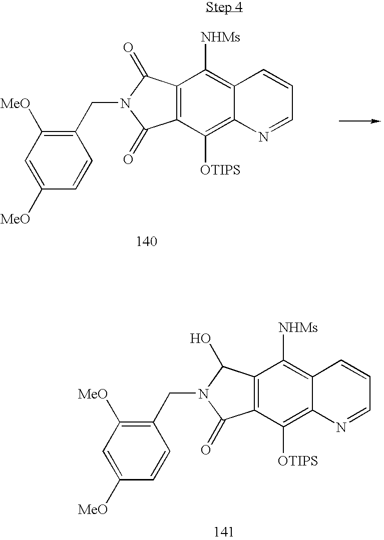

140 (9.2 g, 15.0 mmol) in THF-water (15:1, 160 mL) was cooled to 0° C. and treated with LiBH₄ (5.89 g, 270 mmol) in 3 portions over 3 h. The reaction mixture stirred while warming to room temperature after each addition (which was done at 0° C.). The reaction mixture was cooled to 0° C., treated with citric acid (10% solution, 250 mL), filtered, and THF was removed in vacuo. The resulting solution was diluted with EtOAc (300 mL), washed with saturated NaHCO₃ and brine. The solution was dried (over Na₂SO₄), filtered and concentrated to afford crude aminal 141 (9.3 g, 100%) as a yellow solid: 300 MHz ¹H NMR (CDCl₃) δ ppm) 8.85 (dd,1H), 8.65 (dd, 1H), 7.55 (m, 1H), 7.40 (m,1H), 6.75 (m,1H), 6.53 (m, 2H), 5.97 (s,1H), 4.95 (d,1H), 4.55 (d, 1H), 3.85 (s, 3H), 3.8 (s, 3H), 3.05 (s, 3H), 1.55 (m, 3H), 1.15 (d, 18H); MS: 616 (M+1).

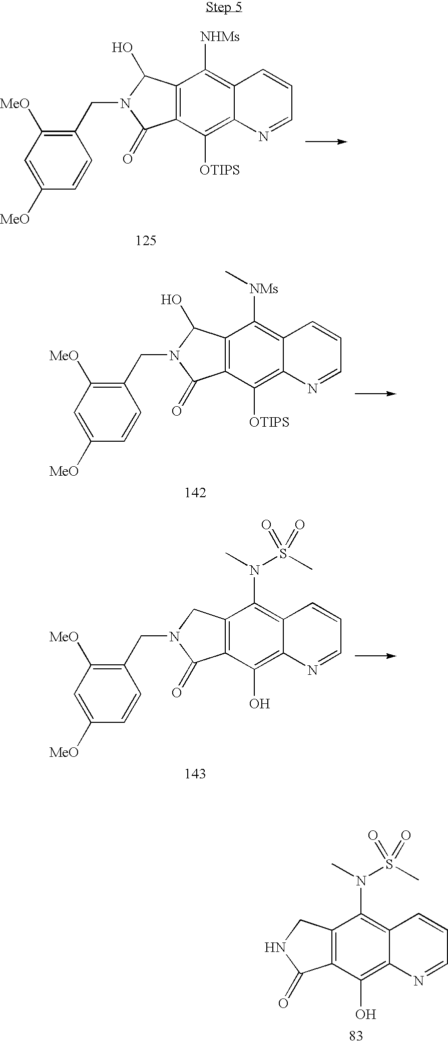

141 (9.9 g, 16.1 mmol) in DMF (161 mL, 0.1 M) was treated with Cs₂CO₃ (5.77 g, 17.7 mmol) and stirred for 5 min. The reaction mixture was treated with MeI (1.1 mL, 17.7 mmol) and stirred for 1.5 h. The reaction mixture was diluted with ethyl acetate then quenched with water. The organic layer was washed with aqueous LiCl (twice), saturated NaHCO₃, and brine. The solution was dried (over Na₂SO₄), filtered and concentrated in vacuo with no further purification to afford the methylated crude product 142 (9.6 g, 95%) as a mixture of diastereomers: MS: 630 (M+1).

A solution of 142 (9.6 g, 15.3 mmol) in CH₂Cl₂ (153 mL) was treated with triethylsilane (48.7 mL) and trifluoroacetic acid (17.6 mL). The reaction mixture was stirred at room temperature under an inert atmosphere for 24 hours. The volatiles were removed in vacuo. Most of the material was carried forward immediately while a small amount of crude product (18 mg) was purified by reverse phase HPLC to afford the desired product 143 (12 mg) as the trifluroacetate salt: 300 MHz ¹H NMR (CDCl₃) δ (ppm) 9.1 (dd, 1H), 8.40 (dd, 1H), 7.71 (m, 1H), 6.49 (m, 2H), 4.9-4.4 (m, 4H), 3.87 (s, 3H), 3.82 (s, 3H), 3.33 (s, 3H), 3.07 (s, 3H); 300 MHz ¹⁹F NMR (CDCl₃) δ (ppm) −76.24; MS: 458 (M+1).

To a solution of the mesylate 143 (15.3 mmol) dissolved in trifluoroacetic acid (153 mL) was added triethylsilane (5 mL). The reaction mixture was heated to 75° C. and stirred for 5 hours upon which the mixture was azeotroped with toluene/THF repeatedly. The crude residue was suspended in dichloromethane and washed thoroughly via trituration. Sonication was used to aid this washing. The solid was filtered on a sintered funnel and air dried thoroughly. An off-white brownish solid 83 (same as example 25) (6.05 g, 94% %) was obtained as the TFA salt; 300 MHz ¹H NMR (DMSO) δ (ppm) 8.95 (dd, 1H), 8.55 (bs, 1H), 8.45 (dd, 1H), 7.77 (m,1H), 4.53 (s, 2H), 3.28 (s, 3H), 3.25 (s, 3H); 300 MHz ¹⁹F NMR (DMSO) δ (ppm) −75.37; MS: 308 (M+1).

### EXAMPLE 49

### Preparation of N-(7-(2,4-difluorobenzyl)-9-hydroxy-8-oxo-7,8-dihydro-6H-pyrrolo[3,4-g]quinolin-5-yl)-N-methylmethanesulfonamide

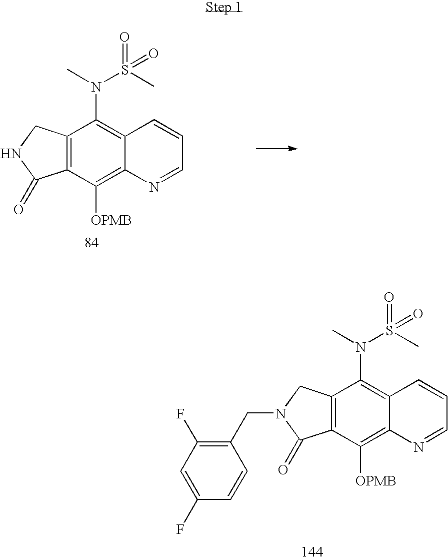

The compound was made in a similar fashion as compound 85 in example 25 to afford the desired product 144 (19 mg, 65%): 300 MHz ¹H NMR (CDCl₃) δ (ppm) 9.05 (dd, 1H), 8.25 (dd, 1H), 7.6 (m, 3H), 7.45 (m, 1H), 6.85 (m, 4H), 5.77 (m, 2H), 4.95 (d, 1H), 4.7 (m, 2H), 4.4 (d, 1H), 3.8 (s, 3H), 3.35 (s, 3H), 3.1 (s, 3H); MS: 554 (M+1).

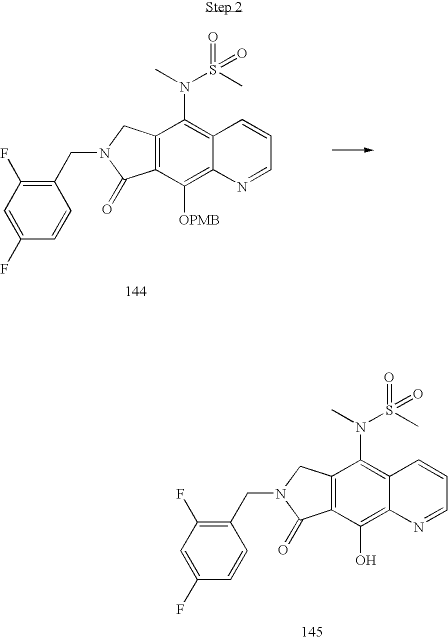

The compound was made in a similar fashion as compound 88 in example 25 to afford the desired product 145 (8 mg, 60%) as the TFA salt: 300 MHz ¹H NMR (CDCl₃) δ (ppm) 9.17 (dd, 1H), 8.50 (dd, 1H), 7.8 (dd, 1H), 7.42 (m, 1H), 6.90 (m, 2H), 4.95-4.47 (m, 4H), 3.35 (s, 3H), 3.11 (s, 3H); 3.00 MHz ¹⁹F NMR (CDCl₃) δ (ppm) −76.28, −109.47, −113.25; MS: 434 (M+1).

### EXAMPLE 50

### Preparation of N-(7-(2-chloro-4-fluorobenzyl)-9-hydroxy-8-oxo-7,8-dihydro-6H-pyrrolo[3,4-g]quinolin-5-yl)-N-methylmethanesulfonamide

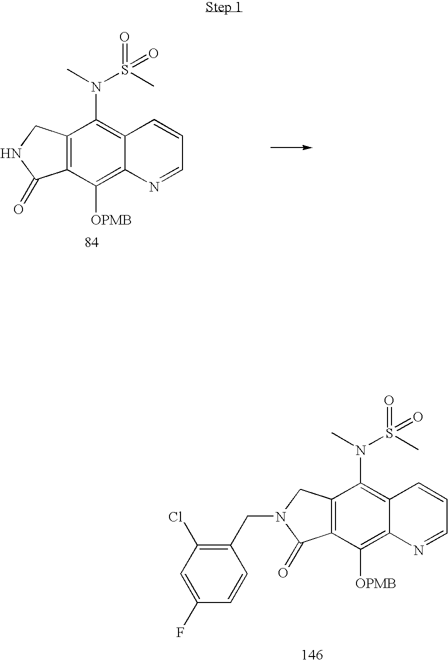

The compound was made in a similar fashion as compound 85 in example 25 to afford the desired product 146 (29 mg, 73%): 300 MHz ¹H NMR (CDCl₃) δ (ppm) 9.07 (dd, 1H), 8.28 (dd, 1H), 7.62 (m, 3H), 7.39 (m, 1H), 7.18 (m, 1H), 7.00 (m, 1H), 6.87 (m, 2H), 5.80 (m, 2H), 5.02 (d, 1H), 4.85 (d, 1H), 4.85 (m, 1H), 4.4 (d, 1H), 3.8 (s, 3H), 3.35 (s, 3H), 3.11 (s, 3H); MS: 570 (M+1).

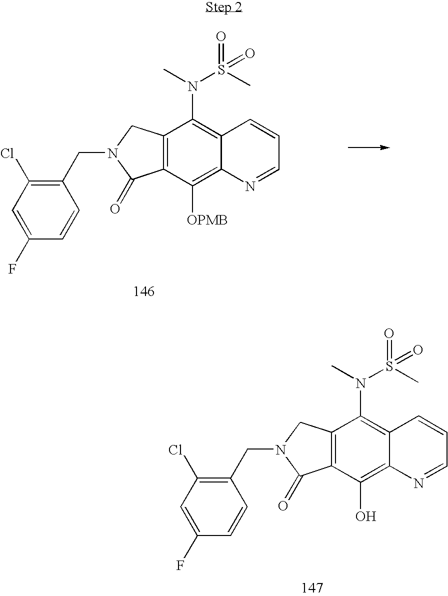

The compound was made in a similar fashion as compound 88 in example 25 to afford the desired product 147 (8 mg, 60%) as the free parent: 300 MHz ¹H NMR (CDCl₃) δ (ppm) 9.00 (dd, 1H), 8.30 (dd, 1H), 7.65 (dd, 1H), 7.43 (m, 1H), 7.18 (m, 1H), 7.01 (m, 1H), 5.03-4.42 (m, 4H), 3.35 (s, 3H), 3.08 (s, 3H); 300 MHz ¹⁹F NMR (CDCl₃) δ (ppm) −111.99; MS: 450 (M+1).

### EXAMPLE 51

### Preparation of N-(7-((5-fluoropyridin-2-yl)methyl)-9-hydroxy-8-oxo-7,8-dihydro-6H-pyrrolo[3,4-g]quinolin-5-yl)-N-methylmethanesulfonamide

Procedure:

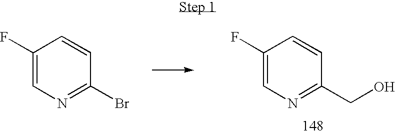

(Related precendent, see Reider et al., Tett. Lett., 41, 2000, 4335-4338). Into a flask containing 2-Bromo-5-fluoro-pyridine (purchased from Aldrich, 2.5 g, 14.30 mmol, 1 eq) was dissolved toluene (143 mL, 0.1 M) and DMF (1.44 mL, 18.58 mmol, 1.3 eq, anhydrous) before being cooled to 78° C. under an inert atmosphere. nBuLi (17.1 mL, 17.14 mmol, 1 M hexanes) was added dropwise via an addition funnel over 15 min and allowed to stir for 90 min. To the mixture was added NaBH₄ (1.08 g, 28.60 mmol, 2 eq) and the reaction allowed to warm up to room temp. The reaction was quenched with saturated NH₄Cl and dissolved in EtOAc (300 mL). It was washed with water (2×100 mL) and brine (100 mL). The organic layer was dried over Na₂SO₄, filtered and concentrated in vacuo. Flash column chromatography was used to purify the product using hexanes/Ethyl acetate (3/7) as eluent to furnish desired alcohol 148.

¹H NMR (300 MHz) CDCl₃ δ: partial 4.75 (s, 2H), 3.67 (bs, 1H). ¹⁹F NMR (300 MHz) CDCl₃ δ: −129.80. MS: 127.96 (M+1).

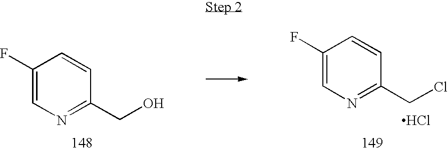

Using standard precedence, alcohol 148 was halogenated to furnish 149 in similar fashion as exemplified above.

¹H NMR (300 MHz) DMSO-d₆ 6: partial 4.79 (s, 2H). ¹⁹F NMR (300 MHz) DMSO-d₆: −128.25. MS: M+absent.

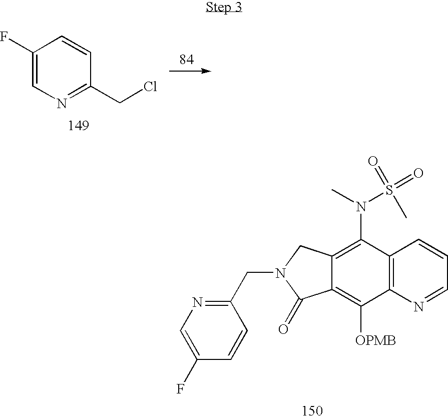

Using standard precedence, alcohol 149 was reacted with 84 to furnish 150 in similar fashion as exemplified above. ¹H NMR (300 MHz) CDCl₃ δ: partial 5.77 (dd, AB, 2H), 3.80 (s, 3H), 3.35 (s, 3H), 3.12 (s, 3H). ¹⁹F NMR (300 MHz) CDCl₃d₆: −128.25. MS: 680.94 (M+1).

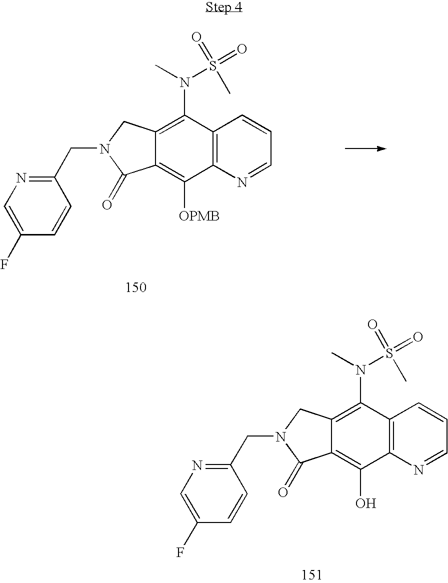

In procedures similar to those exemplified above, compound 151 was prepared.

¹H NMR (300 MHz) CDCl₃ δ: partial 3.56 (s, 3H), 3.10 (s,3H). ¹⁹F NMR (300 MHz) CDCl₃ δ: −128.57. MS: 417.06 (M+1).

### EXAMPLE 52

### Preparation of N-(7-(4-fluorobenzyl)-9-hydroxy-6,8-dioxo-7,8-dihydro-6H-pyrrolo[3,4-g]quinolin-5-yl)-N-methylmethanesulfonamide

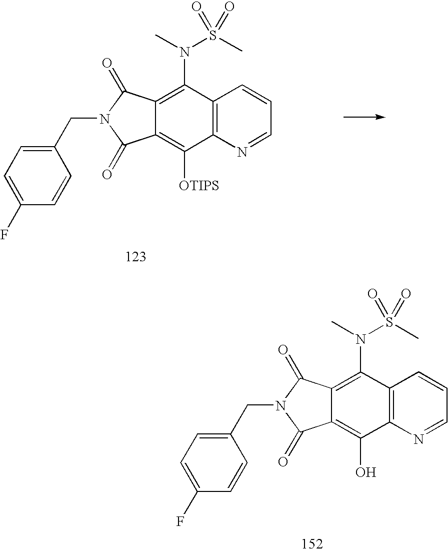

In procedures similar to those exemplified above, compound 152 was prepared from 123 of example 37.

¹H NMR (300 MHz) CDCl₃ δ: partial 4.87 (d, 2H), 3.45 (s, 3H), 3.16 (s, 3H). ¹⁹F NMR (300 MHz) CDCl₃ δ: −114.31. MS: 430.10(M+1).

### EXAMPLE 53

### Preparation of 5-(dimethylamino)-N-(7-(4-fluorobenzyl)-9-hydroxy-8-oxo-7,8-dihydro-6H-pyrrolo[3,4-g]quinolin-5-yl)-N-methylnaphthalene-1-sulfonamide

Procedure:

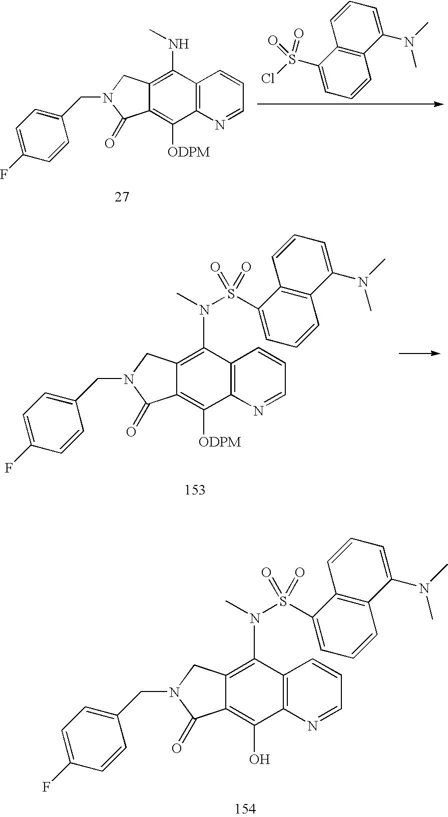

Compound 27 of example 2 (21.5 mg, 42 μmol) was dissolved in 1 mL of pyridine and flashed with nitrogen. It was cold to 0° C. and added sulfonyl chloride (46 mg, 170 μmol) and catalytic amount of DMAP. The mixture was allowed to warm to room temperature and stirred for 20 hours under nitrogen. The reaction was diluted with 10 mL of EtOAc, washed with brine, dried over Na₂SO₄ and concentrated in vacuum to give crude product which was then purified flash chromatography on silica gel (20% to 50% ethyl acetate in hexane) to provide 153 (33 mg,). m/z=737 (M+1).

Compound 153 was dissolved in 1 mL of DCM and treated with TFA (100 μl) and triethylsilane (200 μL). After stirring for 30 minutes at room temperature, the reaction mixture was azeotroped with toluene once. The resulting residue was then purified by reverse-phase prep HPLC to provide 12 mg (42% in yield) of 154 as the TFA salt. 300 MHz ¹H NMR (CDCl₃) δ (ppm): 8.9 (d,1H); 8.7 (d, 1H); 8.3 (d,1H);8.1 (d, 1H); 7.76 (m, 2H); 7.3-7.2 (m, 5H); 7.1 (t, 2H); 4.8 (d, 1H); 4.5 (d, 1H); 4.2 (m, 2H); 3.3 (s, 3H); 3.0 (s, 6H). ¹⁹F NMR (CDCl₃) δ (ppm): −76.2; −114.5. m/z=685 (M+1).

General HPLC conditions: mobile phase A was 0.1% TFA in water, mobile phase b was 0.1% TFA in CH₃CN; gradient from 5% to 60% B in 20 min; flow rate was 20 mL/min; column was Phenomenex, luna 5μ, C18 (2), 150 mm×21.1 mm

### EXAMPLE 54

### Preparation of 2,2,2-trifluoro-N-(7-(4-fluorobenzyl)-9-hydroxy-8-oxo-7,8-dihydro-6H-pyrrolo[3,4-g]quinolin-5-yl)-N-methylethane-1-sulfonamide

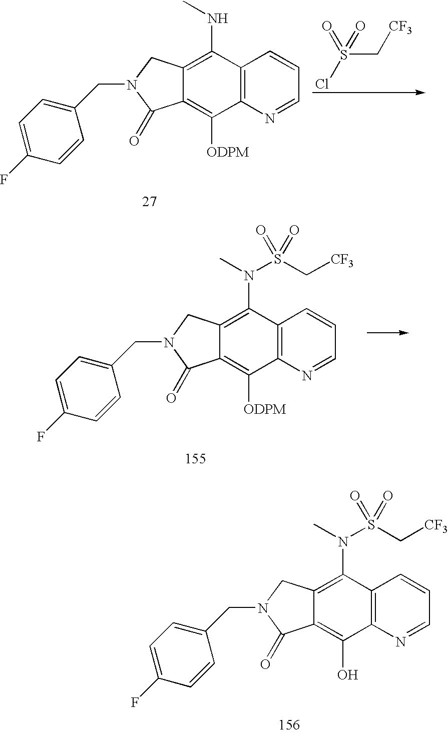

Compound 27 (30 mg, 60 μmol) was dissolved in 1 mL of pyridine and flashed with nitrogen. It was cooled to 0° C. and added sulfonyl chloride (33 μL, 300 μmol). After 10 min in 0° C., the mixture was allowed to warm to room temperature and stirred for 30 min under nitrogen. The reaction was diluted with 10 mL of EtOAc, washed with 0.1N HCl and brine, dried over Na₂SO₄ and concentrated in vacuum to give crude product 155.

The deprotection of DPM group at C8-OH was carried out as in Example 53. The resulting residue was then purified by reverse-phase prep HPLC to provide 25 mg (70% in yield) of 156 as the TFA salt. 300 MHz ¹H NMR (CDCl₃) δ (ppm): 9.1(d, 1H); 8.4 (d, 1H); 7.7 (m, 1H); 7.4-7.3 (m, 2H); 7.0 (t, 2H); 4.9 (d,1H); 4.7 (m, 2H); 4.4 (d, 2H); 3.9 (m, 2H); 3.4 (s, 3H). ¹⁹F NMR (CDCl₃) δ (ppm): −61.7 (t); −76.2; −114.5. m/z=484 (M+1).

### EXAMPLE 55

### Preparation of N-(7-(4-fluorobenzyl)-9-hydroxy-8-oxo-7,8-dihydro-6H-pyrrolo[3,4-g]quinolin-5-yl)-N-methylpyridine-2-sulfonamide

Procedure:

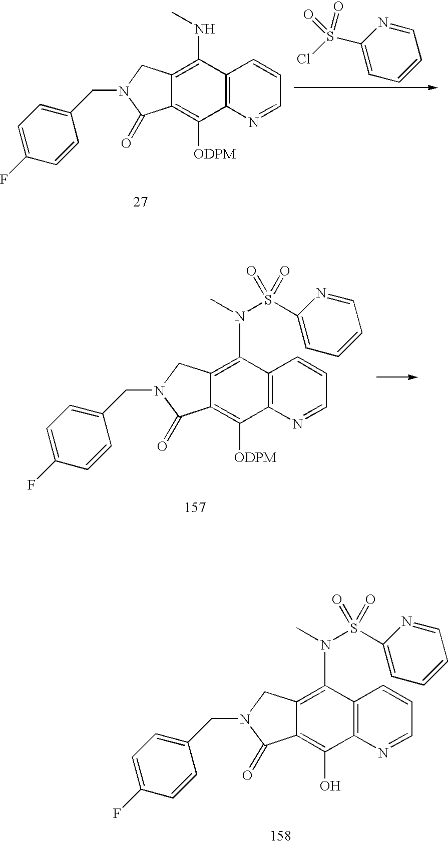

Compound 27 (34 mg, 67 μmol) was dissolved in 1 mL of acetonitrile at room temperature, Potassium carbonate (93 mg, 675 μmol) and 2-pyridinesulfonyl chloride (72 mg, 337 μmol). The mixture was allowed 24 hours under nitrogen. The reaction was diluted with 10 mL of EtOAc, washed with 0.1N HCl and brine, dried over Na₂SO₄ and concentrated in vacuum to give crude product 157.

The deprotection of DPM group at C8-OH was carried out as in Example 53. The resulting residue was then purified by reverse-phase prep HPLC to provide 21.6 mg (45% in yield) of 158 as the bis-TFA salt. 300 MHz ¹H NMR (CDCl₃) δ (ppm): 9.0 (d, 1H); 8.7 (d, 1H); 8.2 (d, 1H); 7.8 (m, 2H); 7.5 (m, 2H); 7.3 (m, 2H); 7.0 (t, 2H); 4.8 (q, 2H); 4.2 (s, 2H); 3.5 (s, 3H). ¹⁹F NMR (CDCl₃) δ (ppm): −76.2; −114.3. m/z=479 (M+1).

### EXAMPLE 56

### Preparation of N-(7-(4-fluorobenzyl)-9-hydroxy-8-oxo-7,8-dihydro-6H-pyrrolo[3,4-g]quinolin-5-yl)-N-methyl-6-morpholinopyridine-3-sulfonamide

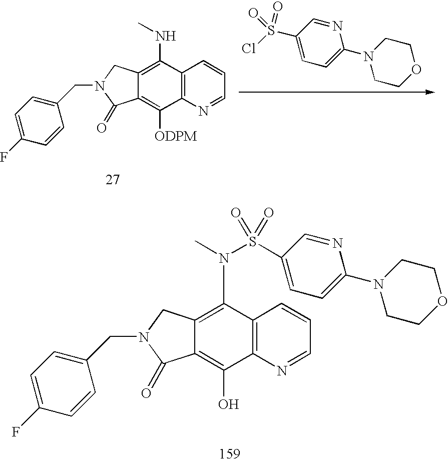

The experiment was carried out as described in Example 55.

The deprotection of DPM group at C8-OH was carried out as in Example 53. The resulting residue was then purified by reverse-phase prep HPLC to provide 13.7 mg of 159 as the bis-TFA salt. 300 MHz ¹H NMR (CDCl₃) δ (ppm): 8.9 (d, 1H); 8.3 (d, 1H); 8.2 (d, 1H); 7.6 (m, 2H); 7.4 (m, 2H); 7.1 (t, 2H);6.8 (d, 1H); 4.7(q, 2H); 4.5-4.2 (q, 2H); 3.8 (m, 4H); 3.6 (m, 4H); 3.3 (s, 3H). ¹⁹F NMR (CD₃OD) δ (ppm): −78.0; −117.1. m/z=564 (M+1).

### EXAMPLE 57

### Preparation of N-(5-(N-(7-(4-fluorobenzyl)-9-hydroxy-8-oxo-7,8-dihydro-6H-pyrrolo[3,4-g]quinolin-5-yl)-N-methylsulfamoyl)-4-methylthiazol-2-yl)acetamide

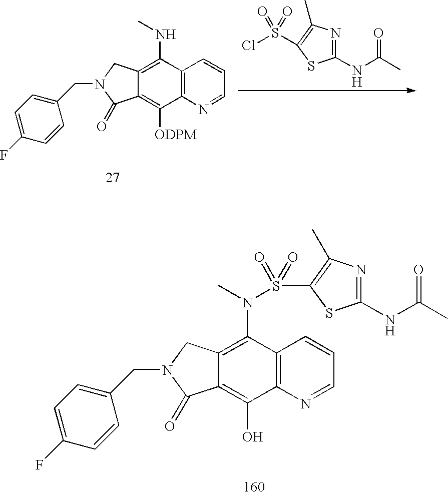

The experiment was carried out as described in Example 55, except that it was heated at 60° C. for 24 h.

The deprotection of DPM group at C8-OH was carried out as in Example 53. The resulting residue was then purified by reverse-phase prep HPLC to provide 24.5 mg of 160 as the TFA salt. 300 MHz ¹H NMR (CD₃OD) δ (ppm): 8.9 (d, 1H); 8.3 (d, 1H); 7.6 (m, 2H); 7.4 (m, 2H); 7.1 (t, 2H); 4.8-4.1 (m, 4H); 3.64 (s, 3H); 2.2 (s., 3H); 2.0 (s, 3H). ¹⁹F NMR (CDCl₃) δ (ppm): −78.0; −117.0. m/z=556 (M+1).

### EXAMPLE 58

### Preparation of 2-(diethylamino)-N-(7-(4-fluorobenzyl)-9-hydroxy-8-oxo-7,8-dihydro-6H-pyrrolo[3,4-g]quinolin-5-yl)-N-methylethanesulfonamide and

### N-(7-(4-fluorobenzyl)-9-hydroxy-8-oxo-7,8-dihydro-6H-pyrrolo[3,4-g]quinolin-5-yl)-N-methyl-2-(piperidin-1-yl)ethanesulfonamide and

### N-(7-(4-fluorobenzyl)-9-hydroxy-8-oxo-7,8-dihydro-6H-pyrrolo[3,4-g]quinolin-5-yl)-N-methyl-2-morpholinoethanesulfonamide and

### N-(7-(4-fluorobenzyl)-9-hydroxy-8-oxo-7,8-dihydro-6H-pyrrolo[3,4-g]quinolin-5-yl)-N-methyl-2-(piperazin-1-yl)ethanesulfonamide

Procedure:

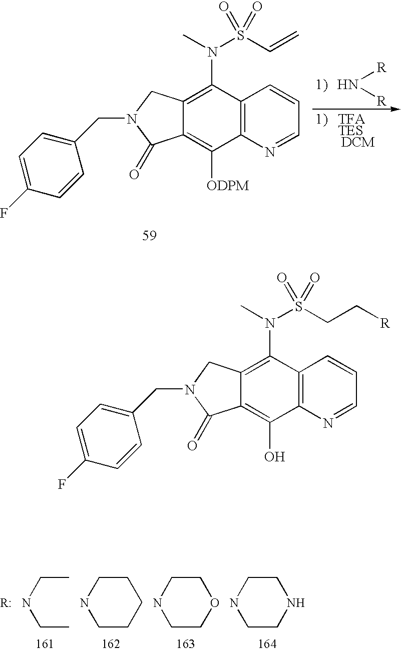

General procedure for the alkylation on terminal amine:

The solid 59 (65 mg, 0.109 mmol) was dissolved in 2 mL of THF. Amine (1 mL) was added. The reaction mixture was stirred at room temperature for 4 hours under nitrogen. The reagent and solvent were removed under reduced pressure evaporation. Re-dissolved in EtOAc, it was washed wit 0.1N HCl and brine, dried over Na₂SO₄ and concentrated in vacuum to give crude product.

The deprotection of DPM group at C8-OH to compounds 161-164 was carried out as in Example 53. The resulting residue was then purified by reverse-phase prep HPLC.

From diethylamine, 58.6 mg (87.5% yield) of 161 was obtained as bis-TFA salt. 300 MHz ¹H NMR (CD₃OD) δ (ppm): 9.0 (d, 1H); 8.6 (d, 1H); 7.8 (m, 1H); 7.4 (m, 2H); 7.1 (t, 2H); 4.8-4.6 (m, 4H); 4.0 (m, 2H); 3.9 (m, 2H); 3.6 (m, 2H); 3.4 (s, 3H); 3.3 (m, 4H); 1.3 (m, 6H). ¹⁹F NMR (CDCl₃) δ (ppm); −78.0; −117.2. m/z=501 (M+1).

From piperidine, 58.6 mg (85.8% yield) of 162 was obtained as bis-TFA salt. 300 MHz ¹H NMR (CD₃OD) δ (ppm): 8.9 (d, 1H); 8.6 (d, 1H); 7.8 (m, 1H); 7.4 (m, 2H); 7.1 (t, 2H); 4.8-4.6 (m, 4H); 4.0 (m, 2H); 3.9 (m, 2H); 3.6 (m, 4H); 3.4 (s, 3H); 3.0 (m, 2H); 2.0-1.4 (m, 6H). ¹⁹F NMR (CDCl₃) δ (ppm); −77.9; −117.2. m/z=513 (M+1).

From morpholine, 24.4 mg (70% yield) of 163 was obtained as bis-TFA salt. 300 MHz ¹H NMR (CD₃OD) δ (ppm): 8.9 (d, 1H); 8.6 (d, 1H); 7.8 (m, 1H); 7.4 (m, 2H); 7.1 (t, 2H); 4.8-4.6 (m, 4H); 4.1-3.6 (m, 8H); 3.4 (m, 4H); 3.3 (s, 3H). ¹⁹F NMR (CDCl₃) δ (ppm): −77.9; −117.2. m/z=515 (M+1).

From N-Boc-piperizine, 44.3 mg (79% yield) of 164 was obtained as tris-TFA salt. 300 MHz ¹H NMR (CD₃OD) δ (ppm): 8.9 (d, 1H); 8.6 (d, 1H); 7.8 (m, 1H); 7.4 (m, 2H); 7.1 (t, 2H); 4.8-4.6 (m, 4H); 3.7-3.6 (m, 2H); 3.3 (s, 3H); 3.2 (m, 2H); 3.0 (m, 2H); 2.9 (m, 4H). ¹⁹F NMR (CDCl₃) δ (ppm): −78.0; −117.2. m/z=514 (M+1).

### EXAMPLE 59

### Preparation of N-(7-(4-fluorobenzyl)-6,9-dihydroxy-8-oxo-7,8-dihydro-6H-pyrrolo[3,4-g]quinolin-5-yl)-N-methylmethanesulfonamide

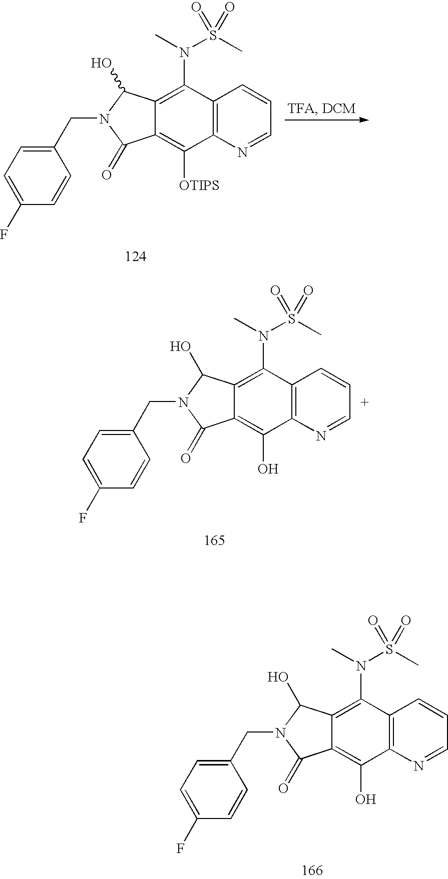

Crude 124-3 (87.7 mg, 0.149 mmol) in CH₂Cl₂ (1.5 mL) was treated with TFA (115 μL, 1.492 mmol) and stirred for 16 h at room temperature. The solvent was removed and the residue was dissolved in DMF (1.5 mL) and purified by reverse-phase HPLC (5-100% MeCN-H₂O gradient) to provide 165 (rₜ=8.88 min (fast), 6.2 mg, 10%) as a white solid: ¹H NMR (CDCl₃, 300 MHz) 8.78 (m, 1H), 8.40 (m, 1H), 7.65 (m, 1H), 7.35 (m, 2H), 6.99 (m, 2H), 5.99 (br s, 1H), 4.99 (d, 1H), 4.96 (d, 1H), 3.43 (s, 3H), 3.08 (s, 3H); ¹⁹(CDCl₃, 282 MHz) −114.0; MS (ESI) m/z 454 [M+Na]⁺ and 166 (rₜ=9.21 min (slow), 5.4 mg, 8%) as a white solid: ¹H NMR (CDCl₃, 300 MHz) 8.50 (m, 1H), 8.45 (m, 1H), 7.48 (m, 1H), 7.35 (m, 2H), 6.99 (m, 2H), 5.67 (br s, 1H), 5.13 (d, 1H), 4.40 (d, 1H), 3.38 (s, 3H), 3.25 (s, 3H); ¹⁹F NMR (CDCl₃, 282 MHz) −114.8; MS (ESI) m/z454 [M+Na]⁺.

### EXAMPLE 60

### Preparation of N-(7-(4-fluorobenzyl)-9-hydroxy-6-oxo-7,8-dihydro-6H-pyrrolo[3,4-g]quinolin-5-yl)-N-methylmethanesulfonamide

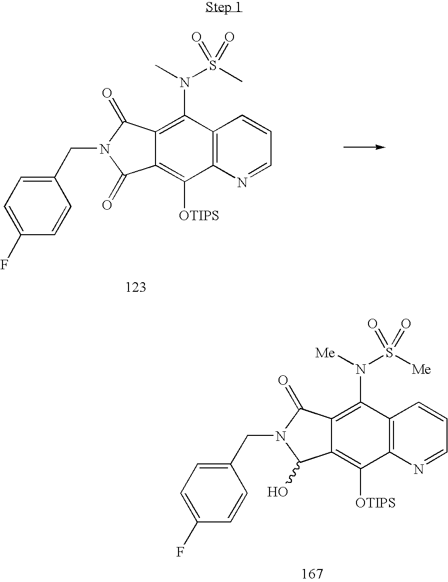

Reduction of purified 123 from example 35 (330.6 mg, 0.564 mmol) was performed with LiBH₄ at room temperature and the reaction mixture was worked up as described in the synthesis of 29 in example 37. The crude product was purified by chromatography to obtain 12.9 mg (4%) of ˜85% pure 167: R_{f}=0.51 (ethyl acetate/hexanes=1/1); ¹H NMR (CDCl₃, 300 MHz) δ 8.89 (dd, 1H, J=4.2 and 1.5 Hz), 8.72 (dd, 1H, J=8.4 and 1.5 Hz), 7.60 (dd, 1H, J=8.4 and 4.2 Hz), 7.42 (appt dd, 2H, J=8.8 and 5.6 Hz), 7.06 (appt t, 2H, J=7.6 Hz), 5.95 (d, 1H, J=9.0 Hz), 5.05 (d, 1H, J=15.0 Hz), 4.52 (d, 1H, J=15.0 Hz), 3.47 (s, 3H), 3.25 (s, 3H), 2.58 (d, 1H, J=9.0 Hz), 1.51 (m, 3H, 7.5 Hz), 1.06 (dd, 18H, J=7.2 and 3.0 Hz); ¹⁹F NMR (CDCl₃, 282 MHz) δ−115.00 (m).

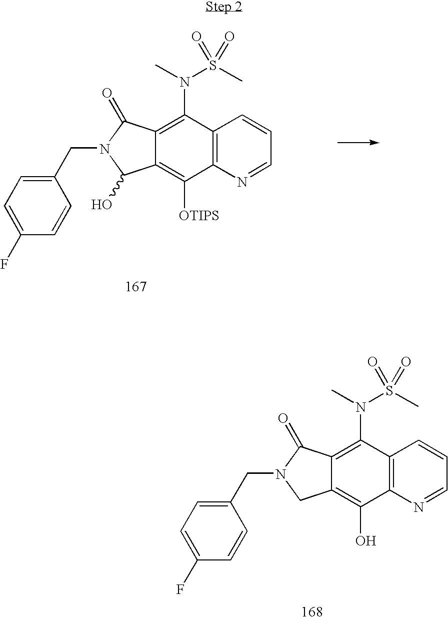

A solution of 12.9 mg (21.9 μmol) of 167, 0.3 mL (1.88 mmol) of Et₃SiH, and 0.15 mL (1.95 mmol) of TFA in 2.25 mL of CH₂Cl₂ was stirred at room temperature for 22 h and the concentrated. The residue was purified by preparative HPLC and freeze-drying of the product containing fractions provided 5.9 mg (65%) of 168 as light yellow powder: ¹H NMR (CDCl₃, 300 MHz) δ 8.89 (br d, 1H, J=1.5 Hz), 8.80 (br d, 1H, J=8.7 Hz), 7.65 (dd, 1H, J=8.7 and 1.5 Hz), 7.33 (br appt dd, 2H, J=8.4 and 5.4 Hz), 7.07 (appt t, 2H, J=8.6 Hz), 4.81 (ABqt, 2H, J=15.9 Hz), 4.45 (s, 1H), 3.53 (s, 3H), 3.26 (s, 3H); ¹⁹F NMR (CDCl₃, 282 MHz) δ−114.47 (m); MS (ESI) m/z416 [M+H]⁺.

### Example 61

### Preparation of 4-nitrophenyl 9-(benzhydryloxy)-7-(4-fluorobenzyl)-7,8-dihydro-8-oxo-6H-pyrrolo[3,4-g]quinolin-5-ylmethylcarbamate

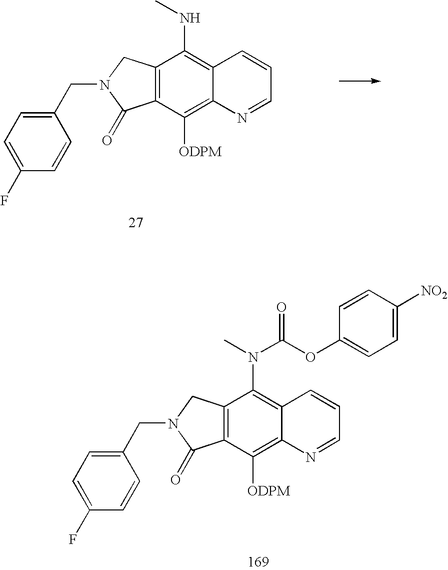

27 (65 mg, 130 μmol) was dissolved in 600 μL of THF. Triethylamine (43 μL, 310 μmol) and para-nitrophenylchloroformate (32 mg, 160 μmol) were then added and the reaction was allowed to stir at room temperature. After stirring at room temperature for 60 minutes, the reaction was diluted with ethyl acetate. The organic was then washed once with 0.25 M citric acid, twice with water, and once with brine. The organic was then dried over Mg₂SO₄ and concentrated in vacuo. The crude residue was then purified by silca gel chromatography (1:1-ethyl acetate:hexane) to afford intermediate 169 (68 mg, 78%).

### Example 62

### Preparation of 1-(7-(4-fluorobenzyl)-9-hydroxy-8-oxo-7,8-dihydro-6H-pyrrolo[3,4-g]quinolin-5-yl)-1,3,3-trimethylurea and 1-(9-(benzhydryloxy)-7-(4-fluorobenzyl)-8-oxo-7,8-dihydro-6H-pyrrolo[3,4-g]quinolin-5-yl)-1,3,3-trimethylurea

Procedure:

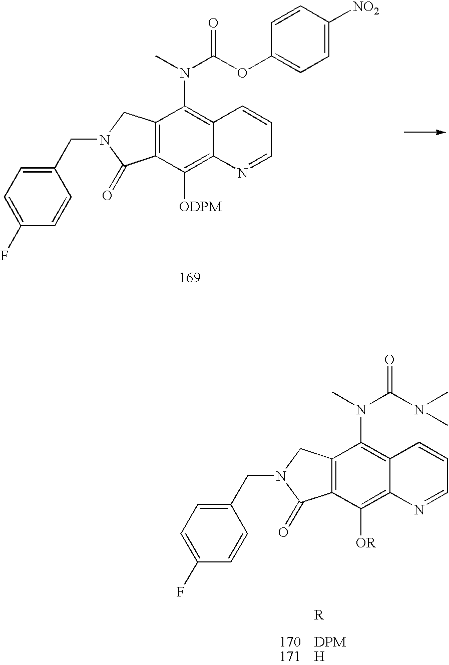

169 (68 mg, 100 μmol) was dissolved in 500 μL of neat dimethylamine, after which the reaction vessel was sealed. After stirring at room temperature overnight, the reaction was diluted with ethyl acetate. The organic was then washed once with 0.25 M citric acid, twice with water, and once with brine. The organic was dried over Mg₂SO₄ and concentrated in vacuo. The crude residue was then purified by silica gel chromatography (neat ethyl acetate) to afford 170 (46 mg, 80%).

170 (46 mg, 80 μmol) was then dissolved in 400 μmol of DCM and treated with 30 μl (400 μmol) of TFA and 31 μl (160 μmol) of triethylsilane. After stirring at room temperature for 30 minutes, the mixture was azeotroped two times with toluene. The residue was then triturated with 3:1-hexane:ether to afford 171 (40 mg, 96%) as the TFA salt. 300 MHz ¹H NMR (CDCl₃) δ (ppm): 8.99 (b, 1H); 8.45 (b, 1H); 8.34 (d,1H); 7.62 (m, 1H); 7.25 (t, 2H); 7.00 (t, 2H); 4.69 (m, 2H); 4.15 (m, 2H); 3.07 (s, 3H); 2.44 (s, 6H); ¹⁹F NMR (CDCl₃) δ (ppm): −75.52; −114.314. MS=409.1 (M+1)

### Example 63

### Preparation of N-(7-(4-fluorobenzyl)-9-hydroxy-8-oxo-7,8-dihydro-6H-pyrrolo[3,4-g]quinolin-5-yl)-N-methylmorpholine-4-carboxamide and

### N-(9-(benzhydryloxy)-7-(4-fluorobenzyl)-8-oxo-7,8-dihydro-6H-pyrrolo[3,4-g]quinolin-5-yl)-N-methylmorpholine-4-carboxamide

Procedure:

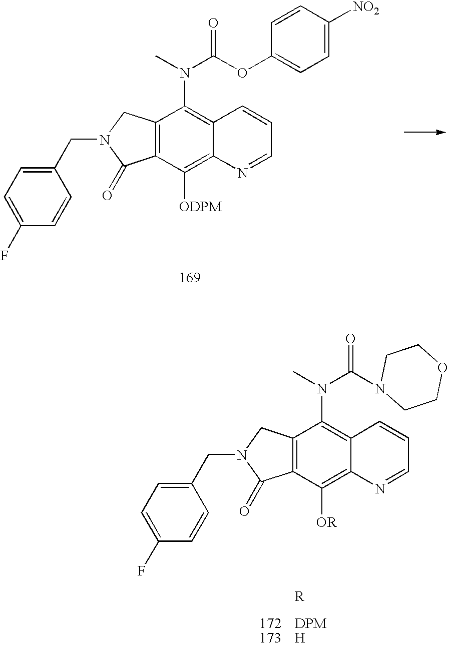

169 (68 mg, 100 μmol) was dissolved in 500 μL of neat morpholine, after which the reaction was heated to 70° C. After stirring at 70° C. for 36 hours, the reaction was diluted with ethyl acetate. The organic was then washed once with 0.25 M citric acid, twice with water, and once with brine. The organic was dried over Mg₂SO₄ and concentrated in vacuo. The crude residue was then purified by silica gel chromatography (neat ethyl acetate) to afford 172 (40 mg, 80%).

172 (46 mg, 80 μmol) was then dissolved in 400 μmol of DCM and treated with 30 μl (400 μmol) of TFA and 31 μl (160 μmol) of triethylsilane. After stirring at room temperature for 30 minutes, the mixture was azeotroped two times with toluene. The residue was then triturated with 3:1-hexane:Ether to afford 173 (23 mg, 78%). 300 MHz ¹H NMR (CDCl₃) δ (ppm): 9.01 (d, 1H); 8.28 (d, 1H); 7.65 (m,1H); 7.31 (t, 2H); 7.06 (t, 2H); 4.70 (m, 2H); 4.23 (m, 2H); 3.17 (m, 7H); 2.97 (m, 4H). MS=451.5 (M+1).

### Example 64

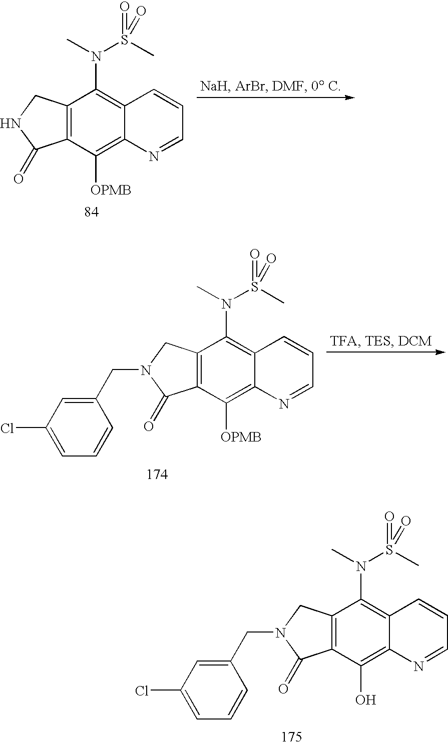

Compound 174: The compound was made in a similar fashion as compound 96 to afford the desired product 174 (32 mg, 83%): 300 MHz ¹H NMR (CDCl₃) δ.(ppm) 9.08 (dd, 1H), 8.28 (dd, 1H), 7.64 (m, 3H), 7.3 (m, 4H), 6.9 (d, 2H), 5.8 (m, 2H), 5.1-4.3 (m, 4H), 3.8 (s, 3H), 3.33 (s, 3H), 3.1 (s, 3H); MS: 552 (M+1).

Compound 175: The compound was made in a similar fashion as compound 97 to afford the desired product 175 (18 mg, 57%) as the TFA salt: 300 MHz ¹H NMR (CDCl₃) δ.(ppm) 9.11 (dd, 1H), 8.40 (dd, 1H), 7.74 (m, 1H), 7.34 (m, 2H), 7.25 (m, 2H), 5.0 (d, 1H), 4.75 (d, 1H), 4.59 (d, 1H), 4.45 (d, 1H), 3.34 (s, 3H), 3.09 (s, 3H); 300 MHz ¹⁹F NMR (CDCl₃) δ.(ppm) −76.25; MS: 432 (M+1).

### Example 65

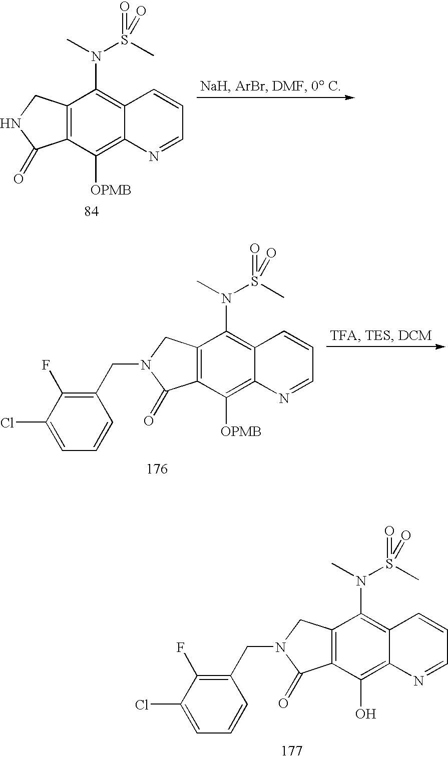

Compound 176: The compound was made in a similar fashion as compound 96 to afford the desired product 176 (31 mg, 78%): 300 MHz ¹H NMR (CDCl₃) δ (ppm) 9.06 (dd, 1H), 8.30 (dd, 1H), 7.61 (m, 3H), 7.32 (m, 2H), 7.09 (t, 1H), 6.87 (d, 2H), 5.78 (m, 2H), 5.1-4.4 (m, 4H), 3.80 (s, 3H), 3.35 (s, 3H), 3.12 (s, 3H); MS: 570 (M+1).

Compound 177: The compound was made in a similar fashion as compound 97 to afford the desired product 177 (25 mg, 82%) as the TFA salt: 300 MHz ¹H NMR (CDCl₃) δ.(ppm) 9.10 (dd, 1H), 8.43 (dd, 1H), 7.75 (m, 1H), 7.34 (m, 2H), 7.12 (t, 1H), 5.01 (d, 1H), 4.8 (m, 2H), 4.54 (d, 1H), 3.36 (s, 3H), 3.11 (s, 3H); 300 MHz ¹⁹F NMR (CDCl₃) δ (ppm) −76.25, −121.09; MS: 450 (M+1).

### Example 66

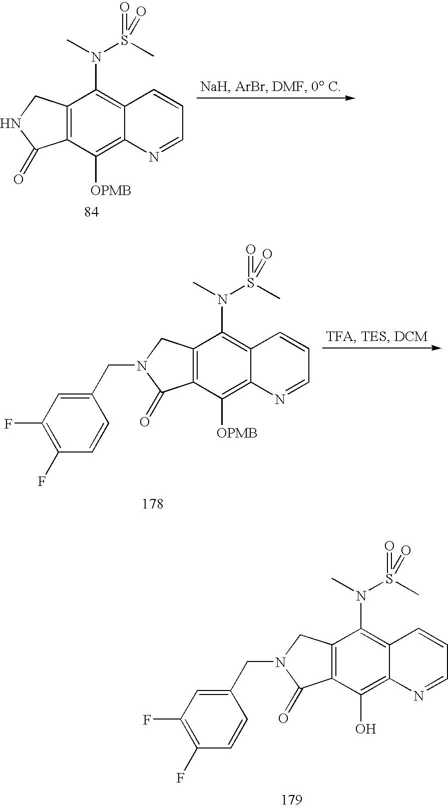

Compound 178: The compound was made in a similar fashion as compound 96 to afford the desired product 178 (30 mg, 77%): 300 MHz ¹H NMR (CDCl₃) δ.(ppm) 9.1 (dd, 1H), 8.26 (dd, 1H), 7.65 (m, 3H), 7.25-7.05 (m, 3H), 6.89 (d, 2H), 5.81 (m, 2H), 5.05-4.3 (m, 4H), 3.80 (s, 3H), 3.34 (s, 3H), 3.11 (s, 3H); MS: 554 (M+1).

Compound 179: The compound was made in a similar fashion as compound 97 to afford the desired product 179 (29 mg, 95%) as the TFA salt: 300 MHz ¹H NMR (CDCl₃) δ.(ppm) 9.12 (dd, 1H), 8.40 (dd, 1H), 7.75 (m, 1H), 7.25-7.05 (m, 3H), 4.95 (d, 1H), 4.75 (m, 1H), 4.57 (d, 1H), 4.42 (d, 1H), 3.34 (s, 3H), 3.09 (s, 3H); 300 MHz ¹⁹F NMR (CDCl₃) δ (ppm) −76.22, −136.7, −138.7; MS: 434 (M+1).

### Example 67

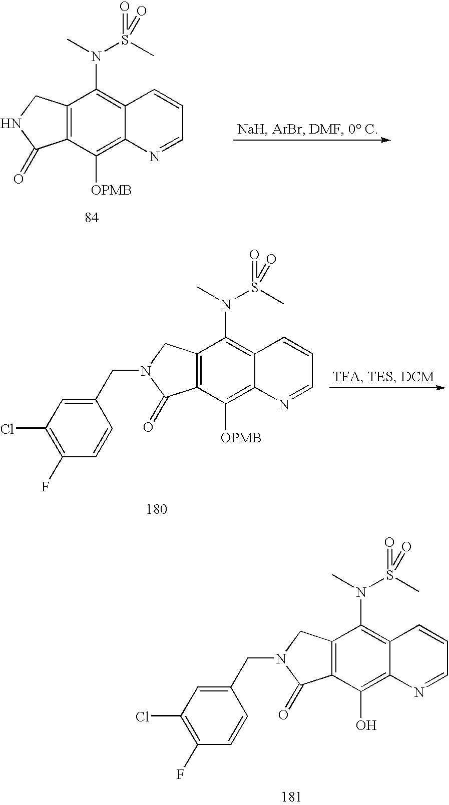

Compound 180: The compound was made in a similar fashion as compound 96 to afford the desired product 180 (365 mg, 91%): 300 MHz ¹H NMR (CDCl₃) δ (ppm) 9.08 (dd, 1H), 8.25 (dd, 1H), 7.64 (m, 3H), 7.43 (dd, 1H), 7.29 (m, 1H), 7.14 (m, 1H), 6.9 (d, 2H), 5.8 (m, 2H), 4.8 (m, 2H), 4.5 (m, 2H), 3.8 (s, 3H), 3.34 (s,.3H), 3.11 (s, 3H); MS: 570 (M+1).

Compound 181: A solution of intermediate 180 (365 mg, 0.64 mmol) in dichloromethane (2 mL) was treated with trifluoroacetic acid (0.3 mL) and triethylsilane (0.3 mL). The reaction mixture was stirred at room temperature under an inert atmosphere overnight upon which the mixture was azeotroped with toluene/THF repeatedly. The solid was triturated in diethyl ether/hexane (1/1) resulting in a yellowish solid, then in ether/methanol (3/1) to afford the desired product 181 (224 mg, 78%) as the parent (white) solid: 300 MHz ¹H NMR (CDCl₃) δ (ppm) 9.00 (dd, 1H), 8.28 (dd, 1H), 7.66 (m, 1H), 7.4 (m, 1H), 7.23 (m, 1H), 7.15 (t, 1H), 4.95 (d, 1H), 4.76 (d, 1H), 4.57 (d, 1H), 4.42 (d, 1H), 3.34 (s, 3H), 3.08 (s, 3H); 300 MHz ¹⁹F NMR (CDCl₃) δ (ppm) −116.71; MS: 450 (M+1).

### Example 68

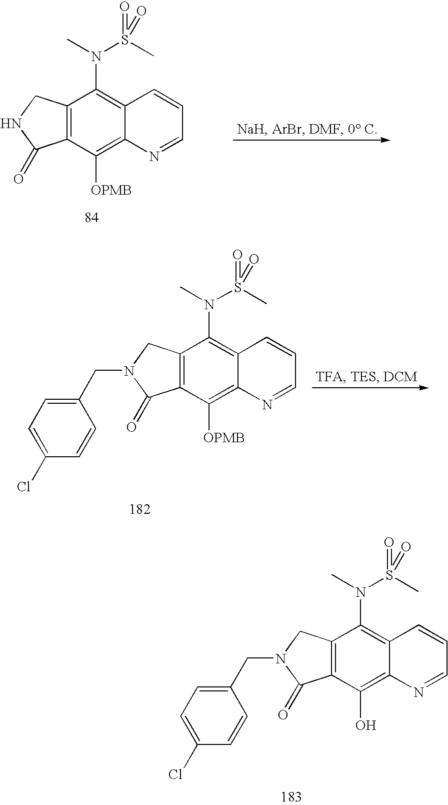

Compound 182: The compound was made in a similar fashion as compound 96 to afford the desired product 182 (30 mg, 77%): 300 MHz ¹H NMR (CDCl₃) δ (ppm) 9.08 (dd, 1H), 8.25 (dd, 1H), 7.65 (m, 3H), 7.35 (m, 4H), 6.9 (m, 2H), 5.8 (m, 2H), 4.8 (m, 2H), 4.5 (m, 2H), 3.8 (s, 3H), 3.34 (s, 3H), 3.1 (s, 3H); MS: 552 (M+1).

Compound 183: The compound was made in a similar fashion as compound 97 except no trifluoroacetic acid (TFA) was added in the reversed phase HPLC purification to afford the desired product 183 (10 mg, 43%) as the free parent: 300 MHz ¹H NMR (CDCl₃) δ.(ppm) 9.02 (dd, 1H), 8.28 (dd, 1H), 7.68 (m, 1H), 7.32 (m, 4H), 5.0 (d, 1H), 4.70(d, 1H), 4.57 (d, 1H), 4.36 (d, 1H), 3.33 (s, 3H), 3.07 (s, 3H); MS: 432 (M+1).

### Example 69

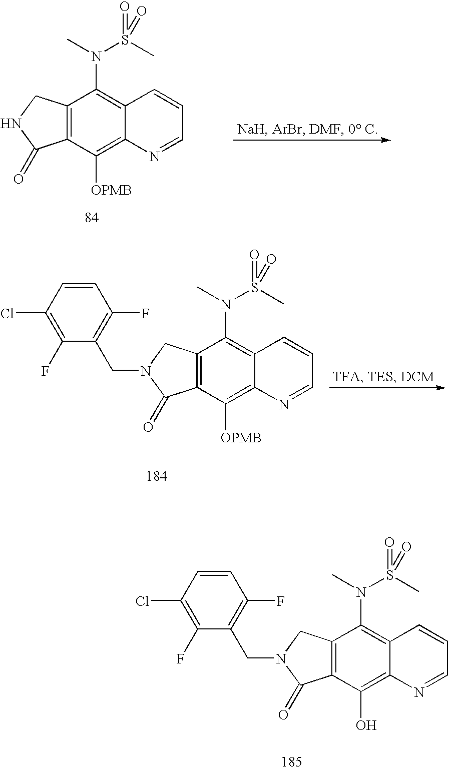

Compound 184: The compound was made in a similar fashion as compound 96 to afford the desired crude product 184 (˜35 mg) with no further characterization: MS: 588 (M+1).

Compound 185: The compound was made in a similar fashion as compound 97 to afford the desired product 185 (22 mg, 67%, 2 steps) as the free parent: 300 MHz ¹H NMR (CDCl₃) δ (ppm) 8.98 (dd, 1H), 8.31 (dd, 1H), 7.65 (m, 1H), 7.41 (m, 1H), 6.91 (m, 1H), 5.02 (d, 1H), 4.85 (d, 1H), 4.75 (d, 1H), 4.45 (d, 1H), 3.36 (s, 3H), 3.09 (s, 3H); 300 MHz ¹⁹F NMR (CDCl₃) δ (ppm) −114.07, −115.22; MS: 468 (M+1).

### Example 70

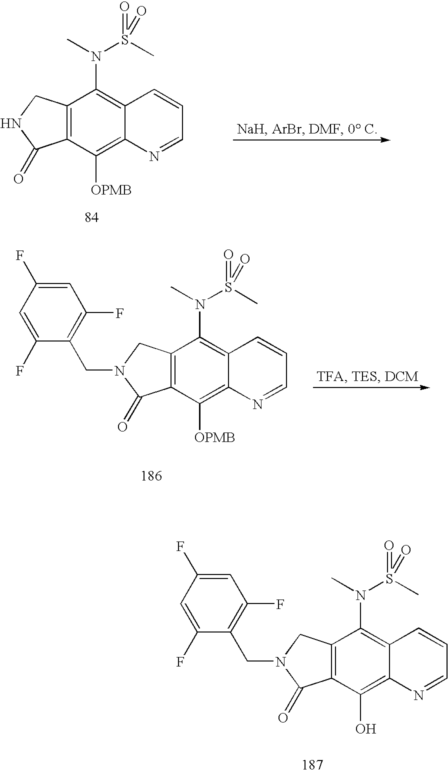

Compound 186: The compound was made in a similar fashion as compound 96 to afford the desired crude product 186 (˜35 mg) with no further characterization: MS: 572 (M+1).

Compound 187: The compound was made in a similar fashion as compound 97 to afford the desired product 187 (22 mg, 69%, 2 steps) as the free parent: 300 MHz ¹H NMR (CDCl₃) δ (ppm) 8.99 (dd, 1H), 8.28 (dd, 1H), 7.65 (m, 1H), 6.75 (t, 2H), 4.95 (d, 1H), 4.79 (m, 2H), 4.45 (d, 1H), 3.36 (s, 3H), 3.08 (s, 3H); 300 MHz ¹⁹F NMR (CDCl₃) δ (ppm) −106.97, −111.18; MS: 452 (M+1).

### Example 71

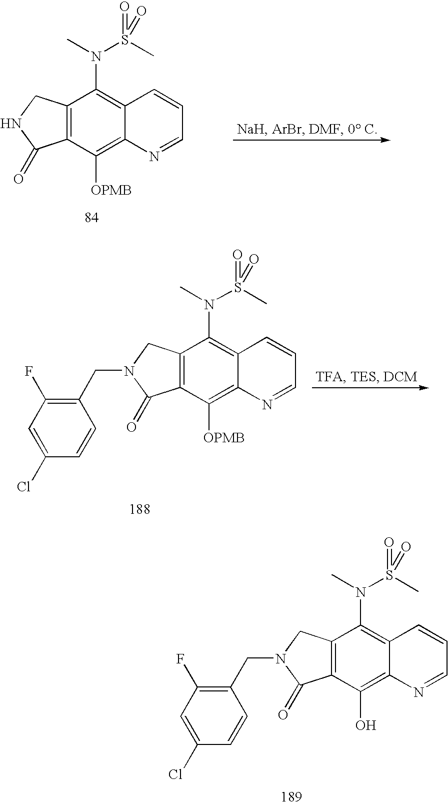

Compound 188: The compound was made in a similar fashion as compound 96 to afford the desired crude product 188 (˜35 mg) with no further characterization: MS: 570 (M+1).

Compound 189: The compound was made in a similar fashion as compound 97 to afford the desired product 189 (16 mg, 51%, 2 steps) as the free parent: 300 MHz ¹H NMR (CDCl₃) δ (ppm) 8.99 (dd, 1H), 8.29 (dd, 1H), 7.65 (m, 1H), 7.39 (t, 1H), 7.15 (m, 2H), 4.92 (d, 1H), 4.75 (m, 2H), 4.45 (d, 1H), 3.35 (s, 3H), 3.09 (s, 3H); 300 MHz ¹⁹F NMR (CDCl₃) δ.(ppm) −116.59; MS: 450 (M+1).

### Example 72

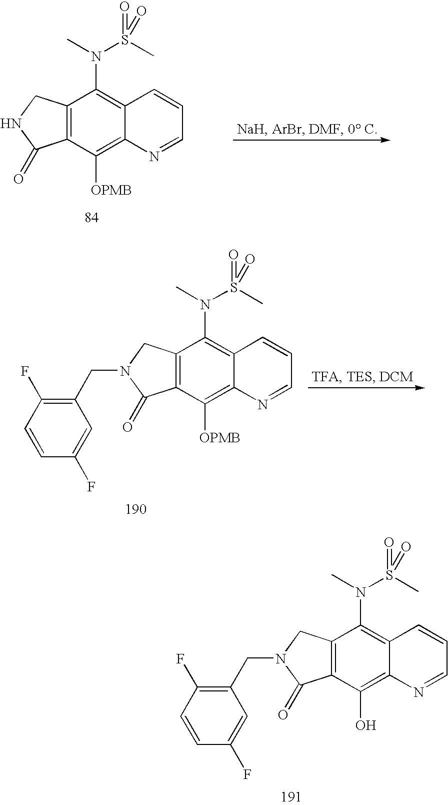

Compound 190: The compound was made in a similar fashion as compound 96 to afford the desired crude product 190 (˜35 mg) with no further characterization: MS: 554 (M+1).

Compound 191: The compound was made in a similar fashion as compound 97 to afford the desired product 191 (16 mg, 53%, 2 steps) as the free parent: 300 MHz ¹H NMR (CDCl₃) δ (ppm) 8.99 (dd, 1H), 8.3 (dd, 1H), 7.65 (m, 1H), 7.3-6.9 (m, 3H), 4.95 (d, 1H), 4.78 (d, 1H), 4.73 (d, 1H), 4.48 (d, 1H), 3.36 (s, 3H), 3.09 (s, 3H); 300 MHz ¹⁹F NMR (CDCl₃) δ (ppm) −118.11, −125.29; MS: 434 (M+1).

### Example 73

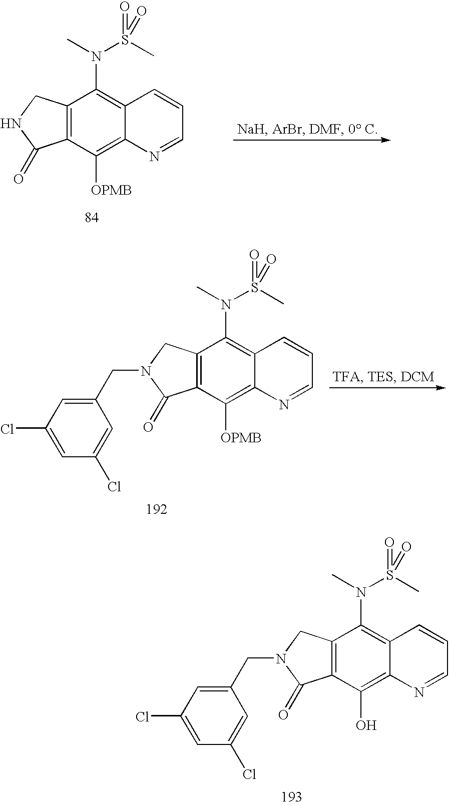

Compound 192: The compound was made in a similar fashion as compound 96 to afford the desired crude product 192 (˜35 mg) with no further characterization: MS: 586 (M+1).

Compound 193: The compound was made in a similar fashion as compound 97 to afford the desired product 193 (16 mg, 49%, 2 steps) as the free parent: 300 MHz ¹H NMR (CDCl₃) δ (ppm) 9.0 (dd, 1H), 8.29 (dd, 1H), 7.67 (m, 1H), 7.3 (m, 1H), 7.26 (m, 2H), 5.0 (d, 1H), 4.73 (d, 1H), 4.5 (d, 1H), 4.41 (d, 1H), 3.35 (s, 3H), 3.09 (s, 3H); MS: 466 (M+1).

### Example 74

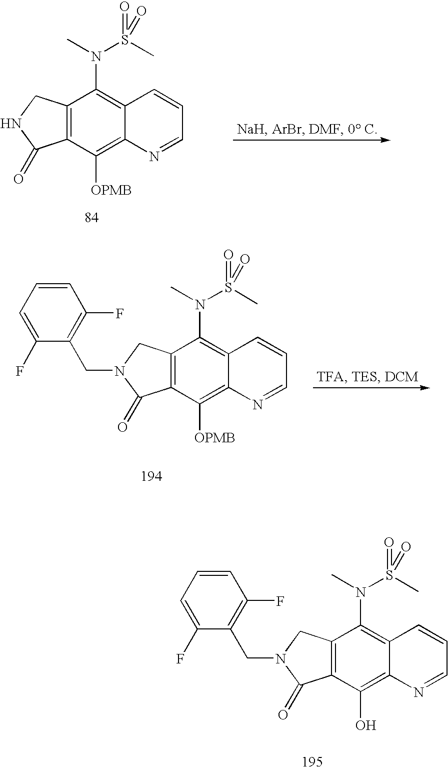

Compound 194: The compound was made in a similar fashion as compound 96 to afford the desired crude product 194 (˜40 mg) with no further characterization: MS: 554 (M+1).

Compound 195: The compound was made in a similar fashion as compound 97 to afford the desired product 195 (20 mg, 66%, 2 steps) as the free parent: 300 MHz ¹H NMR (CDCl₃) δ (ppm) 8.99 (dd, 1H), 8.29 (dd, 1H), 7.63 (m, 1H), 7.33 (m, 1H), 6.98 (m, 2H), 4.99 (d, 1H), 4.85 (d, 1H), 4.75 (d, 1H), 4.45 (d, 1H), 3.35 (s, 3H), 3.07 (s, 3H); 300 MHz ¹⁹F NMR (CDCl₃) δ (ppm) −114.38; MS: 434 (M+1).

### Example 75

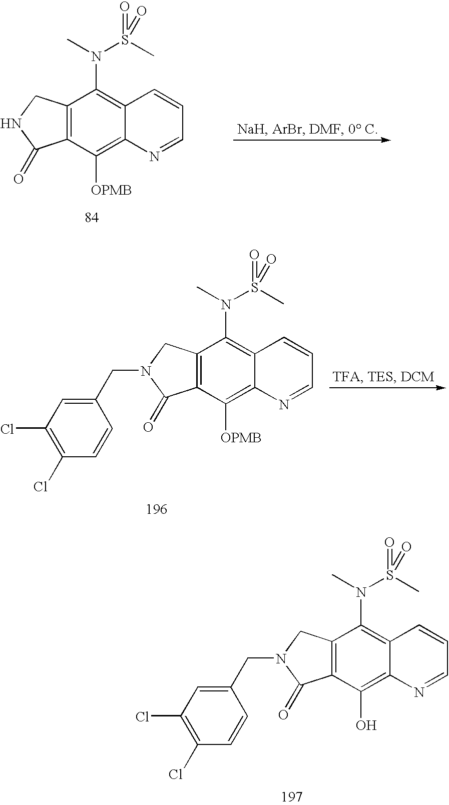

Compound 196: The compound was made in a similar fashion as compound 96 to afford the desired crude product 196 (˜35 mg) with no further characterization: MS: 586 (M+1).

Compound 197: The compound was made in a similar fashion as compound 97 to afford the desired product 197 (20 mg, 61%, 2 steps) as the free parent: 300 MHz ¹H NMR (CDCl₃) δ (ppm) 9.0 (dd, 1H), 8.29 (dd, 1H), 7.67 (m, 1H), 7.45 (m, 2H), 7.20 (m, 1H), 4.97 (d, 1H), 4.73 (d, 1H), 4.53 (d, 1H), 4.41 (d, 1H), 3.34 (s, 3H), 3.08 (s, 3H); MS: 466 (M+1).

### Example 76

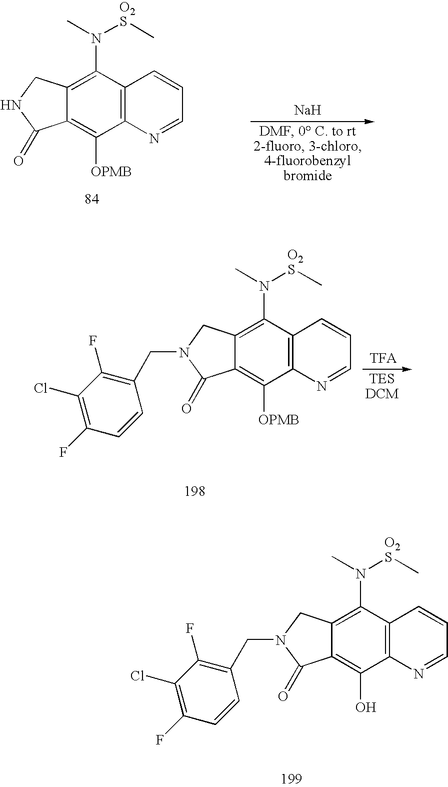

Using the methods of Example 75, compound 84 afforded product 198.

¹H NMR (300 MHz, CD₃CN) shows diagnostic peaks at δ 8.95 (m, 1H), 8.55 (m, 1H) 4.85 (m, 2H), 3.38 (s, 3H) and 3.18 (s, 3H). MS=468.0 (M+H).

### Example 77

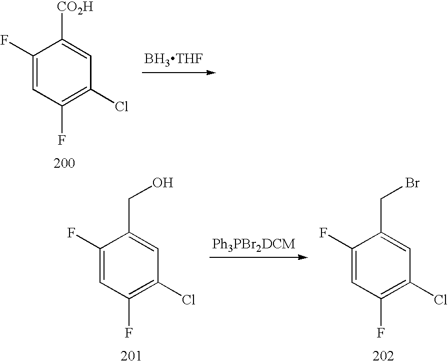

Compound 201: Following the procedure detailed in Journal of Medicinal Chemistry, 2001, Vol. 44, No. 25, pp 4398, the commercially available carboxylic acid 200 (1 g, 5.21 mmol) was dissolved in THF (17.4 mL, 0.3 M), cooled in an ice bath to 0° C. then treated with borane-tetrahydrofuran complex (10.43 mL, 2 equiv, 1M soln THF) added dropwise. The reaction was stirred for 24 hours at room temperature under an inert atmosphere upon which cooled in an ice bath then quenched with 3N HCl (3 mL). The mixture was diluted with ethyl acetate and H₂O was added. The organic layer was washed with H₂O and brine, then dried (over Na₂SO₄), filtered and concentrated in vacuo. The crude residue was purified by chromatography on silica gel (1/9—ethyl acetate/hexane) to afford the desired alcohol 201 (813 mg, 88%): 300 MHz ¹H NMR (CDCl₃) δ (ppm) 7.52 (dd, 1H), 6.92 (dd, 1H), 4.73 (s, 2H), 1.8 (bs, 1H); 300 MHz ¹⁹F NMR (CDCl₃) δ (ppm) −112.65 −117.7.

Compound 202: A solution of the alcohol 201 (200 mg, 1.1 mmol) in dichloromethane (11 mL) cooled in an ice bath to 0° C. was treated with dibromotriphenyl phosphorane (570 mg, 1.3 mmol). After being stirred at room temperature overnight, the reaction had not completed and was treated with dibromotriphenyl phosphorane (150 mg, twice) and allowed to stir again overnight. The reaction mixture was concentrated down in vacuo, then the crude residue was purified by chromatography on silica gel (1/9—ethyl acetate/hexane) to afford the desired bromide 202 (250 mg, 93%): 300 MHz ¹H NMR (CDCl₃) δ.(ppm) 7.46 (dd, 1H), 6.4 (dd, 1H), 4.44 (s, 2H), 1.8 (bs, 1H); 300 MHz ¹⁹F NMR (CDCl₃) δ.(ppm) −109.93 −114.37.

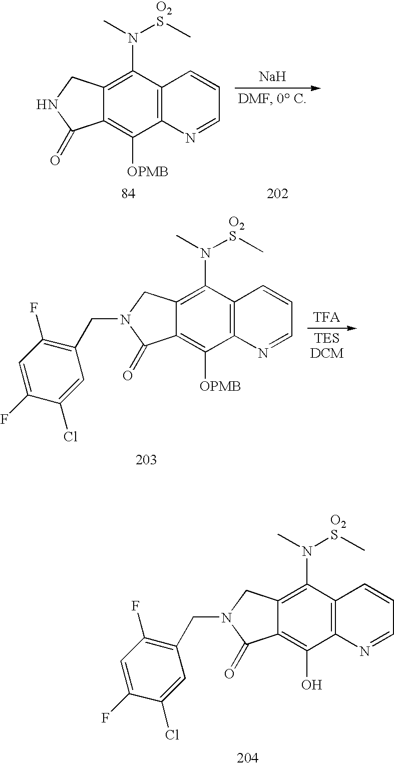

Compound 203: The compound was made in a similar fashion as compound 96 to afford the desired product 203 (349 mg, 92%): 300 MHz ¹H NMR (CDCl₃) δ.(ppm) 9.08 (dd, 1H), 8.27 (dd, 1H), 7.65 (m, 3H), 7.52 (dd, 1H), 6.98 (dd, 1H), 6.9 (d, 2H), 5.8 (m, 2H), 4.8 (m, 2H), 4.55 (m, 2H), 3.8 (s, 3H), 3.36 (s, 3H), 3.12 (s, 3H); MS: 588 (M+1).

Compound 204: The compound was made in a similar fashion as compound 97 to afford the desired product 204 (205 mg, 74%) as the free parent: 300 MHz ¹H NMR (CDCl₃) δ (ppm) 8.99 (dd, 1H), 8.29 (dd, 1H), 7.66 (m, 1H), 7.53 (dd, 1H), 6.98 (dd, 1H), 4.92 (d, 1H), 4.78 (d, 1H), 4.70 (d, 1H), 4.48 (d, 1 H), 3.36 (s, 3H), 3.10 (s, 3H); 300 MHz ¹⁹F NMR (CDCl₃) δ (ppm) −110.99 −116.56; MS: 468 (M-+1).

Compound 204 may also be prepared from the following scheme.

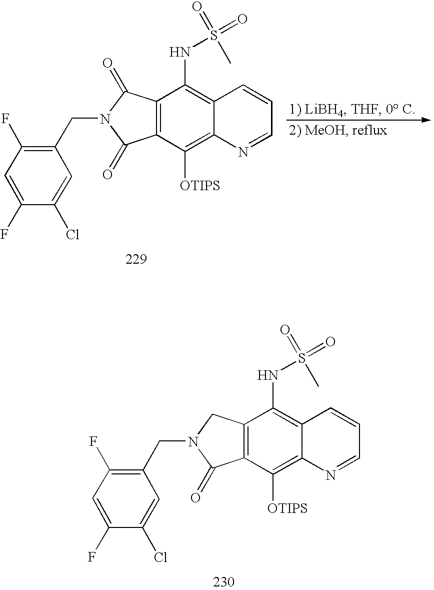

Compound 229 may be prepared through known techniques in the art.

Compound 230 is methylated and deprotected to give compound 204.

### Example 78

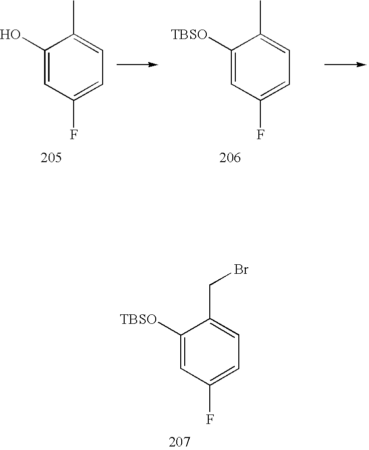

To phenol 205 (7 g, 55.53 mmol, 1 equiv.) was added CH₂Cl₂ (180 mL, 0.3 M) and treated with triethylamine (11.56 mL, 83.30 mmol, 1.5 equiv.) and DMAP (680 mg, 5.56 mmol, 0.5 equiv.). TBSCl (9.16 g, 61.08 mmol, 1.1 equiv.) was slowly added and the reaction mixture was stirred at room temperature for 2 h a under nitrogen atmosphere. The reaction mixture was diluted with CH₂Cl₂ (400 mL) and quenched with water (200 mL). The layers were separated and the aqueous layer was extracted with ethyl acetate (200 mL). The combined organic layer was washed with water and brine then dried (over Na₂SO₄), filtered and concentrated in vacuo to obtain a clear oil of 206 (13.35 g, 100% mass recovery).

300 MHz ¹H NMR (CDCl₃) δ (ppm) 7.27-7.03 (m, 1H), 6.61-6.47 (m, 2H), 2.23 (s, 3H), 1.03 (s, 9H), 0.24 (s, 6H). MS: 430.07 (M+1).

To 206 (13.35 g, 55.59 mmol, 1 equiv.) was added CCl₄ (185 mL, 0.3 M) and to it added N-Bromosuccinimide (11.82 g, 66.71 mmol, 1.2 equiv.) and benzoyl peroxide (1.35 g, 5.56 mmol, 0.1 equiv.). The mixture was stirred under an inert atmosphere, refluxed and a ultra violet lamp shined to the reaction flask. The reaction was cooled and the solid filtered over a sintered funnel and the filtrate concentrated in vacuo. Purification was carried out by ISCO flash column chromatography was carried out with 4/1 EtOAc/ Hexanes to yield 207.

300 MHz ¹H NMR (CDCl₃) δ (ppm) 7.31-7.28 (m, 1H), 6.68-6.62 (m, 1H), 6.56-6.50 (m, 1H), 4.50 (s, 2H), 1.06 (s, 9H), 0.31 (s, 6H). 300 MHz ¹⁹F NMR (CDCl₃) δ (ppm) −110.91

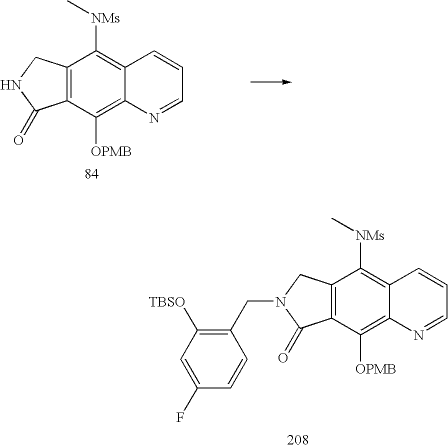

To lactam 84 (120 mg, 0.28 mmol, 1 equiv.) was added DMF (3 mL, 0.1 M) and cooled in an ice bath to 0C before added sodium hydride (13.5 mg, 0.34 mmol, 60% mineral oil, 1.3 equiv.) and stirred for 5 minutes under nitrogen atmosphere. Bromide 207 (107 mg, 0.34 mmol, 1.2 equiv.) was added and the reaction was allowed to stir for 30 minutes at 0° C. The reaction was quenched with water and diluted with Ethyl Acetate. The organic layer was washed with water and brine before being dried over sodium sulfate, filtered and concentrated in vacuo. The crude residue was purified by silica gel chromatography eluting with 4/1 EtOAc/Hexanes to afford the desired product 208 (128 mg, 80%).

300 MHz ¹H NMR (CDCl₃) δ (ppm) 9.05 (s, 1H), 8.28-8.26 (m, 1H), 7.64-7.60 (s, 3H), 6.90-6.85 (m, 2H), 6.68-6.57 (m, 3H), 5.84-5.74 (m, 2H), 5.13 (d, J=14.7 Hz, 1H), 4.88 (d, J=17.1 Hz, 1H), 4.75 (d, J=14.7 Hz, 1H), 4.69 (d, J=17.1 Hz, 1H)), 3.80 (s, 3H), 3.30 (s, 3H), 3.11 (s, 3H), 1.06 (s, 9H), 0.33 (s, 6H). MS: 666.14 (M+1).

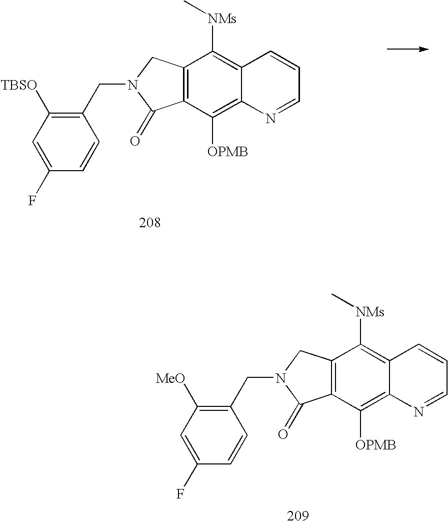

To lactam 208 (50 mg, 0.075 mmol, 1 equiv.) was added THF (7.5 mL) and cooled in an ice bath to 0° C. before added tetra-butylammonium fluoride (491 mg, 0.188 mmol, 2.5 equiv.) was added and stirred for 30 minutes under a nitrogen atmosphere. The reaction was quenched with water and diluted with Ethyl Acetate. The organic layer was washed with water and brine before being dried over sodium sulfate, filtered and concentrated in vacuo to obtain 38 mg of a crude phenol. This phenol (38 mg, 0.008 mmol, 1 equiv.) was stirred in DMF (3 mL) and to it added Cs₂CO₃ (44 mg, 0.17 mmol, 2 equiv.) and iodomethane (20 μL, 0.21 mmol, 2 equiv.) and allowed to stir for 1 hr. The reaction was quenched with water and diluted with Ethyl Acetate. The organic layer was washed with water and brine before being dried over sodium sulfate, filtered and concentrated in vacuo. The crude residue was purified by silica gel chromatography eluting with 4/1 EtOAc/Hexanes to afford the desired product 209.

300 MHz ¹H NMR (CDCl₃) δ (ppm) 9.11-9.05 (m, 2H), 8.72-8.68 (m, 1H), 7.71-7.63 (m, 2H), 7.53 (d, J=8.1 Hz, 1H), 5.86-5.75 (m, 2H), 5.13 (d, J=14.7 Hz, 1H), 4.88 (d, J=17.1 Hz, 1H), 4.75 (d, J=14.7 Hz, 1H), 4.69 (d, J=17.1 Hz, 1H), 3.80 (s, 3H), 3.45 (s, 3H), 3.30 (s, 3H), 3.11 (s, 3H). 300 MHz ¹⁹F NMR (CDCl₃) δ (ppm) −1 17.15, −76.32. MS: 566.04 (M+1).

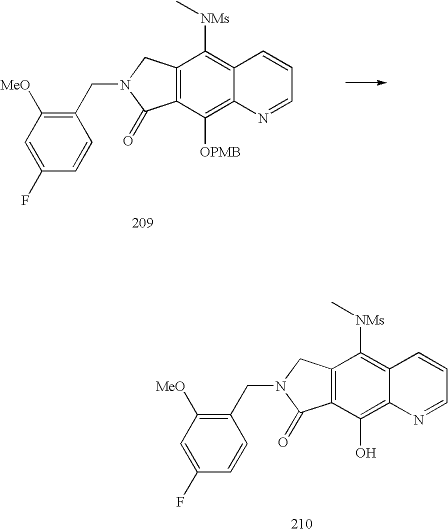

Phenol 210 was made in a similar fashion as described for compound 97. 300 MHz ¹H NMR (CDCl₃) δ (ppm) 9.11 (s, 1H), 8.43(d, J=8.7 Hz, 1H), 7.72-7.21 (m, 1H), 7.34-7.30 (m, 1H), 6.67-6.64 (m, 2H), 4.81 (d, J=15.9 Hz, 1H), 4.72 (d, J=15.9 Hz, 1H), 4.71 (d, J=18.0 Hz, 1H), 4.48 (d, J=18.0 Hz, 1H), 3.88 (s, 3H), 3.34 (s, 3H), 3.08 (s, 3H). 300 MHz ¹⁹F NMR (CDCl₃) δ (ppm) −110.99.15, −76.21 (TFA salt). MS: 446.10 (M+1).

### Example 79

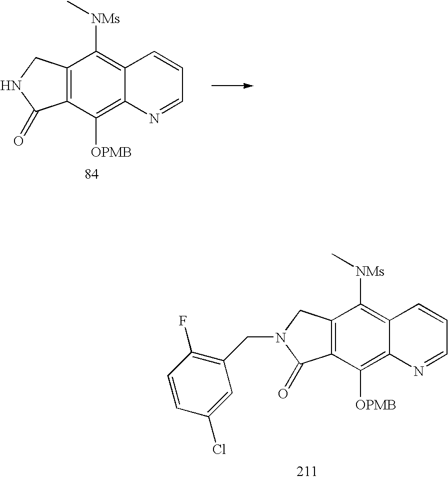

To lactam 84 (29 mg, 0.0.67 mmol, 1 equiv.) was added DMF (1 mL, 0.07 M) and cooled in an ice bath to 0° C. before added sodium hydride (3 mg, 0.075 mmol, 60% mineral oil, 1.1 equiv.) and stirred for 5 minutes under nitrogen atmosphere. 2-Bromomethyl-4-chloro-1-fluoro-benzene (21 μL, 0.10 mmol, 1.5 equiv.) was added and the reaction was allowed to stir for 60 minutes at 0° C. The reaction was quenched with water and diluted with Ethyl Acetate. The organic layer was washed with water and brine before being dried over sodium sulfate, filtered and concentrated in vacuo. The crude residue was purified by silica gel chromatography eluting with 4/1 EtOAc/Hexanes to afford the desired product 211. See below for characterization after PMB deprotection

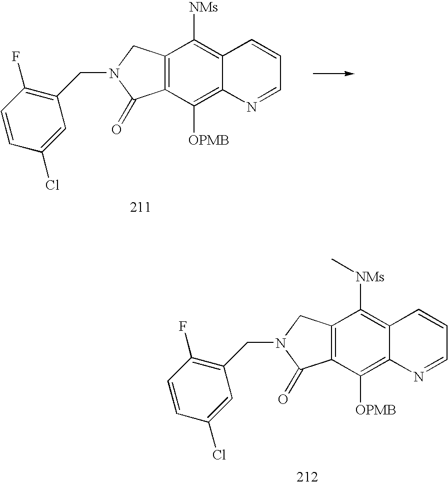

Phenol 212 was made in a similar fashion as described for compound 97 300 MHz ¹H NMR (CDCl₃) δ (ppm) 9.07 (d, J=3.3 Hz, 1H), 8.33 (d, J=8.4 Hz, 1H), 7.71 (dd, J₁=3.3 Hz, J₂=4.5 Hz, 1H), 7.40-7.35 (m, 1H), 7.10-7.04 (m, 2H), 4.96 (d, J=15.3 Hz, 1H), 4.80 (d, J=16.8 Hz, 1H), 4.75 (d, J=17.4 Hz, 1H), 4.51 (d, J=17.4 Hz, 1H), 3.36 (s, 3H), 3.10 (s, 3H). 300 MHz ¹⁹F NMR (CDCl₃) δ.(ppm) −121.84, −76.23 (TFA salt).

MS: 450.05 (M+1).

### Example 80

Representative procedure for the synthesis of compounds 214 and 215

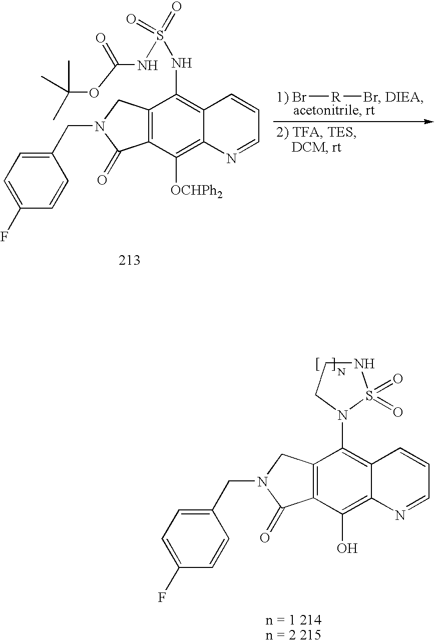

To 200 mg of BOC-protected sulfonyl urea 213 in 8 mL acetonitrile at room temperature was added 262 μL DIEA, followed by 91 μL of 1,3-dibromopropane (0.9 mmol, 3 equiv). After 2 d the reaction was stopped, and the reaction was diluted with 50 mL ethyl acetate. The organics were washed with 25 mL 0.1M HCl, 25 mL water, and then 25 mL aq. brine solution. After drying over Na₂SO₄, solvent was removed by rotary evaporation to give 181 mg of the alkylated sulfonyl urea intermediate. Treatment of this product material with excess TFA and TES in a 1.0M solution of DCM resulted in global deprotection of the BOC and DPM protecting groups. 60 mg (45% yield over 2 steps) of the cyclic sulfonyl urea product 214 as the TFA salt resulted after purification by reverse phase HPLC.

214-: 300MHz ¹H NMR (CD₃CN) δ.(ppm): 8.9(d, 1H), 8.8(d, 1H), 7.8(m, 1H), 7.4(t, 2H), 7.1(t, 2H), 5.5(s, 1H), 4.8(s, 2H), 4.5(dd, 2H), 3.9(t, 1H), 1H), 3.6(m, 3H). m/z=443 (M+H).

215-: 300MHz ¹H NMR (CD₃CN) δ.(ppm): 8.9(d, 1H), 8.8(d, 1H), 7.8(m, 1H), 7.4(t, 2H), 7.1(t, 2H), 5.5(s, 1H), 4.8-4.4(dd, 4H), 3.8(m, 2H), 3.6(m, 2H). m/z=429 (M+H).

### Example 81

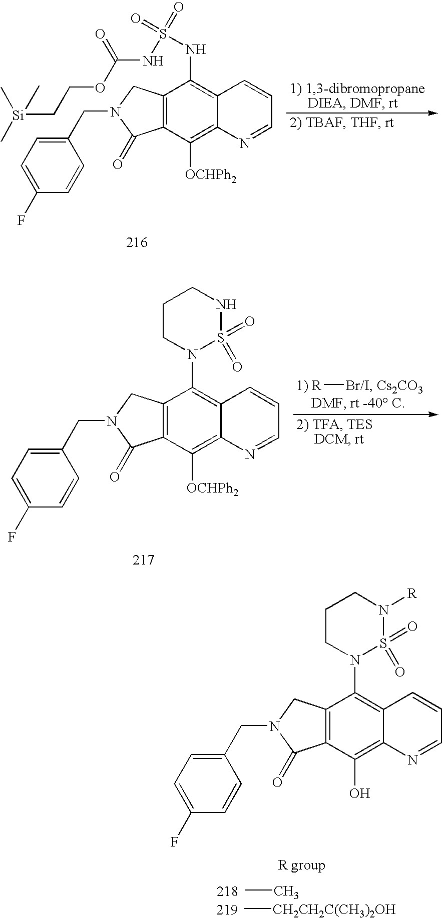

To 900 mg of TEOC protected sulfonyl urea 216, obtained in an analogous manner to that described in Example 42 for the preparation of compound 131, in 30 mL DMF at room temperature was added 1.1 mL DIEA (6.3 mmol, 5 equiv.), followed by 385 μL of 1,3-dibromopropane (3.8 mmol, 3 equiv). After 18 h the reaction was shown to be complete by LC/MS, and the reaction was diluted with 50 mL ethyl acetate. The organics were washed with 25 mL water and then 25 mL aq. brine solution. After drying over Na₂SO₄, solvent was removed by rotary evaporation to give 800 mg of the TEOC protected cyclic sulfonyl urea intermediate. Treatment of this product material with TBAF in THF (0.1M) resulted in deprotection of the TEOC protecting group to give 217. The intermediate was redissolved in DMF and Cs₂CO₃ was added, followed by iodomethane. The reaction mixture was heated to 40° C. overnight in an oil bath and by LC/MS, the reaction was complete. The reaction was diluted with ethyl acetate and washed with water and brine. After drying over Na₂SO₄, the solvent was removed to yield a dark red-orange solid. The solid was then dissolved in DCM, and the DPM protecting group was removed via the previously mentioned route using excess TFA and TES in a 1.0M solution of DCM. 150 mg (25% yield over 4 steps) of the cyclic urea product 218 as the TFA salt were recovered after purification by reverse phase HPLC.

218-: 300 MHz ¹H NMR (CDCl₃) δ (ppm): 8.9 (d, 1H), 8.7 (d, 1H), 8.0 (s, 1H), 7.6 (m, 1H), 7.3 (t, 2H), 7.0 (t, 2H), 4.9-4.7 (dd, 2H), 4.6-4.3 (dd, 2H), 3.9 (q, 2H), 3.5 (m, 2H), 3.0 (s, 3H), 2.2 (m, 1H), 1.7 (m, 2H). m/z=457 (M+H).

219-: 300 MHz ¹H NMR (CD₃CN) δ (ppm): 8.9 (d, 1H), 8.8 (d, 1H), 7.8 (m, 1H), 7.4 (t, 2H), 7.1 (t, 2H), 4.8-4.5 (dd, 4H), 3.7 (m, 4H), 3.5 (m, 2H), 2.1 (m, 1H), 1.7 (m, 3H), 1.1 (m, 6H). m/z=529 (M+H).

### Example 82

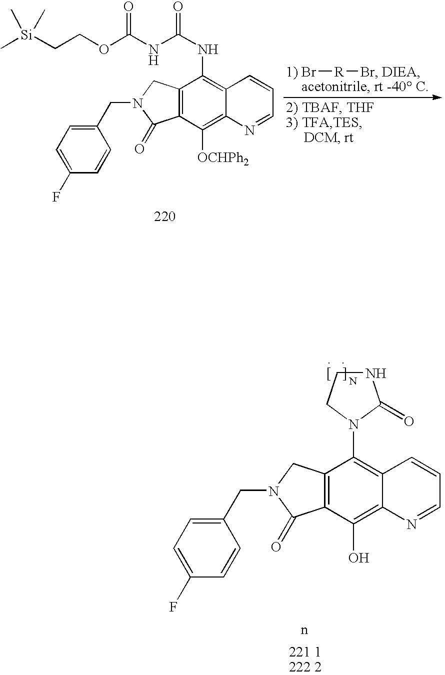

To 200 mg of TEOC-protected urea 220 in 8 mL DMF at 40° C. was added 293 mg Cs₂CO₃, followed by 91 μL of 1,3-dibromopropane (0.9 mmol, 3 equiv). After 1 h the reaction was complete and the mixture diluted with 50 mL ethyl acetate. The organics were washed with 25 mL water and then 25 mL aq. brine solution. After drying over Na₂SO₄, solvent was removed by rotary evaporation to give 216 mg crude of the cyclic urea intermediate. Column chromatography using ISCO Combi-flash instrumentation with Hexanes/Ethyl Acetate (30/70) resulted in 88 mg of pure product. Treatment of this product material with TBAF (0.4 mmol, 3 equiv) in THF (0.1M) resulted in deprotection of the TEOC protecting group. The additional DPM protecting group was removed via the previously mentioned route using excess TFA and TES in a 1.0M solution of DCM. 31 mg (25% yield over 2 steps) of the cyclic urea product 5013a as the TFA salt resulted after purification by reverse phase HPLC.

221-: 300 MHz ¹H NMR (DMSO-d6) δ (ppm): 8.9 (d, 1H), 8.2 (d, 1H), 7.7 (m, 1H), 7.3 (t, 2H), 7.1 (t, 2H), 6.6 (s, 1H), 4.8-4.5(dd, 2H), 4.4-4.1 (dd, 2H), 3.4 (m, 2H), 3.2 (m, 2H), 2.0 (m, 1H), 1.9 (m, 1H). m/z=407 (M+H).

222-: 300 MHz ¹H NMR (CDCl₃) δ (ppm): 9.0 (d, 1H), 8.3 (d, 1H), 7.6 (m, 1H), 7.3 (t, 2H), 7.0 (t, 3H), 5.5 (s, 1H), 4.9-4.6 (dd, 2H), 4.5-4.2 (dd, 2H), 3.9 (m, 2H), 3.7 (m, 2H). m/z=393 (M+H).

### Example 83

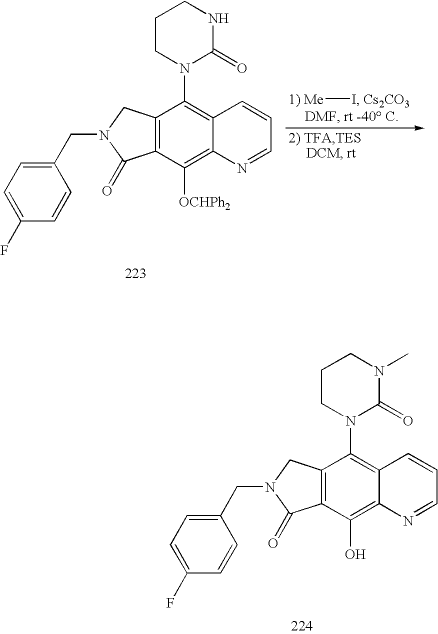

To 40 mg of cyclic urea intermediate 223 from TEOC deprotection dissolved in 2 mL DMF was added Cs₂CO₃, followed by 13 μL methyl iodide. The reaction mixture stirred at 40° C. overnight and was complete by LC/MS. Reaction cooled and diluted with EtOAc. Organics washed with brine and dried over Na₂SO₄. The crude material was carried forward to DPM deprotection. The DPM protecting group was removed via the previously mentioned route using excess TFA and TES in a 1.0M solution of DCM. 31 mg (25% yield over 2 steps) of the cyclic urea product 224 as the TFA salt resulted after purification by reverse phase HPLC.

224-: 300 MHz ¹H NMR (CDCl₃) δ (ppm): 9.0 (d, 1H), 8.3 (d, 1H), 7.6 (m, 1H), 7.3 (t, 2H), 7.0 (t, 3H), 4.8-4.6 (dd, 2H), 4.4-4.1 (dd, 2H), 3.5 (m, 4H), 3.0 (s, 3H), 1.2 (s, 2H). m/z=421 (M+H),

### Example 84

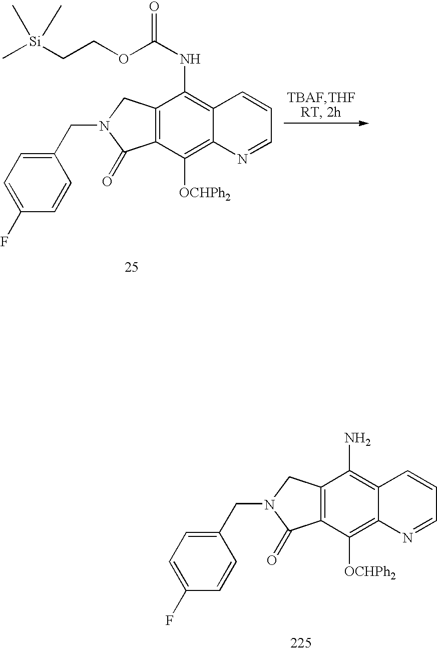

Teoc-protected scaffold 26 (0.5 g, 1.0 eq, 0.8 mmol) was dissolved in THF (8 mL) and solid TBAF (0.6 g, 3.0 eq, 2.4 mmol) was added. The reaction mixture stirred under nitrogen for 2 h and LC/MS indicated that the reaction was complete. Aqueous ammonium chloride was added to quench the reaction and the product was extracted with EtOAc. The organic layer was washed with 0.1% citric acid, water, and brine, dried over Na₂SO₄, and concentrated to a yellow-orange solid as compound 225.

5: 300 MHz ¹H NMR (CDCl₃) δ (ppm): 9.0 (m, 1H), 8.1 (d, 1H), 7.7 (m, 5H), 7.3 (m, 1H), 7.2 (m, 5H), 7.1 (t, 2H), 7.0 (t, 2H), 4.8 (s, 2H), 4.0 (s, 2H). MS: 490 (M+H).

### Example 85

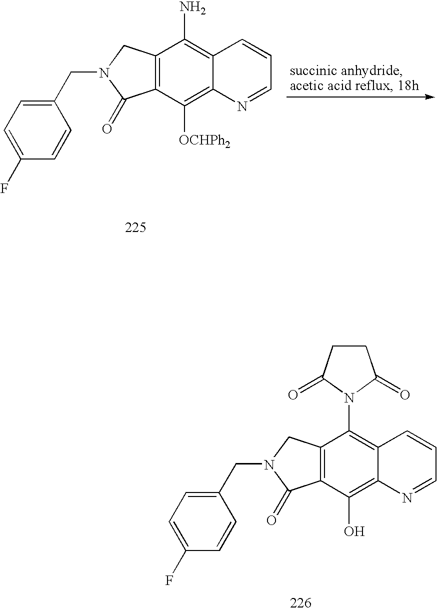

To 50 mg of DPM protected aniline 225 in 1 mL acetic acid (0.1M), was added 10 mg of succinic anhydride. The reaction mixture was fitted with condenser, placed under nitrogen atmosphere, and refluxed for 18 h. LC/MS confirmed desired product mass and the reaction was cooled to room temperature The reaction mixture was filtered and washed with ether/hexanes (50/50) to yield 22 mg of desired imide 226 (51% yield).

226-: 300 MHz ¹H NMR (CD₃OD) δ (ppm): 8.9 (d, 1H), 8.3 (d, 1H), 7.7 (m, 1H), 7.3 (t, 2H), 7.1 (t, 2H), 4.7 (s, 2H), 4.3 (s, 2H), 2.9(qq, 4H). m/z=406 (M+H).

### Example 86

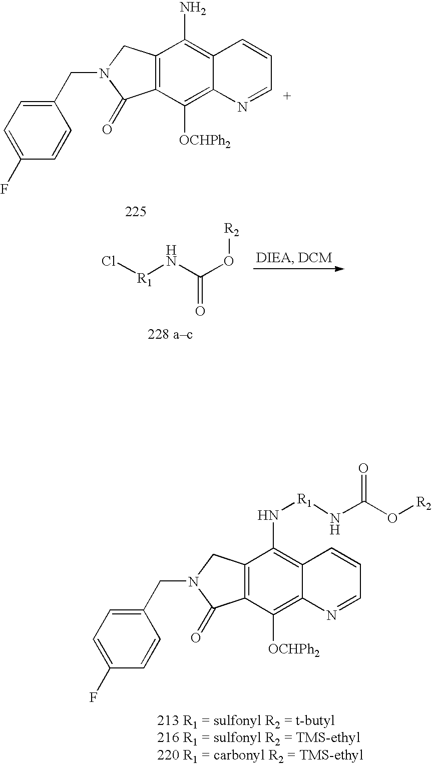

Aniline scaffold 225 (0.6 g, 1.0 eq, 1.2 mmol) was dissolved in DCM (30 mL) and DIEA (210 μL, 1.0 eq, 1.2 mmol) added. The chloride reagent 228a-c reported in Winum et al. Organic Letters 2001, 3, p. 2241-2243 (0.3M solution in DCM) was added drop wise over 10 minutes. The reaction stirred at room temperature under nitrogen for 3 h and LC/MS showed the reaction to be complete. The reaction mixture was diluted with DCM and washed with water and brine. The organics were concentrated to yield the protected urea 213 as a yellow-orange solid (670 mg).

213: 300 MHz ¹H NMR (CDCl₃) δ ppm): 9.0 (m, 1H), 8.7 (d, 1H), 8.1 (s, 1H), 7.8 (d, 5H), 7.4 (m, 1H), 7.2 (m, 5H), 7.1 (t, 2H), 6.9 (t, 2H), 4.8 (s, 2H), 4.5 (s, 2H), 1.4 (s, 9H). MS: 669 (M+H).

216: 300 MHz ¹H NMR (CDCl₃) δ ppm): 9.0 (m, 1H), 8.6 (d, 1H), 8.0 (s, 1H), 7.8 (d, 5H), 7.4 (m, 1H), 7.2 (m, 5H), 7.1 (t, 2H), 6.9 (t, 2H), 4.7 (s, 2H), 4.4 (s, 2H), 4.1 (t, 2H), 1.4 (t, 2H), 0.0 (s, 9H). MS: 713 (M+H).

220: 300 MHz ¹H NMR (CDCl₃) δ ppm): 9.0 (m, 1H), 8.6 (d, 1H), 8.0 (s, 1H), 7.8 (d, 5H), 7.4 (m, 1H), 7.2 (m, 5H), 7.1 (t, 2H), 6.9 (t, 2H), 4.7 (s, 2H), 4.4 (s, 2H), 4.1 (t, 2H), 1.4 (t, 2H), 0.0 (s, 9H). MS: 677 (M+H).

### Example 87

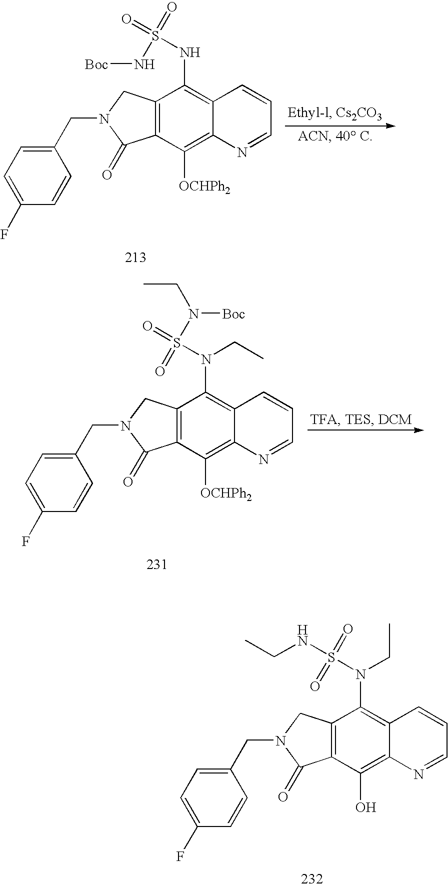

Common intermediate 213 was dissolved in ACN and Cs₂CO₃ was added, followed by ethyl iodide. The reaction mixture was heated to 40° C. overnight and LC/MS showed complete conversion to desired product. The reaction mixture was diluted with EtOAc and washed with water and brine. The organic layer was dried over Na₂SO₄ and concentrated to a red-orange solid. Intermediate 231 was then dissolved in DCM and TES added, followed by TFA. The reaction mixture stirred at room temperature for 30 minutes and LC/MS showed the reaction to be complete. The solvent was removed and the residue azeotroped with THF/Toluene to yield the deprotected product. The crude material was dissolved in MeOH and purified by rpHPLC resulting in pure compound 232 (51 mg).

232 300 MHz ¹H NMR (DMSO-d6) δ (ppm): 9.0 (d, 1H), 8.6 (d, 1H), 7.7 (m, 1H), 7.3 (t, 2H), 7.0 (t, 2H), 4.8-4.6 (dd, 2H), 4.6-4.4(dd, 2H), 3.7 (m, 2H), 3.1 (m, 2H), 1.1 (m, 3H), 1.0 (m, 3H). MS: 459 (M+H).

The above description is not intended to detail all modifications and variations of the invention. It will be appreciated by those skilled in the art that changes can be made to the embodiments described above without departing from the inventive concept. It is understood, therefore, that the invention is not limited to the particular embodiments described above, but is intended to cover modifications that are within the spirit and scope of the invention, as defined by the language of the following claims.

What is claimed is:

1. A compound having the formula A: 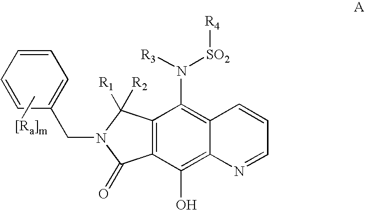
or a pharmaceutically acceptable salt thereof,
where,
each Rₐ is independently selected from the group consisting of hydrogen, chloro, fluoro, CH₃HNC(O)—, (CH₃)₂NC(O)—, (CH₃)₂NS(O)₂—, CH₃S(O)₂—, cyano and amino;
m is zero, one, two, three, four or five;
R₁ and R₂ are independently selected from the group consisting of hydrogen and C₁₋₄ alkyl;
R₃ is selected from from the group consisting of hydrogen, methyl and ethyl; and
R₄ is C₁₋₄ alkyl, N-ethylamino or N,N-dimethylamino; or
R₃ and R₄ are cyclized to form, together with the nitrogen atom pendent to the R₃ group and the SO₂ group pendent to the R₄ group, a heterocyclic or substituted heterocyclic group.

2. The compound of claim 1 which has the formula I or Ia: 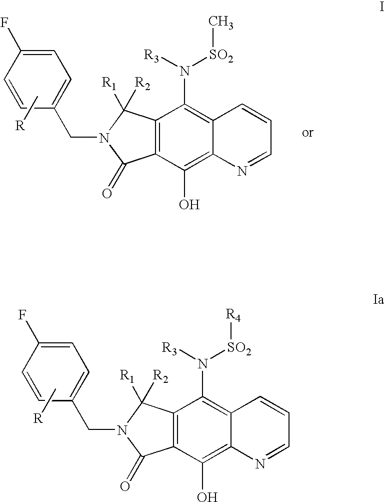
or a pharmaceutically acceptable salt thereof,
where,
R is selected from the group consisting of hydrogen, CH₃HNC(O)—, (CH₃)₂NC(O)—, (CH₃)₂NS(O)₂—, CH₃S(O)₂—, cyano and amino;
R₁ and R₂ are independently selected from the group consisting of hydrogen and methyl;
R₃ is selected from from the group consisting of hydrogen, methyl and ethyl; and
R₄ is N,N-dimethylamino; or
R₃ and R₄ are cyclized to form, together with the nitrogen atom pendent to the R₃ group and the SO₂ group pendent to the R₄ group, a heterocyclic or substituted heterocyclic group.

3. A compound having the formula II: 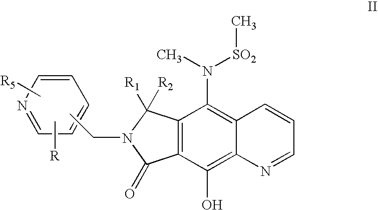
or a pharmaceutically acceptable salt thereof,
where,
R is selected from the group consisting of hydrogen, CH₃HNC(O)—, (CH₃)₂NC(O)—, (CH₃)₂NS(O)₂—, CH₃S(O)₂—, cyano and amino;
R₁ and R₂ are independently selected from the group consisting of hydrogen and methyl; and
R₅ is selected from the group consisting of hydrogen and fluoro.

4. The compound of claim 3 which has the formula IIb: 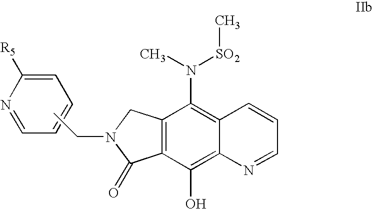
or a pharmaceutically acceptable salt thereof,
where,
R₅ is defined above.

5. A compound having the formula III: 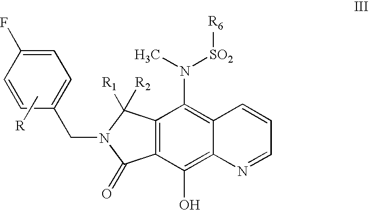
or a pharmaceutically acceptable salt thereof,
where,
R is selected from the group consisting of hydrogen, CH₃HNC(O)—, (CH₃)₂NC(O)—, (CH₃)₂NS(O)₂—, CH₃S(O)₂—, cyano and amino;
R₁ and R₂ are independently selected from the group consisting of hydrogen and methyl; and
R₆ is selected from the group consisting of methyl, ethyl, isopropyl, 1-methylimidazol-4-yl, 2,4-dimethylthiazol-5-yl, 2-(N,N-dimethylamino)eth-1-yl, 2-(N,N-diethylamino)eth-1-yl, 3-cyanoprop-1-yl, 3-(N-morpholino)prop-1-yl, 2-(N-morpholino)eth-1-yl, 3-(N,N-dimethylamino)prop-1-yl, amino, N-methylamino, N,N-dimethylamino, 2-(methylcarbonylamino)-4-methylthiazol-5-yl, 6-(N-morpholino)pyrid-3-yl, pyrid-2-yl, N-methyl-N-(pyrid-4-yl)methylamino, N-methyl-N-benzylamino, 2,2,2-trifluoroeth-1-yl, 2-(piperazin-2-yl)eth-1-yl, 2-(N-piperidinyl)eth-1-yl, 3-(imidazol-1-yl)-prop-1-yl, N-morpholino and 5-N-N-dimethylaminonaphth-1-yl.

6. The compound of claim 5 which has the formula IIIb: 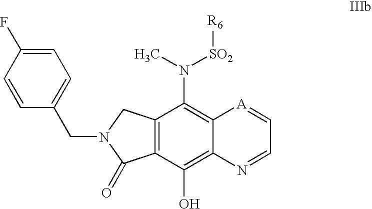
or a pharmaceutically acceptable salt thereof,
where,
R⁶ is defined above.

7. A compound having the formula IV: 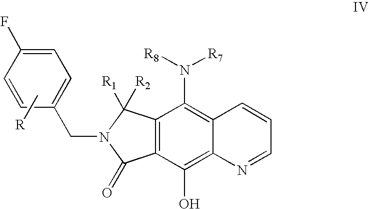
or a pharmaceutically acceptable salt thereof,
where,
R is selected from the group consisting of hydrogen, CH₃HNC(O)—, (CH₃)₂NC(O)—, (CH₃)₂NS(O)₂—, CH₃S(O)₂—, cyano and amino;
R₁ and R₂ are independently selected from the group consisting of hydrogen and methyl;
R₇ is selected from the group consisting of hydrogen and methyl;
R₈ is selected from the group consisting of hydrogen, —C(O)OR₉, —C(O)R₁₀ and —C(O)C(O)NR₁₁R₁₁; or
R₇ and R₈, together with the nitrogen atom pendent thereto, form a heterocyclic or substituted heterocyclic group;
R₉ is selected from the group consisting of hydrogen, C₁-C₄ alkyl, phenyl and substituted phenyl;
R₁₀ is selected from the group consisting of amino, C₁-C₄ alkylamino, [C₁-C₄ alkyl]₂amino, C₁-C₄ alkyl, heterocyclic and substituted heterocyclic; and
each R₁₁ is independently selected from the group consisting of hydrogen and C₁-C₄ alkyl.

8. A compound having the formula V: 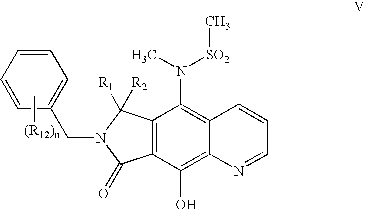
or a pharmaceutically acceptable salt thereof,
where,
R₁ and R₂ are independently selected from the group consisting of hydrogen and methyl;
each R₁₂ is independently selected from the group consisting of halo, C₁-C₄ alkoxy, —C(O)OR₉, —C(O)NR₁₅R₁₆, amino, C₁-C₄ alkylamino, di(C₁-C₄ alkyl)amino, cyano, —SO₂—(C₁-C₄ alkyl) and —SO₂—NR₁₅R₁₆;
R₉ is selected from the group consisting of hydrogen and C₁-C₄ alkyl;
each R₁₅ and R₁₆ is independently selected from the group consisting of hydrogen and C₁-C₄ alkyl; and
n is one, two or three.

9. The compound of claim 8 which has the formula Va: 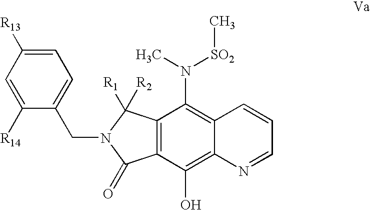
or a pharmaceutically acceptable salt thereof,
where,
R₁₃ and R₁₄ are independently selected from the group consisting of halo, C₁-C₄ alkoxy, —C(O)OR₉, —C(O)NR₁₅R₁₆, amino, C₁-C₄ alkylamino, di(C₁-C₄ alkyl)amino, cyano, —SO₂—(C₁-C₄ alkyl) and —SO₂—NR₁₅R₁₆; and
R₁, R₂, R₉, R₁₅ and R₁₆ are each independently defined above.

10. A compound having the formula VI: 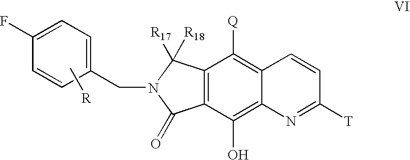
or a pharmaceutically acceptable salt thereof,
where,
R is selected from the group consisting of hydrogen, CH₃HNC(O)—, (CH₃)₂NC(O)—, (CH₃)₂NS(O)₂—, CH₃S(O)₂, cyano and amino;
R₁₇ and R₁₈ are independently selected from the group consisting of hydrogen and hydroxyl, provided that both R₁₇ and R₁₈ are not hydrogen, or
R₁₇ and R₁₈, together with the carbon atom pendent thereto, form a carbonyl group;
Q is selected from the group consisting of amino, hydroxyl, 2-(trimethylsilyl)ethoxy, N-morpholino and —N(CH₃)SO₂CH₃; and
T is selected from the the group consisting of hydrogen, amino and halo.

11. A compound having the formula VII: 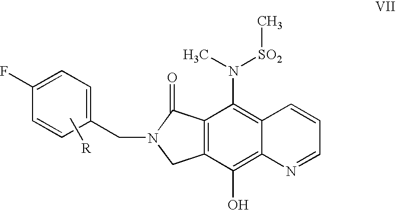
or a pharmaceutically acceptable salt thereof,
where,
R is selected from the group consisting of hydrogen, CH₃HNC(O)—, (CH₃)₂NC(O)—, (CH₃)₂NS(O)₂—, CH₃S(O)₂—, cyano and amino;

12. A compound having the formula XXV: 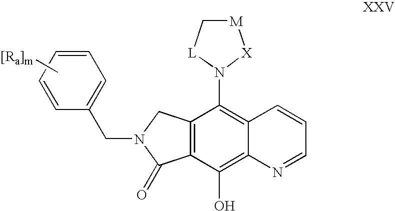
or a pharmaceutically acceptable salt thereof,
where,
L is —CH₂—, —CH₂—CH₂— or —C(O)—;
X is —S(O)₂— or —C(O)—;
M is —N(R₂₀)— or —CH₂—;
R₂₀ is H or —C₁₋₄alkyl;
each Rₐ is independently halo; and
m is zero, one, two, three, four or five.

13. The compound of claim 12 which has the formula XXVa: 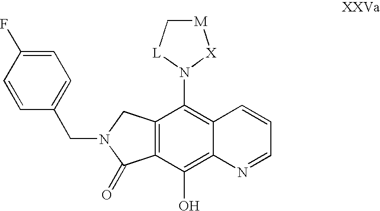
or a pharmaceutically acceptable salt thereof,
where,
L, X and M are independently defined above.

14. A compound having the formula XXIV: 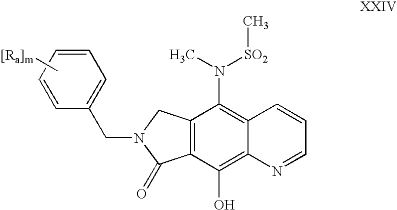
or a pharmaceutically acceptable salt thereof,
where,
each Rₐ is independently halo; and
m is zero, one, two, three, four or five.

15. The compound of claim 14 which has the formula XXIVa: 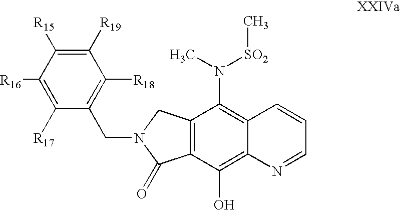
or a pharmaceutically acceptable salt thereof,
where,
R₁₅, R₁₆, R₁₇, R₁₈ and R₁₉ are independently H, Cl or F.

16. The compound of claim 15 which is: 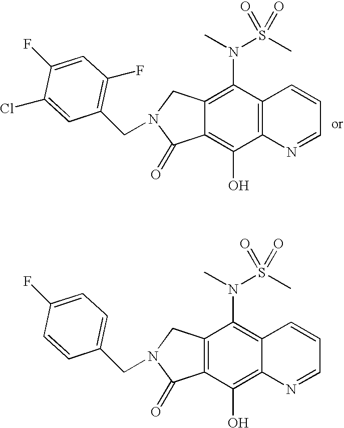
or a pharmaceutically acceptable salt thereof.

17. A compound which is 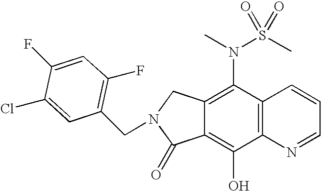
or a pharmaceutically acceptable salt thereof.

18. A prodrug of the compound of claim 17 or a pharmaceutically acceptable salt thereof.

19. A phosphonate of the compound of claim 17 or a pharmaceutically acceptable salt thereof.

20. The phosphonate or pharmaceutically acceptable salt of claim 19 which is a prodrug.

21. The compound or pharmaceutically acceptable salt according to claim 15, where the compound has an IC₅₀ of between>0 μM and about 1 μM.

22. The compound or pharmaceutically acceptable salt according to claim 15, where the compound has an EC₅₀ of between>0 μM and about 1 μM.

23. The compound or pharmaceutically acceptable salt or solvate according to claim 15, where the compound has a IC₅₀ of between>0 nM and about 1 nM and an EC₅₀ of between>0 μM and about 1 μM.

24. A pharmaceutical composition comprising the compound or pharmaceutically acceptable salt according to claim 17 and a pharmaceutically acceptable excipient, diluent or carrier.

25. The pharmaceutical composition of claim 24, further comprising an AIDS treatment agent, an anti-infective agent, an immunomodulator agent, a booster agent or a mixture thereof.

26. The pharmaceutical composition of claim 25, where the AIDS treatment agent is an HIV-protease inhibitor, a nucleoside reverse transcriptase inhibitor, a non-nucleoside reverse transcriptase inhibitor or a mixture thereof.

27. The pharmaceutical composition of claim 24 which is in an oral dosage form.

28. The pharmaceutical composition of claim 26 which is in an oral dosage form.

29. A method of treating the proliferation of HIV virus, treating AIDS, or delaying the onset of AIDS or ARC symptoms, comprising administering to a mammal in need thereof, a thereapeutically effective amount of the compound of claim 17.

30. A method of inhibiting HIV integrase, comprising administering to a mammal in need thereof, a thereapeutically effective amount of the compound of claim 17.

31. The method of claim 29, further comprising administering to a mammal in need thereof, a booster agent, a thereapeutically effective amount of an AIDS treatment agent, a thereapeutically effective amount of an anti-infective agent, a thereapeutically effective amount of an immunomodulator agent, or a mixture thereof.

32. The method of claim 29, where the compound is administered orally.

33. A kit for the treatment of disorders, symptoms and diseases where integrase inhibition plays a role, comprising two or more separate containers in a single package, wherein at least one compound or pharmaceutically acceptable salt of claim 15 is placed in combination with one or more of the following: a pharmaceutically acceptable carrier, a booster agent, a therapeutically effective amount of an AIDS treatment agent, a thereapeutically effective amount of an anti-infective agent or a thereapeutically effective amount of an immunomodulator agent.

34. A compound which is 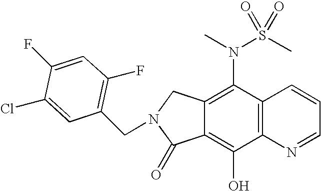
for the use in the treatment of the proliferation of HIV virus, the treatment of AIDS, or delaying the onset of AIDS or ARC symptoms.

35. The compound of claim 34 which is in an oral dosage form.

36. The compound or pharmaceutically acceptable salt of claim 17 for use in the treatment of AIDS.

37. The compound or pharmaceutically acceptable salt of claim 17 for use in therapy.

38. The compound or pharmaceutically acceptable salt of claim 17 for use as a medicament.

39. Use of the compound or pharmaceutically acceptable salt of claim 17 in the manufacture of a medicament for the treatment of HIV.

40. The pharmaceutical composition of claim 24 for use in the treatment of AIDS.

41. The pharmaceutical composition of claim 25 for use in the treatment of AIDS.

42. The compound or pharmaceutically acceptable salt of claim 17 prepared from the following scheme: 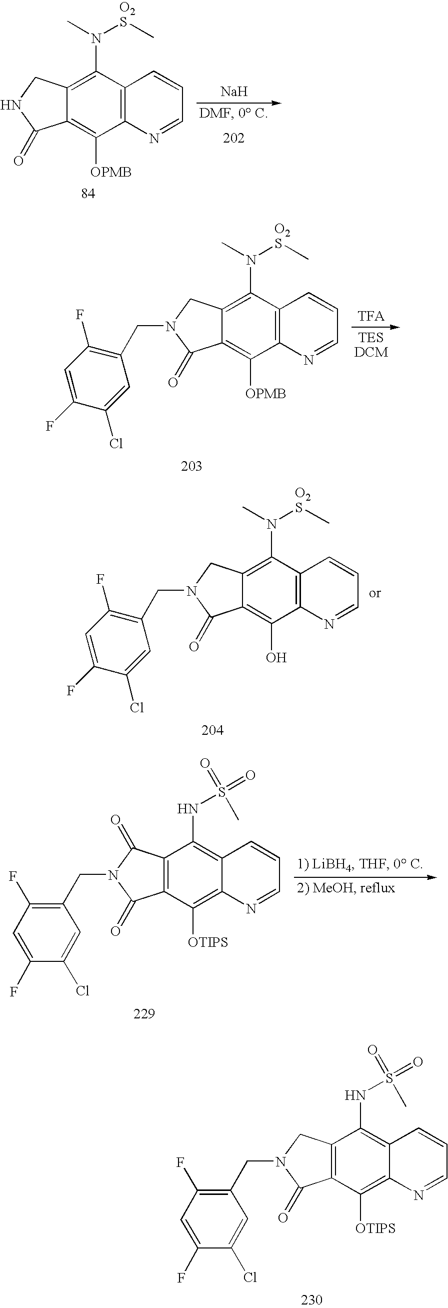
where compound 230 is methylated and deprotected to give compound 204.

43. A compound, pharmaceutically acceptable salt or pharmaceutical composition as described in the description.


SEQUENCE LISTING (4 sequences)

SEQ ID NO: 1 - DNA, 34 bp, Artificial Sequence
  Description of Artificial Sequence Synthetic oligonucleotide
acccttttagtcagtgtggaaaatctctagcagt

SEQ ID NO: 2 - DNA, 31 bp, Artificial Sequence
  Description of Artificial Sequence Synthetic oligonucleotide
actgctagagattttccacactgactaaaag

SEQ ID NO: 3 - DNA, 20 bp, Artificial Sequence
  Description of Artificial Sequence Synthetic oligonucleotide
tgaccaagggctaattcact

SEQ ID NO: 4 - DNA, 20 bp, Artificial Sequence
  Description of Artificial Sequence Synthetic oligonucleotide
agtgaattagcccttggtca